SEQUENCE LISTING (1300 sequences)

SEQ ID NO: 1 - protein, 19 aa, Homo sapiens
  MOD_RES 2: note = Carboxymethylated Cysteine
ACTDNPLEEEYLAQLQEAK

SEQ ID NO: 2 - protein, 11 aa, Homo sapiens
DTLGLPSEEWK

SEQ ID NO: 3 - protein, 10 aa, Homo sapiens
EEEEAPLLPR

SEQ ID NO: 4 - protein, 11 aa, Homo sapiens
ERLDIYQPTAR

SEQ ID NO: 5 - protein, 10 aa, Homo sapiens
  MOD_RES 9: note = Carboxymethylated Cysteine
FDTTPWILCK

SEQ ID NO: 6 - protein, 15 aa, Homo sapiens
GAGMPNLISTDFSPK

SEQ ID NO: 7 - protein, 9 aa, Homo sapiens
LDIYQPTAR

SEQ ID NO: 8 - protein, 22 aa, Homo sapiens
  MOD_RES 15..16: note = Carboxymethylated Cysteine
SMLSGESSTLAAQHCCYDEDSR

SEQ ID NO: 9 - protein, 13 aa, Homo sapiens
  MOD_RES 2: note = Carboxymethylated Cysteine
  MOD_RES 7: note = Carboxymethylated Cysteine
TCDLPSCPGTEDK

SEQ ID NO: 10 - protein, 8 aa, Homo sapiens
TPEVTPLR

SEQ ID NO: 11 - protein, 13 aa, Homo sapiens
TPEVTPLRLELQK

SEQ ID NO: 12 - protein, 14 aa, Homo sapiens
  MOD_RES 4: note = Carboxymethylated Cysteine
  MOD_RES 8: note = Carboxymethylated Cysteine
TRPCGYGCTATETR

SEQ ID NO: 13 - protein, 7 aa, Homo sapiens
YLSQMLR

SEQ ID NO: 14 - protein, 12 aa, Homo sapiens
  SITE 1: note = Pyroglutamic acid
XQNVDHSQEAAK

SEQ ID NO: 15 - protein, 16 aa, Homo sapiens
AAPGQEPPEHMAELQR

SEQ ID NO: 16 - protein, 20 aa, Homo sapiens
AHFSPSNIILDFPAAGSAAR

SEQ ID NO: 17 - protein, 23 aa, Homo sapiens
  MOD_RES 13: note = Carboxymethylated Cysteine
  MOD_RES 22: note = Carboxymethylated Cysteine
AWRPALYLAALDCAEETNSAVCR

SEQ ID NO: 18 - protein, 16 aa, Homo sapiens
  MOD_RES 2: note = Carboxymethylated Cysteine
DCASHFEQMAAASMHR

SEQ ID NO: 19 - protein, 11 aa, Homo sapiens
DFNIPGFPTVR

SEQ ID NO: 20 - protein, 21 aa, Homo sapiens
DVQNVAAAPELAMGALELESR

SEQ ID NO: 21 - protein, 10 aa, Homo sapiens
  MOD_RES 3: note = Carboxymethylated Cysteine
  MOD_RES 6: note = Carboxymethylated Cysteine
ELCSACHNER

SEQ ID NO: 22 - protein, 14 aa, Homo sapiens
  MOD_RES 9: note = Carboxymethylated Cysteine
FGVTDFPSCYLLFR

SEQ ID NO: 23 - protein, 10 aa, Homo sapiens
  MOD_RES 9: note = Carboxymethylated Cysteine
GYVHYFFGCR

SEQ ID NO: 24 - protein, 13 aa, Homo sapiens
IEVGRFPVLEGQR

SEQ ID NO: 25 - protein, 15 aa, Homo sapiens
IYMADLESALHYILR

SEQ ID NO: 26 - protein, 15 aa, Homo sapiens
  MOD_RES 10: note = Carboxymethylated Cysteine
KFGVTDFPSCYLLFR

SEQ ID NO: 27 - protein, 15 aa, Homo sapiens
  MOD_RES 7: note = Carboxymethylated Cysteine
KVNWIGCQGSEPHFR

SEQ ID NO: 28 - protein, 13 aa, Homo sapiens
LAGAPSEDPQFPK

SEQ ID NO: 29 - protein, 16 aa, Homo sapiens
LDVPVWDVEATLNFLK

SEQ ID NO: 30 - protein, 10 aa, Homo sapiens
LEEIDGFFAR

SEQ ID NO: 31 - protein, 33 aa, Homo sapiens
  MOD_RES 16: note = Carboxymethylated Cysteine
LIDALESHHDTWPPACPPLEPAKLEEIDGFFAR

SEQ ID NO: 32 - protein, 18 aa, Homo sapiens
  MOD_RES 1: note = Deamidated Asparagine
NGSGAVFPVAGADVQTLR

SEQ ID NO: 33 - protein, 18 aa, Homo sapiens
NGSGAVFPVAGADVQTLR

SEQ ID NO: 34 - protein, 12 aa, Homo sapiens
ARFEELNADLFR

SEQ ID NO: 35 - protein, 12 aa, Homo sapiens
DAGTIAGLNVLR

SEQ ID NO: 36 - protein, 16 aa, Homo sapiens
IINEPTAAAIAYGLDK

SEQ ID NO: 37 - protein, 9 aa, Homo sapiens
LLQDFFNGK

SEQ ID NO: 38 - protein, 15 aa, Homo sapiens
NQVAMNPTNTVFDAK

SEQ ID NO: 39 - protein, 16 aa, Homo sapiens
NQVAMNPTNTVFDAKR

SEQ ID NO: 40 - protein, 11 aa, Homo sapiens
NSLESYAFNMK

SEQ ID NO: 41 - protein, 27 aa, Homo sapiens
NVLIFDLGGGTFDVSILTIEDGIFEVK

SEQ ID NO: 42 - protein, 12 aa, Homo sapiens
RFDDAVVQSDMK

SEQ ID NO: 43 - protein, 14 aa, Homo sapiens
SQIHDIVLVGGSTR

SEQ ID NO: 44 - protein, 13 aa, Homo sapiens
TTPSYVAFTDTER

SEQ ID NO: 45 - protein, 18 aa, Homo sapiens
TVTNAVVTVPAYFNDSQR

SEQ ID NO: 46 - protein, 12 aa, Homo sapiens
TVTAMDVVYALK

SEQ ID NO: 47 - protein, 13 aa, Homo sapiens
TVTAMDVVYALKR

SEQ ID NO: 48 - protein, 8 aa, Homo sapiens
VFLENVIR

SEQ ID NO: 49 - protein, 27 aa, Homo sapiens
  MOD_RES 13: note = Carboxymethylated Cysteine
  MOD_RES 23: note = Carboxymethylated Cysteine
SVLRPSQFGGQPCTEPLVAFQPCIPSK

SEQ ID NO: 50 - protein, 19 aa, Homo sapiens
EVQLVQSGGGVVQPGGSLR

SEQ ID NO: 51 - protein, 11 aa, Homo sapiens
  MOD_RES 9: note = Carboxymethylated Cysteine
TEDTAVYYCAK

SEQ ID NO: 52 - protein, 9 aa, Homo sapiens
  MOD_RES 3: note = Carboxymethylated Cysteine
LSCVGSGFR

SEQ ID NO: 53 - protein, 22 aa, Homo sapiens
  MOD_RES 20: note = Carboxymethylated Cysteine
SSLYLQMKSLRDEDTAVYYCAR

SEQ ID NO: 54 - protein, 12 aa, Homo sapiens
MVTGVASALSSR

SEQ ID NO: 55 - protein, 18 aa, Homo sapiens
  MOD_RES 1: note = Carboxymethylated Cysteine
CHSANLNGVYYSGPYTAK

SEQ ID NO: 56 - protein, 12 aa, Homo sapiens
GDENTVIDLGSK

SEQ ID NO: 57 - protein, 9 aa, Homo sapiens
HGEYWLGNK

SEQ ID NO: 58 - protein, 11 aa, Homo sapiens
IRPNDFIPNVI

SEQ ID NO: 59 - protein, 14 aa, Homo sapiens
NLHFLTTQEDYTLK

SEQ ID NO: 60 - protein, 13 aa, Homo sapiens
QLLQENEVQFLDK

SEQ ID NO: 61 - protein, 14 aa, Homo sapiens
  MOD_RES 5: note = Carboxymethylated Cysteine
QYADCSEIFNDGYK

SEQ ID NO: 62 - protein, 15 aa, Homo sapiens
  MOD_RES 6: note = Carboxymethylated Cysteine
RQYADCSEIFNDGYK

SEQ ID NO: 63 - protein, 10 aa, Homo sapiens
RSDGSENFNR

SEQ ID NO: 64 - protein, 9 aa, Homo sapiens
SDGSENFNR

SEQ ID NO: 65 - protein, 14 aa, Homo sapiens
DEASSVEVTWPDGK

SEQ ID NO: 66 - protein, 21 aa, Homo sapiens
EHGDPLIEELNPGDALEPEGR

SEQ ID NO: 67 - protein, 8 aa, Homo sapiens
FSMPSPVR

SEQ ID NO: 68 - protein, 22 aa, Homo sapiens
GDGTFVDAAASAGVDDPHQHGR

SEQ ID NO: 69 - protein, 11 aa, Homo sapiens
GNQGFNNNWLR

SEQ ID NO: 70 - protein, 33 aa, Homo sapiens
GTGGVVTDFDGDGMLDLILSHGESMAQPLSVFR

SEQ ID NO: 71 - protein, 9 aa, Homo sapiens
LVNIAVDER

SEQ ID NO: 72 - protein, 16 aa, Homo sapiens
NNRWEDILSDEVNVAR

SEQ ID NO: 73 - protein, 17 aa, Homo sapiens
NVASGEMNSVLEILYPR

SEQ ID NO: 74 - protein, 8 aa, Homo sapiens
SSPYYALR

SEQ ID NO: 75 - protein, 13 aa, Homo sapiens
WEDILSDEVNVAR

SEQ ID NO: 76 - protein, 16 aa, Homo sapiens
IPKPGLDHTEASFSPR

SEQ ID NO: 77 - protein, 13 aa, Homo sapiens
PGLDHTEASFSPR

SEQ ID NO: 78 - protein, 9 aa, Homo sapiens
ATITSLWGK

SEQ ID NO: 79 - protein, 12 aa, Homo sapiens
EFTPEVQASWQK

SEQ ID NO: 80 - protein, 19 aa, Homo sapiens
FFDSFGNLSSASAIMGNPK

SEQ ID NO: 81 - protein, 8 aa, Homo sapiens
GHFTEEDK

SEQ ID NO: 82 - protein, 13 aa, Homo sapiens
  MOD_RES 11: note = Carboxymethylated Cysteine
GTFAQLSELHCDK

SEQ ID NO: 83 - protein, 9 aa, Homo sapiens
LHVDPENFK

SEQ ID NO: 84 - protein, 16 aa, Homo sapiens
LLGNVLVTVLAIHFGK

SEQ ID NO: 85 - protein, 12 aa, Homo sapiens
  MOD_RES 1: note = Oxidized Methionine
MVTAVASALSSR

SEQ ID NO: 86 - protein, 12 aa, Homo sapiens
MVTAVASALSSR

SEQ ID NO: 87 - protein, 10 aa, Homo sapiens
VLTSLGDAIK

SEQ ID NO: 88 - protein, 13 aa, Homo sapiens
VNVEDAGGETLGR

SEQ ID NO: 89 - protein, 9 aa, Homo sapiens
RAEQELAGR

SEQ ID NO: 90 - protein, 13 aa, Homo sapiens
  SITE 1: note = Pyroglutamic acid
XQSEDSTFYLGER

SEQ ID NO: 91 - protein, 9 aa, Homo sapiens
  SITE 1: note = Pyroglutamic acid
XQYTDSTFR

SEQ ID NO: 92 - protein, 14 aa, Homo sapiens
  SITE 1: note = Pyroglutamic acid
XQYTDSTFRVPVER

SEQ ID NO: 93 - protein, 20 aa, Homo sapiens
AEEEHLGILGPQLHADVGDK

SEQ ID NO: 94 - protein, 12 aa, Homo sapiens
AETGDKVYVHLK

SEQ ID NO: 95 - protein, 14 aa, Homo sapiens
  MOD_RES 12: note = Carboxymethylated Cysteine
AGLQAFFQVQECNK

SEQ ID NO: 96 - protein, 12 aa, Homo sapiens
  MOD_RES 5: note = Deamidated Glutamine
ALYLQYTDETFR

SEQ ID NO: 97 - protein, 12 aa, Homo sapiens
ALYLQYTDETFR

SEQ ID NO: 98 - protein, 17 aa, Homo sapiens
ALYLQYTDETFRTTIEK

SEQ ID NO: 99 - protein, 10 aa, Homo sapiens
DDEEFIESNK

SEQ ID NO: 100 - protein, 14 aa, Homo sapiens
  MOD_RES 13: note = Carboxymethylated Cysteine
DIASGLIGPLIICK

SEQ ID NO: 101 - protein, 15 aa, Homo sapiens
  MOD_RES 13: note = Carboxymethylated Cysteine
DIASGLIGPLIICKK

SEQ ID NO: 102 - protein, 11 aa, Homo sapiens
  MOD_RES 10: note = Oxidized Methionine
DIFTGLIGPMK

SEQ ID NO: 103 - protein, 11 aa, Homo sapiens
DIFTGLIGPMK

SEQ ID NO: 104 - protein, 14 aa, Homo sapiens
  MOD_RES 13: note = Carboxymethylated Cysteine
DLYSGLIGPLIVCR

SEQ ID NO: 105 - protein, 10 aa, Homo sapiens
  MOD_RES 6: note = Deamidated Glutamine
DNEDFQESNR

SEQ ID NO: 106 - protein, 10 aa, Homo sapiens
DNEDFQESNR

SEQ ID NO: 107 - protein, 38 aa, Homo sapiens
  MOD_RES 15: note = Oxidized Methionine
  MOD_RES 27: note = Carboxymethylated Cysteine
DTANLFPQTSLTLHMWPDTEGTFNVECLTTDHYTGGMK

SEQ ID NO: 108 - protein, 38 aa, Homo sapiens; identical to SEQ ID NO: 107
  MOD_RES 24: note = Deamidated Asparagine
  MOD_RES 27: note = Carboxymethylated Cysteine

SEQ ID NO: 109 - protein, 38 aa, Homo sapiens; identical to SEQ ID NO: 107
  MOD_RES 27: note = Carboxymethylated Cysteine

SEQ ID NO: 110 - protein, 10 aa, Homo sapiens
  MOD_RES 8: note = Deamidated Asparagine
AQYSWTINGK

SEQ ID NO: 111 - protein, 10 aa, Homo sapiens
AQYSWTINGK

SEQ ID NO: 112 - protein, 31 aa, Homo sapiens
DGTGGVTGHFTFTLHLETPKPSISSSNLNPR

SEQ ID NO: 113 - protein, 20 aa, Homo sapiens
DVLLLVHNLPQNLAGYIWYK

SEQ ID NO: 114 - protein, 10 aa, Homo sapiens
  MOD_RES 7: note = Carboxymethylated Cysteine
DVLTFTCEPK

SEQ ID NO: 115 - protein, 7 aa, Homo sapiens
FQLSGQK

SEQ ID NO: 116 - protein, 19 aa, Homo sapiens
  MOD_RES 3: note = Oxidized Methionine
GQMTYLYHYITSYVVDGQR

SEQ ID NO: 117 - protein, 19 aa, Homo sapiens
GQMTYLYHYITSYVVDGQR

SEQ ID NO: 118 - protein, 10 aa, Homo sapiens
  MOD_RES 7: note = Carboxymethylated Cysteine
HSGLYACSVR

SEQ ID NO: 119 - protein, 10 aa, Homo sapiens
IIYGPAYSGR

SEQ ID NO: 120 - protein, 9 aa, Homo sapiens
LSIPQITTK

SEQ ID NO: 121 - protein, 25 aa, Homo sapiens
SDPVTLNVLYGPDLPSIYPSFTYYR

SEQ ID NO: 122 - protein, 16 aa, Homo sapiens
  MOD_RES 9: note = Carboxymethylated Cysteine
SGENLYLSCFAESNPR

SEQ ID NO: 123 - protein, 11 aa, Homo sapiens
  MOD_RES 8: note = Carboxymethylated Cysteine
YIAGPYECEIR

SEQ ID NO: 124 - protein, 18 aa, Homo sapiens
  MOD_RES 8: note = Carboxymethylated Cysteine
YIAGPYECEIRNPVSASR

SEQ ID NO: 125 - protein, 12 aa, Homo sapiens
  SITE 1: note = Pyroglutamic acid
  MOD_RES 4: note = Deamidated Asparagine
XQLNEINYEDHK

SEQ ID NO: 126 - protein, 12 aa, Homo sapiens
  SITE 1: note = Pyroglutamic acid
XQLNEINYEDHK

SEQ ID NO: 127 - protein, 18 aa, Homo sapiens
  MOD_RES 4: note = Carboxymethylated Cysteine
  MOD_RES 12: note = Deamidated Asparagine
AIHCPRPHDFENGEYWPR

SEQ ID NO: 128 - protein, 18 aa, Homo sapiens
  MOD_RES 4: note = Carboxymethylated Cysteine
AIHCPRPHDFENGEYWPR

SEQ ID NO: 129 - protein, 9 aa, Homo sapiens
ALFVSEEEK

SEQ ID NO: 130 - protein, 10 aa, Homo sapiens
ALFVSEEEKK

SEQ ID NO: 131 - protein, 14 aa, Homo sapiens
  MOD_RES 9: note = Oxidized Methionine
ALHQVFEHMLDVSK

SEQ ID NO: 132 - protein, 14 aa, Homo sapiens
ALHQVFEHMLDVSK

SEQ ID NO: 133 - protein, 14 aa, Homo sapiens
  MOD_RES 10: note = Carboxymethylated Cysteine
ALRLPPTTTCQQQK

SEQ ID NO: 134 - protein, 24 aa, Homo sapiens
  MOD_RES 10: note = Carboxymethylated Cysteine
ALRLPPTTTCQQQKEELLPAQDIK

SEQ ID NO: 135 - protein, 12 aa, Homo sapiens
AVISPGFDVFAK

SEQ ID NO: 136 - protein, 13 aa, Homo sapiens
AVISPGFDVFAKK

SEQ ID NO: 137 - protein, 8 aa, Homo sapiens
  MOD_RES 1: note = Carboxymethylated Cysteine
CLVNLIEK

SEQ ID NO: 138 - protein, 17 aa, Homo sapiens
  MOD_RES 1: note = Carboxymethylated Cysteine
CLVNLIEKVASYGVKPR

SEQ ID NO: 139 - protein, 13 aa, Homo sapiens
  MOD_RES 1: note = Carboxymethylated Cysteine
CSSNLVLTGSSER

SEQ ID NO: 140 - protein, 29 aa, Homo sapiens
  MOD_RES 1: note = Carboxymethylated Cysteine
  MOD_RES 15: note = Carboxymethylated Cysteine
  MOD_RES 28: note = Carboxymethylated Cysteine
CSSNLVLTGSSERECQGNGVWSGTEPICR

SEQ ID NO: 141 - protein, 9 aa, Homo sapiens
  MOD_RES 3: note = Oxidized Methionine
VAMSIDTSK

SEQ ID NO: 142 - protein, 9 aa, Homo sapiens
VAMSIDTSK

SEQ ID NO: 143 - protein, 11 aa, Homo sapiens
  MOD_RES 9: note = Carboxymethylated Cysteine
AEDTALYHCAR

SEQ ID NO: 144 - protein, 23 aa, Homo sapiens
ATGIPDRFSGSGSETDFTLTISR

SEQ ID NO: 145 - protein, 18 aa, Homo sapiens
EIVLTQSPGTLSLFPGER

SEQ ID NO: 146 - protein, 16 aa, Homo sapiens
FSGSGSETDFTLTISR

SEQ ID NO: 147 - protein, 9 aa, Homo sapiens
LLIYGTSSR

SEQ ID NO: 148 - protein, 12 aa, Homo sapiens
  SITE 1: note = N-terminus is acetylated
ASGVAVSDGVIK

SEQ ID NO: 149 - protein, 10 aa, Homo sapiens
  MOD_RES 5: note = Carboxymethylated Cysteine
AVLFCLSEDK

SEQ ID NO: 150 - protein, 12 aa, Homo sapiens
  MOD_RES 7: note = Carboxymethylated Cysteine
HELQANCYEEVK

SEQ ID NO: 151 - protein, 17 aa, Homo sapiens
KEDLVFIFWAPESAPLK

SEQ ID NO: 152 - protein, 8 aa, Homo sapiens
NIILEEGK

SEQ ID NO: 153 - protein, 11 aa, Homo sapiens
YALYDATYETK

SEQ ID NO: 154 - protein, 9 aa, Homo sapiens
LFQGQLVLR

SEQ ID NO: 155 - protein, 15 aa, Homo sapiens
NSETSDTTGTHESDR

SEQ ID NO: 156 - protein, 19 aa, Homo sapiens
  MOD_RES 2: note = Carboxymethylated Cysteine
ACTDNPLEEEYLAQLQEAK

SEQ ID NO: 157 - protein, 11 aa, Homo sapiens
DTLGLPSEEWK

SEQ ID NO: 158 - protein, 10 aa, Homo sapiens
EEEEAPLLPR

SEQ ID NO: 159 - protein, 11 aa, Homo sapiens
ERLDIYQPTAR

SEQ ID NO: 160 - protein, 10 aa, Homo sapiens
  MOD_RES 9: note = Carboxymethylated Cysteine
FDTTPWILCK

SEQ ID NO: 161 - protein, 15 aa, Homo sapiens
GAGMPNLISTDFSPK

SEQ ID NO: 162 - protein, 9 aa, Homo sapiens
LDIYQPTAR

SEQ ID NO: 163 - protein, 22 aa, Homo sapiens
  MOD_RES 15..16: note = Carboxymethylated Cysteine
SMLSGESSTLAAQHCCYDEDSR

SEQ ID NO: 164 - protein, 13 aa, Homo sapiens
  MOD_RES 2: note = Carboxymethylated Cysteine
  MOD_RES 7: note = Carboxymethylated Cysteine
TCDLPSCPGTEDK

SEQ ID NO: 165 - protein, 8 aa, Homo sapiens
TPEVTPLR

SEQ ID NO: 166 - protein, 13 aa, Homo sapiens
TPEVTPLRLELQK

SEQ ID NO: 167 - protein, 14 aa, Homo sapiens
  MOD_RES 4: note = Carboxymethylated Cysteine
  MOD_RES 8: note = Carboxymethylated Cysteine
TRPCGYGCTATETR

SEQ ID NO: 168 - protein, 7 aa, Homo sapiens
YLSQMLR

SEQ ID NO: 169 - protein, 14 aa, Homo sapiens
AGTPYTVTLHGEVR

SEQ ID NO: 170 - protein, 10 aa, Homo sapiens
APTAQVESFR

SEQ ID NO: 171 - protein, 14 aa, Homo sapiens
AVDIPGLEAATPYR

SEQ ID NO: 172 - protein, 28 aa, Homo sapiens
DLAPPSEPSESFQEHTVDGENQIVFTHR

SEQ ID NO: 173 - protein, 14 aa, Homo sapiens
  MOD_RES 9: note = Carboxymethylated Cysteine
DTDSAITNCALSYK

SEQ ID NO: 174 - protein, 11 aa, Homo sapiens
ELEPGYEYFIR

SEQ ID NO: 175 - protein, 17 aa, Homo sapiens
ESNPATINAATELDTPK

SEQ ID NO: 176 - protein, 11 aa, Homo sapiens
ETFTTGLDAPR

SEQ ID NO: 177 - protein, 9 aa, Homo sapiens
FTTDLDSPR

SEQ ID NO: 178 - protein, 12 aa, Homo sapiens
GHEHSIQFAEMK

SEQ ID NO: 179 - protein, 16 aa, Homo sapiens
GLEPGQEYNVLLTAEK

SEQ ID NO: 180 - protein, 9 aa, Homo sapiens
IQALNGPLR

SEQ ID NO: 181 - protein, 10 aa, Homo sapiens
ITAQGQYELR

SEQ ID NO: 182 - protein, 18 aa, Homo sapiens
QTGLAPGQEYEISLHIVK

SEQ ID NO: 183 - protein, 16 aa, Homo sapiens
SQTVSAIATTAMGSPK

SEQ ID NO: 184 - protein, 25 aa, Homo sapiens
TAHISGLPPSTDFIVYLSGLAPSIR

SEQ ID NO: 185 - protein, 12 aa, Homo sapiens
VATYLPAPEGLK

SEQ ID NO: 186 - protein, 8 aa, Homo sapiens
YSIYGYIR

SEQ ID NO: 187 - protein, 15 aa, Homo sapiens
ASQGISNSLAWYQQK

SEQ ID NO: 188 - protein, 8 aa, Homo sapiens
LESGVPSR

SEQ ID NO: 189 - protein, 8 aa, Homo sapiens
LLLYAASR

SEQ ID NO: 190 - protein, 9 aa, Homo sapiens
FSGSILGNK

SEQ ID NO: 191 - protein, 16 aa, Homo sapiens
  MOD_RES 15: note = Deamidated Asparagine
SSGYPDRFSGSILGNK

SEQ ID NO: 192 - protein, 16 aa, Homo sapiens
SSGVPDRFSGSILGNK

SEQ ID NO: 193 - protein, 9 aa, Homo sapiens
  MOD_RES 7: note = Deamidated Asparagine
TLIYSTNTR

SEQ ID NO: 194 - protein, 9 aa, Homo sapiens
TLIYSTNTR

SEQ ID NO: 195 - protein, 11 aa, Homo sapiens
  MOD_RES 1: note = Deamidated Asparagine
NNNRPSGISER

SEQ ID NO: 196 - protein, 11 aa, Homo sapiens
  MOD_RES 3: note = Deamidated Asparagine
NNNRPSGISER

SEQ ID NO: 197 - protein, 11 aa, Homo sapiens
NNNRPSGISER

SEQ ID NO: 198 - protein, 9 aa, Homo sapiens
  MOD_RES 8: note = Deamidated Asparagine
TLIYDTSNK

SEQ ID NO: 199 - protein, 9 aa, Homo sapiens
TLIYDTSNK

SEQ ID NO: 200 - protein, 16 aa, Homo sapiens
  MOD_RES 2: note = Oxidized Methionine
LMIYEVSNRPSGVPDR

SEQ ID NO: 201 - protein, 16 aa, Homo sapiens
  MOD_RES 8: note = Deamidated Asparagine
LMIYEVSNRPSGVPDR

SEQ ID NO: 202 - protein, 16 aa, Homo sapiens
LMIYEYSNRPSGVPDR

SEQ ID NO: 203 - protein, 21 aa, Homo sapiens
LMIYEVSNRPSGVPDRFSGSK

SEQ ID NO: 204 - protein, 13 aa, Homo sapiens
VSWYQQPPGTAPK

SEQ ID NO: 205 - protein, 11 aa, Homo sapiens
  MOD_RES 3: note = Deamidated Asparagine
DSNRPSGIPER

SEQ ID NO: 206 - protein, 11 aa, Homo sapiens
DSNRPSGIPER

SEQ ID NO: 207 - protein, 8 aa, Homo sapiens
NVHWYQQK

SEQ ID NO: 208 - protein, 19 aa, Homo sapiens
NVHWYQQKPGQAPVLVIYR

SEQ ID NO: 209 - protein, 11 aa, Homo sapiens
PGQAPVLVIYR

SEQ ID NO: 210 - protein, 11 aa, Homo sapiens
  MOD_RES 9: note = Carboxymethylated Cysteine
AEDMAVYYCAR

SEQ ID NO: 211 - protein, 11 aa, Homo sapiens
NTLYLQMGSLR

SEQ ID NO: 212 - protein, 21 aa, Homo sapiens
GLEWIGEIYHSGSTNYNPSLK

SEQ ID NO: 213 - protein, 9 aa, Homo sapiens
SRVTISVDK

SEQ ID NO: 214 - protein, 15 aa, Homo sapiens
PGQPPQLLIYEVSNR

SEQ ID NO: 215 - protein, 19 aa, Homo sapiens
APELLIYAASTLQSGVPSR

SEQ ID NO: 216 - protein, 15 aa, Homo sapiens
ASQSVSSYLAWYQQK

SEQ ID NO: 217 - protein, 21 aa, Homo sapiens
ASQSVSSYLAWYQQKPGQAPR

SEQ ID NO: 218 - protein, 16 aa, Homo sapiens
LLIYDASNRATGIPAR

SEQ ID NO: 219 - protein, 11 aa, Homo sapiens
  MOD_RES 9: note = Carboxymethylated Cysteine
SEDTAMYYCAR

SEQ ID NO: 220 - protein, 15 aa, Homo sapiens
ASQSIGSSLHWYQQK

SEQ ID NO: 221 - protein, 21 aa, Homo sapiens
ASQSIGSSLHWYQQKPDQSPK

SEQ ID NO: 222 - protein, 12 aa, Homo sapiens
YASQSISGVPSR

SEQ ID NO: 223 - protein, 7 aa, Homo sapiens
GLEWLGR

SEQ ID NO: 224 - protein, 9 aa, Homo sapiens
ITINPDTSK

SEQ ID NO: 225 - protein, 22 aa, Homo sapiens
  MOD_RES 20: note = Carboxymethylated Cysteine
NQFSLQLNSVTPEDTAVYYCAR

SEQ ID NO: 226 - protein, 11 aa, Homo sapiens
SRITINPDTSK

SEQ ID NO: 227 - protein, 10 aa, Homo sapiens
WYNDYAVSVK

SEQ ID NO: 228 - protein, 14 aa, Homo sapiens
  MOD_RES 11: note = Deamidated Asparagine
ALEWLAHIFSNDEK

SEQ ID NO: 229 - protein, 14 aa, Homo sapiens
ALEWLAHIFSNDEK

SEQ ID NO: 230 - protein, 7 aa, Homo sapiens
MGVSWIR

SEQ ID NO: 231 - protein, 13 aa, Homo sapiens
ANSYATAYAASVK

SEQ ID NO: 232 - protein, 11 aa, Homo sapiens
  MOD_RES 7: note = Oxidized Methionine
NTAYLQMNSLK

SEQ ID NO: 233 - protein, 11 aa, Homo sapiens
  MOD_RES 8: note = Deamidated Asparagine
NTAYLQMNSLK

SEQ ID NO: 234 - protein, 11 aa, Homo sapiens
NTAYLQMNSLK

SEQ ID NO: 235 - protein, 15 aa, Homo sapiens
DNAKNTLYLQMNSLR

SEQ ID NO: 236 - protein, 7 aa, Homo sapiens
GLVWVSR

SEQ ID NO: 237 - protein, 15 aa, Homo sapiens
INSDGSSTSYADSVK

SEQ ID NO: 238 - protein, 22 aa, Homo sapiens
  MOD_RES 20: note = Carboxymethylated Cysteine
NTLYLQMNSLRAEDTAVYYCAR

SEQ ID NO: 239 - protein, 12 aa, Homo sapiens
QAPGKGLVWVSR

SEQ ID NO: 240 - protein, 19 aa, Homo sapiens
  MOD_RES 2: note = Carboxymethylated Cysteine
ACTDNPLEEEYLAQLQEAK

SEQ ID NO: 241 - protein, 11 aa, Homo sapiens
DTLGLPSEEWK

SEQ ID NO: 242 - protein, 10 aa, Homo sapiens
EEEEAPLLPR

SEQ ID NO: 243 - protein, 11 aa, Homo sapiens
ERLDIYQPTAR

SEQ ID NO: 244 - protein, 10 aa, Homo sapiens
  MOD_RES 9: note = Carboxymethylated Cysteine
FDTTPWILCK

SEQ ID NO: 245 - protein, 15 aa, Homo sapiens
GAGMPNLISTDFSPK

SEQ ID NO: 246 - protein, 9 aa, Homo sapiens
LDIYQPTAR

SEQ ID NO: 247 - protein, 22 aa, Homo sapiens
  MOD_RES 15..16: note = Carboxymethylated Cysteine
SMLSGESSTLAAQHCCYDEDSR

SEQ ID NO: 248 - protein, 13 aa, Homo sapiens
  MOD_RES 2: note = Carboxymethylated Cysteine
  MOD_RES 7: note = Carboxymethylated Cysteine
TCDLPSCPGTEDK

SEQ ID NO: 249 - protein, 8 aa, Homo sapiens
TPEVTPLR

SEQ ID NO: 250 - protein, 13 aa, Homo sapiens
TPEVTPLRLELQK

SEQ ID NO: 251 - protein, 14 aa, Homo sapiens
  MOD_RES 4: note = Carboxymethylated Cysteine
  MOD_RES 8: note = Carboxymethylated Cysteine
TRPCGYGCTATETR

SEQ ID NO: 252 - protein, 7 aa, Homo sapiens
YLSQMLR

SEQ ID NO: 253 - protein, 18 aa, Homo sapiens
  MOD_RES 10: note = Carboxymethylated Cysteine
DVPASFAPACLSHEIIIR

SEQ ID NO: 254 - protein, 23 aa, Homo sapiens
  MOD_RES 2: note = Carboxymethylated Cysteine
  MOD_RES 10: note = Carboxymethylated Cysteine
  MOD_RES 15: note = Carboxymethylated Cysteine
  MOD_RES 19: note = Carboxymethylated Cysteine
GCPVHLVDSCPWPHCNPSCPTVR

SEQ ID NO: 255 - protein, 12 aa, Homo sapiens
GLADSGWFLDNK

SEQ ID NO: 256 - protein, 17 aa, Homo sapiens
  MOD_RES 4: note = Carboxymethylated Cysteine
  MOD_RES 10: note = Carboxymethylated Cysteine
HTDCVDTITCAPTEAIR

SEQ ID NO: 257 - protein, 9 aa, Homo sapiens
LGYPAIQVR

SEQ ID NO: 258 - protein, 8 aa, Homo sapiens
LYIQNLGR

SEQ ID NO: 259 - protein, 15 aa, Homo sapiens
  MOD_RES 10: note = Carboxymethylated Cysteine
QFQEGEEWNCFFGYK

SEQ ID NO: 260 - protein, 19 aa, Homo sapiens
  MOD_RES 9: note = Carboxymethylated Cysteine
SLAQSLYPCSAQQLNEDLR

SEQ ID NO: 261 - protein, 9 aa, Homo sapiens
YWNGVVPER

SEQ ID NO: 262 - protein, 12 aa, Homo sapiens
  SITE 1: note = Pyroglutamic acid
XQNVDHSQEAAK

SEQ ID NO: 263 - protein, 16 aa, Homo sapiens
AAPGQEPPEHMAELQR

SEQ ID NO: 264 - protein, 20 aa, Homo sapiens
AHFSPSNIILDFPAAGSAAR

SEQ ID NO: 265 - protein, 23 aa, Homo sapiens
  MOD_RES 13: note = Carboxymethylated Cysteine
  MOD_RES 22: note = Carboxymethylated Cysteine
AWRPALYLAALDCAEETNSAVCR

SEQ ID NO: 266 - protein, 16 aa, Homo sapiens
  MOD_RES 2: note = Carboxymethylated Cysteine
DCASHFEQMAAASMHR

SEQ ID NO: 267 - protein, 11 aa, Homo sapiens
DFNIPGFPTVR

SEQ ID NO: 268 - protein, 21 aa, Homo sapiens
DVQNVAAAPELAMGALELESR

SEQ ID NO: 269 - protein, 10 aa, Homo sapiens
  MOD_RES 3: note = Carboxymethylated Cysteine
  MOD_RES 6: note = Carboxymethylated Cysteine
ELCSACHNER

SEQ ID NO: 270 - protein, 14 aa, Homo sapiens
  MOD_RES 9: note = Carboxymethylated Cysteine
FGVTDFPSCYLLFR

SEQ ID NO: 271 - protein, 10 aa, Homo sapiens
  MOD_RES 9: note = Carboxymethylated Cysteine
GYVHYFFGCR

SEQ ID NO: 272 - protein, 13 aa, Homo sapiens
IEVGRFPVLEGQR

SEQ ID NO: 273 - protein, 15 aa, Homo sapiens
IYMADLESALHYILR

SEQ ID NO: 274 - protein, 15 aa, Homo sapiens
  MOD_RES 10: note = Carboxymethylated Cysteine
KFGVTDFPSCYLLFR

SEQ ID NO: 275 - protein, 15 aa, Homo sapiens
  MOD_RES 7: note = Carboxymethylated Cysteine
KVNWIGCQGSEPHFR

SEQ ID NO: 276 - protein, 13 aa, Homo sapiens
LAGAPSEDPQFPK

SEQ ID NO: 277 - protein, 16 aa, Homo sapiens
LDVPVWDVEATLNFLK

SEQ ID NO: 278 - protein, 10 aa, Homo sapiens
LEEIDGFFAR

SEQ ID NO: 279 - protein, 33 aa, Homo sapiens; identical to SEQ ID NO: 31
  MOD_RES 16: note = Carboxymethylated Cysteine

SEQ ID NO: 280 - protein, 18 aa, Homo sapiens
  MOD_RES 1: note = Deamidated Asparagine
NGSGAVFPVAGADVQTLR

SEQ ID NO: 281 - protein, 18 aa, Homo sapiens
NGSGAVFPVAGADVQTLR

SEQ ID NO: 282 - protein, 8 aa, Homo sapiens
DQVETALK

SEQ ID NO: 283 - protein, 9 aa, Homo sapiens
IMGITLVSK

SEQ ID NO: 284 - protein, 9 aa, Homo sapiens
LLGLSLAGK

SEQ ID NO: 285 - protein, 9 aa, Homo sapiens
  MOD_RES 1: note = Deamidated Asparagine
NGVGVLIWK

SEQ ID NO: 286 - protein, 9 aa, Homo sapiens
NGVGVLIWK

SEQ ID NO: 287 - protein, 12 aa, Homo sapiens
ARFEELNADLFR

SEQ ID NO: 288 - protein, 12 aa, Homo sapiens
DAGTIAGLNVLR

SEQ ID NO: 289 - protein, 16 aa, Homo sapiens
IINEPTAAAIAYGLDK

SEQ ID NO: 290 - protein, 9 aa, Homo sapiens
LLQDFFNGK

SEQ ID NO: 291 - protein, 15 aa, Homo sapiens
NQVAMNPTNTVFDAK

SEQ ID NO: 292 - protein, 16 aa, Homo sapiens
NQVAMNPTNTVFDAKR

SEQ ID NO: 293 - protein, 11 aa, Homo sapiens
NSLESYAFNMK

SEQ ID NO: 294 - protein, 27 aa, Homo sapiens
NVLIFDLGGGTFDVSILTIEDGIFEVK

SEQ ID NO: 295 - protein, 12 aa, Homo sapiens
RFDDAVVQSDMK

SEQ ID NO: 296 - protein, 14 aa, Homo sapiens
SQIHDIVLVGGSTR

SEQ ID NO: 297 - protein, 13 aa, Homo sapiens
TTPSYVAFTDTER

SEQ ID NO: 298 - protein, 18 aa, Homo sapiens
TVTNAVVTVPAYFNDSQR

SEQ ID NO: 299 - protein, 15 aa, Homo sapiens
  MOD_RES 12: note = Oxidized Methionine
DGAVESISVPDMVDK

SEQ ID NO: 300 - protein, 15 aa, Homo sapiens
DGAVESISVPDMVDK

SEQ ID NO: 301 - protein, 8 aa, Homo sapiens
FQWVDGSR

SEQ ID NO: 302 - protein, 10 aa, Homo sapiens
  MOD_RES 4: note = Carboxymethylated Cysteine
  MOD_RES 8: note = Carboxymethylated Cysteine
GGHCVALCTR

SEQ ID NO: 303 - protein, 14 aa, Homo sapiens
  MOD_RES 8: note = Deamidated Asparagine
GNLVSIHNFNINYR

SEQ ID NO: 304 - protein, 14 aa, Homo sapiens
GNLVSIHNFNINYR

SEQ ID NO: 305 - protein, 18 aa, Homo sapiens
  MOD_RES 3: note = Carboxymethylated Cysteine
IQCSVSALNQGQVWIGGR

SEQ ID NO: 306 - protein, 16 aa, Homo sapiens
KDGAVESISVPDMVDK

SEQ ID NO: 307 - protein, 12 aa, Homo sapiens
  MOD_RES 4: note = Carboxymethylated Cysteine
NLTCPEEEDTVK

SEQ ID NO: 308 - protein, 9 aa, Homo sapiens
RFQWVDGSR

SEQ ID NO: 309 - protein, 8 aa, Homo sapiens
  MOD_RES 6: note = Carboxymethylated Cysteine
RLPFICSY

SEQ ID NO: 310 - protein, 13 aa, Homo sapiens
  MOD_RES 12: note = Carboxymethylated Cysteine
SLQTFSQAWFTCR

SEQ ID NO: 311 - protein, 14 aa, Homo sapiens
  MOD_RES 12: note = Carboxymethylated Cysteine
SLQTFSQAWFTCRR

SEQ ID NO: 312 - protein, 11 aa, Homo sapiens
  MOD_RES 7: note = Carboxymethylated Cysteine
  MOD_RES 10: note = Carboxymethylated Cysteine
VVGIPGCQTCR

SEQ ID NO: 313 - protein, 14 aa, Homo sapiens
WNFAYWAAHQPWSR

SEQ ID NO: 314 - protein, 13 aa, Homo sapiens
  SITE 1: note = Pyroglutamic acid
XQSEDSTFYLGER

SEQ ID NO: 315 - protein, 9 aa, Homo sapiens
  SITE 1: note = Pyroglutamic acid
XQYTDSTFR

SEQ ID NO: 316 - protein, 14 aa, Homo sapiens
  SITE 1: note = Pyroglutamic acid
XQYTDSTFRVPVER

SEQ ID NO: 317 - protein, 20 aa, Homo sapiens
AEEEHLGILGPQLHADVGDK

SEQ ID NO: 318 - protein, 12 aa, Homo sapiens
AETGDKVYVHLK

SEQ ID NO: 319 - protein, 14 aa, Homo sapiens
  MOD_RES 12: note = Carboxymethylated Cysteine
AGLQAFFQVQECNK

SEQ ID NO: 320 - protein, 12 aa, Homo sapiens
  MOD_RES 5: note = Deamidated Glutamine
ALYLQYTDETFR

SEQ ID NO: 321 - protein, 12 aa, Homo sapiens
ALYLQYTDETFR

SEQ ID NO: 322 - protein, 17 aa, Homo sapiens
ALYLQYTDETFRTTIEK

SEQ ID NO: 323 - protein, 10 aa, Homo sapiens
DDEEFIESNK

SEQ ID NO: 324 - protein, 14 aa, Homo sapiens
  MOD_RES 13: note = Carboxymethylated Cysteine
DIASGLIGPLIICK

SEQ ID NO: 325 - protein, 15 aa, Homo sapiens
  MOD_RES 13: note = Carboxymethylated Cysteine
DIASGLIGPLIICKK

SEQ ID NO: 326 - protein, 11 aa, Homo sapiens
  MOD_RES 10: note = Oxidized Methionine
DIFTGLIGPMK

SEQ ID NO: 327 - protein, 11 aa, Homo sapiens
DIFTGLIGPMK

SEQ ID NO: 328 - protein, 14 aa, Homo sapiens
  MOD_RES 13: note = Carboxymethylated Cysteine
DLYSGLIGPLIVCR

SEQ ID NO: 329 - protein, 10 aa, Homo sapiens
  MOD_RES 6: note = Deamidated Glutamine
DNEDFQESNR

SEQ ID NO: 330 - protein, 10 aa, Homo sapiens
DNEDFQESNR

SEQ ID NO: 331 - protein, 38 aa, Homo sapiens; identical to SEQ ID NO: 107
  MOD_RES 15: note = Oxidized Methionine
  MOD_RES 27: note = Carboxymethylated Cysteine

SEQ ID NO: 332 - protein, 38 aa, Homo sapiens; identical to SEQ ID NO: 107
  MOD_RES 24: note = Deamidated Asparagine
  MOD_RES 27: note = Carboxymethylated Cysteine

SEQ ID NO: 333 - protein, 38 aa, Homo sapiens; identical to SEQ ID NO: 107
  MOD_RES 27: note = Carboxymethylated Cysteine

SEQ ID NO: 334 - protein, 13 aa, Homo sapiens
AGVFSDLSNQELK

SEQ ID NO: 335 - protein, 10 aa, Homo sapiens
  MOD_RES 1: note = Carboxymethylated Cysteine
CLAFTDVAPR

SEQ ID NO: 336 - protein, 9 aa, Homo sapiens
DNGPNYVQR

SEQ ID NO: 337 - protein, 8 aa, Homo sapiens
DTVIVWPR

SEQ ID NO: 338 - protein, 10 aa, Homo sapiens
FYGSPEELAR

SEQ ID NO: 339 - protein, 13 aa, Homo sapiens
GDFPSPIHVSGPR

SEQ ID NO: 340 - protein, 17 aa, Homo sapiens
GHDSTEEPPLFSSHKPR

SEQ ID NO: 341 - protein, 20 aa, Homo sapiens
KYADGEVDVVVLEDPLPGGK

SEQ ID NO: 342 - protein, 11 aa, Homo sapiens
LQPSSTTTMAK

SEQ ID NO: 343 - protein, 29 aa, Homo sapiens
PGYQSSWASRPISTAEYALLYHTLQEATK

SEQ ID NO: 344 - protein, 9 aa, Homo sapiens
VDLDVAGTK

SEQ ID NO: 345 - protein, 15 aa, Homo sapiens
VVQPTLEQTQYSWER

SEQ ID NO: 346 - protein, 19 aa, Homo sapiens
YADGEVDVVVLEDPLPGGK

SEQ ID NO: 347 - protein, 12 aa, Homo sapiens
TVTAMDVVYALK

SEQ ID NO: 348 - protein, 13 aa, Homo sapiens
TVTAMDVVYALKR

SEQ ID NO: 349 - protein, 8 aa, Homo sapiens
VFLENVIR

SEQ ID NO: 350 - protein, 12 aa, Homo sapiens
  SITE 1: note = Pyroglutamic acid
  MOD_RES 4: note = Deamidated Asparagine
XQLNEINYEDHK

SEQ ID NO: 351 - protein, 12 aa, Homo sapiens
  SITE 1: note = Pyroglutamic acid
XQLNEINYEDHK

SEQ ID NO: 352 - protein, 18 aa, Homo sapiens
  MOD_RES 4: note = Carboxymethylated Cysteine
  MOD_RES 12: note = Deamidated Asparagine
AIHCPRPHDFENGEYWPR

SEQ ID NO: 353 - protein, 18 aa, Homo sapiens
  MOD_RES 4: note = Carboxymethylated Cysteine
AIHCPRPHDFENGEYWPR

SEQ ID NO: 354 - protein, 9 aa, Homo sapiens
ALFVSEEEK

SEQ ID NO: 355 - protein, 10 aa, Homo sapiens
ALFVSEEEKK

SEQ ID NO: 356 - protein, 14 aa, Homo sapiens
  MOD_RES 9: note = Oxidized Methionine
ALHQVFEHMLDVSK

SEQ ID NO: 357 - protein, 14 aa, Homo sapiens
ALHQVFEHMLDVSK

SEQ ID NO: 358 - protein, 14 aa, Homo sapiens
  MOD_RES 10: note = Carboxymethylated Cysteine
ALRLPPTTTCQQQK

SEQ ID NO: 359 - protein, 24 aa, Homo sapiens
  MOD_RES 10: note = Carboxymethylated Cysteine
ALRLPPTTTCQQQKEELLPAQDIK

SEQ ID NO: 360 - protein, 12 aa, Homo sapiens
AVISPGFDVFAK

SEQ ID NO: 361 - protein, 13 aa, Homo sapiens
AVISPGFDVFAKK

SEQ ID NO: 362 - protein, 8 aa, Homo sapiens
  MOD_RES 1: note = Carboxymethylated Cysteine
CLVNLIEK

SEQ ID NO: 363 - protein, 17 aa, Homo sapiens
  MOD_RES 1: note = Carboxymethylated Cysteine
CLVNLIEKVASYGVKPR

SEQ ID NO: 364 - protein, 13 aa, Homo sapiens
  MOD_RES 1: note = Carboxymethylated Cysteine
CSSNLVLTGSSER

SEQ ID NO: 365 - protein, 29 aa, Homo sapiens
  MOD_RES 1: note = Carboxymethylated Cysteine
  MOD_RES 15: note = Carboxymethylated Cysteine
  MOD_RES 28: note = Carboxymethylated Cysteine
CSSNLVLTGSSERECQGNGVWSGTEPICR

SEQ ID NO: 366 - protein, 10 aa, Homo sapiens
DAQYAPGYDK

SEQ ID NO: 367 - protein, 14 aa, Homo sapiens
DFHINLFQVLPWLK

SEQ ID NO: 368 - protein, 9 aa, Homo sapiens
DGNDHSLWR

SEQ ID NO: 369 - protein, 9 aa, Homo sapiens
DHENELLNK

SEQ ID NO: 370 - protein, 9 aa, Homo sapiens
EDAVSAAFK

SEQ ID NO: 371 - protein, 8 aa, Homo sapiens
FNALQYLR

SEQ ID NO: 372 - protein, 9 aa, Homo sapiens
ILGPLSYSK

SEQ ID NO: 373 - protein, 15 aa, Homo sapiens
  MOD_RES 13: note = Carboxymethylated Cysteine
ISETSLPPDMYECLR

SEQ ID NO: 374 - protein, 10 aa, Homo sapiens
ISNIPDEYFK

SEQ ID NO: 375 - protein, 11 aa, Homo sapiens
ISNIPDEYFKR

SEQ ID NO: 376 - protein, 11 aa, Homo sapiens
LKEDAVSAAFK

SEQ ID NO: 377 - protein, 18 aa, Homo sapiens
LPSGLPVSLLTLYLDNNK

SEQ ID NO: 378 - protein, 21 aa, Homo sapiens
NIPTVNENLENYYLEVNQLEK

SEQ ID NO: 379 - protein, 10 aa, Homo sapiens
NNQIDHIDEK

SEQ ID NO: 380 - protein, 9 aa, Homo sapiens
RFNALQYLR

SEQ ID NO: 381 - protein, 11 aa, Homo sapiens
SLEDLQLTHNK

SEQ ID NO: 382 - protein, 14 aa, Homo sapiens
SLEYLDLSFNQIAR

SEQ ID NO: 383 - protein, 10 aa, Homo sapiens
  MOD_RES 4: note = Oxidized Methionine
SVPMVPPGIK

SEQ ID NO: 384 - protein, 8 aa, Homo sapiens
VANEVTLN

SEQ ID NO: 385 - protein, 15 aa, Homo sapiens
DTGENLETPSSFTIK

SEQ ID NO: 386 - protein, 13 aa, Homo sapiens
DWIWNQMHIDEEK

SEQ ID NO: 387 - protein, 11 aa, Homo sapiens
ELDSTGTPTGK

SEQ ID NO: 388 - protein, 22 aa, Homo sapiens
ESIVQVHIEVLDENDNAPEFAK

SEQ ID NO: 389 - protein, 21 aa, Homo sapiens
GDYQDAFTIETNPAHNEGIIK

SEQ ID NO: 390 - protein, 8 aa, Homo sapiens
HSIGYSIR

SEQ ID NO: 391 - protein, 8 aa, Homo sapiens
KGDIYNEK

SEQ ID NO: 392 - protein, 16 aa, Homo sapiens
KPLIGTVLAMDPDAAR

SEQ ID NO: 393 - protein, 18 aa, Homo sapiens
LDRENISEYHLTAVIVDK

SEQ ID NO: 394 - protein, 13 aa, Homo sapiens
VDAETGDVFAIER

SEQ ID NO: 395 - protein, 16 aa, Homo sapiens
VFRVDAETGDVFAIER

SEQ ID NO: 396 - protein, 14 aa, Homo sapiens
VHDVNDNWPVFTHR

SEQ ID NO: 397 - protein, 16 aa, Homo sapiens
  MOD_RES 11: note = Deamidated Asparagine
VHFLPVVISDNGMPSR

SEQ ID NO: 398 - protein, 16 aa, Homo sapiens
VHFLPVVISDNGMPSR

SEQ ID NO: 399 - protein, 9 aa, Homo sapiens
YMSPPAGNR

SEQ ID NO: 400 - protein, 10 aa, Homo sapiens
YTFVVPEDTR

SEQ ID NO: 401 - protein, 19 aa, Homo sapiens
EVQLVQSGGGVVQPGGSLR

SEQ ID NO: 402 - protein, 11 aa, Homo sapiens
  MOD_RES 9: note = Carboxymethylated Cysteine
TEDTAVYYCAK

SEQ ID NO: 403 - protein, 9 aa, Homo sapiens
RAEQELAGR

SEQ ID NO: 404 - protein, 13 aa, Homo sapiens
DTLVADNLDQATR

SEQ ID NO: 405 - protein, 11 aa, Homo sapiens
PNLGAIAEYKK

SEQ ID NO: 406 - protein, 24 aa, Homo sapiens
  SITE 1: note = Pyroglutamic acid
  MOD_RES 5: note = Oxidized Methionine
XQEDMKGHFSISIPVKSDIAPVAR

SEQ ID NO: 407 - protein, 24 aa, Homo sapiens
  SITE 1: note = Pyroglutamic acid
XQEDMKGHFSISIPVKSDIAPVAR

SEQ ID NO: 408 - protein, 17 aa, Homo sapiens
  SITE 1: note = Pyroglutamic acid
XQFSFPLSSEPFQGSYK

SEQ ID NO: 409 - protein, 11 aa, Homo sapiens
  SITE 1: note = Pyroglutamic acid
XQGIPFFGQVR

SEQ ID NO: 410 - protein, 9 aa, Homo sapiens
  SITE 1: note = Pyroglutamic acid
  MOD_RES 5: note = Deamidated Asparagine
  MOD_RES 7: note = Carboxymethylated Cysteine
XQKDNGCFR

SEQ ID NO: 411 - protein, 9 aa, Homo sapiens
  SITE 1: note = Pyroglutamic acid
  MOD_RES 7: note = Carboxymethylated Cysteine
XQKDNGCFR

SEQ ID NO: 412 - protein, 18 aa, Homo sapiens
  SITE 1: note = Pyroglutamic acid
XQLNYKHYDGSYSTFGER

SEQ ID NO: 413 - protein, 24 aa, Homo sapiens
  SITE 1: note = Pyroglutamic acid
  MOD_RES 4: note = Deamidated Asparagine
XQQNAQGGFSSTQDTVVALHALSK

SEQ ID NO: 414 - protein, 24 aa, Homo sapiens
  SITE 1: note = Pyroglutamic acid
XQQNAQGGFSSTQDTVVALHALSK

SEQ ID NO: 415 - protein, 8 aa, Homo sapiens
  SITE 1: note = Pyroglutamic acid
XQSSEITR

SEQ ID NO: 416 - protein, 11 aa, Homo sapiens
  SITE 1: note = Pyroglutamic acid
XQTVSWAVTPK

SEQ ID NO: 417 - protein, 15 aa, Homo sapiens
AAQVTIQSSGTFSSK

SEQ ID NO: 418 - protein, 17 aa, Homo sapiens
  MOD_RES 11: note = Oxidized Methionine
AFQPFFVELTMPYSVIR

SEQ ID NO: 419 - protein, 24 aa, Homo sapiens
  MOD_RES 11: note = Oxidized Methionine
AFQPFFVELTMPYSVIRGEAFTLK

SEQ ID NO: 420 - protein, 17 aa, Homo sapiens
AFQPFFVELTMPYSVIR

SEQ ID NO: 421 - protein, 24 aa, Homo sapiens
AFQPFFVELTMPYSVIRGEAFTLK

SEQ ID NO: 422 - protein, 21 aa, Homo sapiens
  MOD_RES 5: note = Carboxymethylated Cysteine
AGAFCLSEDAGLGISSTASLR

SEQ ID NO: 423 - protein, 17 aa, Homo sapiens
AHTSFQISLSVSYTGSR

SEQ ID NO: 424 - protein, 11 aa, Homo sapiens
  MOD_RES 6: note = Deamidated Asparagine
AIGYLNTGYQR

SEQ ID NO: 425 - protein, 11 aa, Homo sapiens
AIGYLNTGYQR

SEQ ID NO: 426 - protein, 11 aa, Homo sapiens
  MOD_RES 8: note = Carboxymethylated Cysteine
ALANSLACQGK

SEQ ID NO: 427 - protein, 28 aa, Homo sapiens
  MOD_RES 25: note = Carboxymethylated Cysteine
ALSDHHIYLEGTLLKPNMVTPGHACTQK

SEQ ID NO: 428 - protein, 15 aa, Homo sapiens
FSHEEIAMATVTALR

SEQ ID NO: 429 - protein, 16 aa, Homo sapiens
FSHEEIAMATVTALRR

SEQ ID NO: 430 - protein, 14 aa, Homo sapiens
GILAADESTGSIAK

SEQ ID NO: 431 - protein, 15 aa, Homo sapiens
GILAADESTGSIAKR

SEQ ID NO: 432 - protein, 23 aa, Homo sapiens
GVVPLAGTNGETTTQGLDGLSER

SEQ ID NO: 433 - protein, 20 aa, Homo sapiens
IGEHTPSALAIMENANVLAR

SEQ ID NO: 434 - protein, 14 aa, Homo sapiens
LQSIGTENTEENRR

SEQ ID NO: 435 - protein, 12 aa, Homo sapiens
PYQYPALTPEQK

SEQ ID NO: 436 - protein, 13 aa, Homo sapiens
PYQYPALTPEQKK

SEQ ID NO: 437 - protein, 9 aa, Homo sapiens
QLLLTADDR

SEQ ID NO: 438 - protein, 27 aa, Homo sapiens
  MOD_RES 5: note = Carboxymethylated Cysteine
YASICQQNGIVPIVEPEILPDGDHDLK

SEQ ID NO: 439 - protein, 8 aa, Homo sapiens
  SITE 1: note = Pyroglutamic acid
XQDSISSK

SEQ ID NO: 440 - protein, 14 aa, Homo sapiens
  MOD_RES 7: note = Carboxymethylated Cysteine
AESPEVCFNEESPK

SEQ ID NO: 441 - protein, 7 aa, Homo sapiens
AFSSYQK

SEQ ID NO: 442 - protein, 9 aa, Homo sapiens
AIPVTQYLK

SEQ ID NO: 443 - protein, 8 aa, Homo sapiens
  MOD_RES 1: note = Carboxymethylated Cysteine
CQAYESNR

SEQ ID NO: 444 - protein, 21 aa, Homo sapiens
  MOD_RES 1: note = Carboxymethylated Cysteine
CQAYESNRESLLNHFLYEVAR

SEQ ID NO: 445 - protein, 10 aa, Homo sapiens
DADPDTFFAK

SEQ ID NO: 446 - protein, 8 aa, Homo sapiens
DLSLREGK

SEQ ID NO: 447 - protein, 26 aa, Homo sapiens
  MOD_RES 16..17: note = Carboxymethylated Cysteine
  MOD_RES 24: note = Carboxymethylated Cysteine
ELISLVEDVSSNYDGCCEGDVVQCIR

SEQ ID NO: 448 - protein, 13 aa, Homo sapiens
  MOD_RES 5: note = Deamidated Asparagine
ESLLNHFLYEVAR

SEQ ID NO: 449 - protein, 13 aa, Homo sapiens
ESLLNHFLYEVAR

SEQ ID NO: 450 - protein, 28 aa, Homo sapiens
FIEDNIEYITIIAFAQYVQEATFEEMEK

SEQ ID NO: 451 - protein, 11 aa, Homo sapiens
  MOD_RES 8: note = Carboxymethylated Cysteine
FTDSENVCQER

SEQ ID NO: 452 - protein, 21 aa, Homo sapiens
  MOD_RES 8: note = Carboxymethylated Cysteine
FTDSENVCQERDADPDTFFAK

SEQ ID NO: 453 - protein, 7 aa, Homo sapiens
FTFEYSR

SEQ ID NO: 454 - protein, 9 aa, Homo sapiens
  MOD_RES 3: note = Carboxymethylated Cysteine
  MOD_RES 6: note = Deamidated Asparagine
GQCIINSNK

SEQ ID NO: 455 - protein, 14 aa, Homo sapiens
  MOD_RES 3: note = Carboxymethylated Cysteine
  MOD_RES 8: note = Deamidated Asparagine
GQCIINSNKDDRPK

SEQ ID NO: 456 - protein, 9 aa, Homo sapiens
  MOD_RES 3: note = Carboxymethylated Cysteine
GQCIINSNK

SEQ ID NO: 457 - protein, 14 aa, Homo sapiens
  MOD_RES 3: note = Carboxymethylated Cysteine
GQCIINSNKDDRPK

SEQ ID NO: 458 - protein, 21 aa, Homo sapiens
HELTDEELQSLFTNFANVVDK

SEQ ID NO: 459 - protein, 14 aa, Homo sapiens
  MOD_RES 10: note = Carboxymethylated Cysteine
EVQPVELPNCNLVK

SEQ ID NO: 460 - protein, 32 aa, Homo sapiens
  MOD_RES 21: note = Oxidized Methionine
FDVSSFNPHGISTFTDEDNAMYLLVVNHPDAK

SEQ ID NO: 461 - protein, 32 aa, Homo sapiens; identical to SEQ ID NO: 460

SEQ ID NO: 462 - protein, 24 aa, Homo sapiens
  MOD_RES 14: note = Deamidated Asparagine
GIETGSEDLEILPNGLAFISSGLK

SEQ ID NO: 463 - protein, 24 aa, Homo sapiens
GIETGSEDLEILPNGLAFISSGLK

SEQ ID NO: 464 - protein, 16 aa, Homo sapiens
IFFYDSENPPASEVLR

SEQ ID NO: 465 - protein, 22 aa, Homo sapiens
  MOD_RES 4: note = Oxidized Methionine
ILLMDLNEEDPTVLELGITGSK

SEQ ID NO: 466 - protein, 22 aa, Homo sapiens
ILLMDLNEEDPTVLELGITGSK

SEQ ID NO: 467 - protein, 10 aa, Homo sapiens
IQNILTEEPK

SEQ ID NO: 468 - protein, 9 aa, Homo sapiens
LLIGTVFHK

SEQ ID NO: 469 - protein, 19 aa, Homo sapiens
  MOD_RES 15: note = Carboxymethylated Cysteine
LNALREVQPVELPNCNLVK

SEQ ID NO: 470 - protein, 9 aa, Homo sapiens
SFNPNSPGK

SEQ ID NO: 471 - protein, 30 aa, Homo sapiens
  MOD_RES 24: note = Carboxymethylated Cysteine
  MOD_RES 27: note = Deamidated Asparagine
SLDFNTLVDNISVDPETGDLWVGCHPNGMK

SEQ ID NO: 472 - protein, 30 aa, Homo sapiens; identical to SEQ ID NO: 471
  MOD_RES 24: note = Carboxymethylated Cysteine

SEQ ID NO: 473 - protein, 22 aa, Homo sapiens
VTQVYAENGTVLQGSTVASVYK

SEQ ID NO: 474 - protein, 19 aa, Homo sapiens
  MOD_RES 10: note = Deamidated Asparagine
VVAEGFDFANGINISPDGK

SEQ ID NO: 475 - protein, 19 aa, Homo sapiens
VVAEGFDFANGINISPDGK

SEQ ID NO: 476 - protein, 11 aa, Homo sapiens
YVYIAELLAHK

SEQ ID NO: 477 - protein, 17 aa, Homo sapiens
YVYIAELLAHKIHVYEK

SEQ ID NO: 478 - protein, 12 aa, Homo sapiens
EHALLAYTLGVK

SEQ ID NO: 479 - protein, 11 aa, Homo sapiens
IGGIGTVPVGR

SEQ ID NO: 480 - protein, 12 aa, Homo sapiens
AAAATGTIFTFR

SEQ ID NO: 481 - protein, 34 aa, Homo sapiens
ALASAAPSQSIFFSPVSISMSLAMLSLGAGSSTK

SEQ ID NO: 482 - protein, 11 aa, Homo sapiens
AVVEVDESGTR

SEQ ID NO: 483 - protein, 8 aa, Homo sapiens
DFTFDLYR

SEQ ID NO: 484 - protein, 11 aa, Homo sapiens
FSIEGSYQLEK

SEQ ID NO: 485 - protein, 13 aa, Homo sapiens
GFQQLLQELNQPR

SEQ ID NO: 486 - protein, 16 aa, Homo sapiens
  MOD_RES 3: note = Deamidated Glutamine
GTQEQDFYVTSETVVR

SEQ ID NO: 487 - protein, 16 aa, Homo sapiens
GTQEQDFYVTSETVVR

SEQ ID NO: 488 - protein, 13 aa, Homo sapiens
MQILEGLGLNLQK

SEQ ID NO: 489 - protein, 11 aa, Homo sapiens
  MOD_RES 6: note = Deamidated Asparagine
MQQVENGLSEK

SEQ ID NO: 490 - protein, 11 aa, Homo sapiens
MQQVENGLSEK

SEQ ID NO: 491 - protein, 8 aa, Homo sapiens
QLELYLPK

SEQ ID NO: 492 - protein, 13 aa, Homo sapiens
TLYLADTFPTNFR

SEQ ID NO: 493 - protein, 27 aa, Homo sapiens
  MOD_RES 13: note = Carboxymethylated Cysteine
  MOD_RES 23: note = Carboxymethylated Cysteine
SVLRPSQFGGQPCTEPLVAFQPCIPSK

SEQ ID NO: 494 - protein, 10 aa, Homo sapiens
  MOD_RES 5: note = Carboxymethylated Cysteine
AFIGCPPPPK

SEQ ID NO: 495 - protein, 12 aa, Homo sapiens
QLLTKPQHQFVR

SEQ ID NO: 496 - protein, 11 aa, Homo sapiens
QSLQELPAGSR

SEQ ID NO: 497 - protein, 17 aa, Homo sapiens
ITANLFRLFHSLMVLEK

SEQ ID NO: 498 - protein, 13 aa, Homo sapiens
LLAENNEIISNIR

SEQ ID NO: 499 - protein, 11 aa, Homo sapiens
VLLAPLGDDFR

SEQ ID NO: 500 - protein, 18 aa, Homo sapiens
  SITE 1: note = Pyroglutamic acid
XQPFVQGLALYTPVVLPR

SEQ ID NO: 501 - protein, 14 aa, Homo sapiens
  MOD_RES 3: note = Oxidized Methionine
  MOD_RES 6: note = Oxidized Methionine
AAMVGMLANFLGFR

SEQ ID NO: 502 - protein, 14 aa, Homo sapiens
  MOD_RES 3: note = Oxidized Methionine
AAMVGMLANFLGFR

SEQ ID NO: 503 - protein, 14 aa, Homo sapiens
  MOD_RES 6: note = Oxidized Methionine
AAMVGMLANFLGFR

SEQ ID NO: 504 - protein, 14 aa, Homo sapiens
AAMVGMLANFLGFR

SEQ ID NO: 505 - protein, 24 aa, Homo sapiens
  MOD_RES 4: note = Deamidated Glutamine
ADSQAQLLLSTVVGVFTAPGLHLK

SEQ ID NO: 506 - protein, 24 aa, Homo sapiens
  MOD_RES 6: note = Deamidated Glutamine
ADSQAQLLLSTVVGVFTAPGLHLK

SEQ ID NO: 507 - protein, 24 aa, Homo sapiens
ADSQAQLLLSTVVGVFTAPGLHLK

SEQ ID NO: 508 - protein, 12 aa, Homo sapiens
  MOD_RES 3: note = Deamidated Glutamine
ALQDQLVLVAAK

SEQ ID NO: 509 - protein, 12 aa, Homo sapiens
ALQDQLVLVAAK

SEQ ID NO: 510 - protein, 18 aa, Homo sapiens
ALQDQLVLVAAKLDTEDK

SEQ ID NO: 511 - protein, 20 aa, Homo sapiens
ALQDQLVLVAAKLDTEDKLR

SEQ ID NO: 512 - protein, 12 aa, Homo sapiens
  MOD_RES 10: note = Deamidated Glutamine
DPTFIPAPIQAK

SEQ ID NO: 513 - protein, 12 aa, Homo sapiens
DPTFIPAPIQAK

SEQ ID NO: 514 - protein, 9 aa, Homo sapiens
  MOD_RES 2: note = Oxidized Methionine
FMQAVTGWK

SEQ ID NO: 515 - protein, 9 aa, Homo sapiens
FMQAVTGWK

SEQ ID NO: 516 - protein, 12 aa, Homo sapiens
  MOD_RES 5: note = Oxidized Methionine
IDRFMQAVTGWK

SEQ ID NO: 517 - protein, 12 aa, Homo sapiens
IDRFMQAVTGWK

SEQ ID NO: 518 - protein, 41 aa, Homo sapiens
IYGMHSELWGVVHGATVLSPTAVFGTLASLYLGALDHTADR

SEQ ID NO: 519 - protein, 22 aa, Homo sapiens
LDAHKVLSALQAVQGLLVAQGR

SEQ ID NO: 520 - protein, 12 aa, Homo sapiens
MVTGVASALSSR

SEQ ID NO: 521 - protein, 11 aa, Homo sapiens
ATWSGAVLAGR

SEQ ID NO: 522 - protein, 16 aa, Homo sapiens
ATWSGAVLAGRDAVLR

SEQ ID NO: 523 - protein, 14 aa, Homo sapiens
  MOD_RES 1: note = Carboxymethylated Cysteine
CEGPIPDVTFELLR

SEQ ID NO: 524 - protein, 19 aa, Homo sapiens
  MOD_RES 1: note = Carboxymethylated Cysteine
CEGPIPDVTFELLREGETK

SEQ ID NO: 525 - protein, 9 aa, Homo sapiens
  MOD_RES 1: note = Carboxymethylated Cysteine
CLAPLEGAR

SEQ ID NO: 526 - protein, 8 aa, Homo sapiens
FALVREDR

SEQ ID NO: 527 - protein, 12 aa, Homo sapiens
GVLRGVTFLLRR

SEQ ID NO: 528 - protein, 7 aa, Homo sapiens
GVTFLLR

SEQ ID NO: 529 - protein, 8 aa, Homo sapiens
GVTFLLRR

SEQ ID NO: 530 - protein, 12 aa, Homo sapiens
HQFLLTGDTQGR

SEQ ID NO: 531 - protein, 19 aa, Homo sapiens
  MOD_RES 6: note = Deamidated Asparagine
  MOD_RES 18: note = Carboxymethylated Cysteine
IFFHLNAVALGDGGHYTCR

SEQ ID NO: 532 - protein, 19 aa, Homo sapiens
  MOD_RES 18: note = Carboxymethylated Cysteine
IFFHLNAVALGDGGHYTCR

SEQ ID NO: 533 - protein, 15 aa, Homo sapiens
LELHVDGPPPRPQLR

SEQ ID NO: 534 - protein, 8 aa, Homo sapiens
LLELTGPK

SEQ ID NO: 535 - protein, 11 aa, Homo sapiens
  MOD_RES 3: note = Carboxymethylated Cysteine
LRCLAPLEGAR

SEQ ID NO: 536 - protein, 16 aa, Homo sapiens
  MOD_RES 1: note = Deamidated Asparagine
NGVAQEPVHLDSPAIK

SEQ ID NO: 537 - protein, 16 aa, Homo sapiens
NGVAQEPVHLDSPAIK

SEQ ID NO: 538 - protein, 10 aa, Homo sapiens
RGEKELLVPR

SEQ ID NO: 539 - protein, 12 aa, Homo sapiens
SGLSTGWTQLSK

SEQ ID NO: 540 - protein, 20 aa, Homo sapiens
  MOD_RES 9: note = Oxidized Methionine
SLPAPWLSMAPVSWITPGLK

SEQ ID NO: 541 - protein, 13 aa, Homo sapiens
  MOD_RES 1: note = Deamidated Asparagine
NRNVHSAGAAGSR

SEQ ID NO: 542 - protein, 13 aa, Homo sapiens
  MOD_RES 3: note = Deamidated Asparagine
NRNVHSAGAAGSR

SEQ ID NO: 543 - protein, 13 aa, Homo sapiens
NRNVHSAGAAGSR

SEQ ID NO: 544 - protein, 11 aa, Homo sapiens
NVHSAGAAGSR

SEQ ID NO: 545 - protein, 11 aa, Homo sapiens
  MOD_RES 3: note = Deamidated Glutamine
PGQVDAAMAGR

SEQ ID NO: 546 - protein, 11 aa, Homo sapiens
PGQVDAAMAGR

SEQ ID NO: 547 - protein, 13 aa, Homo sapiens
AHSSMVGVNLPQK

SEQ ID NO: 548 - protein, 17 aa, Homo sapiens
  MOD_RES 2: note = Carboxymethylated Cysteine
  MOD_RES 14..15: note = Carboxymethylated Cysteine
GCITIIGGGDTATCCAK

SEQ ID NO: 549 - protein, 18 aa, Homo sapiens
VSHVSTGGGASLELLEGK

SEQ ID NO: 550 - protein, 15 aa, Homo sapiens
VQLVESGGGVVQPGR

SEQ ID NO: 551 - protein, 34 aa, Homo sapiens
  SITE 1: note = Pyroglutamic acid
  MOD_RES 3: note = Carboxymethylated Cysteine
  MOD_RES 16: note = Carboxymethylated Cysteine
  MOD_RES 23: note = Carboxymethylated Cysteine
XQCRPNGMWDGETAVCDNGAGHCPNPGISLGAVR

SEQ ID NO: 552 - protein, 12 aa, Homo sapiens
  SITE 1: note = Pyroglutamic acid
XHLGDVLNFLPL

SEQ ID NO: 553 - protein, 15 aa, Homo sapiens
  MOD_RES 1: note = Carboxymethylated Cysteine
  MOD_RES 9: note = Deamidated Asparagine
CPAPVSFENGIYTPR

SEQ ID NO: 554 - protein, 15 aa, Homo sapiens
  MOD_RES 1: note = Carboxymethylated Cysteine
CPAPVSFENGIYTPR

SEQ ID NO: 555 - protein, 8 aa, Homo sapiens
DFHINLFR

SEQ ID NO: 556 - protein, 15 aa, Homo sapiens
  MOD_RES 2: note = Oxidized Methionine
DMTEVISSLENANYK

SEQ ID NO: 557 - protein, 15 aa, Homo sapiens
  MOD_RES 11: note = Deamidated Asparagine
DMTEVISSLENANYK

SEQ ID NO: 558 - protein, 15 aa, Homo sapiens
DMTEVISSLENANYK

SEQ ID NO: 559 - protein, 9 aa, Homo sapiens
ESASLMVDR

SEQ ID NO: 560 - protein, 31 aa, Homo sapiens
  MOD_RES 9: note = Carboxymethylated Cysteine
  MOD_RES 19: note = Carboxymethylated Cysteine
EVVTDQFLCSGTQEDESPCKGESGGAVFLER

SEQ ID NO: 561 - protein, 21 aa, Homo sapiens
  MOD_RES 15: note = Carboxymethylated Cysteine
FFQVGLVSWGLYNPCLGSADK

SEQ ID NO: 562 - protein, 23 aa, Homo sapiens
  MOD_RES 17: note = Carboxymethylated Cysteine
FRFFQVGLVSWGLYNPCLGSADK

SEQ ID NO: 563 - protein, 11 aa, Homo sapiens
GESGGAVFLER

SEQ ID NO: 564 - protein, 33 aa, Homo sapiens
  MOD_RES 2: note = Carboxymethylated Cysteine
  MOD_RES 5: note = Deamidated Asparagine
  MOD_RES 15: note = Carboxymethylated Cysteine
  MOD_RES 22: note = Carboxymethylated Cysteine
QCRPNGMWDGETAVCDNGAGHCPNPGISLGAVR

SEQ ID NO: 565 - protein, 33 aa, Homo sapiens; identical to SEQ ID NO: 564
  MOD_RES 2: note = Carboxymethylated Cysteine
  MOD_RES 15: note = Carboxymethylated Cysteine
  MOD_RES 17: note = Deamidated Asparagine
  MOD_RES 22: note = Carboxymethylated Cysteine

SEQ ID NO: 566 - protein, 33 aa, Homo sapiens; identical to SEQ ID NO: 564
  MOD_RES 2: note = Carboxymethylated Cysteine
  MOD_RES 15: note = Carboxymethylated Cysteine
  MOD_RES 22: note = Carboxymethylated Cysteine

SEQ ID NO: 567 - protein, 12 aa, Homo sapiens
QHLGDVLNFLPL

SEQ ID NO: 568 - protein, 15 aa, Homo sapiens
SKVPPPRDFHINLFR

SEQ ID NO: 569 - protein, 11 aa, Homo sapiens
  MOD_RES 3: note = Oxidized Methionine
VLMSVLNDNSR

SEQ ID NO: 570 - protein, 11 aa, Homo sapiens
  MOD_RES 7: note = Deamidated Asparagine
VLMSVLNDNSR

SEQ ID NO: 571 - protein, 10 aa, Homo sapiens
LHMLQISYFR

SEQ ID NO: 572 - protein, 9 aa, Homo sapiens
TLAFPLTIR

SEQ ID NO: 573 - protein, 9 aa, Homo sapiens
  MOD_RES 3: note = Carboxymethylated Cysteine
LSCVGSGFR

SEQ ID NO: 574 - protein, 22 aa, Homo sapiens
  MOD_RES 20: note = Carboxymethylated Cysteine
SSLYLQMKSLRDEDTAVYYCAR

SEQ ID NO: 575 - protein, 25 aa, Homo sapiens
  SITE 1: note = Pyroglutamic acid
XQGPVNLLSDPEQGVEVTGQYEREK

SEQ ID NO: 576 - protein, 21 aa, Homo sapiens
  SITE 1: note = Pyroglutamic acid
XQLGLPGPPDVPDHAAYHPFR

SEQ ID NO: 577 - protein, 22 aa, Homo sapiens
  SITE 1: note = Pyroglutamic acid
XQLGLPGPPDVPDHAAYHPFRR

SEQ ID NO: 578 - protein, 13 aa, Homo sapiens
AEAQAQYSAAVAK

SEQ ID NO: 579 - protein, 20 aa, Homo sapiens
AFITNFSMNIDGMTYPGIIK

SEQ ID NO: 580 - protein, 11 aa, Homo sapiens
AGFSWIEYTFK

SEQ ID NO: 581 - protein, 28 aa, Homo sapiens
AGFSWIEVTFKNPLVWVHASPEHYYYTR

SEQ ID NO: 582 - protein, 28 aa, Homo sapiens
AISGGSIQIENGYFVHYFAPEGLTTMPK

SEQ ID NO: 583 - protein, 15 aa, Homo sapiens
  MOD_RES 11: note = Oxidized Methionine
ANTVQEATFQMELPK

SEQ ID NO: 584 - protein, 16 aa, Homo sapiens
  MOD_RES 11: note = Oxidized Methionine
ANTVQEATFQMELPKK

SEQ ID NO: 585 - protein, 15 aa, Homo sapiens
ANTVQEATFQMELPK

SEQ ID NO: 586 - protein, 16 aa, Homo sapiens
ANTVQEATFQMELPKK

SEQ ID NO: 587 - protein, 16 aa, Homo sapiens
DQFNLIVFSTEATQWR

SEQ ID NO: 588 - protein, 29 aa, Homo sapiens
DQFNLIVFSTEATQWRPSLVPASAENVNK

SEQ ID NO: 589 - protein, 32 aa, Homo sapiens
DTDRFSSHVGGTLGQFYQEVLWGSPAASDDGR

SEQ ID NO: 590 - protein, 33 aa, Homo sapiens
DTDRFSSHVGGTLGQFYQEVLWGSPAASDDGRR

SEQ ID NO: 591 - protein, 15 aa, Homo sapiens
EKAEAQAQYSAAVAK

SEQ ID NO: 592 - protein, 11 aa, Homo sapiens
  MOD_RES 7: note = Oxidized Methionine
ETLFSVMPGLK

SEQ ID NO: 593 - protein, 11 aa, Homo sapiens
ETLFSVMPGLK

SEQ ID NO: 594 - protein, 9 aa, Homo sapiens
FAHTVVTSR

SEQ ID NO: 595 - protein, 9 aa, Homo sapiens
AVPVAADQR

SEQ ID NO: 596 - protein, 16 aa, Homo sapiens
DAQGQPQAYPVSGDLR

SEQ ID NO: 597 - protein, 8 aa, Homo sapiens
DLQFSEIR

SEQ ID NO: 598 - protein, 11 aa, Homo sapiens
EVTVQGLEPSR

SEQ ID NO: 599 - protein, 19 aa, Homo sapiens
FGVPSPSTLEPHPRPLLQR

SEQ ID NO: 600 - protein, 9 aa, Homo sapiens
FLLYGLSGR

SEQ ID NO: 601 - protein, 13 aa, Homo sapiens
ILISGLEPSTPYR

SEQ ID NO: 602 - protein, 15 aa, Homo sapiens
LGYLTVTDTTPDSMR

SEQ ID NO: 603 - protein, 9 aa, Homo sapiens
LQGLIPGAR

SEQ ID NO: 604 - protein, 11 aa, Homo sapiens
LQVVPVAANQR

SEQ ID NO: 605 - protein, 15 aa, Homo sapiens
LSQLSYTDYTTSSLR

SEQ ID NO: 606 - protein, 16 aa, Homo sapiens
RLGPISADSTTAPLEK

SEQ ID NO: 607 - protein, 10 aa, Homo sapiens
TLSPVLESPR

SEQ ID NO: 608 - protein, 10 aa, Homo sapiens
TSTIFLNGNR

SEQ ID NO: 609 - protein, 11 aa, Homo sapiens
TVTVEDLEPGK

SEQ ID NO: 610 - protein, 9 aa, Homo sapiens
VNWMPPPSR

SEQ ID NO: 611 - protein, 21 aa, Homo sapiens
YSYQLADGGEPQSYQYDGQAR

SEQ ID NO: 612 - protein, 9 aa, Homo sapiens
YEVTVVSVR

SEQ ID NO: 613 - protein, 10 aa, Homo sapiens
DYVASLVSTR

SEQ ID NO: 614 - protein, 11 aa, Homo sapiens
EHNLQYLGLVK

SEQ ID NO: 615 - protein, 8 aa, Homo sapiens
LIVAGLPR

SEQ ID NO: 616 - protein, 12 aa, Homo sapiens
NEEALDTESSER

SEQ ID NO: 617 - protein, 18 aa, Homo sapiens
TFTPFYFLVEPVDTLSYR

SEQ ID NO: 618 - protein, 15 aa, Homo sapiens
YLPDPEYISDLVFLK

SEQ ID NO: 619 - protein, 9 aa, Homo sapiens
AKLPDATPK

SEQ ID NO: 620 - protein, 12 aa, Homo sapiens
  MOD_RES 1..2: note = Carboxymethylated Cysteine
  MOD_RES 9: note = Carboxymethylated Cysteine
  MOD_RES 10: note = Oxidized Methionine
CCESASEDCMAK

SEQ ID NO: 621 - protein, 12 aa, Homo sapiens
  MOD_RES 1..2: note = Carboxymethylated Cysteine
  MOD_RES 9: note = Carboxymethylated Cysteine
CCESASEDCMAK

SEQ ID NO: 622 - protein, 9 aa, Homo sapiens
  MOD_RES 3: note = Carboxymethylated Cysteine
DVCDPGNTK

SEQ ID NO: 623 - protein, 13 aa, Homo sapiens
EDFTSLSLVLYSR

SEQ ID NO: 624 - protein, 14 aa, Homo sapiens
EDFTSLSLVLYSRK

SEQ ID NO: 625 - protein, 20 aa, Homo sapiens
EFSHLGKEDFTSLSLYLYSR

SEQ ID NO: 626 - protein, 8 aa, Homo sapiens
ELPEHTVK

SEQ ID NO: 627 - protein, 8 aa, Homo sapiens
ELSSFIDK

SEQ ID NO: 628 - protein, 22 aa, Homo sapiens
  MOD_RES 9..10: note = Carboxymethylated Cysteine
  MOD_RES 18: note = Carboxymethylated Cysteine
EYVSLTEACCAEGADPDCYDTR

SEQ ID NO: 629 - protein, 27 aa, Homo sapiens
EYANQFMWEYSTNYGQAPLSLLYSYTK

SEQ ID NO: 630 - protein, 8 aa, Homo sapiens
  MOD_RES 4..5: note = Carboxymethylated Cysteine
FEDCCQEK

SEQ ID NO: 631 - protein, 14 aa, Homo sapiens
FPSGTFEQVSQLVK

SEQ ID NO: 632 - protein, 17 aa, Homo sapiens
  MOD_RES 5: note = Carboxymethylated Cysteine
GQELCADYSENTFTEYK

SEQ ID NO: 633 - protein, 18 aa, Homo sapiens
  MOD_RES 5: note = Carboxymethylated Cysteine
GQELCADYSENTFTEYKK

SEQ ID NO: 634 - protein, 11 aa, Homo sapiens
  MOD_RES 10: note = Deamidated Asparagine
HLSLLTTLSNR

SEQ ID NO: 635 - protein, 11 aa, Homo sapiens
HLSLLTTLSNR

SEQ ID NO: 636 - protein, 23 aa, Homo sapiens
  MOD_RES 14: note = Deamidated Asparagine
  MOD_RES 18: note = Carboxymethylated Cysteine
HQPQEFPTYVEPTNDEICEAFRK

SEQ ID NO: 637 - protein, 22 aa, Homo sapiens
  MOD_RES 18: note = Carboxymethylated Cysteine
HQPQEFPTYYEPTNDEICEAFR

SEQ ID NO: 638 - protein, 23 aa, Homo sapiens
  MOD_RES 18: note = Carboxymethylated Cysteine
HQPQEFPTYYEPTNDEICEAFRK

SEQ ID NO: 639 - protein, 8 aa, Homo sapiens
  MOD_RES 7: note = Carboxymethylated Cysteine
ALEVEECR

SEQ ID NO: 640 - protein, 11 aa, Homo sapiens
  MOD_RES 2: note = Oxidized Methionine
AMDFNGILTIR

SEQ ID NO: 641 - protein, 11 aa, Homo sapiens
ASYAQQPAESR

SEQ ID NO: 642 - protein, 17 aa, Homo sapiens
  MOD_RES 1: note = Carboxymethylated Cysteine
  MOD_RES 15: note = Carboxymethylated Cysteine
CAPGYYGNPSQGQPCQR

SEQ ID NO: 643 - protein, 24 aa, Homo sapiens
  MOD_RES 1: note = Carboxymethylated Cysteine
CSATGSPTPTLEWTGGPGGQLPAK

SEQ ID NO: 644 - protein, 16 aa, Homo sapiens
EHLLMALADLDELLIR

SEQ ID NO: 645 - protein, 16 aa, Homo sapiens
EHLLMALAGIDTLLIR

SEQ ID NO: 646 - protein, 13 aa, Homo sapiens
GHTPTQPGALNQR

SEQ ID NO: 647 - protein, 22 aa, Homo sapiens
HLISTHFAPGDFQGFALYNPQR

SEQ ID NO: 648 - protein, 13 aa, Homo sapiens
IAHYELADAGQYR

SEQ ID NO: 649 - protein, 17 aa, Homo sapiens
IQYYVLSASDASPPPVK

SEQ ID NO: 650 - protein, 15 aa, Homo sapiens
  MOD_RES 2: note = Carboxymethylated Cysteine
  MOD_RES 5: note = Carboxymethylated Cysteine
LCNECADGSFHLSTR

SEQ ID NO: 651 - protein, 15 aa, Homo sapiens
  MOD_RES 14: note = Carboxymethylated Cysteine
LHQMSVADSGEYVCR

SEQ ID NO: 652 - protein, 15 aa, Homo sapiens
  MOD_RES 14: note = Carboxymethylated Cysteine
LPAVEPTDQAQYLCR

SEQ ID NO: 653 - protein, 17 aa, Homo sapiens
  MOD_RES 16: note = Carboxymethylated Cysteine
LYIFQASPADAGQYVCR

SEQ ID NO: 654 - protein, 10 aa, Homo sapiens
SPAYTLVWTR

SEQ ID NO: 655 - protein, 7 aa, Homo sapiens
VQVSPER

SEQ ID NO: 656 - protein, 14 aa, Homo sapiens
GNALALLVLPDPGK

SEQ ID NO: 657 - protein, 12 aa, Homo sapiens
HQLSEPAPAYHR

SEQ ID NO: 658 - protein, 11 aa, Homo sapiens
ITPTITNFALR

SEQ ID NO: 659 - protein, 9 aa, Homo sapiens
QESFFVDER

SEQ ID NO: 660 - protein, 12 aa, Homo sapiens
QVEAALQPQTLR

SEQ ID NO: 661 - protein, 12 aa, Homo sapiens
SLLHTLALPSPK

SEQ ID NO: 662 - protein, 16 aa, Homo sapiens
WGQLLLPSLLDLHLPR

SEQ ID NO: 663 - protein, 11 aa, Homo sapiens
  MOD_RES 6: note = Deamidated Glutamine
NSVFLQMNSLR

SEQ ID NO: 664 - protein, 11 aa, Homo sapiens
  MOD_RES 8: note = Deamidated Asparagine
NSVFLQMNSLR

SEQ ID NO: 665 - protein, 11 aa, Homo sapiens
NSVFLQMNSLR

SEQ ID NO: 666 - protein, 21 aa, Homo sapiens
  MOD_RES 3: note = Oxidized Methionine
  MOD_RES 18: note = Oxidized Methionine
ALMLQGVDLLADAVAVTMGPK

SEQ ID NO: 667 - protein, 25 aa, Homo sapiens
LVQDVANNTNEEAGDGTTTATVLAR

SEQ ID NO: 668 - protein, 11 aa, Homo sapiens
NTLFLQLSSLR

SEQ ID NO: 669 - protein, 14 aa, Homo sapiens
  SITE 1: note = Pyroglutamic acid
  MOD_RES 6: note = Deamidated Glutamine
XQLQLQESGPGLVK

SEQ ID NO: 670 - protein, 14 aa, Homo sapiens
  SITE 1: note = Pyroglutamic acid
XQLQLQESGPGLVK

SEQ ID NO: 671 - protein, 16 aa, Homo sapiens
  MOD_RES 14: note = Carboxymethylated Cysteine
LTSVTAADTAVYYCAR

SEQ ID NO: 672 - protein, 13 aa, Homo sapiens
  MOD_RES 1: note = Deamidated Glutamine
QLQLQESGPGLVK

SEQ ID NO: 673 - protein, 13 aa, Homo sapiens
QLQLQESGPGLVK

SEQ ID NO: 674 - protein, 9 aa, Homo sapiens
SRVTISVDR

SEQ ID NO: 675 - protein, 9 aa, Homo sapiens
  MOD_RES 5: note = Carboxymethylated Cysteine
ADQVCINLR

SEQ ID NO: 676 - protein, 11 aa, Homo sapiens
  MOD_RES 1: note = Carboxymethylated Cysteine
  MOD_RES 3: note = Carboxymethylated Cysteine
  MOD_RES 10: note = Carboxymethylated Cysteine
CVCPVSNAMCR

SEQ ID NO: 677 - protein, 13 aa, Homo sapiens
  MOD_RES 1: note = Carboxymethylated Cysteine
  MOD_RES 10: note = Carboxymethylated Cysteine
CVNHYGGYLCLPK

SEQ ID NO: 678 - protein, 12 aa, Homo sapiens
  MOD_RES 5: note = Carboxymethylated Cysteine
  MOD_RES 11: note = Carboxymethylated Cysteine
DIDECDNPDACK

SEQ ID NO: 679 - protein, 17 aa, Homo sapiens
EHNDLEMLTVSSIGTFR

SEQ ID NO: 680 - protein, 8 aa, Homo sapiens
ELPQSNYK

SEQ ID NO: 681 - protein, 13 aa, Homo sapiens
  MOD_RES 3: note = Carboxymethylated Cysteine
  MOD_RES 5: note = Carboxymethylated Cysteine
FSCMCPQGYQVVR

SEQ ID NO: 682 - protein, 8 aa, Homo sapiens
IPSNPSHR

SEQ ID NO: 683 - protein, 29 aa, Homo sapiens
  MOD_RES 3: note = Carboxymethylated Cysteine
  MOD_RES 15: note = Carboxymethylated Cysteine
  MOD_RES 21: note = Carboxymethylated Cysteine
  MOD_RES 28: note = Carboxymethylated Cysteine
IQCAAGYEQSEHNVCQDIDECTAGTHNCR

SEQ ID NO: 684 - protein, 10 aa, Homo sapiens
  MOD_RES 3: note = Carboxymethylated Cysteine
  MOD_RES 9: note = Carboxymethylated Cysteine
LNCEDIDECR

SEQ ID NO: 685 - protein, 15 aa, Homo sapiens
NPADPQRIPSNPSHR

SEQ ID NO: 686 - protein, 14 aa, Homo sapiens
  MOD_RES 3: note = Carboxymethylated Cysteine
NPCQDPYILTPENR

SEQ ID NO: 687 - protein, 20 aa, Homo sapiens
  MOD_RES 5: note = Carboxymethylated Cysteine
  MOD_RES 11: note = Carboxymethylated Cysteine
  MOD_RES 17: note = Carboxymethylated Cysteine
RGEQCVDIDECTIPPYCHQR

SEQ ID NO: 688 - protein, 8 aa, Homo sapiens
RNPADPQR

SEQ ID NO: 689 - protein, 16 aa, Homo sapiens
RNPADPQRIPSNPSHR

SEQ ID NO: 690 - protein, 11 aa, Homo sapiens
  MOD_RES 5: note = Deamidated Asparagine
SGNENGEFYLR

SEQ ID NO: 691 - protein, 11 aa, Homo sapiens
SGNENGEFYLR

SEQ ID NO: 692 - protein, 23 aa, Homo sapiens
SVPSDIFQIQATTIYANTINTFR

SEQ ID NO: 693 - protein, 21 aa, Homo sapiens
  MOD_RES 5: note = Carboxymethylated Cysteine
TQTVCNFTDGALVQHQEWDGK

SEQ ID NO: 694 - protein, 12 aa, Homo sapiens
  SITE 1: note = Pyroglutamic acid
XQVHPDTGISSK

SEQ ID NO: 695 - protein, 15 aa, Homo sapiens
  MOD_RES 2: note = Oxidized Methionine
AMGIMNSFVNDIFER

SEQ ID NO: 696 - protein, 9 aa, Homo sapiens
LLLPGELAK

SEQ ID NO: 697 - protein, 11 aa, Homo sapiens
PEPAKSAPAPK

SEQ ID NO: 698 - protein, 17 aa, Homo sapiens
  MOD_RES 5: note = Carboxymethylated Cysteine
  MOD_RES 12: note = Carboxymethylated Cysteine
ALAQCAPPPAVCAELVR

SEQ ID NO: 699 - protein, 7 aa, Homo sapiens
  MOD_RES 1: note = Carboxymethylated Cysteine
  MOD_RES 4: note = Carboxymethylated Cysteine
CEPCDAR

SEQ ID NO: 700 - protein, 18 aa, Homo sapiens
  MOD_RES 1: note = Carboxymethylated Cysteine
CQPSPDEARPLQALLDGR

SEQ ID NO: 701 - protein, 10 aa, Homo sapiens
EMEDTLNHLK

SEQ ID NO: 702 - protein, 24 aa, Homo sapiens
  MOD_RES 4: note = Carboxymethylated Cysteine
  MOD_RES 6..7: note = Carboxymethylated Cysteine
  MOD_RES 10: note = Carboxymethylated Cysteine
  MOD_RES 18: note = Carboxymethylated Cysteine
EPGCGCCLTCALSEGQPCGIYTER

SEQ ID NO: 703 - protein, 8 aa, Homo sapiens
  MOD_RES 7: note = Carboxymethylated Cysteine
ETEYGPCR

SEQ ID NO: 704 - protein, 8 aa, Homo sapiens
FLNVLSPR

SEQ ID NO: 705 - protein, 11 aa, Homo sapiens
  MOD_RES 3: note = Carboxymethylated Cysteine
GLCVNASAVSR

SEQ ID NO: 706 - protein, 9 aa, Homo sapiens
  MOD_RES 8: note = Carboxymethylated Cysteine
RETEYGPCR

SEQ ID NO: 707 - protein, 15 aa, Homo sapiens
SAGSVESPSVSSTHR

SEQ ID NO: 708 - protein, 11 aa, Homo sapiens
YGQPLPGYTTK

SEQ ID NO: 709 - protein, 15 aa, Homo sapiens
  SITE 1: note = Pyroglutamic acid
XQVRPIVGPFHAVLK

SEQ ID NO: 710 - protein, 23 aa, Homo sapiens
AITPPHPASQANIIFDITEGNLR

SEQ ID NO: 711 - protein, 31 aa, Homo sapiens
AITPPHPASQANIIFDITEGNLRDSFDIIKR

SEQ ID NO: 712 - protein, 7 aa, Homo sapiens
DSFDIIK

SEQ ID NO: 713 - protein, 8 aa, Homo sapiens
DSFDIIKR

SEQ ID NO: 714 - protein, 12 aa, Homo sapiens
EFTRPEEIIFLR

SEQ ID NO: 715 - protein, 14 aa, Homo sapiens
LEMNYVVGGVVSHR

SEQ ID NO: 716 - protein, 12 aa, Homo sapiens
RYMDGMTVGVVR

SEQ ID NO: 717 - protein, 9 aa, Homo sapiens
SAATLQQEK

SEQ ID NO: 718 - protein, 28 aa, Homo sapiens
  MOD_RES 2: note = Carboxymethylated Cysteine
  MOD_RES 5: note = Deamidated Asparagine
  MOD_RES 9: note = Carboxymethylated Cysteine
SCRPNDVTCVFDPVHTISHTVISLPTFR

SEQ ID NO: 719 - protein, 28 aa, Homo sapiens
  MOD_RES 2: note = Carboxymethylated Cysteine
  MOD_RES 9: note = Carboxymethylated Cysteine
SCRPNDVTCVFDPVHTISHTVISLPTFR

SEQ ID NO: 720 - protein, 11 aa, Homo sapiens
YMDGMTVGVVR

SEQ ID NO: 721 - protein, 10 aa, Homo sapiens
GLYDVVSVLR

SEQ ID NO: 722 - protein, 10 aa, Homo sapiens
DVVASLVSTR

SEQ ID NO: 723 - protein, 11 aa, Homo sapiens
EHNLQVLGLVK

SEQ ID NO: 724 - protein, 7 aa, Homo sapiens
LNAGLPR

SEQ ID NO: 725 - protein, 12 aa, Homo sapiens
NEEALDTESSER

SEQ ID NO: 726 - protein, 18 aa, Homo sapiens
TFTPFYFLVEPVDTLSVR

SEQ ID NO: 727 - protein, 15 aa, Homo sapiens
VLPDPEVISDLVFLK

SEQ ID NO: 728 - protein, 8 aa, Homo sapiens
RPSGVSSR

SEQ ID NO: 729 - protein, 18 aa, Homo sapiens
YAASSYLSLTPEQWESHR

SEQ ID NO: 730 - protein, 15 aa, Homo sapiens
ASQGISNSLAWYQQK

SEQ ID NO: 731 - protein, 8 aa, Homo sapiens
LESGVPSR

SEQ ID NO: 732 - protein, 8 aa, Homo sapiens
LLLYAASR

SEQ ID NO: 733 - protein, 12 aa, Homo sapiens
  SITE 1: note = Pyroglutamic acid
XQNVDHSQEAAK

SEQ ID NO: 734 - protein, 16 aa, Homo sapiens
AAPGQEPPEHMAELQR

SEQ ID NO: 735 - protein, 20 aa, Homo sapiens
AHFSPSNIILDFPAAGSAAR

SEQ ID NO: 736 - protein, 23 aa, Homo sapiens
  MOD_RES 13: note = Carboxymethylated Cysteine
  MOD_RES 22: note = Carboxymethylated Cysteine
AWRPALYLAALDCAEETNSAVCR

SEQ ID NO: 737 - protein, 16 aa, Homo sapiens
  MOD_RES 2: note = Carboxymethylated Cysteine
DCASHFEQMAAASMHR

SEQ ID NO: 738 - protein, 11 aa, Homo sapiens
DFNIPGFPTVR

SEQ ID NO: 739 - protein, 21 aa, Homo sapiens
DVQNVAAAPELAMGALELESR

SEQ ID NO: 740 - protein, 10 aa, Homo sapiens
  MOD_RES 3: note = Carboxymethylated Cysteine
  MOD_RES 6: note = Carboxymethylated Cysteine
ELCSACHNER

SEQ ID NO: 741 - protein, 14 aa, Homo sapiens
  MOD_RES 9: note = Carboxymethylated Cysteine
FGVTDFPSCYLLFR

SEQ ID NO: 742 - protein, 10 aa, Homo sapiens
  MOD_RES 9: note = Carboxymethylated Cysteine
GYVHYFFGCR

SEQ ID NO: 743 - protein, 13 aa, Homo sapiens
IEVGRFPVLEGQR

SEQ ID NO: 744 - protein, 15 aa, Homo sapiens
IYMADLESALHYILR

SEQ ID NO: 745 - protein, 15 aa, Homo sapiens
  MOD_RES 10: note = Carboxymethylated Cysteine
KFGVTDFPSCYLLFR

SEQ ID NO: 746 - protein, 15 aa, Homo sapiens
  MOD_RES 7: note = Carboxymethylated Cysteine
KVNWIGCQGSEPHFR

SEQ ID NO: 747 - protein, 13 aa, Homo sapiens
LAGAPSEDPQFPK

SEQ ID NO: 748 - protein, 16 aa, Homo sapiens
LDVPVWDVEATLNFLK

SEQ ID NO: 749 - protein, 10 aa, Homo sapiens
LEEIDGFFAR

SEQ ID NO: 750 - protein, 33 aa, Homo sapiens; identical to SEQ ID NO: 31
  MOD_RES 16: note = Carboxymethylated Cysteine

SEQ ID NO: 751 - protein, 18 aa, Homo sapiens
  MOD_RES 1: note = Deamidated Asparagine
NGSGAVFPVAGADVQTLR

SEQ ID NO: 752 - protein, 18 aa, Homo sapiens
NGSGAVFPVAGADVQTLR

SEQ ID NO: 753 - protein, 19 aa, Homo sapiens
  MOD_RES 2: note = Carboxymethylated Cysteine
ACTDNPLEEEYLAQLQEAK

SEQ ID NO: 754 - protein, 11 aa, Homo sapiens
DTLGLPSEEWK

SEQ ID NO: 755 - protein, 10 aa, Homo sapiens
EEEEAPLLPR

SEQ ID NO: 756 - protein, 11 aa, Homo sapiens
ERLDIYQPTAR

SEQ ID NO: 757 - protein, 10 aa, Homo sapiens
  MOD_RES 9: note = Carboxymethylated Cysteine
FDTTPWILCK

SEQ ID NO: 758 - protein, 15 aa, Homo sapiens
GAGMPNLISTDFSPK

SEQ ID NO: 759 - protein, 9 aa, Homo sapiens
LDIYQPTAR

SEQ ID NO: 760 - protein, 22 aa, Homo sapiens
  MOD_RES 15..16: note = Carboxymethylated Cysteine
SMLSGESSTLAAQHCCYDEDSR

SEQ ID NO: 761 - protein, 13 aa, Homo sapiens
  MOD_RES 2: note = Carboxymethylated Cysteine
  MOD_RES 7: note = Carboxymethylated Cysteine
TCDLPSCPGTEDK

SEQ ID NO: 762 - protein, 8 aa, Homo sapiens
TPEVTPLR

SEQ ID NO: 763 - protein, 13 aa, Homo sapiens
TPEVTPLRLELQK

SEQ ID NO: 764 - protein, 14 aa, Homo sapiens
  MOD_RES 4: note = Carboxymethylated Cysteine
  MOD_RES 8: note = Carboxymethylated Cysteine
TRPCGYGCTATETR

SEQ ID NO: 765 - protein, 7 aa, Homo sapiens
YLSQMLR

SEQ ID NO: 766 - protein, 15 aa, Homo sapiens
  MOD_RES 7: note = Deamidated Asparagine
ASQSVSNYLAWYQQK

SEQ ID NO: 767 - protein, 15 aa, Homo sapiens
ASQSVSNYLAWYQQK

SEQ ID NO: 768 - protein, 21 aa, Homo sapiens
ASQSVSNYLAWYQQKPGQAPR

SEQ ID NO: 769 - protein, 12 aa, Homo sapiens
  MOD_RES 2: note = Deamidated Asparagine
SNWPITFGQGTR

SEQ ID NO: 770 - protein, 12 aa, Homo sapiens
SNWPITFGQGTR

SEQ ID NO: 771 - protein, 10 aa, Homo sapiens
  SITE 1: note = Pyroglutamic acid
XQFPILLDFK

SEQ ID NO: 772 - protein, 12 aa, Homo sapiens
  SITE 1: note = Pyroglutamic acid
XQFPILLDFKTK

SEQ ID NO: 773 - protein, 21 aa, Homo sapiens
  MOD_RES 12: note = Oxidized Methionine
  MOD_RES 17: note = Carboxymethylated Cysteine
DALENIDPATQMMILNCIYFK

SEQ ID NO: 774 - protein, 21 aa, Homo sapiens
  MOD_RES 17: note = Carboxymethylated Cysteine
DALENIDPATQMMILNCIYFK

SEQ ID NO: 775 - protein, 28 aa, Homo sapiens
DQVNTFDNIFIAPVGISTAMGMISLGLK

SEQ ID NO: 776 - protein, 23 aa, Homo sapiens
ENTVTNDWIPEGEEDDDYLDLEK

SEQ ID NO: 777 - protein, 20 aa, Homo sapiens
EYYFAEAQIADFSDPAFISK

SEQ ID NO: 778 - protein, 7 aa, Homo sapiens
FAFNLYR

SEQ ID NO: 779 - protein, 14 aa, Homo sapiens
FKLEKNYNLVESLK

SEQ ID NO: 780 - protein, 12 aa, Homo sapiens
  MOD_RES 5: note = Oxidized Methionine
FPVEMTHNHNFR

SEQ ID NO: 781 - protein, 12 aa, Homo sapiens
FPVEMTHNHNFR

SEQ ID NO: 782 - protein, 15 aa, Homo sapiens
FTVDRPFLFLIYEHR

SEQ ID NO: 783 - protein, 15 aa, Homo sapiens
GETHEQVHSILIIFK

SEQ ID NO: 784 - protein, 18 aa, Homo sapiens
  MOD_RES 16: note = Deamidated Asparagine
GGETAQSADPQWEQLNNK

SEQ ID NO: 785 - protein, 18 aa, Homo sapiens
GGETAQSADPQWEQLNNK

SEQ ID NO: 786 - protein, 18 aa, Homo sapiens
GSWVNKFPVEMTHNHNFR

SEQ ID NO: 787 - protein, 30 aa, Homo sapiens
HQGTITVNEEGTQATTVTTVGFMPLSTQVR

SEQ ID NO: 788 - protein, 16 aa, Homo sapiens
  MOD_RES 6: note = Deamidated Asparagine
MLFDKNGNMAGISDQR

SEQ ID NO: 789 - protein, 16 aa, Homo sapiens
MLFDKNGNMAGISDQR

SEQ ID NO: 790 - protein, 11 aa, Homo sapiens
  MOD_RES 1: note = Deamidated Asparagine
  MOD_RES 4: note = Oxidized Methionine
NGNMAGISDQR

SEQ ID NO: 791 - protein, 16 aa, Homo sapiens
  MOD_RES 8: note = Deamidated Asparagine
LLIYSASNLQSGVPSR

SEQ ID NO: 792 - protein, 16 aa, Homo sapiens
LLIYSASNLQSGVPSR

SEQ ID NO: 793 - protein, 14 aa, Homo sapiens
AGTPYTVTLHGEVR

SEQ ID NO: 794 - protein, 10 aa, Homo sapiens
APTAQVESFR

SEQ ID NO: 795 - protein, 14 aa, Homo sapiens
AVDIPGLEAATPYR

SEQ ID NO: 796 - protein, 28 aa, Homo sapiens
DLAPPSEPSESFQEHTVDGENQIVFIBR

SEQ ID NO: 797 - protein, 14 aa, Homo sapiens
  MOD_RES 9: note = Carboxymethylated Cysteine
DTDSAITNCALSYK

SEQ ID NO: 798 - protein, 11 aa, Homo sapiens
ELEPGVEYFIR

SEQ ID NO: 799 - protein, 17 aa, Homo sapiens
ESNPATINAATELDTPK

SEQ ID NO: 800 - protein, 11 aa, Homo sapiens
ETFTTGLDAPR

SEQ ID NO: 801 - protein, 9 aa, Homo sapiens
FTTDLDSPR

SEQ ID NO: 802 - protein, 12 aa, Homo sapiens
GHEHSIQFAEMK

SEQ ID NO: 803 - protein, 16 aa, Homo sapiens
GLEPGQEYNVLLTAEK

SEQ ID NO: 804 - protein, 9 aa, Homo sapiens
IQALNGPLR

SEQ ID NO: 805 - protein, 10 aa, Homo sapiens
ITAQGQYELR

SEQ ID NO: 806 - protein, 18 aa, Homo sapiens
QTGLAPGQEYEISLHIVK

SEQ ID NO: 807 - protein, 16 aa, Homo sapiens
SQTVSAIATTAMGSPK

SEQ ID NO: 808 - protein, 25 aa, Homo sapiens
TAHISGLPPSTDFIVYLSGLAPSIR

SEQ ID NO: 809 - protein, 12 aa, Homo sapiens
VATYLPAPEGLK

SEQ ID NO: 810 - protein, 8 aa, Homo sapiens
VSIYGVIR

SEQ ID NO: 811 - protein, 21 aa, Homo sapiens
GGTLSTPQTGSENDALYEYLR

SEQ ID NO: 812 - protein, 9 aa, Homo sapiens
LFQGQLVLR

SEQ ID NO: 813 - protein, 15 aa, Homo sapiens
NSETSDTTGTHESDR

SEQ ID NO: 814 - protein, 18 aa, Homo sapiens
  MOD_RES 10: note = Carboxymethylated Cysteine
DVPASFAPACLSHEIIIR

SEQ ID NO: 815 - protein, 23 aa, Homo sapiens
  MOD_RES 2: note = Carboxymethylated Cysteine
  MOD_RES 10: note = Carboxymethylated Cysteine
  MOD_RES 15: note = Carboxymethylated Cysteine
  MOD_RES 19: note = Carboxymethylated Cysteine
GCPVHLVDSCPWPHCNPSCPTVR

SEQ ID NO: 816 - protein, 12 aa, Homo sapiens
GLADSGWFLDNK

SEQ ID NO: 817 - protein, 17 aa, Homo sapiens
  MOD_RES 4: note = Carboxymethylated Cysteine
  MOD_RES 10: note = Carboxymethylated Cysteine
HTDCVDTITCAPTEAIR

SEQ ID NO: 818 - protein, 9 aa, Homo sapiens
LGYPAIQVR

SEQ ID NO: 819 - protein, 8 aa, Homo sapiens
LYIQNLGR

SEQ ID NO: 820 - protein, 15 aa, Homo sapiens
  MOD_RES 10: note = Carboxymethylated Cysteine
QFQEGEEWNCFFGYK

SEQ ID NO: 821 - protein, 19 aa, Homo sapiens
  MOD_RES 9: note = Carboxymethylated Cysteine
SLAQSLYPCSAQQLNEDLR

SEQ ID NO: 822 - protein, 9 aa, Homo sapiens
YWNGVVPER

SEQ ID NO: 823 - protein, 15 aa, Homo sapiens
VQLVESGGGVVQPGR

SEQ ID NO: 824 - protein, 14 aa, Homo sapiens
DLGEENFKALAVAR

SEQ ID NO: 825 - protein, 19 aa, Homo sapiens
  MOD_RES 1: note = Carboxymethylated Cysteine
  MOD_RES 8: note = Carboxymethylated Cysteine
  MOD_RES 14: note = Carboxymethylated Cysteine
CQPNEHNCLGTELCVPMSR

SEQ ID NO: 826 - protein, 10 aa, Homo sapiens
EDVVTNGIGR

SEQ ID NO: 827 - protein, 10 aa, Homo sapiens
  MOD_RES 3: note = Carboxymethylated Cysteine
FVCPPNRPFR

SEQ ID NO: 828 - protein, 10 aa, Homo sapiens
GIALDPAMGK

SEQ ID NO: 829 - protein, 9 aa, Homo sapiens
  MOD_RES 7: note = Carboxymethylated Cysteine
GVLFQPCER

SEQ ID NO: 830 - protein, 9 aa, Homo sapiens
  MOD_RES 8: note = Carboxymethylated Cysteine
ILQDDLTCR

SEQ ID NO: 831 - protein, 19 aa, Homo sapiens
  MOD_RES 2: note = Carboxymethylated Cysteine
  MOD_RES 8: note = Carboxymethylated Cysteine
  MOD_RES 18: note = Carboxymethylated Cysteine
LCNGVQDCMDGSDEGPHCR

SEQ ID NO: 832 - protein, 9 aa, Homo sapiens
  MOD_RES 5: note = Carboxymethylated Cysteine
LDGLCIPLR

SEQ ID NO: 833 - protein, 8 aa, Homo sapiens
LSVIGSIR

SEQ ID NO: 834 - protein, 19 aa, Homo sapiens
NAVVQGLEQPHGLVVHPLR

SEQ ID NO: 835 - protein, 14 aa, Homo sapiens
  MOD_RES 3: note = Carboxymethylated Cysteine
  MOD_RES 10: note = Carboxymethylated Cysteine
PRCPLNYFACPSGR

SEQ ID NO: 836 - protein, 14 aa, Homo sapiens
  MOD_RES 2: note = Carboxymethylated Cysteine
  MOD_RES 9: note = Carboxymethylated Cysteine
SCEGVTHVCDPSVK

SEQ ID NO: 837 - protein, 14 aa, Homo sapiens
SLDPFKPFIIFSNR

SEQ ID NO: 838 - protein, 12 aa, Homo sapiens
TTLLAGDIEHPR

SEQ ID NO: 839 - protein, 16 aa, Homo sapiens
VPDEHMIPIENLMNPR

SEQ ID NO: 840 - protein, 12 aa, Homo sapiens
YVVISQGLDKPR

SEQ ID NO: 841 - protein, 15 aa, Homo sapiens
DTSISTAYMELSSLR

SEQ ID NO: 842 - protein, 22 aa, Homo sapiens
  MOD_RES 11: note = Carboxymethylated Cysteine
DQDNDLNTGNCAVMFQGAWWYK

SEQ ID NO: 843 - protein, 13 aa, Homo sapiens
  MOD_RES 8: note = Deamidated Asparagine
GTHGSFANGINWK

SEQ ID NO: 844 - protein, 13 aa, Homo sapiens
GTHGSFANGINWK

SEQ ID NO: 845 - protein, 23 aa, Homo sapiens
LGEFWLGNDNIHALTAQGTSELR

SEQ ID NO: 846 - protein, 10 aa, Homo sapiens
  MOD_RES 2: note = Carboxymethylated Cysteine
NCHVSNLNGR

SEQ ID NO: 847 - protein, 10 aa, Homo sapiens
RVDGSVDFYR

SEQ ID NO: 848 - protein, 16 aa, Homo sapiens
RVDGSVDFYRDWATYK

SEQ ID NO: 849 - protein, 9 aa, Homo sapiens
VDGSVDFYR

SEQ ID NO: 850 - protein, 15 aa, Homo sapiens
VDGSVDFYRDWATYK

SEQ ID NO: 851 - protein, 14 aa, Homo sapiens
VDLVDFEDNYQFAK

SEQ ID NO: 852 - protein, 12 aa, Homo sapiens
  MOD_RES 6: note = Oxidized Methionine
EFTPQMQAAYQK

SEQ ID NO: 853 - protein, 12 aa, Homo sapiens
EFTPQMQAAYQK

SEQ ID NO: 854 - protein, 19 aa, Homo sapiens
  MOD_RES 15: note = Oxidized Methionine
FFESFGDLSSPDAVMGNPK

SEQ ID NO: 855 - protein, 19 aa, Homo sapiens
FFESFGDLSSPDAVMGNPK

SEQ ID NO: 856 - protein, 13 aa, Homo sapiens
  MOD_RES 11: note = Carboxymethylated Cysteine
GTFSQLSELHCDK

SEQ ID NO: 857 - protein, 12 aa, Homo sapiens
  MOD_RES 8: note = Carboxymethylated Cysteine
LLGNVLVCVLAR

SEQ ID NO: 858 - protein, 16 aa, Homo sapiens
NFGKEFTPQMQAAYQK

SEQ ID NO: 859 - protein, 9 aa, Homo sapiens
TAVNALWGK

SEQ ID NO: 860 - protein, 13 aa, Homo sapiens
VNVDAVGGEALGR

SEQ ID NO: 861 - protein, 22 aa, Homo sapiens
  MOD_RES 7: note = Deamidated Asparagine
AGTELVNFLSYFVELGTQPATQ

SEQ ID NO: 862 - protein, 22 aa, Homo sapiens
AGTELVNFLSYFVELGTQPATQ

SEQ ID NO: 863 - protein, 20 aa, Homo sapiens
  MOD_RES 3: note = Carboxymethylated Cysteine
  MOD_RES 10: note = Deamidated Glutamine
EPCVESLVSQYFQTVTDYGK

SEQ ID NO: 864 - protein, 20 aa, Homo sapiens
  MOD_RES 3: note = Carboxymethylated Cysteine
EPCVESLVSQYFQTVTDYGK

SEQ ID NO: 865 - protein, 25 aa, Homo sapiens
  MOD_RES 3: note = Carboxymethylated Cysteine
EPCVESLVSQYFQTVTDYGKDLMEK

SEQ ID NO: 866 - protein, 8 aa, Homo sapiens
EQLTPLIK

SEQ ID NO: 867 - protein, 9 aa, Homo sapiens
EQLTPLIKK

SEQ ID NO: 868 - protein, 23 aa, Homo sapiens
  MOD_RES 8: note = Deamidated Asparagine
KAGTELVNFLSYFVELGTQPATQ

SEQ ID NO: 869 - protein, 23 aa, Homo sapiens
KAGTELVNFLSYFVELGTQPATQ

SEQ ID NO: 870 - protein, 10 aa, Homo sapiens
SKEQLTPLIK

SEQ ID NO: 871 - protein, 11 aa, Homo sapiens
SKEQLTPLIKK

SEQ ID NO: 872 - protein, 9 aa, Homo sapiens
  MOD_RES 5: note = Deamidated Glutamine
SPELQAEAK

SEQ ID NO: 873 - protein, 9 aa, Homo sapiens
SPELQAEAK

SEQ ID NO: 874 - protein, 14 aa, Homo sapiens
SPELQAEAKSYFEK

SEQ ID NO: 875 - protein, 11 aa, Homo sapiens
VKSPELQAEAK

SEQ ID NO: 876 - protein, 11 aa, Homo sapiens
ASVGQDSPEPR

SEQ ID NO: 877 - protein, 17 aa, Homo sapiens
EIPVLVTQISSTNHPVK

SEQ ID NO: 878 - protein, 8 aa, Homo sapiens
IQQIPNVR

SEQ ID NO: 879 - protein, 10 aa, Homo sapiens
IYGPSDSASR

SEQ ID NO: 880 - protein, 12 aa, Homo sapiens
VGLSDAFVVVHR

SEQ ID NO: 881 - protein, 20 aa, Homo sapiens
  SITE 1: note = Pyroglutamic acid
XQPVLTQPSSHSASSGASVR

SEQ ID NO: 882 - protein, 9 aa, Homo sapiens
GQGSGVPSR

SEQ ID NO: 883 - protein, 14 aa, Homo sapiens
  SITE 1: note = Pyroglutamic acid
  MOD_RES 6: note = Deamidated Glutamine
XQLQLQESGPGLVK

SEQ ID NO: 884 - protein, 14 aa, Homo sapiens
  SITE 1: note = Pyroglutamic acid
XQLQLQESGPGLVK

SEQ ID NO: 885 - protein, 16 aa, Homo sapiens
  MOD_RES 14: note = Carboxymethylated Cysteine
LTSVTAADTAVYYCAR

SEQ ID NO: 886 - protein, 13 aa, Homo sapiens
  MOD_RES 1: note = Deamidated Glutamine
QLQLQESGPGLVK

SEQ ID NO: 887 - protein, 13 aa, Homo sapiens
QLQLQESGPGLVK

SEQ ID NO: 888 - protein, 9 aa, Homo sapiens
SRVTISVDR

SEQ ID NO: 889 - protein, 26 aa, Homo sapiens
AHYDLRHTFMGVVSLGSPSGEVSHPR

SEQ ID NO: 890 - protein, 24 aa, Homo sapiens
  MOD_RES 21: note = Carboxymethylated Cysteine
AQLVPLPPSTYVEFTVSGTDCVAK

SEQ ID NO: 891 - protein, 13 aa, Homo sapiens
  MOD_RES 1: note = Carboxymethylated Cysteine
CDSSPDSAEDVRK

SEQ ID NO: 892 - protein, 7 aa, Homo sapiens
  MOD_RES 1: note = Carboxymethylated Cysteine
CNLLAEK

SEQ ID NO: 893 - protein, 14 aa, Homo sapiens
  MOD_RES 8: note = Carboxymethylated Cysteine
  MOD_RES 11: note = Deamidated Glutamine
EHAVEGDCDFQLLK

SEQ ID NO: 894 - protein, 14 aa, Homo sapiens
  MOD_RES 8: note = Carboxymethylated Cysteine
EHAVEGDCDFQLLK

SEQ ID NO: 895 - protein, 7 aa, Homo sapiens
FSVVYAK

SEQ ID NO: 896 - protein, 20 aa, Homo sapiens
  MOD_RES 4: note = Oxidized Methionine
HTFMGVVSLGSPSGEVSHPR

SEQ ID NO: 897 - protein, 20 aa, Homo sapiens
HTFMGVVSLGSPSGEVSHPR

SEQ ID NO: 898 - protein, 21 aa, Homo sapiens
HTFMGVVSLGSPSGEVSHPRK

SEQ ID NO: 899 - protein, 10 aa, Homo sapiens
  MOD_RES 4: note = Deamidated Asparagine
HTLNQIDEVK

SEQ ID NO: 900 - protein, 10 aa, Homo sapiens
  MOD_RES 5: note = Deamidated Glutamine
HTLNQIDEVK

SEQ ID NO: 901 - protein, 10 aa, Homo sapiens
HTLNQIDEVK

SEQ ID NO: 902 - protein, 29 aa, Homo sapiens
  MOD_RES 4: note = Carboxymethylated Cysteine
  MOD_RES 21: note = Deamidated Asparagine
QPNCDDPETEEAALVAIDYINQNLPWGYK

SEQ ID NO: 903 - protein, 29 aa, Homo sapiens
  MOD_RES 4: note = Carboxymethylated Cysteine
QPNCDDPETEEAALVAIDYINQNLPWGYK

SEQ ID NO: 904 - protein, 21 aa, Homo sapiens
  MOD_RES 18: note = Carboxymethylated Cysteine
TVVQPSVGAAAGPVVPPCPGR

SEQ ID NO: 905 - protein, 32 aa, Homo sapiens
  MOD_RES 22: note = Carboxymethylated Cysteine
VWPQQPSGELFEIEIDTLETTCHVLDPTPVAR

SEQ ID NO: 906 - protein, 13 aa, Homo sapiens
  SITE 1: note = Pyroglutamic acid
XQVQLVQSGAEVR

SEQ ID NO: 907 - protein, 14 aa, Homo sapiens
  SITE 1: note = Pyroglutamic acid
XQVQLVQSGAEVRK

SEQ ID NO: 908 - protein, 12 aa, Homo sapiens
  MOD_RES 11: note = Carboxymethylated Cysteine
AEWQEKDEFICR

SEQ ID NO: 909 - protein, 14 aa, Homo sapiens
AVHEAASPSQTVQR

SEQ ID NO: 910 - protein, 17 aa, Homo sapiens
GVSAYLSRPSPFDLFIR

SEQ ID NO: 911 - protein, 12 aa, Homo sapiens
  MOD_RES 1: note = Deamidated Glutamine
  MOD_RES 3: note = Deamidated Glutamine
QVQLVQSGAEVR

SEQ ID NO: 912 - protein, 12 aa, Homo sapiens
  MOD_RES 1: note = Deamidated Glutamine
QVQLVQSGAEVR

SEQ ID NO: 913 - protein, 13 aa, Homo sapiens
AGVFSDLSNQELK

SEQ ID NO: 914 - protein, 10 aa, Homo sapiens
  MOD_RES 1: note = Carboxymethylated Cysteine
CLAFTDVAPR

SEQ ID NO: 915 - protein, 9 aa, Homo sapiens
DNGPNYVQR

SEQ ID NO: 916 - protein, 7 aa, Homo sapiens
DTVNWPR

SEQ ID NO: 917 - protein, 10 aa, Homo sapiens
FYGSPEELAR

SEQ ID NO: 918 - protein, 13 aa, Homo sapiens
GDFPSPIHVSGPR

SEQ ID NO: 919 - protein, 17 aa, Homo sapiens
GHDSTEEPPLFSSHKPR

SEQ ID NO: 920 - protein, 20 aa, Homo sapiens
KYADGEVDVVVLEDPLPGGK

SEQ ID NO: 921 - protein, 11 aa, Homo sapiens
LQPSSTTTMAK

SEQ ID NO: 922 - protein, 29 aa, Homo sapiens
PGYQSSWASRPISTAEYALLYHTLQEATK

SEQ ID NO: 923 - protein, 9 aa, Homo sapiens
VDLDVAGTK

SEQ ID NO: 924 - protein, 15 aa, Homo sapiens
VVQPTLEQTQYSWER

SEQ ID NO: 925 - protein, 19 aa, Homo sapiens
YADGEVDVVVLEDPLPGGK

SEQ ID NO: 926 - protein, 9 aa, Homo sapiens
ASEQAELPR

SEQ ID NO: 927 - protein, 17 aa, Homo sapiens
FWLEQGVDSSVFEALPK

SEQ ID NO: 928 - protein, 27 aa, Homo sapiens
  MOD_RES 9: note = Carboxymethylated Cysteine
GQSQFQALCFVTQLQHNEIIPSEAMAK

SEQ ID NO: 929 - protein, 14 aa, Homo sapiens
KPDGTLVSFTADFK

SEQ ID NO: 930 - protein, 15 aa, Homo sapiens
KPDGTLVSFTADFKK

SEQ ID NO: 931 - protein, 11 aa, Homo sapiens
SYSFDFYVPQR

SEQ ID NO: 932 - protein, 13 aa, Homo sapiens
ASLEGNLAETENR

SEQ ID NO: 933 - protein, 13 aa, Homo sapiens
LLEGEDAHLTQYK

SEQ ID NO: 934 - protein, 9 aa, Homo sapiens
TNEEVQDGK

SEQ ID NO: 935 - protein, 23 aa, Homo sapiens
  MOD_RES 2: note = Carboxymethylated Cysteine
YCVQLSQIQGLIGSVEEQLAQLR

SEQ ID NO: 936 - protein, 11 aa, Homo sapiens
  MOD_RES 1: note = Deamidated Asparagine
NNNRPSGISER

SEQ ID NO: 937 - protein, 11 aa, Homo sapiens
  MOD_RES 3: note = Deamidated Asparagine
NNNRPSGISER

SEQ ID NO: 938 - protein, 11 aa, Homo sapiens
NNNRPSGISER

SEQ ID NO: 939 - protein, 19 aa, Homo sapiens
APNLLIYAASTLQGGVPSR

SEQ ID NO: 940 - protein, 18 aa, Homo sapiens
DIRMTQSPSSLSASVGDR

SEQ ID NO: 941 - protein, 15 aa, Homo sapiens
  MOD_RES 1: note = Oxidized Methionine
MTQSPSSLSASVGDR

SEQ ID NO: 942 - protein, 15 aa, Homo sapiens
MTQSPSSLSASVGDR

SEQ ID NO: 943 - protein, 14 aa, Homo sapiens
AQIHDLVLVGGSTR

SEQ ID NO: 944 - protein, 16 aa, Homo sapiens
ATAGDTHLGGEDFDNR

SEQ ID NO: 945 - protein, 12 aa, Homo sapiens
DAGVIAGLNVLR

SEQ ID NO: 946 - protein, 15 aa, Homo sapiens
NQVALNPQNTVFDAK

SEQ ID NO: 947 - protein, 23 aa, Homo sapiens
SINPDEAVAYGAAVQAAILMGDK

SEQ ID NO: 948 - protein, 8 aa, Homo sapiens
  MOD_RES 7: note = Carboxymethylated Cysteine
ALEVEECR

SEQ ID NO: 949 - protein, 11 aa, Homo sapiens
  MOD_RES 2: note = Oxidized Methionine
AMDFNGILTIR

SEQ ID NO: 950 - protein, 11 aa, Homo sapiens
ASYAQQPAESR

SEQ ID NO: 951 - protein, 17 aa, Homo sapiens
  MOD_RES 1: note = Carboxymethylated Cysteine
  MOD_RES 15: note = Carboxymethylated Cysteine
CAPGYYGNPSQGQPCQR

SEQ ID NO: 952 - protein, 24 aa, Homo sapiens
  MOD_RES 1: note = Carboxymethylated Cysteine
CSATGSPTPTLEWTGGPGGQLPAK

SEQ ID NO: 953 - protein, 16 aa, Homo sapiens
EHLLMALADLDELLIR

SEQ ID NO: 954 - protein, 16 aa, Homo sapiens
EHLLMALAGIDTLLIR

SEQ ID NO: 955 - protein, 13 aa, Homo sapiens
GHTPTQPGALNQR

SEQ ID NO: 956 - protein, 22 aa, Homo sapiens
HLISTHFAPGDFQGFALVNPQR

SEQ ID NO: 957 - protein, 13 aa, Homo sapiens
IAHVELADAGQYR

SEQ ID NO: 958 - protein, 17 aa, Homo sapiens
IQVVVLSASDASPPPVK

SEQ ID NO: 959 - protein, 15 aa, Homo sapiens
  MOD_RES 2: note = Carboxymethylated Cysteine
  MOD_RES 5: note = Carboxymethylated Cysteine
LCNECADGSFHLSTR

SEQ ID NO: 960 - protein, 15 aa, Homo sapiens
  MOD_RES 14: note = Carboxymethylated Cysteine
LHQMSVADSGEYVCR

SEQ ID NO: 961 - protein, 15 aa, Homo sapiens
  MOD_RES 14: note = Carboxymethylated Cysteine
LPAVEPTDQAQYLCR

SEQ ID NO: 962 - protein, 17 aa, Homo sapiens
  MOD_RES 16: note = Carboxymethylated Cysteine
LYIFQASPADAGQYVCR

SEQ ID NO: 963 - protein, 10 aa, Homo sapiens
SPAYTLVWTR

SEQ ID NO: 964 - protein, 7 aa, Homo sapiens
VQVSPER

SEQ ID NO: 965 - protein, 16 aa, Homo sapiens
  SITE 1: note = Pyroglutamic acid
  MOD_RES 5: note = Carboxymethylated Cysteine
XQDACQGDSGGVFAVR

SEQ ID NO: 966 - protein, 28 aa, Homo sapiens
  SITE 1: note = Pyroglutamic acid
  MOD_RES 20: note = Carboxymethylated Cysteine
XQGYQLIEGNQVLHSFTAVCQDDGTWHR

SEQ ID NO: 967 - protein, 27 aa, Homo sapiens
  SITE 1: note = Pyroglutamic acid
XQRPPDLDTSSNAVDLLFFTDESGDSR

SEQ ID NO: 968 - protein, 30 aa, Homo sapiens
  SITE 1: note = Pyroglutamic acid
XQRPPDLDTSSNAVDLLFFIDESGDSRGWK

SEQ ID NO: 969 - protein, 15 aa, Homo sapiens
  MOD_RES 1: note = Carboxymethylated Cysteine
  MOD_RES 5: note = Carboxymethylated Cysteine
CLPVCGKPVNPVEQR

SEQ ID NO: 970 - protein, 21 aa, Homo sapiens
  MOD_RES 19: note = Carboxymethylated Cysteine
DPNTDRWVATGIVSWGIGCSR

SEQ ID NO: 971 - protein, 8 aa, Homo sapiens
  MOD_RES 7: note = Carboxymethylated Cysteine
DYFIATCK

SEQ ID NO: 972 - protein, 8 aa, Homo sapiens
  MOD_RES 3: note = Oxidized Methionine
EFMSQGNK

SEQ ID NO: 973 - protein, 8 aa, Homo sapiens
EFMSQGNK

SEQ ID NO: 974 - protein, 16 aa, Homo sapiens
  MOD_RES 9: note = Carboxymethylated Cysteine
ESEQGVYTCTAQGIWK

SEQ ID NO: 975 - protein, 15 aa, Homo sapiens
  MOD_RES 2: note = Carboxymethylated Cysteine
FCGQLGSPLGNPPGK

SEQ ID NO: 976 - protein, 16 aa, Homo sapiens
  MOD_RES 2: note = Carboxymethylated Cysteine
FCGQLGSPLGNPPGKK

SEQ ID NO: 977 - protein, 27 aa, Homo sapiens
  MOD_RES 15: note = Carboxymethylated Cysteine
FLEPFDIDDHQQVHCPYDQLQIYANGK

SEQ ID NO: 978 - protein, 17 aa, Homo sapiens
  MOD_RES 12: note = Carboxymethylated Cysteine
FVRLPVANPQACENWLR

SEQ ID NO: 979 - protein, 17 aa, Homo sapiens
  MOD_RES 14: note = Carboxymethylated Cysteine
GFLAYYQAVDLDECASR

SEQ ID NO: 980 - protein, 9 aa, Homo sapiens
GGGALLGDR

SEQ ID NO: 981 - protein, 21 aa, Homo sapiens
GGGALLGDRWILTAAHTLYPK

SEQ ID NO: 982 - protein, 26 aa, Homo sapiens
  MOD_RES 3: note = Carboxymethylated Cysteine
  MOD_RES 7: note = Carboxymethylated Cysteine
HSCQAECSSELYTEASGYISSLEYPR

SEQ ID NO: 983 - protein, 11 aa, Homo sapiens
  MOD_RES 5: note = Carboxymethylated Cysteine
IQYYCHEPYYK

SEQ ID NO: 984 - protein, 12 aa, Homo sapiens
LFGEVTSPLFPK

SEQ ID NO: 985 - protein, 16 aa, Homo sapiens
AGLAASLAGPHSIVGR

SEQ ID NO: 986 - protein, 13 aa, Homo sapiens
AVVVHAGEDDLGR

SEQ ID NO: 987 - protein, 11 aa, Homo sapiens
VTEIWQEVMQR

SEQ ID NO: 988 - protein, 9 aa, Homo sapiens
  MOD_RES 3: note = Carboxymethylated Cysteine
LSCVGSGFR

SEQ ID NO: 989 - protein, 22 aa, Homo sapiens
  MOD_RES 20: note = Carboxymethylated Cysteine
SSLYLQMKSLRDEDTAVYYCAR

SEQ ID NO: 990 - protein, 10 aa, Homo sapiens
TYFPHFDLSH

SEQ ID NO: 991 - protein, 24 aa, Homo sapiens
  SITE 1: note = Pyroglutamic acid
  MOD_RES 5: note = Oxidized Methionine
XQEDMKGHFSISIPVKSDIAPVAR

SEQ ID NO: 992 - protein, 24 aa, Homo sapiens
  SITE 1: note = Pyroglutamic acid
XQEDMKGHFSISIPVKSDIAPVAR

SEQ ID NO: 993 - protein, 17 aa, Homo sapiens
  SITE 1: note = Pyroglutamic acid
XQFSFPLSSEPFQGSYK

SEQ ID NO: 994 - protein, 11 aa, Homo sapiens
  SITE 1: note = Pyroglutamic acid
XQGIPFFGQVR

SEQ ID NO: 995 - protein, 9 aa, Homo sapiens
  SITE 1: note = Pyroglutamic acid
  MOD_RES 5: note = Deamidated Asparagine
  MOD_RES 7: note = Carboxymethylated Cysteine
XQKDNGCFR

SEQ ID NO: 996 - protein, 9 aa, Homo sapiens
  SITE 1: note = Pyroglutamic acid
  MOD_RES 7: note = Carboxymethylated Cysteine
XQKDNGCFR

SEQ ID NO: 997 - protein, 18 aa, Homo sapiens
  SITE 1: note = Pyroglutamic acid
XQLNYKHYDGSYSTFGER

SEQ ID NO: 998 - protein, 24 aa, Homo sapiens
  SITE 1: note = Pyroglutamic acid
  MOD_RES 4: note = Deamidated Asparagine
XQQNAQGGFSSTQDTVVALHALSK

SEQ ID NO: 999 - protein, 24 aa, Homo sapiens
  SITE 1: note = Pyroglutamic acid
XQQNAQGGFSSTQDTVVALHALSK

SEQ ID NO: 1000 - protein, 8 aa, Homo sapiens
  SITE 1: note = Pyroglutamic acid
XQSSEITR

SEQ ID NO: 1001 - protein, 11 aa, Homo sapiens
  SITE 1: note = Pyroglutamic acid
XQTVSWAVTPK

SEQ ID NO: 1002 - protein, 15 aa, Homo sapiens
AAQVTIQSSGTFSSK

SEQ ID NO: 1003 - protein, 17 aa, Homo sapiens
  MOD_RES 11: note = Oxidized Methionine
AFQPFFVELTMPYSVIR

SEQ ID NO: 1004 - protein, 24 aa, Homo sapiens
  MOD_RES 11: note = Oxidized Methionine
AFQPFFVELTMPYSVIRGEAFTLK

SEQ ID NO: 1005 - protein, 17 aa, Homo sapiens
AFQPFFVELTMPYSVIR

SEQ ID NO: 1006 - protein, 24 aa, Homo sapiens
AFQPFFVELTMPYSVIRGEAFTLK

SEQ ID NO: 1007 - protein, 21 aa, Homo sapiens
  MOD_RES 5: note = Carboxymethylated Cysteine
AGAFCLSEDAGLGISSTASLR

SEQ ID NO: 1008 - protein, 17 aa, Homo sapiens
AHTSFQISLSVSYTGSR

SEQ ID NO: 1009 - protein, 11 aa, Homo sapiens
  MOD_RES 6: note = Deamidated Asparagine
AIGYLNTGYQR

SEQ ID NO: 1010 - protein, 11 aa, Homo sapiens
AIGYLNTGYQR

SEQ ID NO: 1011 - protein, 17 aa, Homo sapiens
  MOD_RES 1: note = Carboxymethylated Cysteine
CAFSSQEPYFSYSGAFK

SEQ ID NO: 1012 - protein, 9 aa, Homo sapiens
EDAIWNLLR

SEQ ID NO: 1013 - protein, 26 aa, Homo sapiens
  MOD_RES 21: note = Carboxymethylated Cysteine
ESTVFEDLSDEAERDEYELLCPDNTR

SEQ ID NO: 1014 - protein, 12 aa, Homo sapiens
  MOD_RES 6: note = Carboxymethylated Cysteine
FFSASCVPGADK

SEQ ID NO: 1015 - protein, 12 aa, Homo sapiens
  MOD_RES 8: note = Carboxymethylated Cysteine
LADFALLCLDGK

SEQ ID NO: 1016 - protein, 13 aa, Homo sapiens
LRPVAAEVYGTER

SEQ ID NO: 1017 - protein, 13 aa, Homo sapiens
  MOD_RES 10: note = Carboxymethylated Cysteine
NLLFNDNTECLAR

SEQ ID NO: 1018 - protein, 14 aa, Homo sapiens
YLGPQYVAGITNLK

SEQ ID NO: 1019 - protein, 9 aa, Homo sapiens
YYGYTGAFR

SEQ ID NO: 1020 - protein, 12 aa, Homo sapiens
  SITE 1: note = Pyroglutamic acid
  MOD_RES 3: note = Carboxymethylated Cysteine
  MOD_RES 7: note = Deamidated Asparagine
XQCYHGNGQSYR

SEQ ID NO: 1021 - protein, 11 aa, Homo sapiens
  SITE 1: note = Pyroglutamic acid
  MOD_RES 2: note = Carboxymethylated Cysteine
XCYHGNGQSYR

SEQ ID NO: 1022 - protein, 19 aa, Homo sapiens
  MOD_RES 9: note = Carboxymethylated Cysteine
ATTVTGTPCQEWAAQEPHR

SEQ ID NO: 1023 - protein, 23 aa, Homo sapiens
  MOD_RES 1: note = Carboxymethylated Cysteine
  MOD_RES 9: note = Carboxymethylated Cysteine
CPGSIVGGCVAHPHSWPWQVSLR

SEQ ID NO: 1024 - protein, 16 aa, Homo sapiens
  MOD_RES 9: note = Deamidated Asparagine
  MOD_RES 12: note = Carboxymethylated Cysteine
EAQLLVIENEVCNHYK

SEQ ID NO: 1025 - protein, 16 aa, Homo sapiens
  MOD_RES 12: note = Carboxymethylated Cysteine
  MOD_RES 13: note = Deamidated Asparagine
EAQLLVIENEVCNHYK

SEQ ID NO: 1026 - protein, 16 aa, Homo sapiens
  MOD_RES 12: note = Carboxymethylated Cysteine
EAQLLVIENEVCNHYK

SEQ ID NO: 1027 - protein, 10 aa, Homo sapiens
  MOD_RES 8: note = Oxidized Methionine
FVTWIEGMMR

SEQ ID NO: 1028 - protein, 10 aa, Homo sapiens
FVTWIEGMMR

SEQ ID NO: 1029 - protein, 10 aa, Homo sapiens
GISSTTVTGR

SEQ ID NO: 1030 - protein, 10 aa, Homo sapiens
GSFSTTVTGR

SEQ ID NO: 1031 - protein, 18 aa, Homo sapiens
  MOD_RES 5: note = Carboxymethylated Cysteine
  MOD_RES 15: note = Carboxymethylated Cysteine
GTDSCQGDSGGPLVCFEK

SEQ ID NO: 1032 - protein, 10 aa, Homo sapiens
GTFSTTVTGR

SEQ ID NO: 1033 - protein, 10 aa, Homo sapiens
GTLSTTITGR

SEQ ID NO: 1034 - protein, 10 aa, Homo sapiens
GTYSTTVTGR

SEQ ID NO: 1035 - protein, 21 aa, Homo sapiens
  MOD_RES 3: note = Carboxymethylated Cysteine
  MOD_RES 19: note = Carboxymethylated Cysteine
HFCGGTLISPEWVLTAAHCLK

SEQ ID NO: 1036 - protein, 10 aa, Homo sapiens
HSTFIPGTNK

SEQ ID NO: 1037 - protein, 12 aa, Homo sapiens
IPLYYPNAGLTR

SEQ ID NO: 1038 - protein, 24 aa, Homo sapiens
  MOD_RES 2: note = Carboxymethylated Cysteine
  MOD_RES 10: note = Carboxymethylated Cysteine
KCPGSIVGGCVAHPHSWPWQVSLR

SEQ ID NO: 1039 - protein, 20 aa, Homo sapiens
  MOD_RES 6: note = Carboxymethylated Cysteine
  MOD_RES 11: note = Carboxymethylated Cysteine
  MOD_RES 18: note = Carboxymethylated Cysteine
KLFDYCDIPLCASSSFDCGK

SEQ ID NO: 1040 - protein, 25 aa, Homo sapiens
  MOD_RES 3: note = Carboxymethylated Cysteine
TGCSLMGASVDSTLAFNTYVHFQGK

SEQ ID NO: 1041 - protein, 13 aa, Homo sapiens
ALGQNPTNAEVLK

SEQ ID NO: 1042 - protein, 13 aa, Homo sapiens
DQGTYEDYVEGLR

SEQ ID NO: 1043 - protein, 8 aa, Homo sapiens
EAFQLFDR

SEQ ID NO: 1044 - protein, 9 aa, Homo sapiens
HVLVTLGEK

SEQ ID NO: 1045 - protein, 11 aa, Homo sapiens
  MOD_RES 6: note = Carboxymethylated Cysteine
ILYSQCGDVMR

SEQ ID NO: 1046 - protein, 16 aa, Homo sapiens
VLDFEHFLPMLQTVAK

SEQ ID NO: 1047 - protein, 14 aa, Homo sapiens
  SITE 1: note = Pyroglutamic acid
  MOD_RES 2..3: note = Deamidated Glutamine
  MOD_RES 8: note = Carboxymethylated Cysteine
  MOD_RES 9: note = Oxidized Methionine
  MOD_RES 13: note = Deamidated Asparagine
XQQNVAHCMLVSNR

SEQ ID NO: 1048 - protein, 14 aa, Homo sapiens
PGQPPQLLNEVSNR

SEQ ID NO: 1049 - protein, 15 aa, Homo sapiens
ASQSISSYLNWYQQK

SEQ ID NO: 1050 - protein, 18 aa, Homo sapiens
ASQSISSYLNWYQQLPGK

SEQ ID NO: 1051 - protein, 12 aa, Homo sapiens
AKFEELNMDLFR

SEQ ID NO: 1052 - protein, 12 aa, Homo sapiens
DAGTIAGLNVMR

SEQ ID NO: 1053 - protein, 18 aa, Homo sapiens
DNHLLGTFDLTGIPPAPR

SEQ ID NO: 1054 - protein, 12 aa, Homo sapiens
ELEEIVQPIISK

SEQ ID NO: 1055 - protein, 10 aa, Homo sapiens
FEELNMDLFR

SEQ ID NO: 1056 - protein, 17 aa, Homo sapiens
IINEPTAAAIAYGLDKR

SEQ ID NO: 1057 - protein, 9 aa, Homo sapiens
ITITNDQNR

SEQ ID NO: 1058 - protein, 14 aa, Homo sapiens
ITPSYVAFTPEGER

SEQ ID NO: 1059 - protein, 15 aa, Homo sapiens
KSDIDEIVLVGGSTR

SEQ ID NO: 1060 - protein, 18 aa, Homo sapiens
KVTHAVVTVPAYFNDAQR

SEQ ID NO: 1061 - protein, 14 aa, Homo sapiens
  MOD_RES 1: note = Deamidated Asparagine
NGRVEIIANDQGNR

SEQ ID NO: 1062 - protein, 11 aa, Homo sapiens
NELESYAYSLK

SEQ ID NO: 1063 - protein, 14 aa, Homo sapiens
NGRVEIIANDQGNR

SEQ ID NO: 1064 - protein, 15 aa, Homo sapiens
NQLTSNPENTVFDAK

SEQ ID NO: 1065 - protein, 16 aa, Homo sapiens
NQLTSNPENTVFDAKR

SEQ ID NO: 1066 - protein, 14 aa, Homo sapiens
SDIDEIVLVGGSTR

SEQ ID NO: 1067 - protein, 14 aa, Homo sapiens
TFAPEEISAMVLTK

SEQ ID NO: 1068 - protein, 15 aa, Homo sapiens
TKPYIQVDIGGGQTK

SEQ ID NO: 1069 - protein, 12 aa, Homo sapiens
TWNDPSVQQDIK

SEQ ID NO: 1070 - protein, 17 aa, Homo sapiens
VTHAVVTVPAYFNDAQR

SEQ ID NO: 1071 - protein, 10 aa, Homo sapiens
  MOD_RES 1: note = Oxidized Methionine
MYYSAVEPTK

SEQ ID NO: 1072 - protein, 10 aa, Homo sapiens
MYYSAVEPTK

SEQ ID NO: 1073 - protein, 21 aa, Homo sapiens
MYYSAVEPTKDIFTGLIGPMK

SEQ ID NO: 1074 - protein, 36 aa, Homo sapiens
  MOD_RES 9: note = Carboxymethylated Cysteine
TPGIWLLHCHVTDHIHAGMETTYTVLQNEGEYPDTK

SEQ ID NO: 1075 - protein, 9 aa, Homo sapiens
EEALALMNR

SEQ ID NO: 1076 - protein, 9 aa, Homo sapiens
FGQTPVQER

SEQ ID NO: 1077 - protein, 19 aa, Homo sapiens
LFSLKPTSGPVLTVTLFGR

SEQ ID NO: 1078 - protein, 14 aa, Homo sapiens
YQYNTDVVFDSQGK

SEQ ID NO: 1079 - protein, 22 aa, Homo sapiens
PGEAAIFIIQEATTLVPGIPPR

SEQ ID NO: 1080 - protein, 8 aa, Homo sapiens
EDFQIQPR

SEQ ID NO: 1081 - protein, 8 aa, Homo sapiens
DQVETALK

SEQ ID NO: 1082 - protein, 9 aa, Homo sapiens
IMGITLVSK

SEQ ID NO: 1083 - protein, 9 aa, Homo sapiens
LLGLSLAGK

SEQ ID NO: 1084 - protein, 9 aa, Homo sapiens
  MOD_RES 1: note = Deamidated Asparagine
NGVGVLIWK

SEQ ID NO: 1085 - protein, 9 aa, Homo sapiens
NGVGVLIWK

SEQ ID NO: 1086 - protein, 8 aa, Homo sapiens
AEIEYLEK

SEQ ID NO: 1087 - protein, 11 aa, Homo sapiens
  MOD_RES 5: note = Carboxymethylated Cysteine
NKEDCVEIYIK

SEQ ID NO: 1088 - protein, 18 aa, Homo sapiens
SLTEEAENWGDGEPNNKK

SEQ ID NO: 1089 - protein, 8 aa, Homo sapiens
SYYWIGIR

SEQ ID NO: 1090 - protein, 18 aa, Homo sapiens
  MOD_RES 3: note = Carboxymethylated Cysteine
  MOD_RES 16: note = Carboxymethylated Cysteine
TICESSGIWSNPSPICQK

SEQ ID NO: 1091 - protein, 11 aa, Homo sapiens
  MOD_RES 1: note = Deamidated Asparagine
NNNRPSGISER

SEQ ID NO: 1092 - protein, 11 aa, Homo sapiens
  MOD_RES 3: note = Deamidated Asparagine
NNNRPSGISER

SEQ ID NO: 1093 - protein, 11 aa, Homo sapiens
NNNRPSGISER

SEQ ID NO: 1094 - protein, 12 aa, Homo sapiens
FPTDQLTPDQER

SEQ ID NO: 1095 - protein, 10 aa, Homo sapiens
IANVFTNAFR

SEQ ID NO: 1096 - protein, 10 aa, Homo sapiens
  MOD_RES 3: note = Carboxymethylated Cysteine
IPCFLAGDTR

SEQ ID NO: 1097 - protein, 10 aa, Homo sapiens
QALAQISLPR

SEQ ID NO: 1098 - protein, 12 aa, Homo sapiens
VVLEGGIDPILR

SEQ ID NO: 1099 - protein, 13 aa, Homo sapiens
AGVFSDLSNQELK

SEQ ID NO: 1100 - protein, 10 aa, Homo sapiens
  MOD_RES 1: note = Carboxymethylated Cysteine
CLAFTDVAPR

SEQ ID NO: 1101 - protein, 9 aa, Homo sapiens
DNGPNYVQR

SEQ ID NO: 1102 - protein, 8 aa, Homo sapiens
DTVIVWPR

SEQ ID NO: 1103 - protein, 10 aa, Homo sapiens
FYGSPEELAR

SEQ ID NO: 1104 - protein, 13 aa, Homo sapiens
GDFPSPIHVSGPR

SEQ ID NO: 1105 - protein, 17 aa, Homo sapiens
GHDSTEEPPLFSSHKPR

SEQ ID NO: 1106 - protein, 20 aa, Homo sapiens
KYADGEVDVVVLEDPLPGGK

SEQ ID NO: 1107 - protein, 11 aa, Homo sapiens
LQPSSTTTMAK

SEQ ID NO: 1108 - protein, 29 aa, Homo sapiens
PGYQSSWASRPISTAEYALLYHTLQEATK

SEQ ID NO: 1109 - protein, 9 aa, Homo sapiens
VDLDVAGTK

SEQ ID NO: 1110 - protein, 15 aa, Homo sapiens
VVQPTLEQTQYSWER

SEQ ID NO: 1111 - protein, 19 aa, Homo sapiens
YADGEVDVVVLEDPLPGGK

SEQ ID NO: 1112 - protein, 16 aa, Homo sapiens
  MOD_RES 5: note = Carboxymethylated Cysteine
  MOD_RES 12: note = Carboxymethylated Cysteine
ALSMCPPSPLGCELVK

SEQ ID NO: 1113 - protein, 9 aa, Homo sapiens
  MOD_RES 7: note = Carboxymethylated Cysteine
AVYLPNCDR

SEQ ID NO: 1114 - protein, 10 aa, Homo sapiens
  MOD_RES 7: note = Carboxymethylated Cysteine
AVYLPNCDRK

SEQ ID NO: 1115 - protein, 12 aa, Homo sapiens
FVGGAENTAHPR

SEQ ID NO: 1116 - protein, 8 aa, Homo sapiens
IISAPEMR

SEQ ID NO: 1117 - protein, 10 aa, Homo sapiens
EAFNMIDQNR

SEQ ID NO: 1118 - protein, 12 aa, Homo sapiens
EDLHDMLASLGK

SEQ ID NO: 1119 - protein, 10 aa, Homo sapiens
GNFNYVEFTR

SEQ ID NO: 1120 - protein, 11 aa, Homo sapiens
LNGTDPEDVIR

SEQ ID NO: 1121 - protein, 20 aa, Homo sapiens
  MOD_RES 5: note = Carboxymethylated Cysteine
NAFACFDEEASGFIHEDHLR

SEQ ID NO: 1122 - protein, 15 aa, Homo sapiens
ALSVAELTQQMFDAR

SEQ ID NO: 1123 - protein, 15 aa, Homo sapiens
AVLVDLEPGTMDSIR

SEQ ID NO: 1124 - protein, 12 aa, Homo sapiens
EVDQQLLSVQTR

SEQ ID NO: 1125 - protein, 18 aa, Homo sapiens
FWEMIGEEHGIDLAGSDR

SEQ ID NO: 1126 - protein, 9 aa, Homo sapiens
GASALQLER

SEQ ID NO: 1127 - protein, 18 aa, Homo sapiens
GHYTEGAELIENVLEVVR

SEQ ID NO: 1128 - protein, 12 aa, Homo sapiens
IMNSFSVMPSPK

SEQ ID NO: 1129 - protein, 11 aa, Homo sapiens
ISVYYNEAYGR

SEQ ID NO: 1130 - protein, 23 aa, Homo sapiens
LGALFQPDSFVHGNSGAGNNWAK

SEQ ID NO: 1131 - protein, 20 aa, Homo sapiens
LHFFMPGFAPLTAQGSQQYR

SEQ ID NO: 1132 - protein, 25 aa, Homo sapiens
LTTPTYGDLNHLVSLTMSGITTSLR

SEQ ID NO: 1133 - protein, 12 aa, Homo sapiens
ARPEDVVNLDLK

SEQ ID NO: 1134 - protein, 13 aa, Homo sapiens
DAFDTLFDHAPDK

SEQ ID NO: 1135 - protein, 14 aa, Homo sapiens
LNVAEVTQSEIGQK

SEQ ID NO: 1136 - protein, 15 aa, Homo sapiens
LQTVLEAVHDLLRPR

SEQ ID NO: 1137 - protein, 14 aa, Homo sapiens
QLEEDLYDGQVLQK

SEQ ID NO: 1138 - protein, 14 aa, Homo sapiens
VLLDWINDVLVEER

SEQ ID NO: 1139 - protein, 10 aa, Homo sapiens
AGGAAGLFAK

SEQ ID NO: 1140 - protein, 23 aa, Homo sapiens
AQTVFEDLNQELLEELPILYNSR

SEQ ID NO: 1141 - protein, 11 aa, Homo sapiens
LNHNLYEVMSK

SEQ ID NO: 1142 - protein, 9 aa, Homo sapiens
SLVISPPVR

SEQ ID NO: 1143 - protein, 11 aa, Homo sapiens
AHSQTDRENLR

SEQ ID NO: 1144 - protein, 14 aa, Homo sapiens
FISVGYVDDTQFVR

SEQ ID NO: 1145 - protein, 15 aa, Homo sapiens
  MOD_RES 9: note = Oxidized Methionine
FNKPFVFLMIDQNTK

SEQ ID NO: 1146 - protein, 22 aa, Homo sapiens
  MOD_RES 9: note = Oxidized Methionine
  MOD_RES 20: note = Oxidized Methionine
FNKPFVFLMIDQNTKSPLFMGK

SEQ ID NO: 1147 - protein, 22 aa, Homo sapiens
  MOD_RES 9: note = Oxidized Methionine
FNKPFVFLMIDQNTKSPLFMGK

SEQ ID NO: 1148 - protein, 15 aa, Homo sapiens
FNKPFVFLMIDQNTK

SEQ ID NO: 1149 - protein, 22 aa, Homo sapiens
FNKPFVFLMIDQNTKSPLFMGK

SEQ ID NO: 1150 - protein, 12 aa, Homo sapiens
  MOD_RES 6: note = Oxidized Methionine
PFVFLMIDQNTK

SEQ ID NO: 1151 - protein, 12 aa, Homo sapiens
  MOD_RES 10: note = Deamidated Asparagine
PFVFLMIDQNTK

SEQ ID NO: 1152 - protein, 12 aa, Homo sapiens
PFVFLMIDQNTK

SEQ ID NO: 1153 - protein, 20 aa, Homo sapiens
  SITE 1: note = Pyroglutamic acid
XQPVLTQPTSLSASPGASAR

SEQ ID NO: 1154 - protein, 16 aa, Homo sapiens
  MOD_RES 5: note = Carboxymethylated Cysteine
  MOD_RES 11: note = Carboxymethylated Cysteine
EVEDCVTNRPCGSQVR

SEQ ID NO: 1155 - protein, 20 aa, Homo sapiens
  SITE 1: note = Pyroglutamic acid
XQPVLTQSSSASASLGSSVK

SEQ ID NO: 1156 - protein, 10 aa, Homo sapiens
FSGSSSGADR

SEQ ID NO: 1157 - protein, 17 aa, Homo sapiens
GSGVPDRFSGSSSGADR

SEQ ID NO: 1158 - protein, 15 aa, Homo sapiens
ALSVAELTQQMFDAR

SEQ ID NO: 1159 - protein, 15 aa, Homo sapiens
AVLVDLEPGTMDSIR

SEQ ID NO: 1160 - protein, 12 aa, Homo sapiens
EVDQQLLSVQTR

SEQ ID NO: 1161 - protein, 18 aa, Homo sapiens
FWEMIGEEHGIDLAGSDR

SEQ ID NO: 1162 - protein, 9 aa, Homo sapiens
GASALQLER

SEQ ID NO: 1163 - protein, 18 aa, Homo sapiens
GHYTEGAELIENVLEVVR

SEQ ID NO: 1164 - protein, 12 aa, Homo sapiens
IMNSFSVMPSPK

SEQ ID NO: 1165 - protein, 11 aa, Homo sapiens
ISVYYNEAYGR

SEQ ID NO: 1166 - protein, 23 aa, Homo sapiens
LGALFQPDSFVHGNSGAGNNWAK

SEQ ID NO: 1167 - protein, 20 aa, Homo sapiens
LHFFMPGFAPLTAQGSQQYR

SEQ ID NO: 1168 - protein, 25 aa, Homo sapiens
LTTPTYGDLNHLVSLTMSGITTSLR

SEQ ID NO: 1169 - protein, 11 aa, Homo sapiens
  MOD_RES 1: note = Deamidated Asparagine
NNNRPSGISER

SEQ ID NO: 1170 - protein, 11 aa, Homo sapiens
  MOD_RES 3: note = Deamidated Asparagine
NNNRPSGISER

SEQ ID NO: 1171 - protein, 11 aa, Homo sapiens
NNNRPSGISER

SEQ ID NO: 1172 - protein, 15 aa, Homo sapiens
  MOD_RES 9: note = Oxidized Methionine
FNKPFVFLMIDQNTK

SEQ ID NO: 1173 - protein, 22 aa, Homo sapiens
  MOD_RES 9: note = Oxidized Methionine
  MOD_RES 20: note = Oxidized Methionine
FNKPFVFLMIDQNTKSPLFMGK

SEQ ID NO: 1174 - protein, 22 aa, Homo sapiens
  MOD_RES 9: note = Oxidized Methionine
FNKPFVFLMIDQNTKSPLFMGK

SEQ ID NO: 1175 - protein, 15 aa, Homo sapiens
FNKPFVFLMIDQNTK

SEQ ID NO: 1176 - protein, 22 aa, Homo sapiens
FNKPFVFLMIDQNTKSPLFMGK

SEQ ID NO: 1177 - protein, 12 aa, Homo sapiens
  MOD_RES 6: note = Oxidized Methionine
PFVFLMIDQNTK

SEQ ID NO: 1178 - protein, 12 aa, Homo sapiens
  MOD_RES 10: note = Deamidated Asparagine
PFVFLMIDQNTK

SEQ ID NO: 1179 - protein, 12 aa, Homo sapiens
PFVFLMIDQNTK

SEQ ID NO: 1180 - protein, 12 aa, Homo sapiens
FPTDQLTPDQER

SEQ ID NO: 1181 - protein, 10 aa, Homo sapiens
IANVFTNAFR

SEQ ID NO: 1182 - protein, 10 aa, Homo sapiens
  MOD_RES 3: note = Carboxymethylated Cysteine
IPCFLAGDTR

SEQ ID NO: 1183 - protein, 10 aa, Homo sapiens
QALAQISLPR

SEQ ID NO: 1184 - protein, 12 aa, Homo sapiens
VVLEGGIDPILR

SEQ ID NO: 1185 - protein, 16 aa, Homo sapiens
  MOD_RES 5: note = Carboxymethylated Cysteine
  MOD_RES 12: note = Carboxymethylated Cysteine
ALSMCPPSPLGCELVK

SEQ ID NO: 1186 - protein, 9 aa, Homo sapiens
  MOD_RES 7: note = Carboxymethylated Cysteine
AVYLPNCDR

SEQ ID NO: 1187 - protein, 10 aa, Homo sapiens
  MOD_RES 7: note = Carboxymethylated Cysteine
AVYLPNCDRK

SEQ ID NO: 1188 - protein, 12 aa, Homo sapiens
FVGGAENTAHPR

SEQ ID NO: 1189 - protein, 8 aa, Homo sapiens
IISAPEMR

SEQ ID NO: 1190 - protein, 13 aa, Homo sapiens
AGVFSDLSNQELK

SEQ ID NO: 1191 - protein, 10 aa, Homo sapiens
  MOD_RES 1: note = Carboxymethylated Cysteine
CLAFTDVAPR

SEQ ID NO: 1192 - protein, 9 aa, Homo sapiens
DNGPNYVQR

SEQ ID NO: 1193 - protein, 8 aa, Homo sapiens
DTVIVWPR

SEQ ID NO: 1194 - protein, 10 aa, Homo sapiens
FYGSPEELAR

SEQ ID NO: 1195 - protein, 13 aa, Homo sapiens
GDFPSPIHVSGPR

SEQ ID NO: 1196 - protein, 17 aa, Homo sapiens
GHDSTEEPPLFSSHKPR

SEQ ID NO: 1197 - protein, 20 aa, Homo sapiens
KYADGEVDVVVLEDPLPGGK

SEQ ID NO: 1198 - protein, 11 aa, Homo sapiens
LQPSSTTTMAK

SEQ ID NO: 1199 - protein, 29 aa, Homo sapiens
PGYQSSWASRPISTAEYALLYHTLQEATK

SEQ ID NO: 1200 - protein, 9 aa, Homo sapiens
VDLDVAGTK

SEQ ID NO: 1201 - protein, 15 aa, Homo sapiens
VVQPTLEQTQYSWER

SEQ ID NO: 1202 - protein, 19 aa, Homo sapiens
YADGEVDVVVLEDPLPGGK

SEQ ID NO: 1203 - protein, 16 aa, Homo sapiens
  MOD_RES 5: note = Carboxymethylated Cysteine
  MOD_RES 11: note = Carboxymethylated Cysteine
EVEDCVTNRPCGSQVR

SEQ ID NO: 1204 - protein, 11 aa, Homo sapiens
AHSQTDRENLR

SEQ ID NO: 1205 - protein, 14 aa, Homo sapiens
FISVGYVDDTQFVR

SEQ ID NO: 1206 - protein, 12 aa, Homo sapiens
ARPEDVVNLDLK

SEQ ID NO: 1207 - protein, 13 aa, Homo sapiens
DAFDTLFDHAPDK

SEQ ID NO: 1208 - protein, 14 aa, Homo sapiens
LNVAEVTQSEIGQK

SEQ ID NO: 1209 - protein, 15 aa, Homo sapiens
LQTVLEAVHDLLRPR

SEQ ID NO: 1210 - protein, 14 aa, Homo sapiens
QLEEDLYDGQVLQK

SEQ ID NO: 1211 - protein, 14 aa, Homo sapiens
VLLDWINDVLVEER

SEQ ID NO: 1212 - protein, 20 aa, Homo sapiens
  SITE 1: note = Pyroglutamic acid
XQPVLTQSSSASASLGSSVK

SEQ ID NO: 1213 - protein, 10 aa, Homo sapiens
FSGSSSGADR

SEQ ID NO: 1214 - protein, 17 aa, Homo sapiens
GSGVPDRFSGSSSGADR

SEQ ID NO: 1215 - protein, 20 aa, Homo sapiens
  SITE 1: note = Pyroglutamic acid
XQPVLTQPTSLSASPGASAR

SEQ ID NO: 1216 - protein, 10 aa, Homo sapiens
EAFNMIDQNR

SEQ ID NO: 1217 - protein, 12 aa, Homo sapiens
EDLHDMLASLGK

SEQ ID NO: 1218 - protein, 10 aa, Homo sapiens
GNFNYVEFTR

SEQ ID NO: 1219 - protein, 11 aa, Homo sapiens
LNGTDPEDVIR

SEQ ID NO: 1220 - protein, 20 aa, Homo sapiens
  MOD_RES 5: note = Carboxymethylated Cysteine
NAFACFDEEASGFIHEDHLR

SEQ ID NO: 1221 - protein, 10 aa, Homo sapiens
AGGAAGLFAK

SEQ ID NO: 1222 - protein, 23 aa, Homo sapiens
AQTVFEDLNQELLEELPILYNSR

SEQ ID NO: 1223 - protein, 11 aa, Homo sapiens
LNHNLYEVMSK

SEQ ID NO: 1224 - protein, 9 aa, Homo sapiens
SLVISPPVR

SEQ ID NO: 1225 - protein, 12 aa, Homo sapiens
ALAAGGVGSIVR

SEQ ID NO: 1226 - protein, 19 aa, Homo sapiens
GFESPSDNSSAMLLQWHEK

SEQ ID NO: 1227 - protein, 17 aa, Homo sapiens
AEDGSVIDYELIDQDAR

SEQ ID NO: 1228 - protein, 10 aa, Homo sapiens
DALNIETAIK

SEQ ID NO: 1229 - protein, 14 aa, Homo sapiens
GVDEVTIVNILTNR

SEQ ID NO: 1230 - protein, 18 aa, Homo sapiens
LSLEGDHSTPPSAYGSVK

SEQ ID NO: 1231 - protein, 18 aa, Homo sapiens
RAEDGSVIDYELIDQDAR

SEQ ID NO: 1232 - protein, 10 aa, Homo sapiens
TNQELQEINR

SEQ ID NO: 1233 - protein, 11 aa, Homo sapiens
DIDDLELTLAK

SEQ ID NO: 1234 - protein, 17 aa, Homo sapiens
DLEEATLQHEATAAALR

SEQ ID NO: 1235 - protein, 11 aa, Homo sapiens
INATLETKQPR

SEQ ID NO: 1236 - protein, 12 aa, Homo sapiens
LAEQELIETSER

SEQ ID NO: 1237 - protein, 13 aa, Homo sapiens
LASADIETYLLEK

SEQ ID NO: 1238 - protein, 20 aa, Homo sapiens
LELQSALEEAEASLEHEEGK

SEQ ID NO: 1239 - protein, 15 aa, Homo sapiens
RVIQYFASIAAIGDR

SEQ ID NO: 1240 - protein, 20 aa, Homo sapiens
AWDLTAPVAAVQAAVSNLVR

SEQ ID NO: 1241 - protein, 13 aa, Homo sapiens
AVAGNISDPGLQK

SEQ ID NO: 1242 - protein, 15 aa, Homo sapiens
DPSASPGDAGEQAIR

SEQ ID NO: 1243 - protein, 8 aa, Homo sapiens
DYLIDGSR

SEQ ID NO: 1244 - protein, 11 aa, Homo sapiens
ELTPQVVSAAR

SEQ ID NO: 1245 - protein, 12 aa, Homo sapiens
ETVQTTEDQILK

SEQ ID NO: 1246 - protein, 8 aa, Homo sapiens
GLVAEGHR

SEQ ID NO: 1247 - protein, 18 aa, Homo sapiens
LVQAAQMLQSDPYSVPAR

SEQ ID NO: 1248 - protein, 13 aa, Homo sapiens
MLGQMTDQVADLR

SEQ ID NO: 1249 - protein, 10 aa, Homo sapiens
MSAEINEIIR

SEQ ID NO: 1250 - protein, 12 aa, Homo sapiens
MTGLVDEAIDTK

SEQ ID NO: 1251 - protein, 9 aa, Homo sapiens
NQGIEEALK

SEQ ID NO: 1252 - protein, 12 aa, Homo sapiens
SLLDASEEAIKK

SEQ ID NO: 1253 - protein, 10 aa, Homo sapiens
TISPMVMDAK

SEQ ID NO: 1254 - protein, 11 aa, Homo sapiens
VMLVNSMNTVK

SEQ ID NO: 1255 - protein, 10 aa, Homo sapiens
  MOD_RES 5: note = Carboxymethylated Cysteine
AFIGCPPPPK

SEQ ID NO: 1256 - protein, 12 aa, Homo sapiens
QLLTKPQHQFVR

SEQ ID NO: 1257 - protein, 11 aa, Homo sapiens
QSLQELPAGSR

SEQ ID NO: 1258 - protein, 18 aa, Homo sapiens
  MOD_RES 10: note = Carboxymethylated Cysteine
DVPASFAPACLSHEIIIR

SEQ ID NO: 1259 - protein, 23 aa, Homo sapiens
  MOD_RES 2: note = Carboxymethylated Cysteine
  MOD_RES 10: note = Carboxymethylated Cysteine
  MOD_RES 15: note = Carboxymethylated Cysteine
  MOD_RES 19: note = Carboxymethylated Cysteine
GCPVHLVDSCPWPHCNPSCPTVR

SEQ ID NO: 1260 - protein, 12 aa, Homo sapiens
GLADSGWFLDNK

SEQ ID NO: 1261 - protein, 17 aa, Homo sapiens
  MOD_RES 4: note = Carboxymethylated Cysteine
  MOD_RES 10: note = Carboxymethylated Cysteine
HTDCVDTITCAPTEAIR

SEQ ID NO: 1262 - protein, 9 aa, Homo sapiens
LGYPAIQVR

SEQ ID NO: 1263 - protein, 8 aa, Homo sapiens
LYIQNLGR

SEQ ID NO: 1264 - protein, 15 aa, Homo sapiens
  MOD_RES 10: note = Carboxymethylated Cysteine
QFQEGEEWNCFFGYK

SEQ ID NO: 1265 - protein, 19 aa, Homo sapiens
  MOD_RES 9: note = Carboxymethylated Cysteine
SLAQSLYPCSAQQLNEDLR

SEQ ID NO: 1266 - protein, 9 aa, Homo sapiens
YWNGVVPER

SEQ ID NO: 1267 - protein, 10 aa, Homo sapiens
  MOD_RES 1: note = Oxidized Methionine
MYYSAVEPTK

SEQ ID NO: 1268 - protein, 10 aa, Homo sapiens
MYYSAVEPTK

SEQ ID NO: 1269 - protein, 21 aa, Homo sapiens
MYYSAVEPTKDIFTGLIGPMK

SEQ ID NO: 1270 - protein, 36 aa, Homo sapiens; identical to SEQ ID NO: 1074
  MOD_RES 9: note = Carboxymethylated Cysteine

SEQ ID NO: 1271 - protein, 10 aa, Homo sapiens
  MOD_RES 5: note = Carboxymethylated Cysteine
AFIGCPPPPK

SEQ ID NO: 1272 - protein, 12 aa, Homo sapiens
QLLTKPQHQFVR

SEQ ID NO: 1273 - protein, 11 aa, Homo sapiens
QSLQELPAGSR

SEQ ID NO: 1274 - protein, 12 aa, Homo sapiens
ALAAGGVGSIVR

SEQ ID NO: 1275 - protein, 19 aa, Homo sapiens
GFESPSDNSSAMLLQWHEK

SEQ ID NO: 1276 - protein, 21 aa, Homo sapiens
AIPDLTAPVAAVQAAVSNLVR

SEQ ID NO: 1277 - protein, 13 aa, Homo sapiens
AVAGNISDPGLQK

SEQ ID NO: 1278 - protein, 15 aa, Homo sapiens
DPSASPGDAGEQAIR

SEQ ID NO: 1279 - protein, 8 aa, Homo sapiens
DYLIDGSR

SEQ ID NO: 1280 - protein, 11 aa, Homo sapiens
ELTPQVVSAAR

SEQ ID NO: 1281 - protein, 12 aa, Homo sapiens
ETVQTTEDQILK

SEQ ID NO: 1282 - protein, 8 aa, Homo sapiens
GLVAEGHR

SEQ ID NO: 1283 - protein, 18 aa, Homo sapiens
LVQAAQMLQSDPYSVPAR

SEQ ID NO: 1284 - protein, 13 aa, Homo sapiens
MLGQMTDQVADLR

SEQ ID NO: 1285 - protein, 10 aa, Homo sapiens
MSAEINEIIR

SEQ ID NO: 1286 - protein, 12 aa, Homo sapiens
MTGLVDEAIDTK

SEQ ID NO: 1287 - protein, 9 aa, Homo sapiens
NQGIEEALK

SEQ ID NO: 1288 - protein, 12 aa, Homo sapiens
SLLDASEEAIKK

SEQ ID NO: 1289 - protein, 10 aa, Homo sapiens
TISPMVMDAK

SEQ ID NO: 1290 - protein, 11 aa, Homo sapiens
VMLVNSMNTVK

SEQ ID NO: 1291 - protein, 10 aa, Homo sapiens
  MOD_RES 1: note = Oxidized Methionine
MYYSAVEPTK

SEQ ID NO: 1292 - protein, 10 aa, Homo sapiens
MYYSAVEPTK

SEQ ID NO: 1293 - protein, 21 aa, Homo sapiens
MYYSAVEPTKDIFTGLIGPMK

SEQ ID NO: 1294 - protein, 36 aa, Homo sapiens; identical to SEQ ID NO: 1074
  MOD_RES 9: note = Carboxymethylated Cysteine

SEQ ID NO: 1295 - protein, 17 aa, Homo sapiens
AEDGSVIDYELIDQDAR

SEQ ID NO: 1296 - protein, 10 aa, Homo sapiens
DALNIETAIK

SEQ ID NO: 1297 - protein, 14 aa, Homo sapiens
GVDEVTIVNILTNR

SEQ ID NO: 1298 - protein, 18 aa, Homo sapiens
LSLEGDHSTPPSAYGSVK

SEQ ID NO: 1299 - protein, 18 aa, Homo sapiens
RAEDGSVIDYELIDQDAR

SEQ ID NO: 1300 - protein, 10 aa, Homo sapiens
TNQELQEINR

(19) United States
(12) Patent Application Publication
(10) Pub. No.: US 2025/0102482 A1
(43) Pub. Date: Mar. 27, 2025

(54) METHODS OF PREPARING AND ANALYZING SAMPLES FOR BIOMARKERS ASSOCIATED WITH PLACENTA ACCRETA

(71) Applicants: NX PRENATAL INC., Bellaire, TX (US); The Brigham and Women’s Hospital, Inc., Boston, MA (US)

(72) Inventors: Thomas F. McELRATH, Dover, MA (US); Kevin P. ROSENBLATT, Bellaire, TX (US); Brian D. BROHMAN, Louisville, KY (US); Prem P. GURNANI, Bellaire, TX (US)

(21) Appl. No.: 18/709,475

(22) PCT Filed: Nov. 14, 2022
(86) PCT No.: PCT/US2022/079835

§ 371 Date: May 10, 2024

Related U.S. Application Data
(60) Provisional application No. 63/278,456, filed on Nov. 11, 2021.

(51) Int. Cl. G01N 30/88; A61B 34/10; G01N 30/72; G01N 33/573; G01N 33/68; G16H 10/40; G16H 50/30

(52) U.S. Cl. CPC G01N 30/88; A61B 34/10; G01N 33/573; G01N 33/6848; G01N 33/6893; G16H 10/40; G16H 50/30; G01N 30/7233; G01N 2030/8831; G01N 2333/912; G01N 2333/948; G01N 2800/368

(57) ABSTRACT

Disclosed herein are methods of preparing and analyzing samples for biomarkers associated with placenta accreta in pregnant subjects. Also disclosed are method of assessing the risk of placenta accreta in pregnant subjects.

### CROSS REFERENCE TO RELATED APPLICATIONS

This application claims priority to U.S. provisional patent application No. 63/278,456, filed on Nov. 11, 2021, the contents of which are incorporated by reference herein in their entirety.

### BACKGROUND

Placenta accreta is a serious pregnancy condition that occurs when the placenta grows too deeply into the uterine wall. Typically, the placenta detaches from the uterine wall after childbirth. With placenta accreta, part or all of the placenta remains attached. This can cause severe blood loss after delivery. It is also possible for the placenta to invade the muscles of the uterus (placenta increta) or grow through the uterine wall (placenta percreta).

Placenta accreta is considered a high-risk pregnancy complication. If the condition is diagnosed during pregnancy, there is increases likelihood that a woman will require cesarian delivery followed by hysterectomy.

The incidence of placenta accreta spectrum has increased by a factor of approximately 8 since the 1970s, probably owing to increases in cesarean delivery. The incidence of cesarean birth is much higher in women with placenta accreta than those without. For example, in a first pregnancy, the incidence of cesarian birth in women without placenta accreta is 0.03%, while in women with placenta accreta, the incidence is 100-fold higher, or 3%.

Women with major risk factors, such as placenta previa, previous cesarean delivery, endometrial ablation, or other uterine surgery, should undergo obstetrical sonography in the middle-to-late second trimester to assess for possible placenta accreta spectrum. Patients with suspected placenta accreta spectrum should be referred to a center with multidisciplinary expertise and experience.

Present diagnosis of placenta accreta involves ultrasound, the specificity and sensitivity of which relies on knowledge of patient's clinical status (e.g., clinical suspicion for accreta, prior knowledge of risk factors). Needed are methods of more sensitive ways of predicting and detecting placenta accreta in a pregnant woman. Provided herein are methods and compositions that address this need.

### SUMMARY

Disclosed herein are circulating microparticle (CMP)-associated proteins, useful for the prediction and detection of plasma accreta. In some embodiments, the CMP-associated proteins are collected from around about 20 weeks to around about 37 weeks of gestation, that can be used to assess risk of placenta accreta. The biomarkers are presented in Tables 1-6, and Table 9, the tables of FIGS. 1A-1D, FIGS. 2A-2C, FIGS. 3A-3K, FIGS. 4A-4J, FIGS. 5A-5C, FIGS. 6A-6F, as well as tables in Example 1. Also provided are surrogates useful for the detection of the biomarkers, presented in FIGS. 10A-10C, FIGS. 11A-11C, FIGS. 12A-12H, FIGS. 13A-13H, FIGS. 14A-14D, and FIGS. 15A-15B. Panels of biomarkers are also presented.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIGS. 1A-1D depict Table 1 comprising median 24-week samples. The markers are ranked using Lasso regression (alpha=1). The top 20 markers are displayed. This technique optimizes proteins that may interact and “collaborate” in panels rather than isolated markers. Individual markers are ranked by frequency of utility in multimarker panels.

FIGS. 2A-2C depict Table 2 comprising median 34-week samples. The markers are ranked using Lasso regression (alpha=1). The top 20 markers are displayed and ranked by frequency of utility in multimarker panels.

FIGS. 3A-3K. depict Table 3 comprising the top 50 protein markers at median 24 weeks using an ensemble feature selection routine that selects the top individual markers that distinguish case from control. The protein markers that overlap with the top 20 of the Lasso regression of Table 1 are indicated with asterisk. Individual markers are ranked by performance in distinguishing case/control.

FIGS. 4A-4J depict Table 4 comprising the top 50 protein markers at median 34 weeks using an ensemble feature selection routine that selects the top individual markers that distinguish case from control. The protein markers that overlap with the top 20 of the Lasso regression (Table 2) are indicated with an asterisk. Individual markers are ranked by performance in distinguishing case/control.

FIGS. 5A-5C depict Table 5 comprising 24-week markers. Individual markers ranked by performance in distinguishing case/control.

FIGS. 6A-6F depict Table 6 comprising 34-week markers. Individual markers ranked by performance in distinguishing case/control.

FIG. 7 depicts Table 7 comprising 24-week markers. Top performing multiplex panels are ranked by average AUC of an iterative cross-validation procedure.

FIG. 8 depicts Table 8 comprising 34-week markers. Top performing multiplex panels are ranked by average AUC of an iterative cross-validation procedure.

FIG. 9 illustrates a schematic of a protocol for identifying predictive circulating microparticle protein panels for placenta accreta.

FIGS. 10A-10C provide surrogate peptides, useful for the detection of the biomarkers of Table 1.

FIGS. 11A-11C provide surrogate peptides, useful for the detection of the biomarkers of Table 2.

FIGS. 12A-12H provide surrogate peptides, useful for the detection of the biomarkers of Table 3.

FIGS. 13A-13H provide surrogate peptides, useful for the detection of the biomarkers of Table 4.

FIGS. 14A-14D provide surrogate peptides, useful for the detection of the biomarkers of Table 5.

FIGS. 15A-15B provide surrogate peptides, useful for the detection of the biomarkers of Table 6.

FIG. 16A is a density plot of protein versus permuted with a first shaded area represents actual protein AUC and a second shaded area represents AUC from randomly permuting the sample labels (placenta accreta spectrum vs. control) for the second trimester (e.g., 24 weeks).

FIG. 16B is a density plot of protein versus permuted with a first shaded area represents actual protein AUC and a second shaded area represents AUC from randomly permuting the sample labels (placenta accreta spectrum vs. control) for the third trimester (e.g., 37 weeks).

FIG. 17 depicts a schematic of exemplary canonical pathways, upstream regulators and molecular and cellular function analyses proposed in the second and third trimester leading to morbid placental adherence.

### DETAILED DESCRIPTION

### I. Introduction

Disclosed herein are methods, compositions, systems and articles of manufacture useful in preparing a sample for the detection of biomarkers useful in determining risk of developing, and for treating, placenta accreta. In some embodiments, determination involves detection of placenta accreta biomarkers found in microparticle-enriched fractions from the blood of pregnant women. Exemplary biomarkers useful for the detection of placenta accreta in either or both the second and third trimester are presented in Table 1, Table 2, Table 3, Table 4, Table 5, Table 6, and Table 9. As discussed below, Table 7 and Table 8 present biomarker panels for placenta accreta. Additional marker sets are also presented herein.

### II. Subjects

Subjects for providing samples for prediction and treatment of placenta accreta are pregnant human females. The stage of pregnancy can be calculated from the first day of the last normal menstrual period of the pregnant subject.

In some embodiments, the pregnant women may be about 20 weeks to about 37 weeks of pregnancy. In some embodiments, the pregnant woman may be about 20 weeks of pregnancy, about 21 weeks of pregnancy, about 22 weeks of pregnancy, about 23 weeks of pregnancy, about 24 weeks of pregnancy, about 25 weeks of pregnancy, about 26 weeks of pregnancy, about 27 weeks of pregnancy, about 28 weeks of pregnancy, about 29 weeks of pregnancy, about 30 weeks of pregnancy, about 31 weeks of pregnancy, about 32 weeks of pregnancy, about 33 weeks of pregnancy, about 34 weeks of pregnancy, about 35 weeks of pregnancy, about 36 weeks of pregnancy, or about 37 weeks of pregnancy.

Pregnant subjects of the methods described herein can belong to one or more classes including primiparous (no previous child brought to delivery, interchangeably referred to herein as nulliparous or parity-0) or multiparous (at least one previous child brought to at least 20 weeks of gestation, referred to interchangeably herein as parity >0, parity ≥1), primigravida (first pregnancy) or multigravida (more than one pregnancy).

In some embodiments, the pregnant human subject is asymptomatic. In some embodiments, the subject may have a risk factor of placenta accreta. The most common risk factor is a previous cesarean delivery, with the incidence of placenta accreta spectrum increasing with the number of prior cesarean deliveries. In a systematic review, the rate of placenta accreta spectrum increased from 0.3% in women with one previous cesarean delivery to 6.74% for women with five or more cesarean deliveries. Additional risk factors include advanced maternal age, multiparity, prior uterine surgeries or curettage, and Asherman syndrome. Placenta previa is another significant risk factor.

### III. Sample Preparation

A sample for use in the methods of the present disclosure is a biological sample obtained from a pregnant subject. In certain embodiments, the sample is collected during a stage of pregnancy described in the preceding section. In some embodiments, the sample is a blood, saliva, tears, sweat, nasal secretions, urine, amniotic fluid or cervicovaginal fluid sample. In some embodiments, the sample is a blood sample. In some embodiments, the sample is a blood plasma sample. In some embodiments, the sample is a blood serum sample. In some embodiments, the sample is a blood product in a different matrix (e.g. Citrate buffer, or Streck tube). In some embodiments, the sample has been stored frozen (e.g., −20° C. or −80° C.).

The term “microparticle” refers to an extracellular microvesicle or lipid raft protein aggregate having a hydrodynamic diameter of about 50 to about 5000 nm. As such, the term microparticle encompasses exosomes (about 50 to about 100 nm), microvesicles (about 100 to about 300 nm), ectosomes (about 50 to about 1000 nm), apoptotic bodies (about 50 to about 5000 nm) and lipid-protein aggregates of the same dimensions.

The term “microparticle-associated protein” refers to a protein or fragment thereof that is detectable in a microparticle-enriched sample from a mammalian (e.g., human) subject. As such the term “microparticle-associated protein” is not restricted to proteins or fragments thereof that are physically associated with microparticles at the time of detection. The term “microparticle-associated peptide” refers to a protein fragment that is detectable in such a sample.

The term “polypeptide” as used herein refers to a polymer of amino acids. This includes oligopeptides (which typically have fewer than 10 amino acids), peptides (which typically have between about 10 and about 50 amino acids), and proteins (which include polypeptides assuming secondary, tertiary, or quaternary structures). Depending on context, the term “protein” may refer to a polypeptide lacking secondary structure.

Biomarkers for placenta accreta can be derived from microparticles. Microparticles can be isolated from blood (e.g., serum or plasma) or other biological samples, by size exclusion chromatography. The mobile phase/elution buffer can be, for example, a buffered solution such as PBS, or a non-buffered solution. Water, as a mobile phase, refers to non-buffered water, e.g., distilled, deionized, or distilled de-ionized water (“ddH₂O”). The high molecular weight fraction can be collected to obtain a microparticle-enriched sample. Proteins within the microparticle-enriched sample are then extracted before digestion with a proteolytic enzyme such as trypsin to obtain a digested sample comprising a plurality of peptides. The digested sample is then subjected to a peptide purification, concentration, and/or a fractionation step before analysis to obtain a proteomic profile of the sample, e.g., by liquid chromatography and mass spectrometry. In some embodiments, the purification/concentration step comprises reverse phase chromatography (e.g., ZipTip® pipette tip with 0.2 μL C18 resin, from Millipore Corporation, Billerica, MA) or ultrafiltration. In some embodiments, the fractionation step may involve the fractionation into 96 fractions with a high pH reverse phase offline HPLC fractionator be with a Mobile phase A is DI H₂O with 20 mM Formic Acetate, pH 9.3; mobile phase B is Acetonitrile (Optima™, LC/MS grade, Fisher Chemical™) with 20 mM Formic Acetate, pH 9.3.

In some embodiments, for example, detection of proteins by mass spectrometry, a method of sample preparation can include fragmenting proteins in a sample. Fragmentation can be accomplished using proteases, such as trypsin. Tryptic fragments can usefully serve as surrogate biomarkers because their unique mass can be associated with the parent protein.

In certain embodiments, the microparticles are placental-derived exosomes or endothelial-derived exosomes. Such exosomes can be isolated using capture agents, such as antibodies, against surface markers for these cells of origin. For example, placental-derived exosomes can be isolated using antibodies directed to PLAP (placental alkaline phosphatase), Klotho, CD34, CD44 or leukemia inhibitory factor (LIF). Endothelial-derived exosomes can be isolated using antibodies directed to ICAM or VCAM.

### IV. Methods of Detection

Biomarkers can be detected and quantified by any method known in the art. This includes, without limitation, immunoassay, chromatography, mass spectrometry, electrophoresis and surface plasmon resonance.

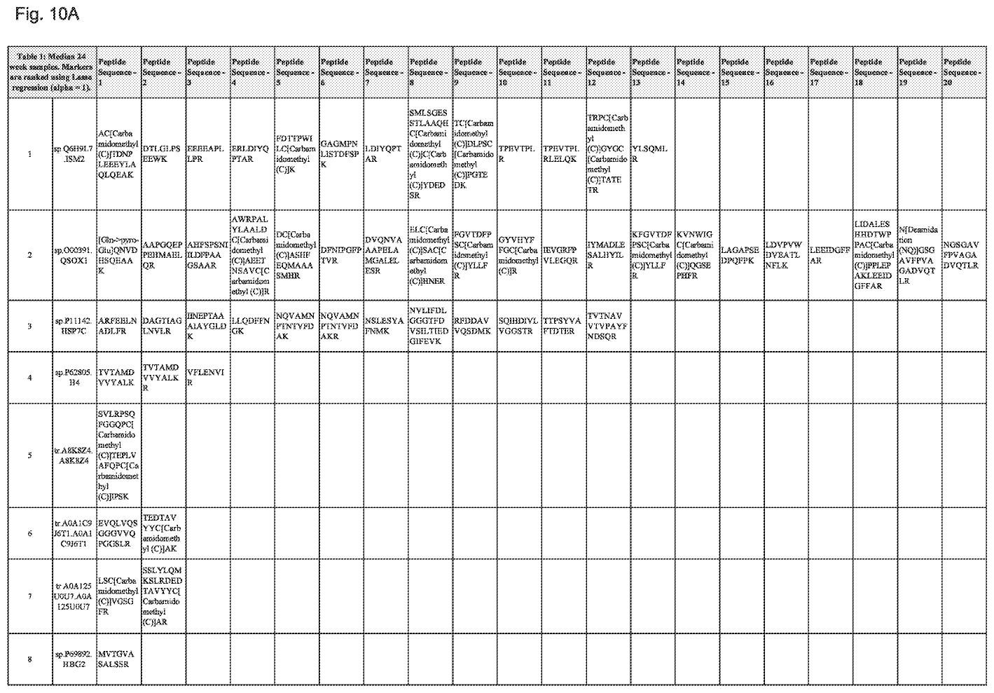

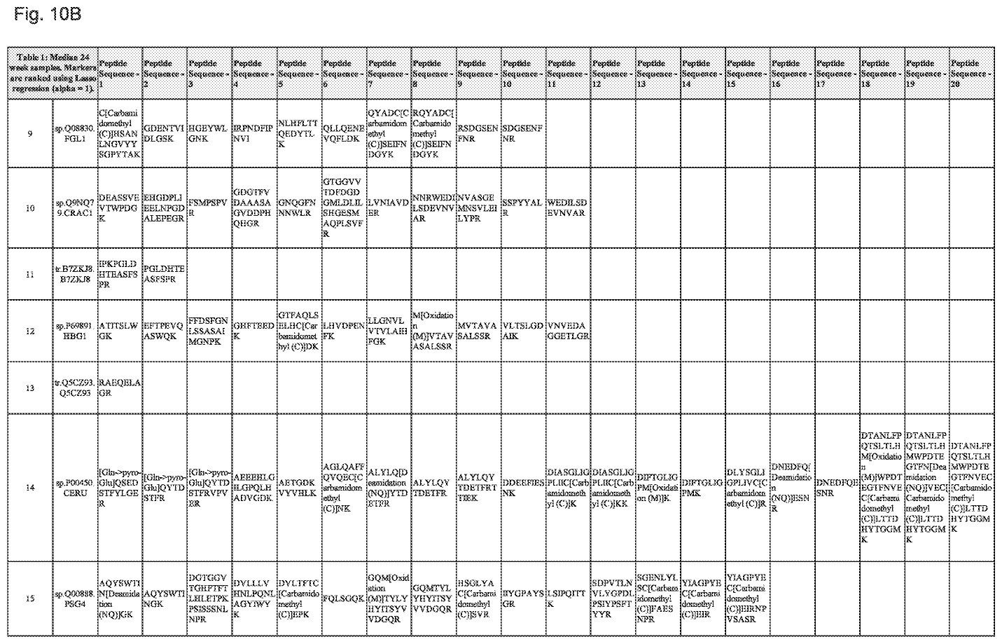

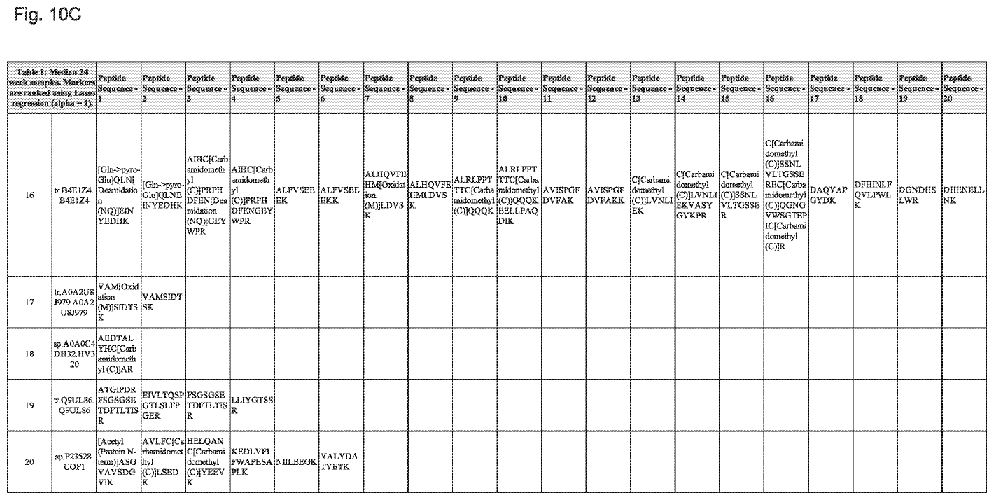

Detection of a biomarker includes detection of an intact protein, or detection of surrogate for the protein, such as a fragment. Exemplary fragments are provided in FIG. 10A to FIG. 15B.

Immunoassay methods include, for example, radioimmunoassay, enzyme-linked immunosorbent assay (ELISA), sandwich assays and Western blot, immunoprecipitation, immunohistochemistry, immunofluorescence, antibody microarray, dot blotting, and FACS.

Chromatographic methods include, for example, affinity chromatography, ion exchange chromatography, size exclusion chromatography/gel filtration chromatography, hydrophobic interaction chromatography and reverse phase chromatography, including, e.g., HPLC.

### 1. Mass Spectrometry

In some embodiments, detecting the level (e.g., including detecting the presence) of a microparticle-associated protein is accomplished using a liquid chromatography/mass spectrometry (LC/MS)-based proteomic analysis. In an exemplary embodiment, the method involves subjecting a sample to size exclusion chromatography and collecting the high molecular weight fraction (e.g., by size-exclusion chromatography) to obtain a microparticle-enriched sample. In some embodiments, the size exclusion chromatography includes a size-exclusion column comprising an agarose solid phase and an aqueous liquid phase. The microparticle-enriched sample is then disrupted (using, for example, chaotropic agents, denaturing agents, reducing agents and/or alkylating agents) and the released contents subjected to proteolysis. The disrupted microsome preparation, containing a plurality of peptides, is then processed using the tandem column system described herein prior to peptide analysis by mass spectrometry, to provide a proteomic profile of the sample. The methods disclosed herein avoid the necessity of protein concentration/purification, buffer exchange and liquid chromatography steps associated with previous methods.

Proteins in a sample can be detected by mass spectrometry. Mass spectrometers typically include an ion source to ionize analytes, and one or more mass analyzers to determine mass. Mass analyzers can be used together in tandem mass spectrometers. Ionization methods include, among others, electrospray or laser desorption methods. Mass analyzers include quadrupoles, ion traps, time-of-flight instruments and magnetic or electric sector instruments. In certain embodiments, the mass spectrometer is a tandem mass spectrometer (e.g., “MS-MS”) that uses a first mass analyzer to select ions of a certain mass and a second mass analyzer to analyze the selected ions. One example of a tandem mass spectrometer is a triple quadrupole instrument, the first and third quadrupoles act as mass filters, and an intermediate quadrupole functions as a collision cell. Mass spectrometry also can be coupled with up-stream separation techniques, such as liquid chromatography or gas chromatography. So, for example, liquid chromatography coupled with tandem mass spectrometry can be referred to as “LC-MS-MS”.

Mass spectrometers useful for the analyses described herein include, without limitation, Altis™ quadrupole, Quantis™ quadrupole, Quantiva™ or Fortis™ triple quadrupole from ThermoFisher Scientific, the 8050 or 8060 triple quadruploes from Shimadzu, the Xevo TQ-XS™ triple quadrupole from Waters, QSight™ Triple Quad LC/MS/MS from Perkin Elmer, Thermo Orbitrap Mass Spectrometer Tribrid Eclipse with a Thermo Fisher Scientific Nanospray Flex™ Ion Source, and others.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods and compositions disclosed herein. In some embodiments, any MS technique can provide process information on the mass of peptides wherein the mass comprises more than 100, more than 1000, more than 10,000, more than 100,000 peptides from a biological sample. Suitable peptide MS and MS/MS techniques and systems are known in the art (see, e.g., Methods in Molecular Biology, vol. 146: “Mass Spectrometry of Proteins and Peptides”, by Chapman, ed., Humana Press 2000; Kassel & Biemann (1990) Anal. Chem. 62:1691-1695; Methods Enzymol 193:455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more peptides. Such quantitative methods can be performed in an automated (Villanueva, et al., Nature Protocols (2006) 1 (2): 880-891) or semi-automated format. In particular embodiments, MS can be operably linked to a liquid chromatography device (LC-MS/MS or LC-MS) or gas chromatography device (GC-MS or GC-MS/MS).

Selected reaction monitoring is a mass spectrometry method in which a first mass analyzer selects a protein of interest (precursor), a collision cell fragments the protein into product fragments and one or more of the fragments is detected in a second mass analyzer. The precursor and product ion pair is called an SRM “transition”. The method is typically performed in a triple quadrupole instrument. When multiple fragments of a protein are analyzed, the method is referred to as Multiple Reaction Monitoring Mass Spectrometry (“MRM-MS”).

Typically, protein samples are digested with a proteolytic enzyme, such as trypsin, to produce peptide fragments. Heavy isotope labeled analogues of certain of these peptides are synthesized as standards. These standards are referred to as Stable Isotopic Standards or “SIS”. SIS peptides are mixed with a protease-treated sample. The mixture is subjected to triple quadrupole mass spectrometry. Peptides corresponding to the daughter ions of the SIS standards and the target peptides are detected with high accuracy, in either the time domain or the mass domain. Usually, a plurality of the daughter ions is used to unambiguously identify the presence of a parent ion, and one of the daughter ions, usually the most abundant, is used for quantification. SIS peptides can be synthesized to order or can be available as commercial kits from vendors such as, for example, e.g., Thermo Fisher Scientific (Waltham, MA) or Biognosys AG (Zurich, Switzerland).

As used herein, the terms “multiple reaction monitoring (MRM)” or “selected reaction monitoring (SRM)” refer to a MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment. A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” or “dynamic” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, the assay can include standards that correspond to the analytes of interest (e.g., peptides having the same amino acid sequence as that of analyte peptides), but differ by the inclusion of stable isotopes. Stable isotopic standards (SIS) can be incorporated into the assay at precise levels and used to quantify the corresponding unknown analyte. Additional levels of specificity are contributed by the co-elution of the unknown analyte and its corresponding SIS, and by the properties of their transitions (e.g., the similarity in the ratio of the level of two transitions of the analyte and the ratio of the two transitions of its corresponding SIS).

Accordingly, detection of a protein target by MRM-MS involves detection of one or more peptide fragments of the protein, typically through detection of a stable isotope standard peptide against which the peptide fragment is compared. Typically, an SIS will, itself, be fragmented in a collision cell as the original digested fragment, and one or more of these fragments is detected by the mass spectrometer.

Mass spectrometry assays, instruments and systems suitable for biomarker peptide analysis can include, without limitation, matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS) n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS) n; ion mobility spectrometry (IMS); inductively coupled plasma mass spectrometry (ICP-MS) atmospheric pressure photoionization mass spectrometry (APPI-MS); APPI-MS/MS; and APPI-(MS) n. Peptide ion fragmentation in tandem MS (MS/MS) arrangements can be achieved using techniques known in the art, such as, e.g., collision induced dissociation (CID). As described herein, detection and quantification of biomarkers by mass spectrometry can involve multiple reaction monitoring (MRM), such as described, inter alia, by Kuhn et al. (2004) Proteomics 4:1175-1186. Scheduled multiple-reaction-monitoring (Scheduled MRM) mode acquisition during LC-MS/MS analysis enhances the sensitivity and accuracy of peptide quantitation. Anderson and Hunter (2006) Mol. Cell. Proteomics 5 (4): 573-588. Mass spectrometry-based assays can be advantageously combined with upstream peptide or protein separation or fractionation methods, such as, for example, with the tandem column system described herein.

In another embodiment, useful in biomarker discovery, fractionated samples are analyzed by nano flow HPLC (e.g., Ultimate 3000, Thermo Fisher Scientific) followed by Thermo Orbitrap Mass Spectrometer (Tribrid Eclipse). The ion source can be a Nanospray Flex™ Ion Source (Thermo Fisher Scientific) equipped with Column Oven (PRSO-V2, Sonation) to heat up the nano column (PicoFrit, 100 μm×250 mm×15 μm tip, New Objective) for peptide separation. Peptides can be engaged on a trap column and then were delivered to the separation nano column by the mobile phase.

### V. Biomarkers

As used herein, the term “biomarker” refers to a biological molecule, the presence, form or amount of which exhibits a statistically significant difference between two states. Accordingly, biomarkers are useful, alone or in combination, for classifying a subject into one of a plurality of groups. Biomarkers may be naturally occurring or non-naturally occurring. For example, a biomarker may be a naturally occurring protein or a non-naturally occurring fragment of a protein. Fragments of a protein can function as a proxy or surrogate peptide for the protein or as stand-alone biomarkers.

Provided herein are compositions of matter comprising one or a plurality of placenta accreta biomarkers in substantially pure form. The biomarkers can be mixed in a container, or can be physically separated, for example, through attachment to solid supports at different addressable locations. As used herein, a chemical entity, such as a polynucleotide or polypeptide, is “substantially pure” if it is the predominant chemical entity of its kind in a composition. This includes the chemical entity representing more than 50%, more than 80%, more than 90% or more than 95% or of the chemical entities of its kind in the composition. A chemical entity is “essentially pure” if it represents more than 98%, more than 99%, more than 99.5%, more than 99.9%, or more than 99.99% of the chemical entities of its kind in the composition. Chemical entities which are essentially pure are also substantially pure.

### 1. Protein Biomarkers

Provided herein are one or more protein biomarkers associated with increased risk of placenta accreta. Exemplary Biomarkers for inferring placenta accreta in the second trimester are presented in Table 1 (24 weeks+/−2 weeks), Table 3 (24 weeks+/−2 weeks), and Table 5 (24 weeks+/−2 weeks). Biomarkers for inferring placenta accreta in the third trimester are presented in Table 2 (35 weeks+/−2 weeks), Table 4 (35 weeks+/−2 weeks), and Table 6 (35 weeks+/−2 weeks). Although the data collected for the biomarkers of these tables are from pregnant subject at 24 weeks+/−2 weeks gestation or 35 weeks+/−2 weeks gestation, the biomarkers may be relevant for assessment at gestational ages that fall outside this range. As discussed below, Table 7 and Table 8 present panels of biomarker for inferring placenta accreta at around 24 and around 35 weeks, respectively. In some embodiments, the one or more protein biomarkers associated with increased risk of placenta accreta include a plurality of protein biomarkers.

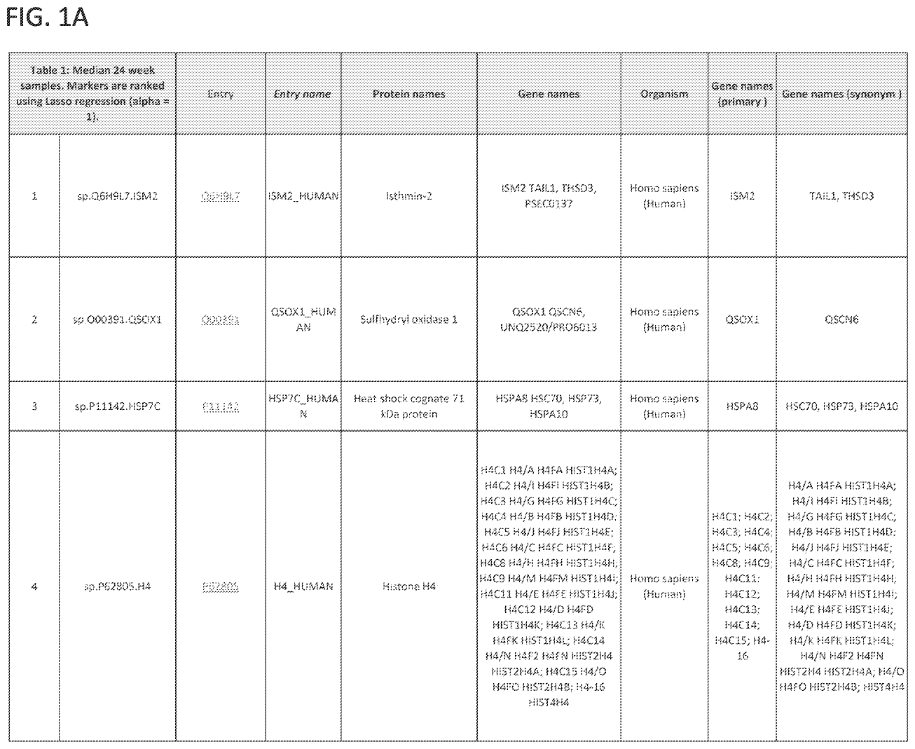

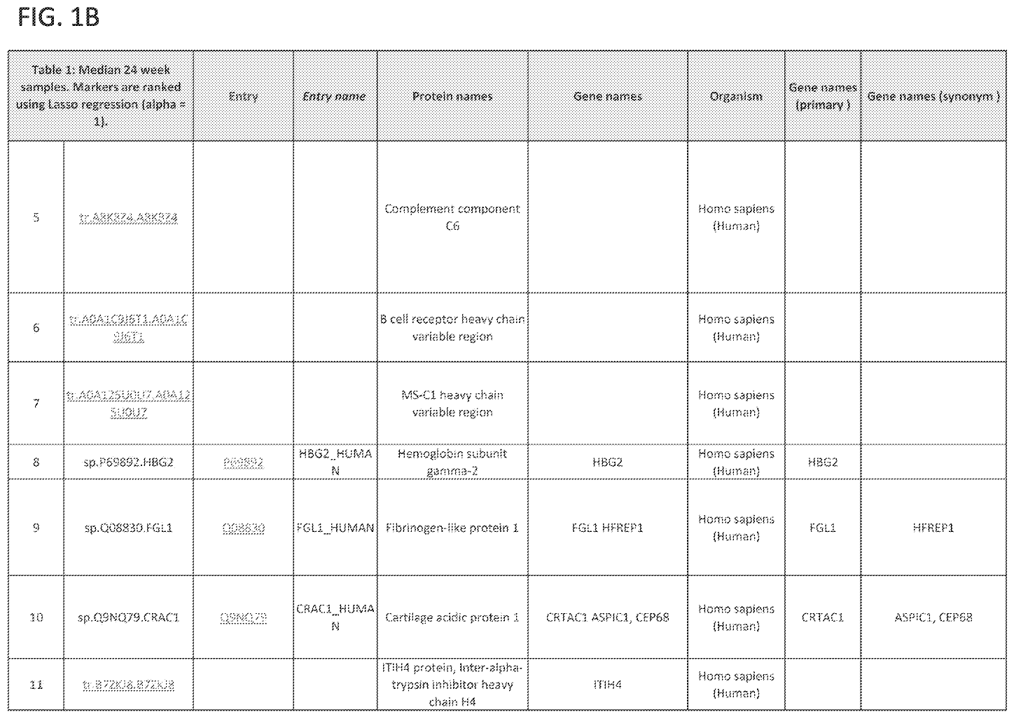

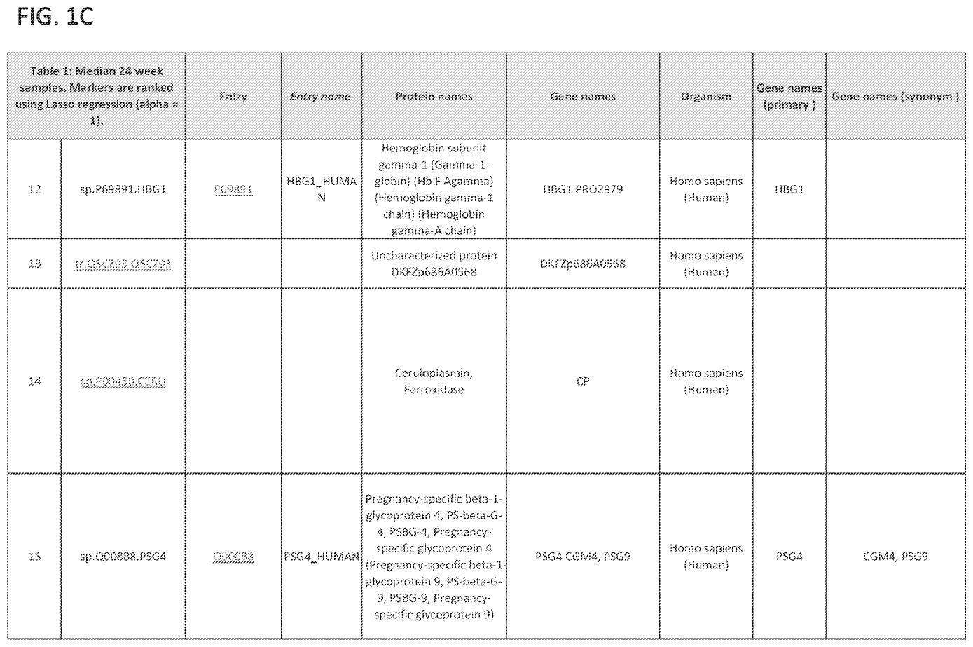

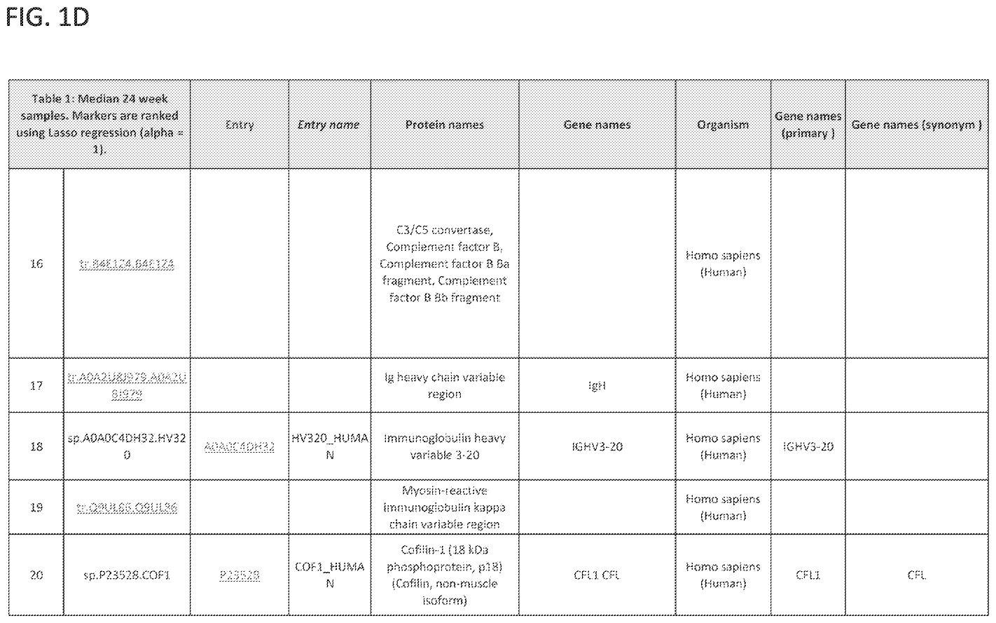

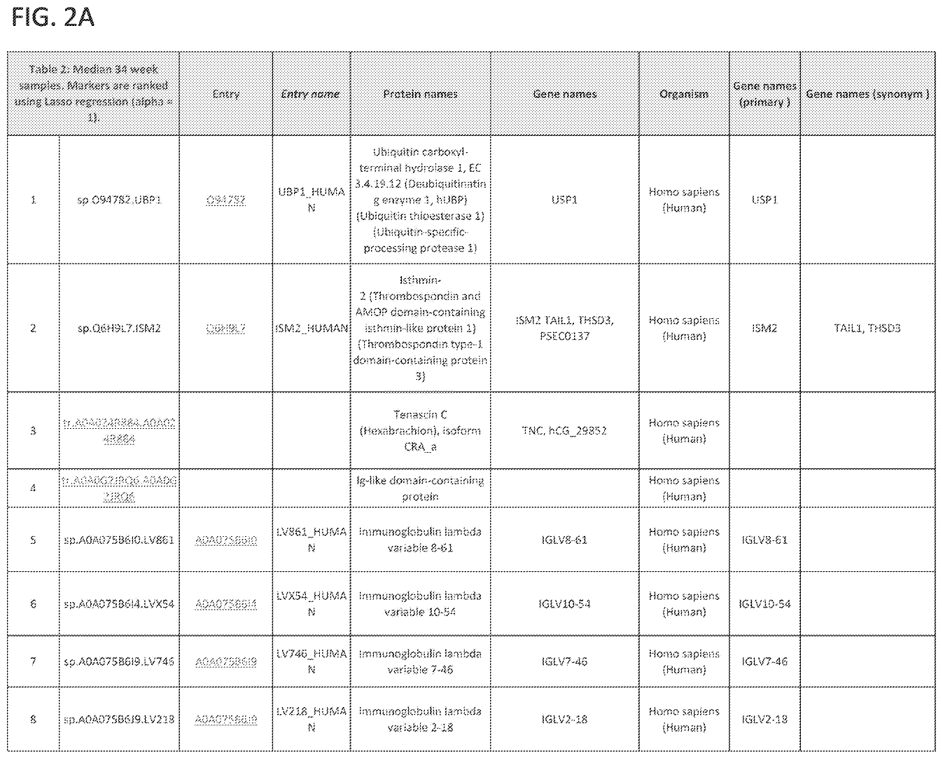

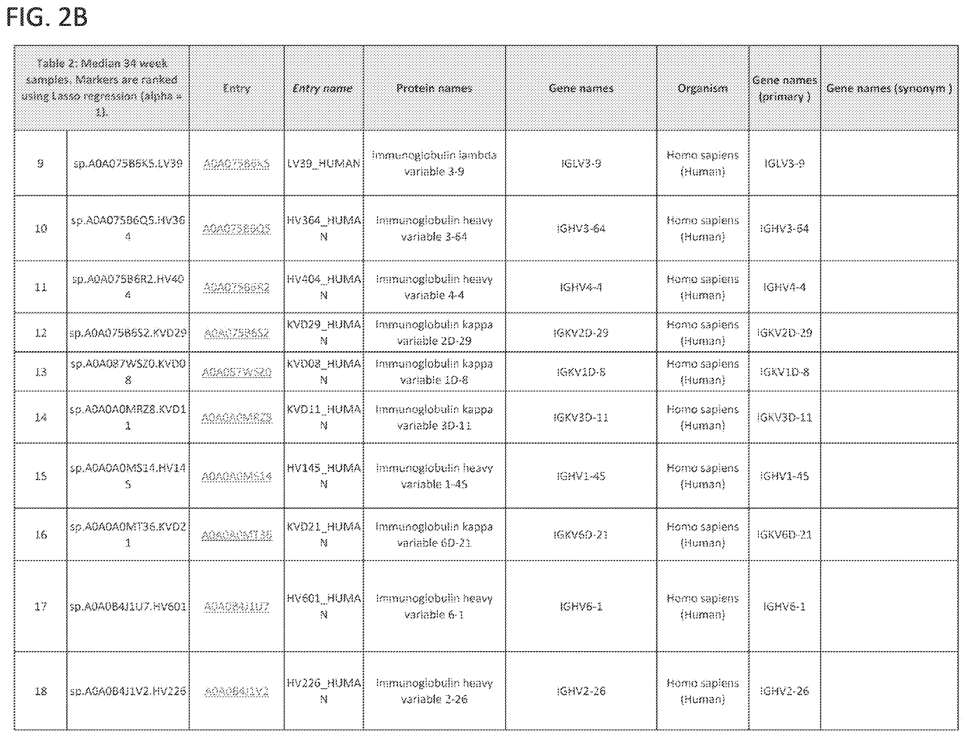

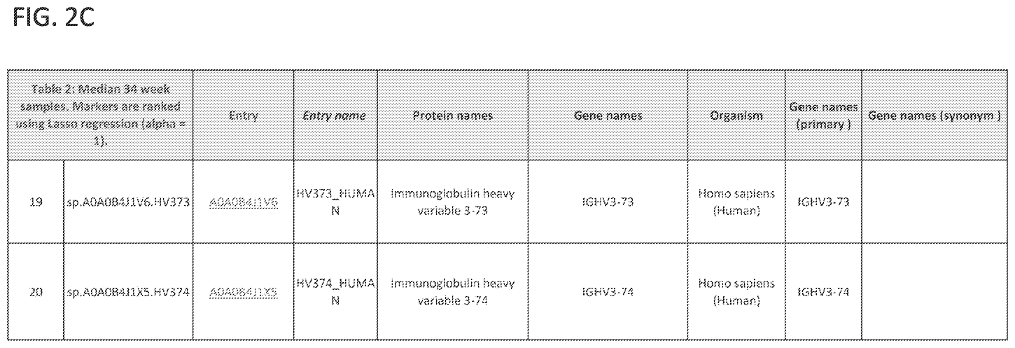

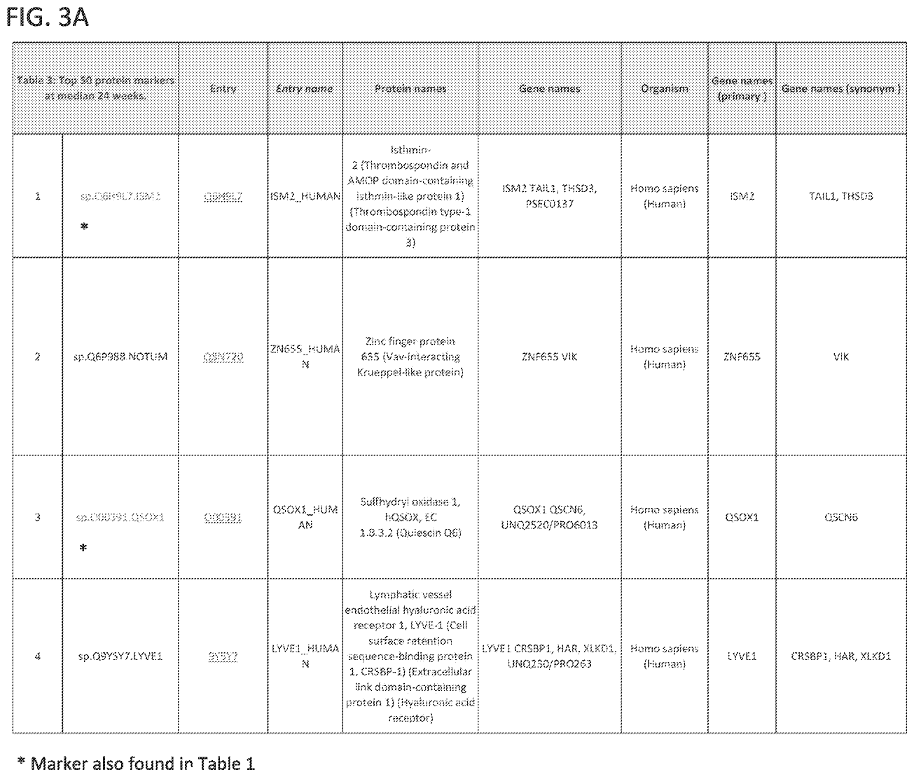

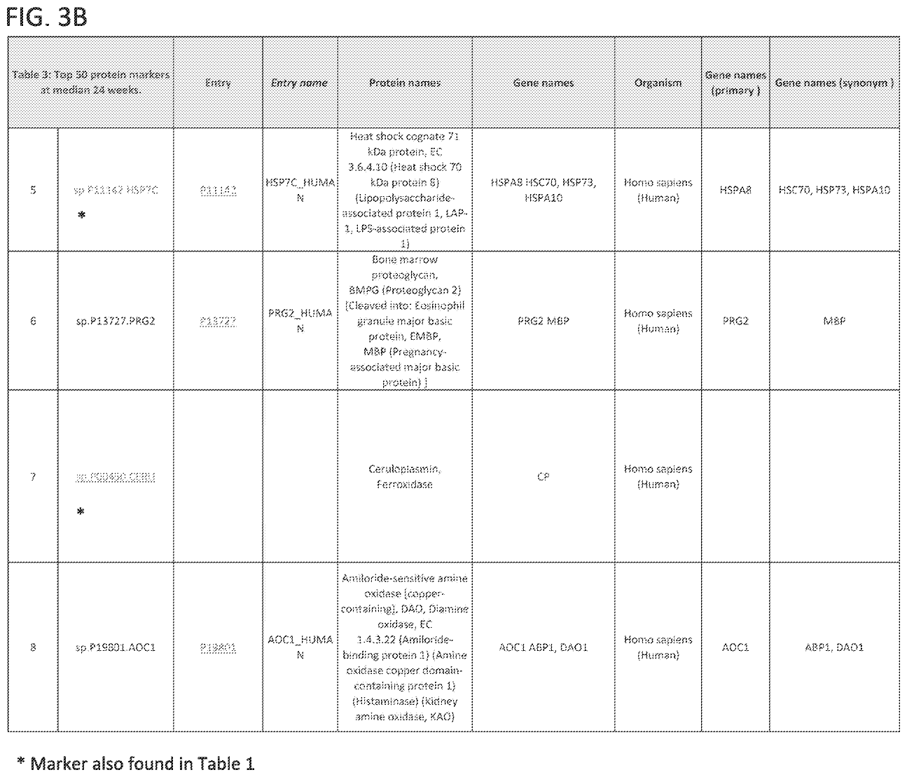

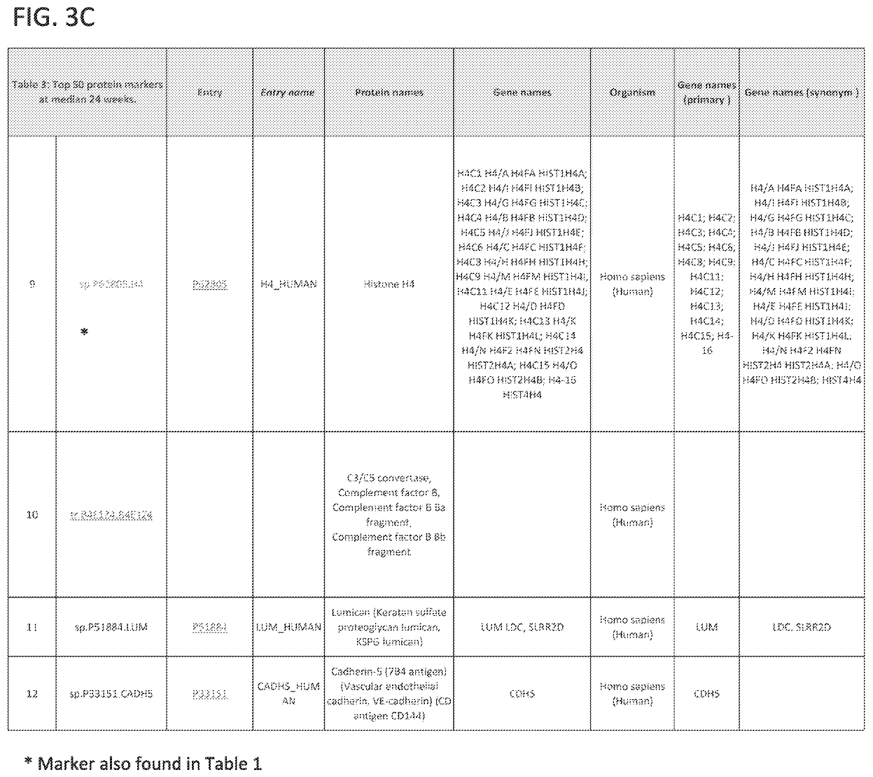

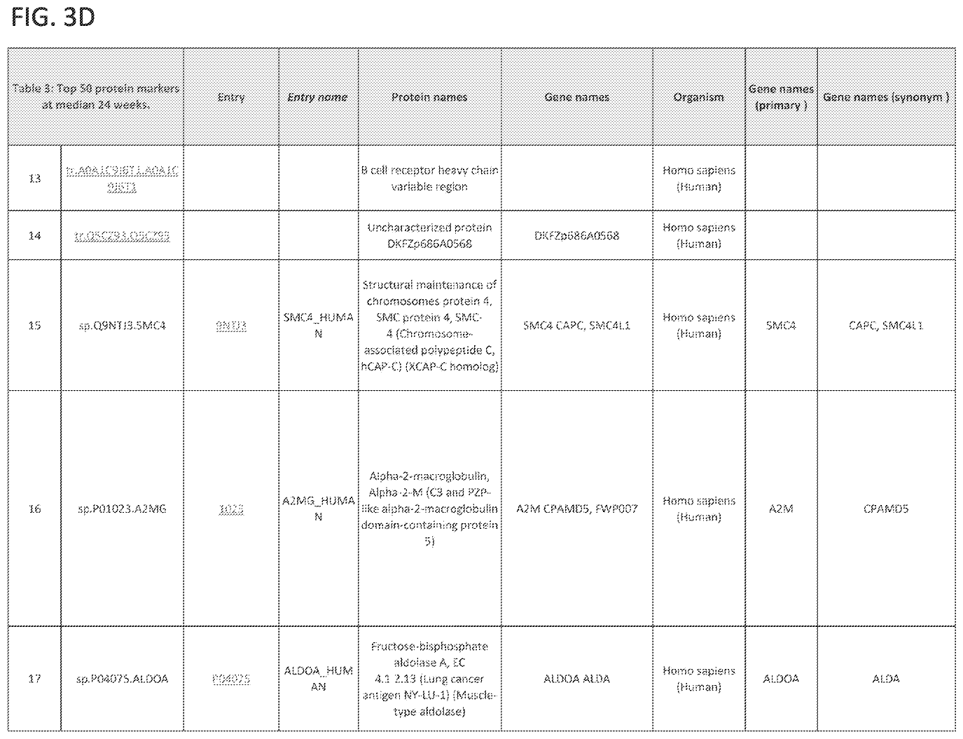

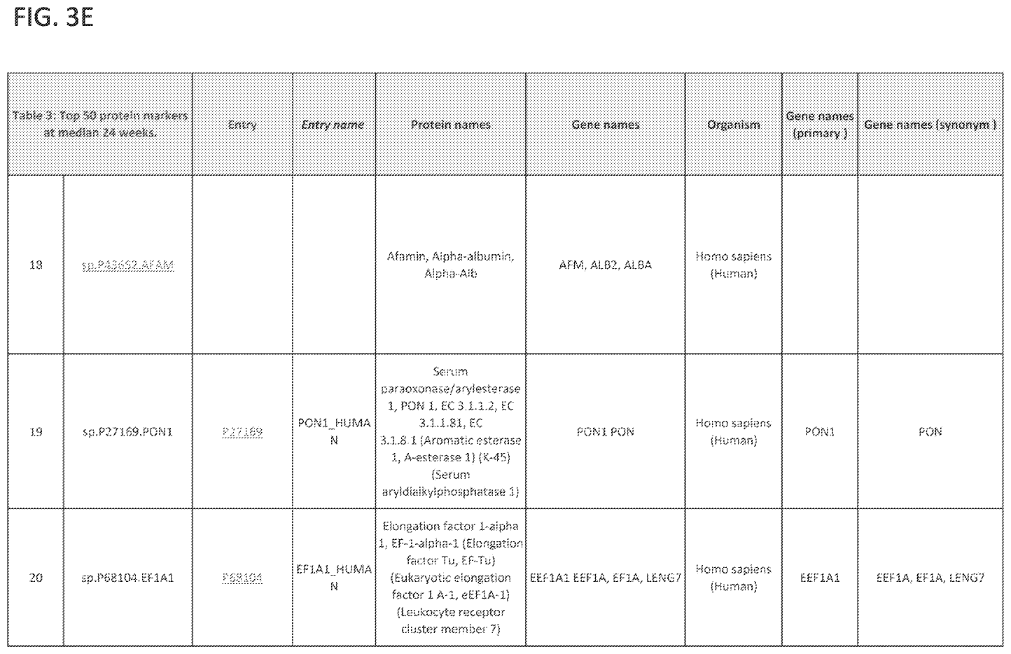

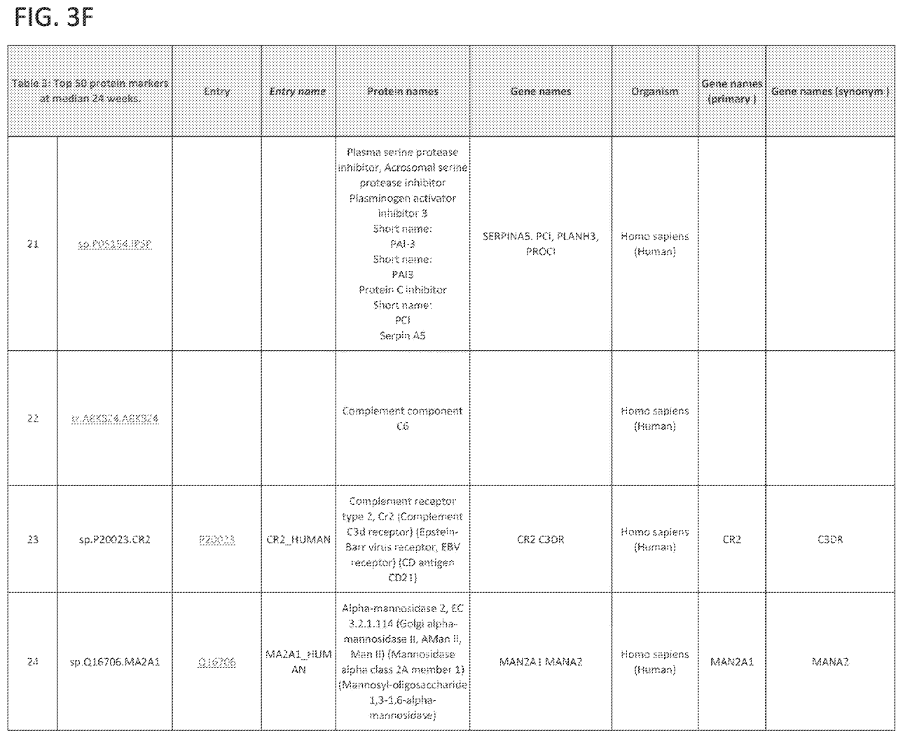

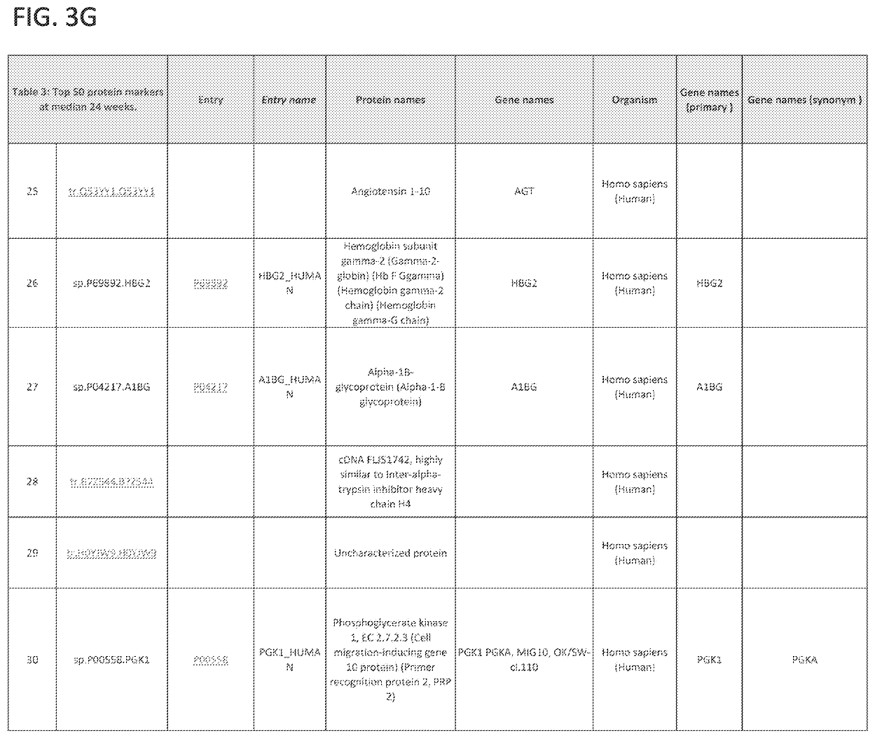

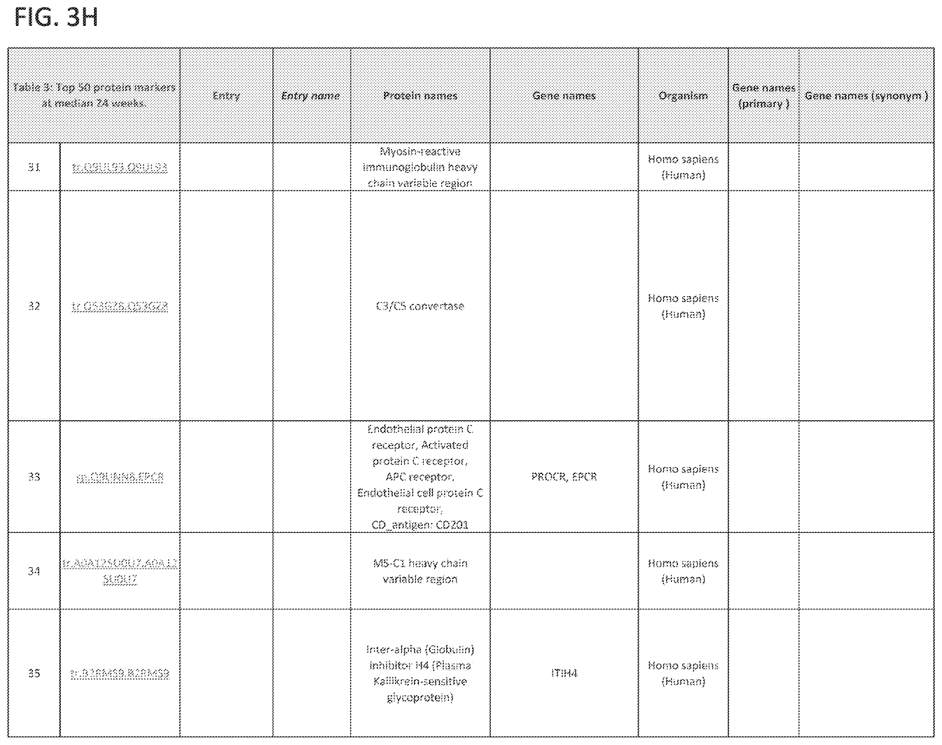

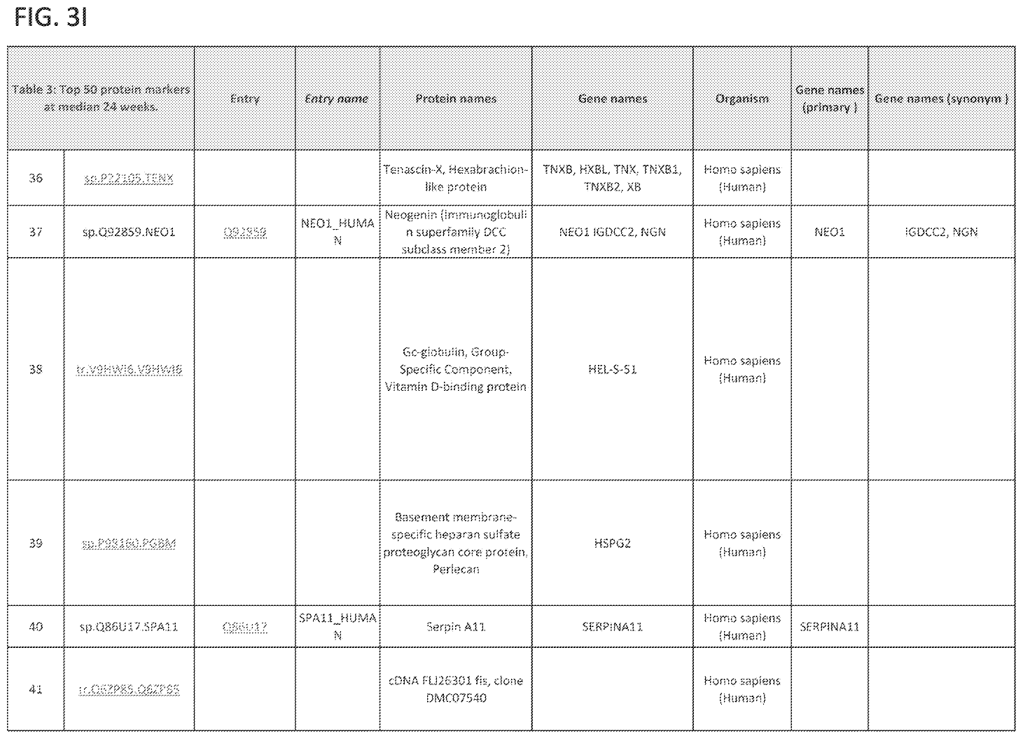

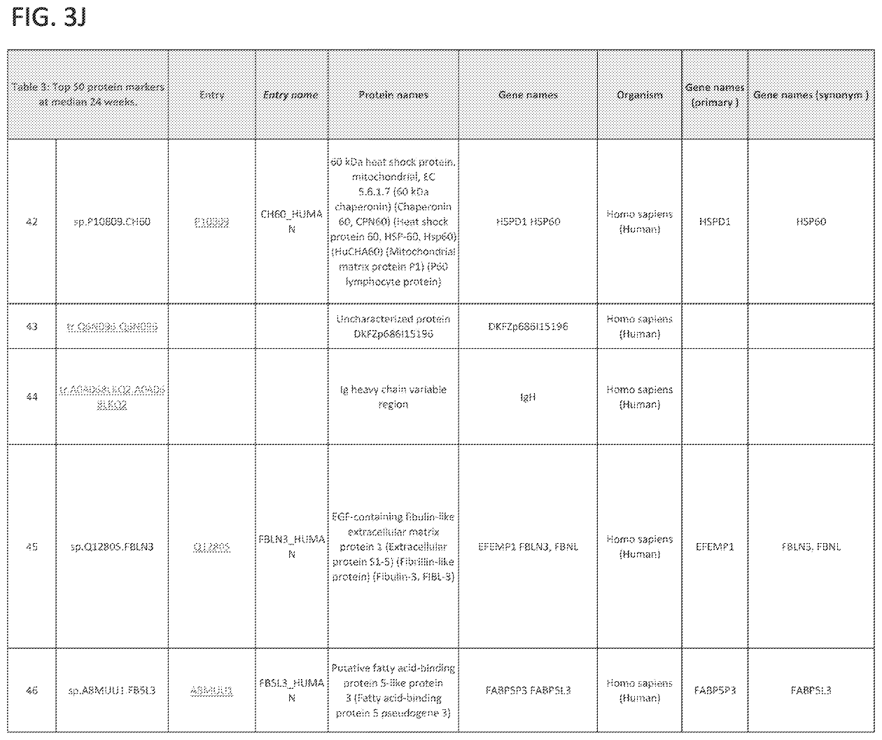

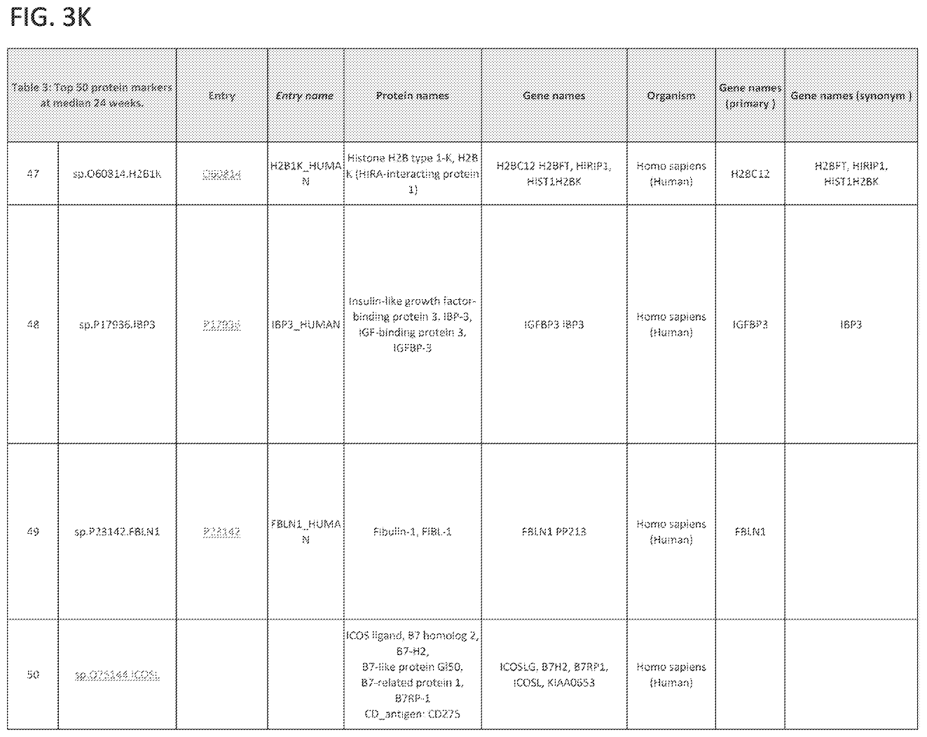

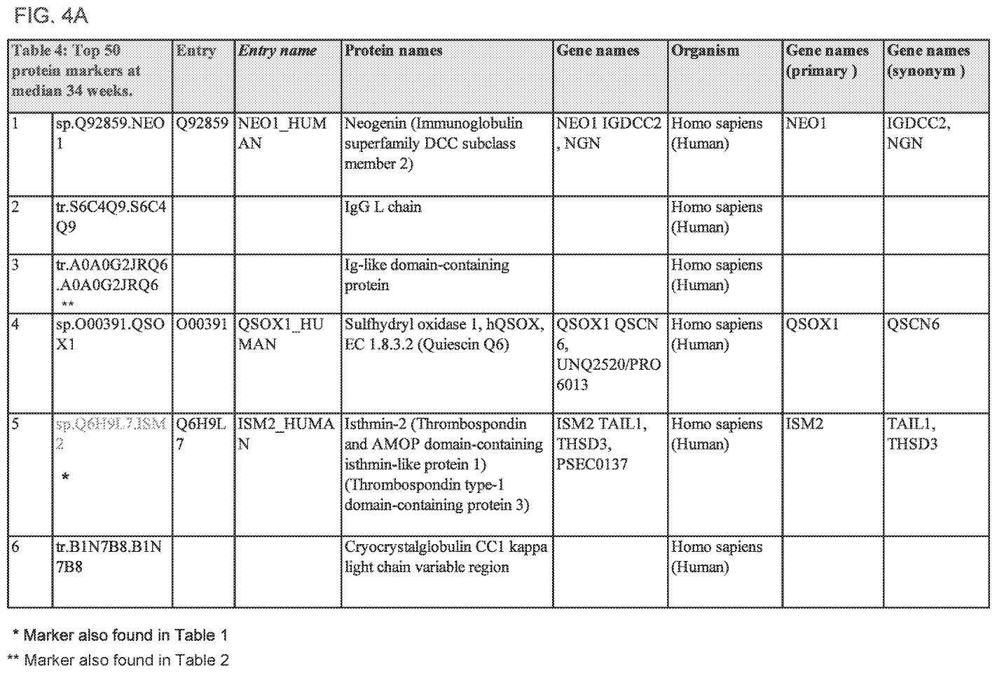

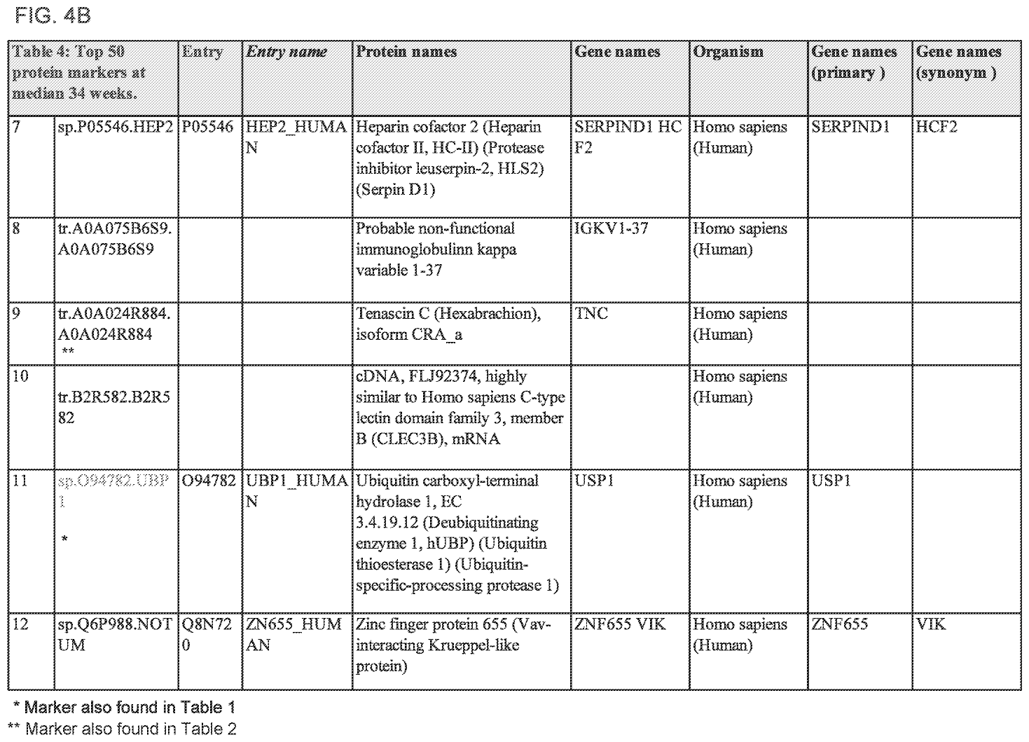

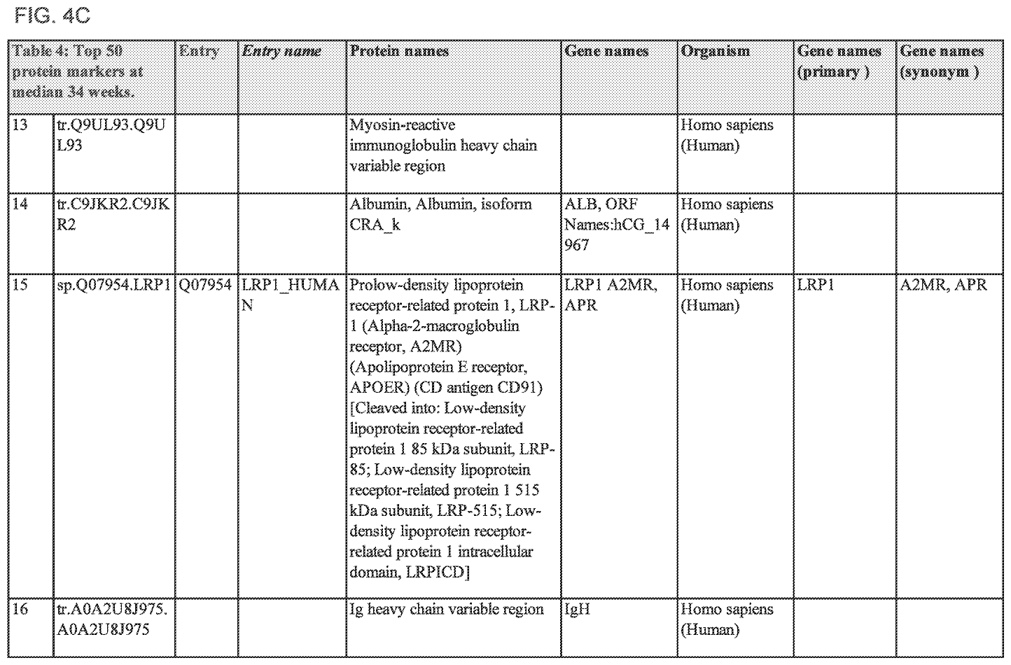

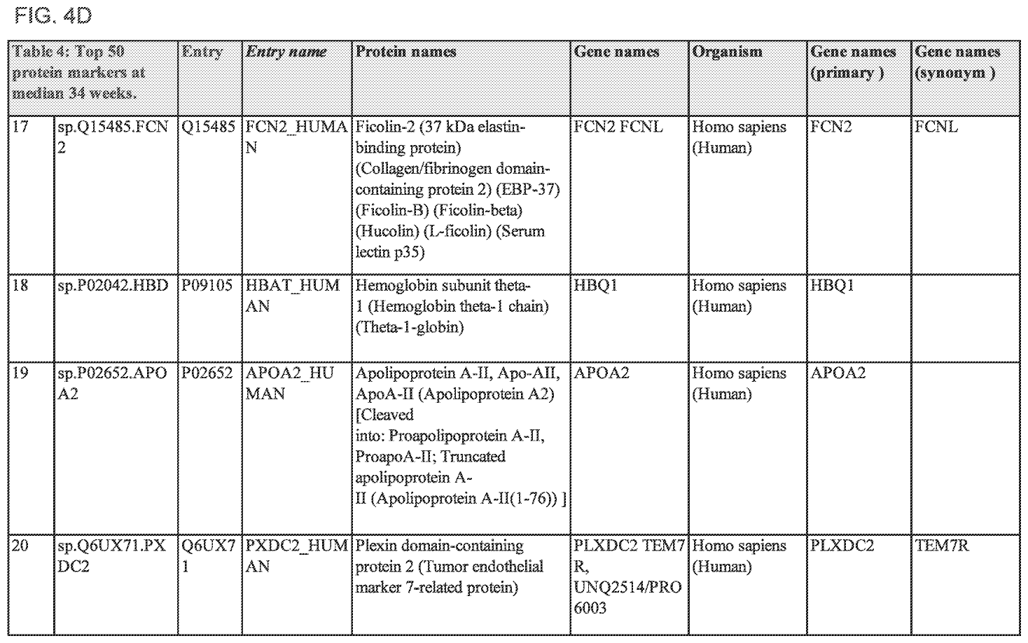

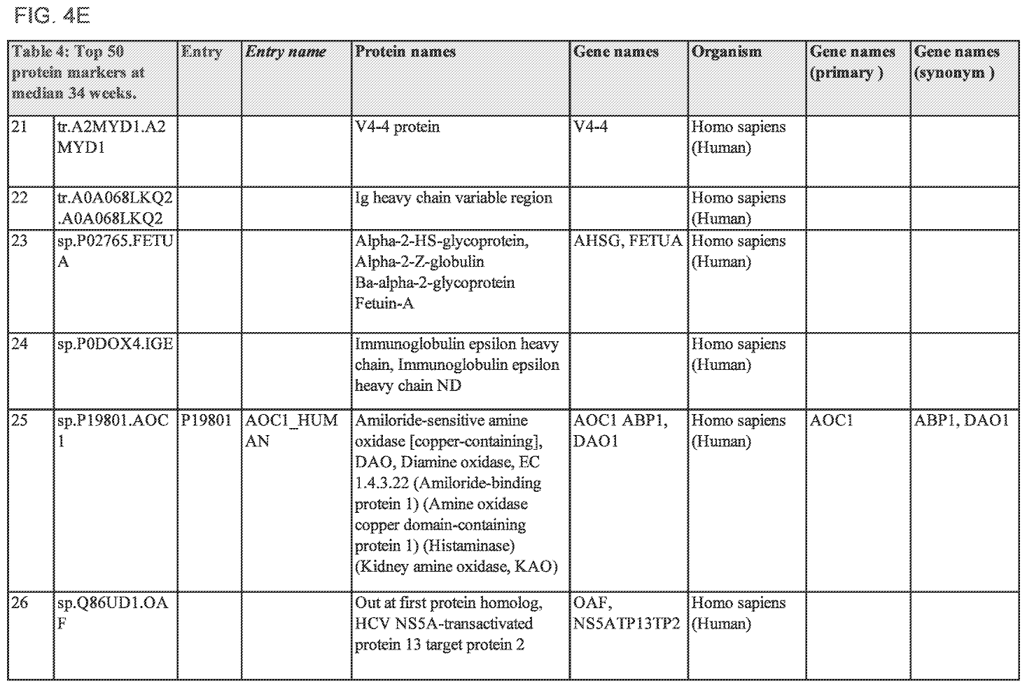

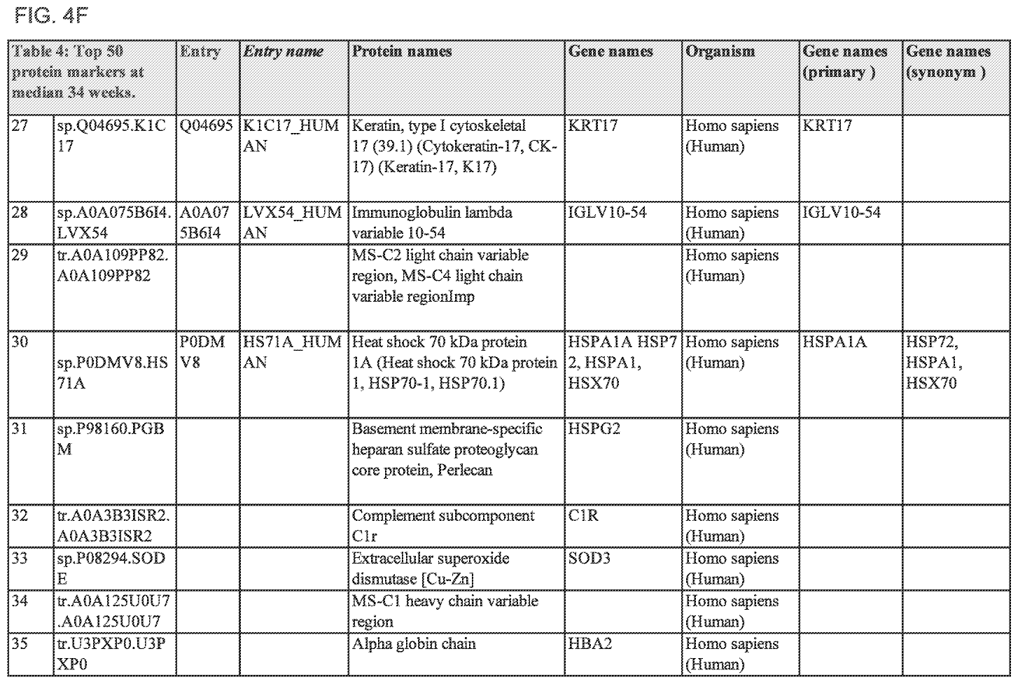

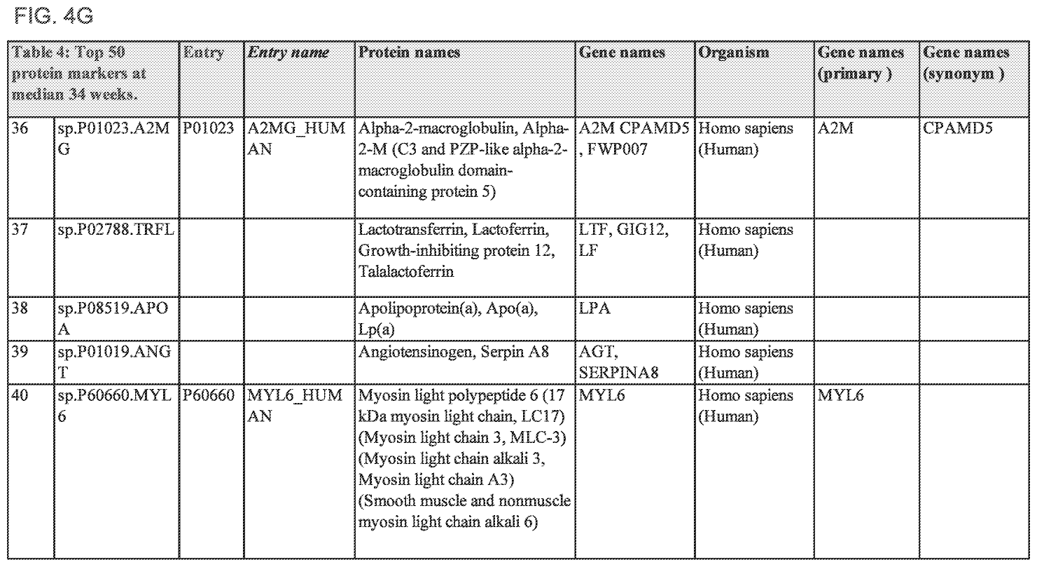

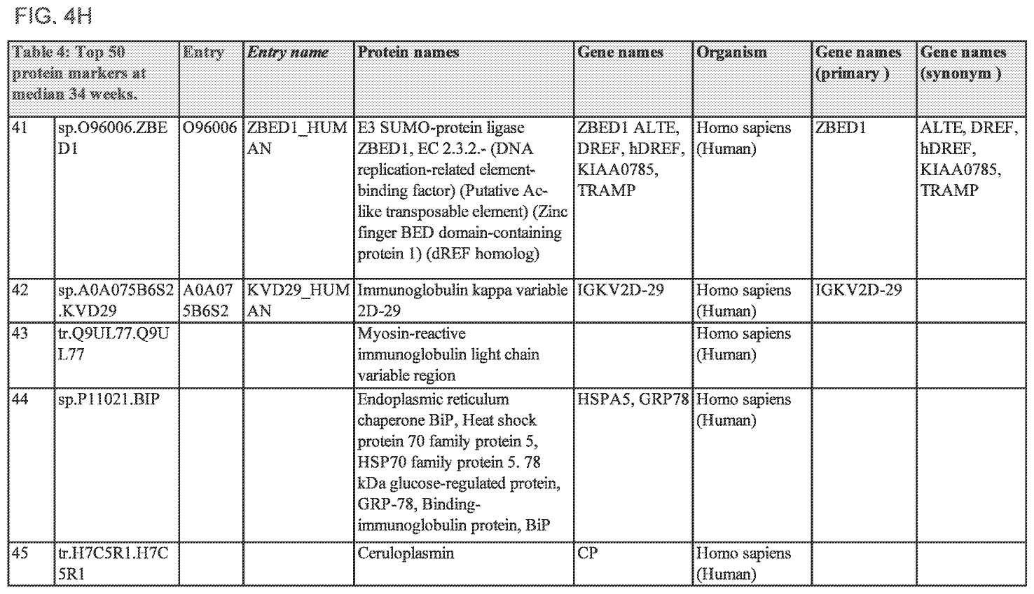

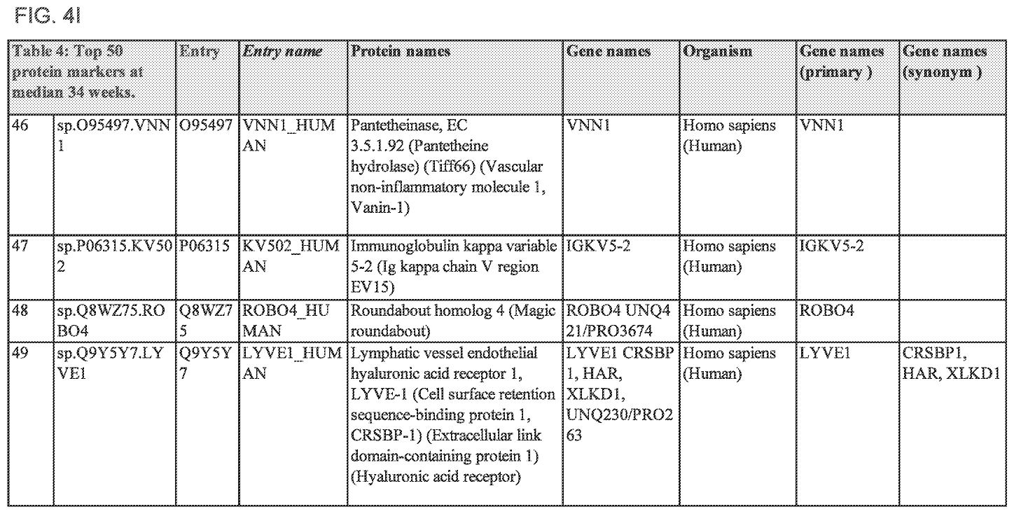

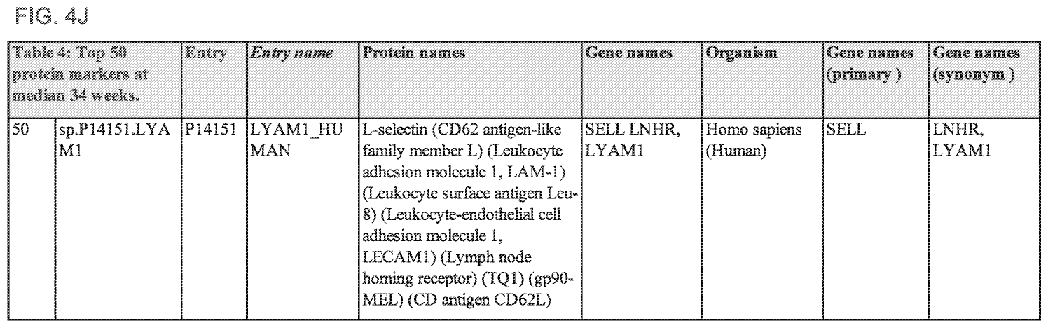

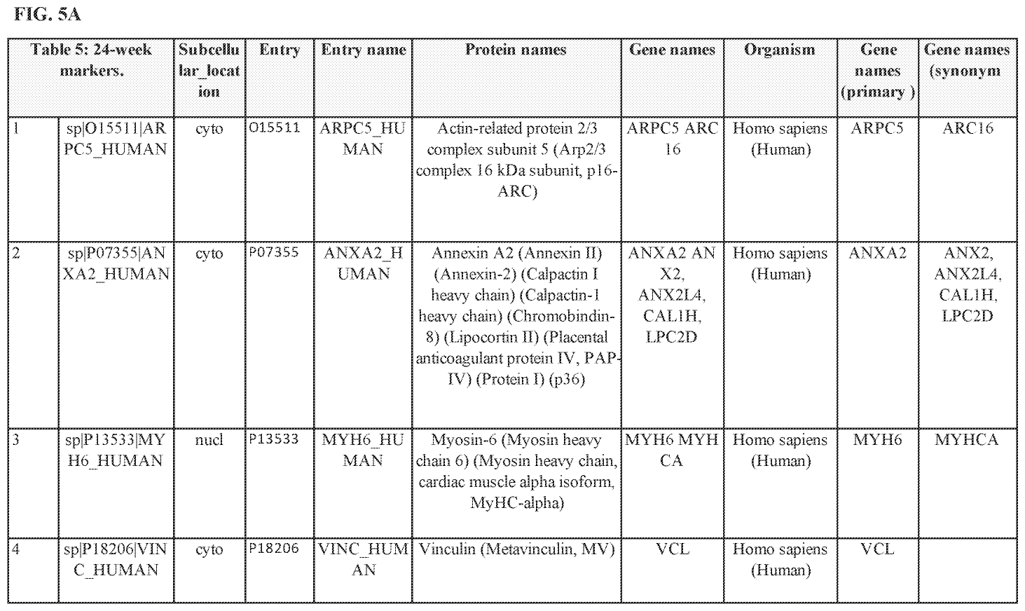

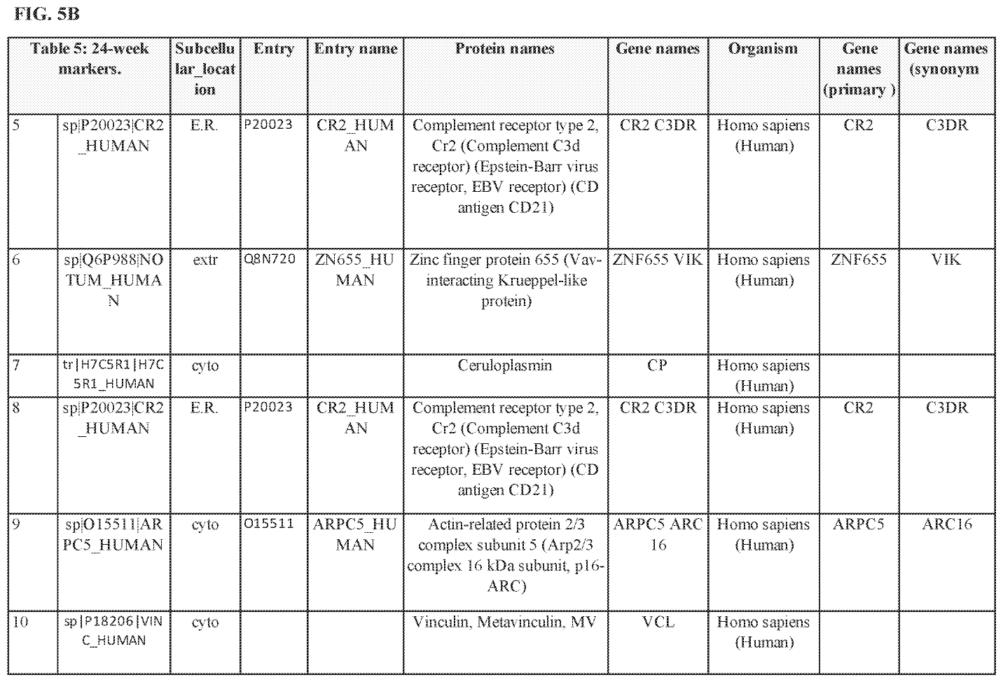

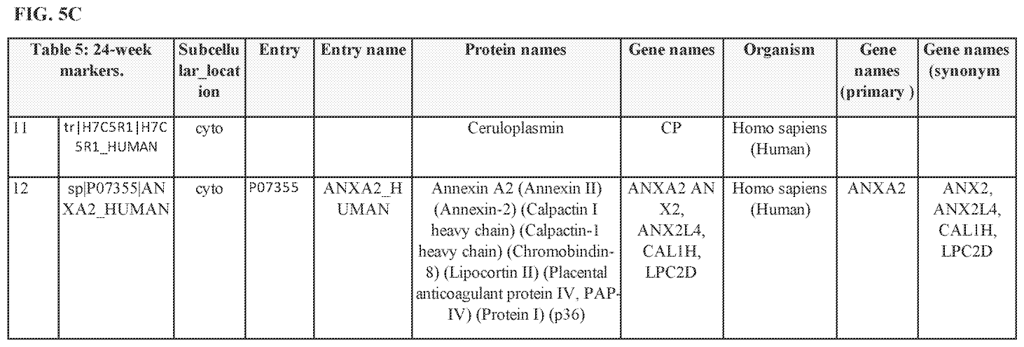

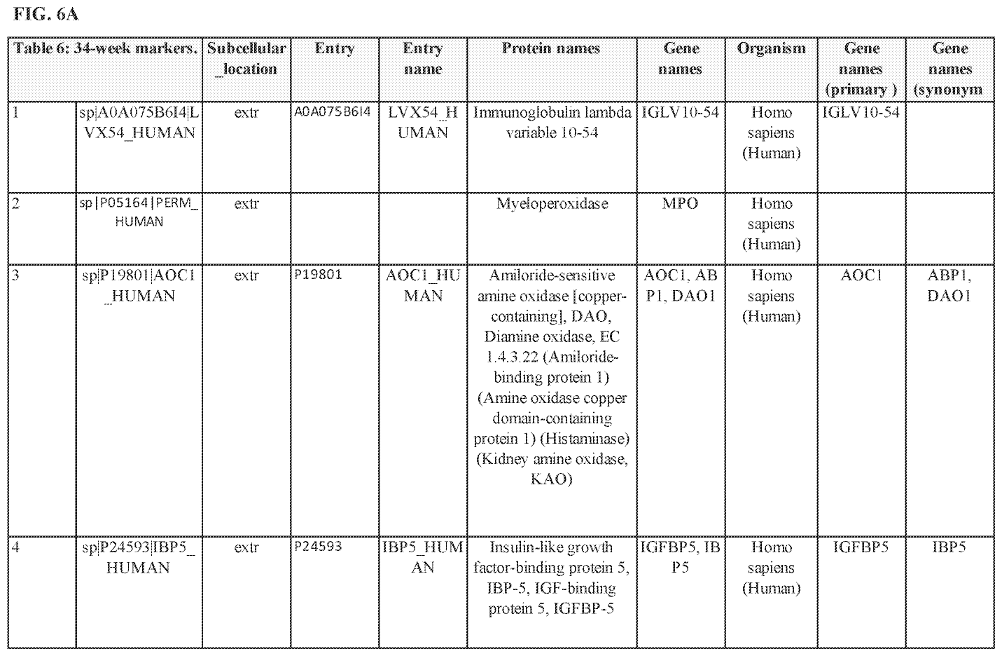

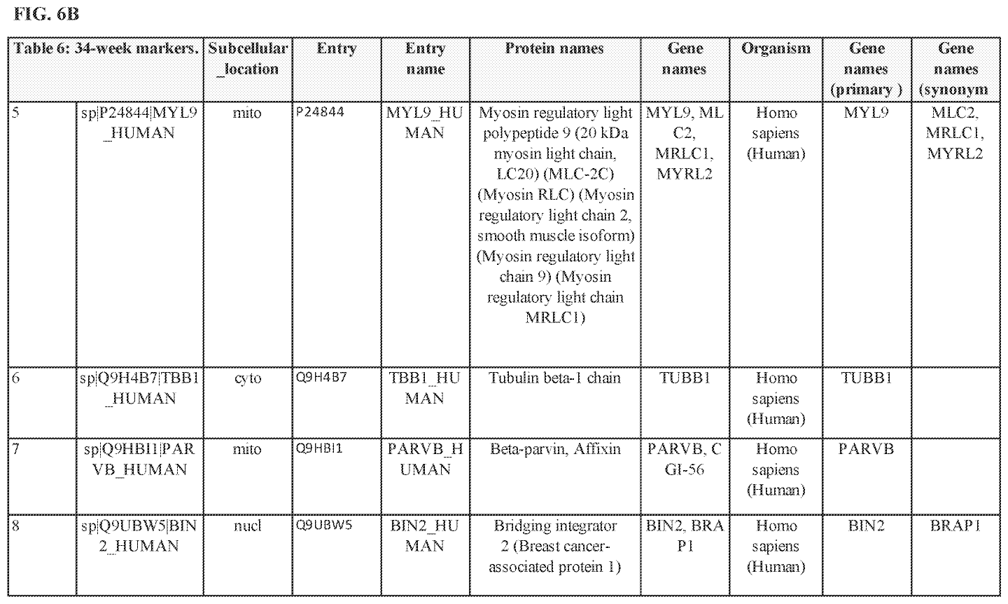

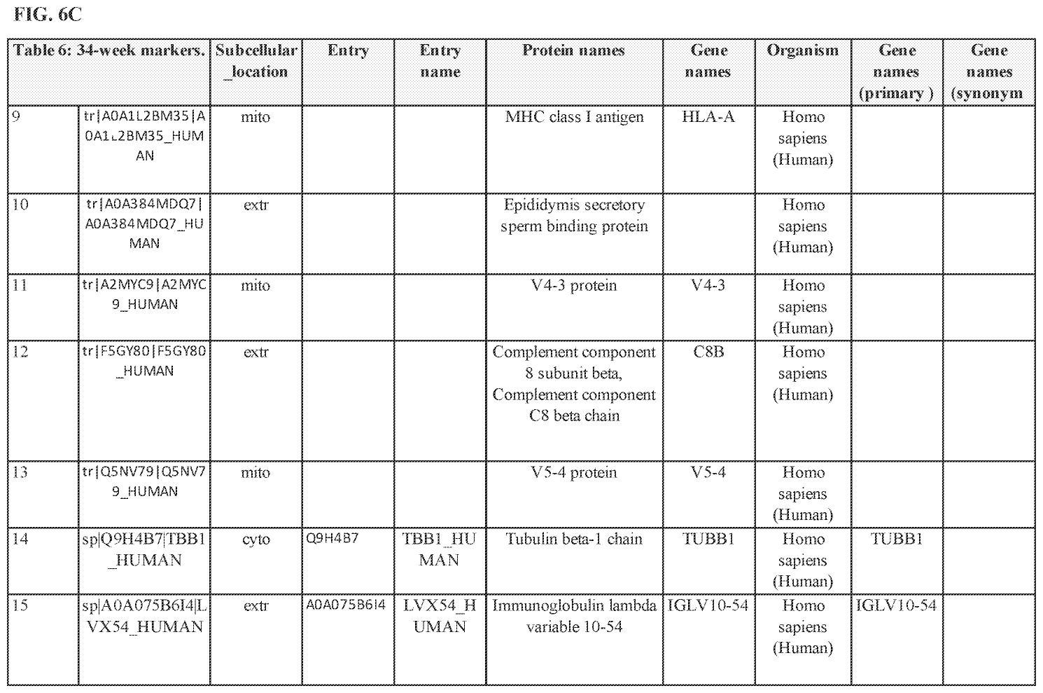

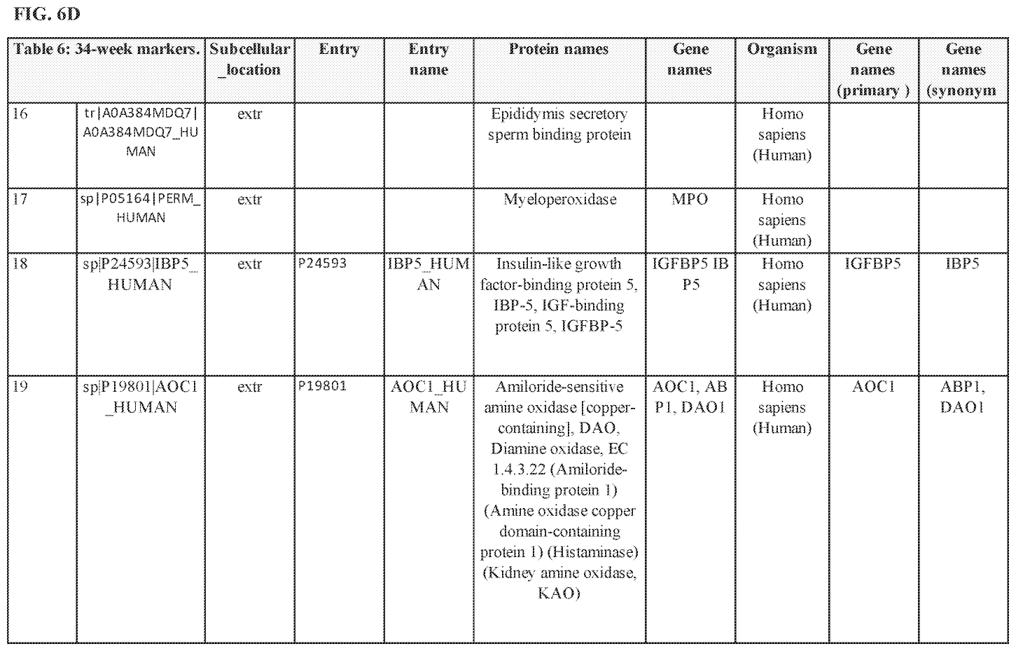

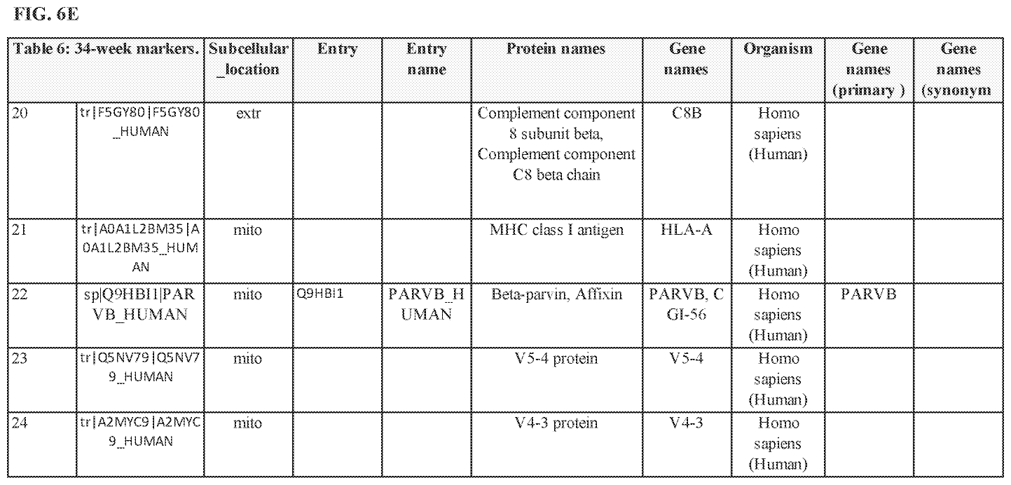

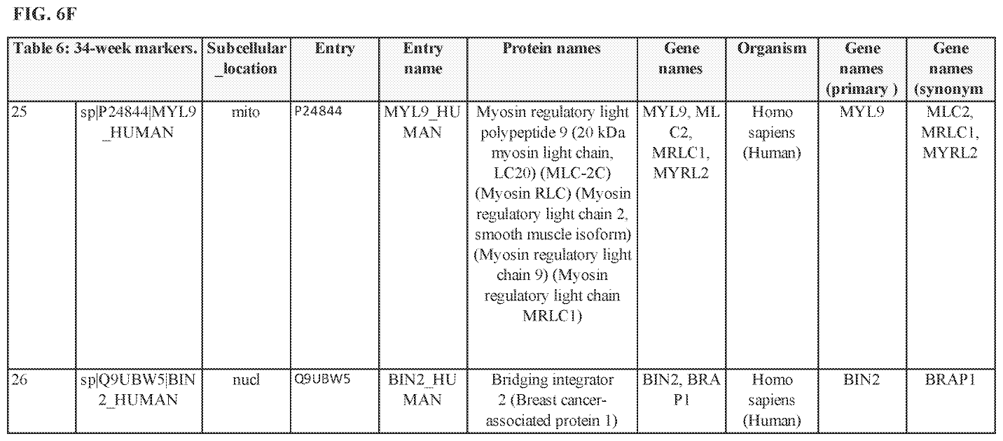

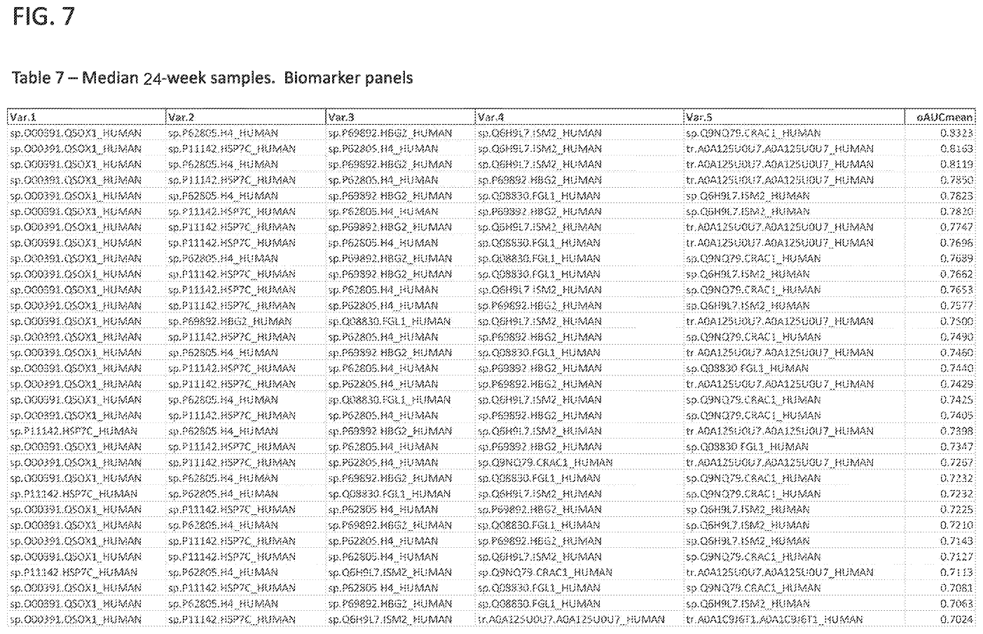

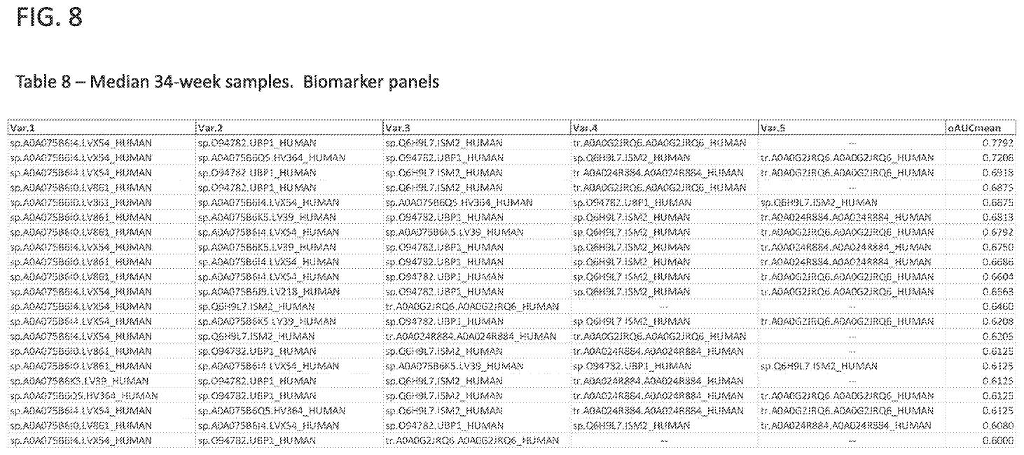

The tables in the drawings provide the UniProt entry number and entry name, protein names, gene names, organism (all Homo sapiens), primary gene names and gene name synonyms. The contents of Tables 1, 2, 3, 4, 5, 6, 7, and 8 of FIGS. 1A-1D, FIGS. 2A-2C, FIGS. 3A-3K, FIGS. 4A-4J, FIGS. 5A-5C, FIGS. 6A-6F, and FIGS. 10A-10C, FIGS. 11A-11C, FIGS. 12A-12H, FIGS. 13A-13H, FIGS. 14A-14D, FIGS. 15A-15B including their protein name, gene name (primary and synonym), and/or peptide sequence are hereby incorporated in their entirety into the detailed description as if they were provided herein. For the purpose of inclusion into the detailed description. Table 9 provides exemplary biomarkers of the disclosure, useful for the prediction of plasma accreta (protein name and synonyms provided).

TABLE 9
Exemplary biomarkers

lsthmin-2
Sulfhydryl oxidase 1
Heat shock cognate 71 kDA protein
Histone H4
Complement component C6
B cell receptor heavy chain variable region
MS-Cl heavy chain variable region
Hemoglobin subunit gamma-2
Fibrinogen-like protein 1
Cartilage acidic protein 1
ITIH4 protein, Inter-alpha-trypsin inhibitor heavy chain H4
Hemoglobin subunit gamma-1 (Gamma-1-globin) (Hb F Agamma) (Hemoglobin gamma-
1chain) (Hemoglobin gamma-A chain)
Uncharacterized protein DKFZp686A0568
Ceruloplasmin, Ferroxidase
Pregnancy-specific beta-1-glycoprotein 4, PS-beta-G-4, PSBG-4, Pregnancy-specific
glycoprotein 4
(Pregnancy-specific beta-1 glycoprotein 9, PS-beta-G-9, PSBG-9, Pregnancy specific
glycoprotein 9)
C3/CS convertase, Complement factor B, Complement factor B Ba fragment, Complement
factor B Bb fragment
lg heavy chain variable region
lmmunoglobulin heavy variable 3-20
Myosin-reactive mmunoglobulin kappa chain variable region
Cofilin-1 (18 kDa phosphoprotein, p18) (Cofilin, non-muscle isoform)
Ubiquitin carboxylterminal hydrolase 1, EC 3.4.19.12 (Deubiquitinating enzyme 1, hUBP)
Ubiquitin thioesterase 1) (Ubiquitin-specific processing protease 1)
lsthmin- 2 (Thrombospondin and AMOP domain-containing isthmin-like protein 1)
(Thrombospondin type-1 domain-containing protein 3)
Tenascin C (Hexabrachion), isoform CRA a
lg-like domain-containing protein
lmmunoglobulin lambda variable 8-61
lmmunoglobulin lambda variable variable 10-54
lmmunoglobulin lambda variable 7-46
lmmunoglobulin lambda variable 2-18
lmmunoglobulin lambda variable 3-9
lmmunoglobulin heavy variable variable 3-64
lmmunoglobulin heavy variable 4-4
lmmunoglobulin kappa variable 2D-29
lmmunoglobulin kappa variable 1D-8
lmmunoglobulin kappa variable 3D-11
lmmunoglobulin heavy variable 1-45
lmmunoglobulin kappa variable 6D-21
lmmunoglobulin heavy variable 6-1
lmmunoglobulin heavy variable 2-26
lmmunoglobulin heavy variable 3-73
lmmunoglobulin heavy variable 3-74
lsthmin-2 (Thrombospondin and AMOP domain-containing isthmin-like protein 1)
(Thrombospondin type-1 domain-containing protein 3)
Zinc finger protein 655 (Vav-interacting Krueppel-like protein)
Sulfhydryl oxidase 1 hQSOX, EC 1.8.3.2 (Quiescin Q6)
Lymphatic vessel endothelial hyaluronic acid receptor 1, LYVE-1 (Cell surface retention
sequence-binding protein 1, CRSBP-1) (Extracellular link domain-containing protein 1)
(Hyaluronic acid receptor)
Heat shock cognate 71 kDa protein, EC 3.6.4.10 (Heat shock 70 kDa protein 8)
(Lipopolysaccharide-associated protein 1, LAP-1, LPS-associated protein 1)
Bone marrow proteoglycan, BMPG (Proteoglycan 2) [Cleaved into: Eosinophil granule major
basic protein, EMBP, MBP (Pregnancy associated major basic protein)]
Ceruloplasmin, Ferroxidase
Amiloride-sensitive amine oxidase [copper containing], DAO, Diamine oxidase, EC 1.4.3.22
(Ami loride-binding protein 1) (Amine oxidase copper domain containing protein 1)
(Histaminase) (Kidney amine oxidase, KAO)
Histone H4
C3/CS convertase, Complement factor B, Complement factor B Ba fragment, Complement
factor B Bb fragment
Lumican (Keratan sulfate proteoglycan lumican, KSPG lumican)
Cad herin-5 (7B4 antigen) (Vascular endothelial cadherin, VE-cadheri n) {CD antigen
CD144)
B cell receptor heavy chain variable region
Uncharacterized protein DKFZp686A0568
Structural maintenance of chromosomes protein 4 SMC protein 4, SMC 4 (Chromosome-
associated polypeptide C, hCAP-C) (XCAP-C homolog)
Alpha-2-macroglobulin, Alpha-2-M (C3 and PZP like alpha-2-macroglobulin domain-
containing protein 5)
Fructose-bisphosphate aldolase A, EC 4.1.2.13 (Lung cancer antigen NY-LU-1) (Muscle-type
aldolase)
Afamin, Alpha-albumin Alpha-Alb
Serum paraoxonase/arylesterase 1, PON 1, EC 3.1.1.2, EC 3.1.1.81, EC 3.1.8.1 (Aromatic
esterase 1, A-esterase 1) (K-45) (Serum aryldialkylphosphatase 1)
Elongation factor 1-alpha 1, EF-1-alpha-1 (Elongation factor Tu, EF-Tu) (Eukaryotic
elongation factor 1 A-1, eEFIA-1) (Leukocyte receptor cluster member 7)
Plasma serine protease inhibitor, Acrosoma 1 serine protease inhibitor Plasminogen activator
inhibitor 3 Short name: PA1-3 Short name: PA13 Protein C inhibitor Short name: PCI Serpin AS
Complement component C6
Complement receptor type 2, Cr2 (Complement C3d receptor) (Epstein-Barr virus receptor,
EBV receptor) (CD antigen CD21)
Alpha-mannosidase 2, EC 3.2.1.114 (Golgi alphamannosidase II, AMan II, Man II)
(Mannosidase alpha class 2A member 1) (Mannosyl-o ligosaccharide 1,3-1,6-
alphamannosidase)
Angiotensin 1-10
Hemoglobin subunit gamma-2 (Gamma-2-globin) (Hb F Ggamma) (Hemoglobin gamma-2
chain) (Hemoglobin gamma-G chain)
Alpha-1B glycoprotein (Alpha-1-B glycoprotein)
cDNA FU51742, highly similar to Inter-alpha-trypsin inhibitor heavy chain H4
Uncharacterized protein
Phosphoglycerate kinase 1, EC 2.7.2.3 (Cell migration-inducing gene 10 protein) (Primer
recognition protein 2, PRP 2)
Myosin-reactive immunoglobulin heavy chain variable region
C3/CS convertase
Endothelial protein C receptor, Activated protein C receptor, APC receptor Endothelial cell
protein C receptor, CD antigen: CD201
MS-Cl heavy chain variable region
Inter-alpha (Globulin) inhibitor H4 (Plasma Kallikrein-sensitive glycoprotein)
Tenascin-X, Hexabrachion like protein
Neogenin (Immunoglobulin superfamily DCC subclass member 2)
Ge-globulin, Group-Specific Component Vitamin D-binding protein
Basement membrane-specific heparan sulfate proteoglycan core protein Perlecan
Serpin All
cDNA FLJ26301 fis, clone DMC07540
60 kDa heat shock protein, mitochondrial, EC 5.6.1.7 (60 kDa chaperonin) (Chaperonin
60, CPN60)(Heatshock protein 60, HSP-60, Hsp60 (HuCHA60) (Mitochondrial matrix protein
P1) (P60 lymphocyte protein)
Uncharacterized protein DKFZp686115196
lg heavy chain variable region
EGF-containing fibulin-like extracellular matrix protein 1 (Extracellular protein S1-5)
(Fibrillin-like protein) (Fibulin-3, FIBL-3)
Putative fatty acid-binding protein 5-like protein 3 (Fatty acid-binding protein 5 pseudogene 3)
Histone H2B type 1-K, H2B K (HIRA-interacting protein 1)
Insulin-like growth factor-binding protein 3, IBP-3, IGF-binding protein 3, IGFBP-3
Fibulin-1, FIBL-1
ICOS ligand, 87 homolog 2, B7-H2, 87-like protein GIS0, 87-related protein 1, B7RP-1
CD_antigen: CD275
Neogenin (Immunoglobulin superfamily DCC subclass member 2)
IgG L chain
lg-like domain-containing protein
Sulfhydryl oxidase 1, hQSOX EC 1.8.3.2 (Quiescin Q6)
Isthmin-2 (Thrombospondin and AMOP domain-containing isthmin-like protein 1)
(Thrombospondin type-I domain-containing protein 3)
Cryocrystalglobulin CC 1 kappa light chain variable region
Heparin cofactor 2 (Heparin cofactor II, HC-11) (Protease inhibitor leuserpin-2, HLS2)
(Serpin D1)
Probable non-functional immunoglobulinn kappa variable 1-37
Tenascin C (Hexabrachion), isoform CRA_a isoform CRA a
cDNA, FLJ92374, highly similar to Homo sapiens C-type lectin domain family 3, member B
(CLEC3B), mRNA
Ubiquitin car boxy I-terminal hydrolase 1, EC 3.4.19.12 (Deubiquitinating enzyme 1,
hUBP) (Ubiquitin thioesterase 1) (Ubiquitinspecific- processing protease 1)
Zinc finger protein 655 (Vav-interacting Krueppel-like protein)
Myosin-reactive immunoglobulin heavy chain variable region
Albumin, Albumin, isoform CRA k
Prolow-density lipoprotein receptor-related protein 1, LRP-1 (Alpha-2-macroglobulin
receptor, A2MR) (Apolipoprotein E receptor, APOER) (CD antigen CD91) [Cleaved into:
Low-density lipoprotein receptor-related protein 1 85 kDa subunit, LRP-85; Low-density
lipoprotein receptor-related protein 1 515 kDa subunit, LRP-515; Low density lipoprotein
receptor related protein 1 intracellular domain, LRPICD]
lg heavy chain variable region
Ficolin-2 (37 kDa elastin-binding protein) (Collagen/fibrinogen domain containing protein 2)
(EBP-37) (Ficolin-B) (Ficolin-beta) (Hucolin) (L-ficolin) (Serum lectin p35)
Hemoglobin subunit theta-1 (Hemoglobin theta-I chain) (Theta-1-globin)
Apolipoprotein A-II, Apo-All, ApoA-11 (Apolipoprotein A2) [Cleaved into:
Proapolipoprotein A-II, ProapoA-11; Truncated apolipoprotein A-II (Apolipoprotein A-11(1-76))]
Plexin domain-containing protein 2 (Tumor endothelial marker 7-related protein)
V 4-4 protein
lg heavy chain variable region
Alpha-2-HS-glycoprotein, Alpha-2-Z-globulin Ba-alpha-2-glycoprotein Fetuin-A
Immunoglobulin epsilon heavy chain, Immunoglobulin epsilon heavy chain ND
Amiloride-sensitive amine oxidase [copper-containing], DAO, Diamine oxidase, EC
1.4.3.22 (Amiloride-binding protein 1) (Amine oxidase copper domain-containing
protein 1) (Histaminase) (Kidney amine oxidase, KAO)
Out at first protein homolog, HCV NS5A-transactivated protein 13 target protein 2
Keratin, type I cytoskeletal 17 (39.1) (Cytokeratin-17, CK-17) (Keratin-17, K17)
Immunoglobulin lambda variable 10-54
MS-C2 light chain variable region, MS-C4 light chain variable regionlmp
Heat shock 70 kDa protein IA (Heat shock 70 kDa protein 1, HSP70-1, HSP70.1)
Basement membrane-specific heparan sulfate proteoglycan core protein Perlecan
Complement subcomponent Clr
Extracellular superoxide dismutase [Cu—Zn]
MS-Cl heavy chain variable region
Alpha globin chain
Alpha-2-macroglobulin, Alpha-2-M (C3 and PZP-like alpha-2-macroglobulin domain
containing protein 5)
Lactotransferrin, Lactoferrin, Growth-inhibiting protein 12, Talalactoferrin
Apolipoprotein(a), Apo(a), Lp(a)
Angiotensinogen, Serpin A8
Myosin light polypeptide 6 (17 kDa myosin light chain, LCl 7) (Myosin light chain 3, MLC-3)
(Myosin light chain alkali 3, Myosin light chain A3)
(Smooth muscle and nonmuscle myosin light chain alkali 6)
E3 SUMO-protein ligase ZBEDI, EC 2.3.2.- (DNA replication-related element-binding factor)
(Putative Ac-like transposable element) (Zinc finger BED domain-containing protein 1)
(dREF homo log)
Immunoglobulin kappa variable 2D-29
Myosin-reactive immunoglobulin light chain variable region
Endoplasmic reticulum chaperone BiP, Heat shock protein 70 family protein 5, HSP70 family
protein 5.78 kDa glucose-regulated protein, GRP-78, Bindingimmunoglobulin protein, BiP
Ceruloplasmin
Pantetheinase, EC 3.5.1.92 (Pantetheine hydrolase) (Tiff66) (Vascular non-inflammatory
molecule 1, Vanin-1)
Immunoglobulin kappa variable 5-2 (1g kappa chain V region EV15)
Roundabout homolog 4 (Magic roundabout)
Lymphatic vessel endothelial hyaluronic acid receptor 1, LYVE-1 (Cell surface retention
sequence-binding protein 1, CRSBP-1) (Extracellular link domain-containing protein 1)
(Hyaluronic acid receptor)
L-selectin (CD62 antigen-like family member L) (Leukocyte adhesion molecule 1, LAM-1)
(Leukocyte surface antigen Leu-8) (Leukocyte-endothelial cell adhesion molecule 1,
LECAM 1) (Lymph node homing receptor) (TQ 1) (gp90- MEL) (CD antigen CD62L)
Actin-related protein 2/3 complex subunit 5 (Arp2/3 complex 16 kDa subunit, p16-ARC)
Annexin A2 (Annexin II) (Annexin-2( (Calpactin I heavy chain) (Calpactin-1 hevay chain)
(Chromobindin-8) (Lipocortin II) (Placental anticoagulant protein IV, PAP-IV) (Protein I)
(p36)
Myosin-6 (Myosin heavy chain 6) (Myosin heavy chain, cardiac muscle alpha isoform,
MyHC-alpha)
Vinculin (Metavinculin, MV)
Complement receptor type 2, Cr2 (Complement C3d receptor) (Epstein-Barr virus receptor, EBV
receptor) (CD antigen CD21)
Zinc finger protein 655 (Vav-interacting Krueppel-like protein)
Ceruloplasmin
Complement receptor type 2, Cr2 (Complement C3d receptor) (Epstein-Barr virus receptor,
EBV receptor) (CD antigen CD21)
Actin-related protein 2/3 complex subunit 5 (Arp2/3 complex 16 kDa subunit, p16-ARC)
Vinculin (Metavinculin, MV)
Ceruloplasmin
Annexin A2 (Annexin II) (Annexin-2( (Calpactin I heavy chain) (Calpactin-1 hevay chain)
(Chromobindin-8) (Lipocortin II) (Placental anticoagulant protein IV, PAP-IV) (Protein I)
(p36)
Immunoglobulin lambda variable 10-54
Myeloperoxidase
Amiloride-sensitive amine oxidase [copper-containing], DAO, Diamine oxidase, EC
1.4.3.22 (Amiloride-binding protein 1) (Amine oxidase copper domain-containing protein 1)
(Histaminase) (Kidney amine oxidase, KAO)
Insulin-like growth factor-binding protein 5, IBP-5, IGF-binding protein 5, IGFBP-5
Myosin regulatory light polypeptide 9 (20 kDA myosin light chain, LC20) (MLC-2C)
(Myosin RLC) (Myosin regulatory light chain 2, smooth muscle isoform) (Myosin regularoty
light chain 9) (myosin regulatory light chain MRLC1)
Tubulin beta-1 chain
Beta-parvin, Affixin
Bridging integrator 2 (Breast cancer-associated protein 1)
MHC class I antigen
Epididymis secretory sperm binding protein
V4-3 protein
Complement component 8 subunit beta, complement component c8 beta chain
V5-4 protein
Tubulin beta-1 chain
Immunoglobulin lambda variable 10-54
Epididymis secretory sperm binding protein
Myeloperoxidase
Insulin-like growth factor binding protein 5, IBP-5, IGF-binding protein 5, IGFBP-5
Amiloride-sensitive amine oxidase [copper-containing], DAO, Diamine oxidase, EC
1.4.3.22 (Amiloride-binding protein 1) (Amine oxidase copper domain-containing
protein 1) (Histaminase) (Kidney amine oxidase, KAO)
Complement component 8 subunit beta, complement component c8 beta chain
MHC class I antigen
Beta-parvin, Affixin
V5-4 protein
V4-3 protein
Myosin regulatory light polypeptide 9 (20 kDA myosin light chain, LC20) (MLC-2C)
(Myosin RLC) (Myosin regulatory light chain 2, smooth muscle isoform) (Myosin regularoty
light chain 9) (myosin regulatory light chain MRLC1)
Bridging integrator 2 (Breast cancer-associated protein 1)

Also provided for each biomarker is one or more peptide fragments from the protein that function as surrogate markers. A surrogate marker can be used as a measure of the protein for purposes of the models described herein. Accordingly, in some embodiments, the detection of one or more peptide fragments of a protein biomarker serves to detect the protein biomarker. Peptides useful as surrogates for biomarkers are presented in FIGS. 10A-10C, FIGS. 11A-11C, FIGS. 12A-12H, FIGS. 13A-13H, FIGS. 14A-14D, and FIGS. 15A-15B.

The biomarkers can be detected using de novo sequencing of proteins from microparticles isolated from a sample (e.g., blood) taken from a pregnant woman. Proteins can be sequenced by mass spectrometry, e.g., single or double (MS/MS) mass spectrometry. Both parent proteins (such as those provided in Tables 1-6, and Table 9; or the panels of Tables 7 and 8) and peptide fragments of the parent proteins (such as those described above in FIGS. 10A-10C, FIGS. 11A-11C, FIGS. 12A-12H, FIGS. 13A-13H, FIGS. 14A-14D, and FIGS. 15A-15B) are useful as biomarkers of placenta accreta. Accordingly, in some embodiments, detection of a named protein biomarker encompasses detection by a surrogate, e.g., one or more fragments of the protein.

Proteins, e.g., peptides, detected by mass spectrometry are analyzed to identify those that are up-regulated (increased in amounts) or down-regulated (decreased in amounts) compared with controls. Proteins showing statistically significant differential expression are further analyzed to identify the parent protein. Such proteins can be identified in a protein database such as SwissProt.

In certain embodiments, biomarkers are in a composition in which the peptide biomarker is paired with a stable isotopic standard of the peptide. In some embodiments, the composition may include one pair of a peptide biomarker and stable isotopic standard of the peptide or a plurality of pairs with each pair comprising a peptide biomarker and a stable isotopic standard of the peptide. The peptide biomarker may include a surrogate biomarker as is described in more detail herein. Such compositions are useful for detection in multiple reaction monitoring mass spectrometry.

For purposes of mass spectrometry, proteins can be detected intact, or through fragmentation, e.g., LCMS or in multiple reaction monitoring (MRM). In such cases, proteins can be fragmented proteolytically before analysis. Proteolytic fragmentation includes both chemical and enzymatic fragmentation. Chemical fragmentation includes, for example, treatment with cyanogen bromide. Enzymatic fragmentation includes, for example, digestion with proteases such as trypsin, chymotrypsin, LysC, ArgC, GluC, LysN and AspN. Detection of these protein fragments, or fragmented forms of them produced in mass spectrometry, can function as surrogates for the full protein.

### 2. Biomarker Panels

In certain embodiments, biomarkers are analyzed as a panel. A panel is a plurality of biomarkers used in an algorithm to make a prediction or inference. As used herein, a panel comprising a group of identified biomarkers includes at least those identified biomarkers. A panel consisting of a group of identified biomarkers includes only the identified biomarkers. A panel consisting essentially of a group of identified biomarkers includes the identified biomarkers and no more than one or two other biomarkers. For example, a panel consisting essentially of four identified biomarkers can include up to six total biomarkers. A panel can exist as a conceptual grouping, as a composition of matter (e.g., comprising purified biomarkers, or as an article, such as solid support attached to a capture reagent such as an antibody, further bound to the biomarker. The solid support can be, for example, one or more solid particles, such as beads, or a chip in which biomarkers are attached in an array format.

Exemplary panels of biomarkers for assessing placenta accreta at around 24 weeks are presented in Table 7.

Exemplary panels of biomarkers for assessing placenta accreta at around 34 weeks are presented in Table 8.

### VI. Data Analysis

As used herein, the term “analysis” refers to any algorithm that transforms inputs into outputs. Analyses include, without limitation, statistical analyses, machine learning analyses and neural net analyses. The term “data” may include data received from various data sources, metadata associated with the data, and/or a combination of both data and metadata.

### A. Measurements

A measurement of a variable, such as sequencing reads mapping to a position, can be any combination of numbers and words. A measure can be any scale, including nominal (e.g., name or category), ordinal (e.g., hierarchical order of categories), interval (distance between members of an order), ratio (interval compared to a meaningful “0”), or a cardinal number measurement that counts the number of things in a set. Measurements of a variable on a nominal scale indicate a name or category, e.g., category into which the sequencing read is classified. Measurements of a variable on an ordinal scale produce a ranking, such as “first”, “second”, “third”. Measurements on a ratio scale include, for example, any measure on a pre-defined scale, absolute number of reads, normalized or estimated numbers, as well as statistical measurements such as frequency, mean, median, standard deviation, or quantile. Measurements that involve quantification are typically determined at the ratio scale level.

### B. Analysis

In some embodiments, analysis statistical analysis of a sufficiently large number of samples to provide statistically meaningful results. Any statistical method known in the art can be used for this purpose. Exemplary methods, or tools, include, without limitation, correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression, stepwise regression, ridge regression, lasso regression, elastic net regression) or non-parametric analysis (e.g., Wilcoxon rank-sum test, Wilcoxon sign-rank test, sign test). Such tools are included in commercially available statistical packages such as MATLAB, JMP Statistical Software and SAS. Such methods produce models or classifiers which one can use to classify a particular biomarker profile into a particular state.

In statistics and machine learning, lasso (least absolute shrinkage and selection operator; also Lasso or LASSO) is a regression analysis method that performs both variable selection and regularization in order to enhance the prediction accuracy and interpretability of the resulting statistical model.

Statistical analysis can be operator implemented or implemented by machine learning.

Certain classifiers, such as cut-offs, can be executed by human inspection. Other classifiers, such as multivariate classifiers, can require a computer to execute the classification algorithm.

### 1. Machine Learning

In some variations, analysis may involve implementing machine learning techniques including linear and non-linear models, e.g., processes such as CART-classification and regression trees), artificial neural networks such as back propagation networks, discriminant analyses (e.g., Bayesian classifier or Fischer analysis), logistic classifiers, and support vector classifiers (e.g., support vector machines).

Classification rules, algorithms, also referred to as models, can be generated by mathematical analysis, including by machine learning techniques that perform analysis of datasets of biomarker measurements derived from subjects classed into one or another group. In some embodiments, one or more classification rules or algorithms may employ one or more of the following: cut-off, linear regression including multiple linear regression, partial least squares regression, principal components regression, binary decision trees including recursive partitioning processes further including classification and regression trees, artificial neural networks including back propagation networks, discriminant analyses further including Bayesian classifier or Fischer analysis, logistic classifiers, and support vector classifiers including support vector machines. In some variations, these datasets of biomarker measurements may comprise more than 100, more than 1000, more than 10,000, or more than 100,000 data entries. In some variations, machine learning techniques that perform analysis of datasets of biomarker measurements derived from subjects classed into one or another group may access test data from the subjects and execute the one or more classification rules on the test data.

Diagnostic tests are characterized by sensitivity (percentage classified as positive that are true positives) and specificity (percentage classified as negative that are true negatives). The relative sensitivity and specificity of a diagnostic test can involve a trade-off-higher sensitivity can mean lower specificity, while higher specificity can mean lower sensitivity. These relative values can be displayed on a receiver operating characteristic (ROC) curve. The diagnostic power of a set of variables, such as biomarkers, is reflected by the area under the curve (AUC) of an ROC curve.

In some embodiments, the classifiers of this disclosure have a sensitivity value, a specificity value, a positive predictive value, or a negative predictive value of at least 85%, at least 90%, at least 95%, at least 98%, or at least 99%. Classifiers of this disclosure have an AUC of at least 0.6, at least 0.7, at least 0.8, at least 0.9 or at least 0.95.

Classification can be based on a measurement of a biomarker being above or below a selected cutoff level or value or threshold level or value. In certain embodiments, a cutoff value is obtained by measuring biomarker levels in a plurality of positive and negative reference samples, e.g., at least 10, 20, 50, 100 or 200 samples of each type (e.g., samples from control subjects and test subjects). A cutoff value can be established with respect to a measure of central tendency, such as mean, median or mode in the negative samples. A measure of deviation from this measure of central tendency can be used to set the cutoff. For example, the cutoff can be set based on variance or standard deviation. For example, the cutoff can be based on Z score, that is, a number of standard deviations above a mean of normal samples, for example one standard deviation, two standard deviations, three standard deviations or four standard deviations. For example, cutoff values can be selected so that the diagnostic test has at least an 80%, a 90%, a 95%, a 98%, a 99%, a 99.5%, or a 99.9% sensitivity value, specificity value and/or positive predictive value.

Numerically, an increased risk is associated with an odds ratio of over 1.0, preferably over 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 for placenta accreta.

### VII. Methods of Assessing Risk of Placenta Accreta

The phrase “increased risk” of a condition, as used herein, indicates that a subject has a greater likelihood of developing the condition than a general population of subjects. So, for example, a subject who is at “increased risk of placenta accreta” has a greater likelihood of developing placenta accreta than a general population of subjects at the same stage of pregnancy, optionally compared with a population sharing one or more demographic or risk factors. These may include, for example, age, placenta previa, previous cesarean delivery, endometrial ablation, in vitro fertilization, prior uterine infection, or other uterine surgery. For example, a test may indicate that a woman at around 24 weeks or around 34 weeks of pregnancy has a higher risk of developing placenta accreta than a general or control population of woman at around 24 weeks or around 34 weeks pregnancy.

Classifying can employ a classification rule, algorithm or model determined by statistical analysis and/or machine learning. In some embodiments, the classification rule may be based on one or more values. The one or more values may include one or more demographic or risk factors of a subject compared to the general population of subjects. The one or more values may also include measured values of one or more protein biomarkers.

### A. Assessing Placenta Accreta

Provided herein are methods of assessing risk for placenta accreta at anywhere between 20 weeks of pregnancy to about 37 weeks of pregnancy, for example, classifying a pregnant human female as being at an increased risk of placenta accreta. The methods can involve determining a measure of one or a plurality of the biomarkers in Table 1, Table 2, Table 3, Table 4, Table 5, Table 6, or Table 9, and associating the measure to risk of placenta accreta. For example, one can use a panel that includes 2, 3, 4, 5, 6, 7, 8, 9, or 10 or more, or, no more than 2, 3, 4, 5, 6, 7, 8, 9, or 10 biomarkers from any one or more of Table 1, Table 2, Table 3, Table 4, Table 5, Table 6, or Table 9. in the determination. In general, an amount of a biomarker that shows a difference compared to a control amount of the biomarker (e.g., a healthy pregnant female or a non-placenta accreta pregnant female), which difference is statistically significant, is associated with increased risk of placenta accreta. The difference can be an up-regulation or a down-regulation, which can be easily determined by the practitioner. Alternatively, determination may be based on a classification algorithm that may employ non-linear and/or hyperdimensional methods.

In some embodiments, pathways can be interrogated to aid the determination of risk of plasma accreta. For example, in some embodiments, one or more canonical pathways may be over-represented by differentially expressed proteins in second trimester placenta accreta cases. Such pathways may be one or more of the erythropoietin signaling pathway; and the iron homeostasis signaling pathway (making reference to Table 1.7 in Example 1).

In some embodiments, certain targets may be activated or inhibited, and may be interrogated to aid the determination of risk of plasma accreta. Exemplary targets are provided in Table 1.8 in Example 1.

In some embodiments, cellular and molecular functions around iron handling and erythrocyte function are over-represented and may be interrogated to aid the determination of risk of plasma accreta. Exemplary targets are provided in Table 1.9 in Example 1.

In some embodiments, one or more of isthmin-2 (ISM2), sulfhydryl oxidase 1 (QSOX1), histone H4 (H4), hemoglobin subunit gamma-2 (HBG2) and cartilage acidic protein 1 (CRAC1) are useful to distinguish plasma accreta from controls. In some embodiments, a biomarker panel of the disclosure comprises one, two, three, four, or all five of isthmin-2 (ISM2), sulfhydryl oxidase 1 (QSOX1), histone H4 (H4), hemoglobin subunit gamma-2 (HBG2) and cartilage acidic protein 1 (CRAC1). In some embodiments one or more of these markers are useful to distinguish plasma accreta from controls in the second trimester.

In some embodiments, one or more of ISM2, ubiquitin carboxyl-terminal hydrolase 1 (UBP1), immunoglobulin lambda variables 10-54 (LVX54) and Ig-like domain-containing protein, are useful to distinguish plasma accreta from controls. In some embodiments, a biomarker panel of the disclosure comprises one, two, three, or all four of ISM2, ubiquitin carboxyl-terminal hydrolase 1 (UBP1), immunoglobulin lambda variables 10-54 (LVX54) and Ig-like domain-containing protein. In some embodiments one or more of these markers are useful to distinguish plasma accreta from controls in the third trimester.

In other embodiments a determination is based on the use of a panel of biomarkers, for example those that are provided in Tables 7 or 8.

### B. Assessing Placenta Accreta-Second Trimester

Provided herein are methods of assessing risk for placenta accreta during the second trimester (e.g. at around 24 weeks of pregnancy), for example, classifying a pregnant human female as being at an increased risk of placenta accreta. The methods can involve determining a measure of one or a plurality of the biomarkers in Table 1, Table 3, Table 5, or Table 9 and associating the measure to risk of placenta accreta. For example, one can use a panel that includes 2, 3, 4, 5, 6, 7, 8, 9, or 10 or more, or, no more than 2, 3, 4, 5, 6, 7, 8, 9, or 10 biomarkers from the tables in the determination. In general, an amount of a biomarker that shows a difference compared to a control amount of the biomarker (e.g., a healthy pregnant female or a non-placenta accreta pregnant female), which difference is statistically significant, is associated with increased risk of placenta accreta. The difference can be an up-regulation or a down-regulation, which can be easily determined by the practitioner. Alternatively, determination may be based on a classification algorithm that may employ non-linear and/or hyperdimensional methods.

A biomarker panel can comprise of any of the biomarker panels presented in Table 7. A biomarker panel can consist essentially of any of the biomarker panels presented in Table 7. A biomarker panel can consist of any of the biomarker panels presented in Table 7. In some embodiments, one or more of isthmin-2 (ISM2), sulfhydryl oxidase 1 (QSOX1), histone H4 (H4), hemoglobin subunit gamma-2 (HBG2) and cartilage acidic protein 1 (CRAC1) are useful to distinguish plasma accreta from controls.

### C. Assessing Placenta Accreta—Third Trimester

Provided herein are methods of assessing risk for placenta accreta during the third trimester (e.g. at around 35 weeks of pregnancy), for example, classifying a pregnant human female as being at increased risk of placenta accreta. The methods can involve determining a measure of one or a plurality of the biomarkers in Table 2, Table 4, Table 6, or Table 9 and associating the measure to risk of placenta accreta. For example, one can use a panel that includes 2, 3, 4, 5, 6, 7, 8, 9, or 10 or more, or, no more than 2, 3, 4, 5, 6, 7, 8, 9, or 10 biomarkers from the tables in the determination. In general, an amount of a biomarker that shows a difference compared to a control amount of the biomarker (e.g., a healthy pregnant female or a non-placenta accreta pregnant female), which difference is statistically significant, is associated with increased risk of placenta accreta. The difference can be an up-regulation or a down-regulation, which can be easily determined by the practitioner. Alternatively, determination may be based on a classification algorithm that may employ non-linear and/or hyperdimensional methods.

A biomarker panel can comprise of any of the biomarker panels presented in Table 8. A biomarker panel can consist essentially of any of the biomarker panels presented in Table 8. A biomarker panel can consist of any of the biomarker panels presented in Table 8.

In some embodiments, one or more of ISM2, ubiquitin carboxyl-terminal hydrolase 1 (UBP1), immunoglobulin lambda variables 10-54 (LVX54) and Ig-like domain-containing protein, are useful to distinguish plasma accreta from controls.

### VIII. Methods of Treating Subjects at Increased Risk of Placenta Accreta

Methods of treating pregnant subjects suffering from or at increased risk of placenta accreta include assessing the risk of placenta accreta in a pregnant subject and administering one or more therapeutic interventions useful in treating placenta accreta, reducing the risk or placenta accreta and/or reducing neonatal complications of placenta accreta. In some embodiments, administering one or more therapeutic interventions includes administering an effective amount of one or more treatments designed to reduce the risk of placenta accreta. In some embodiments, one or more treatments may include performing a Cesarean hysterectomy, recommending bed rest to the subject to prevent preterm labor, performing a prophylactic embolization, leaving a portion of the placenta left in-situ, interesting a uterine balloon tamponade, administering methotrexate, inserting one or more temporal internal iliac occlusion balloon catheters, inserting one or more ureteral stents, or the like.

Patients with suspected placenta accreta spectrum should be referred to a center having advanced multidisciplinary surgical expertise and experience in any of the therapeutic interventions listed above or standard therapeutic interventions in the field.

Surgical planning for uterine conservation would be planned if future fertility was desired or planning for or performing a hysterectomy would be undertaken if fertility was not desired.

This can allow for appropriate planning to ensure access to specialists/surgeons and properly equipped facilities (i.e., blood bank support).

### IX. Kits and Other Articles of Manufacture

In another embodiment, provided herein are articles of manufacture, e.g., kits of reagents useful in detecting in a sample biomarkers, for increased risk of placenta accreta, in particular, placenta accreta. Reagents capable of detecting protein biomarkers include but are not limited to antibodies. Antibodies capable of detecting protein biomarkers are also typically directly or indirectly linked to a molecule such as a fluorophore or an enzyme, which can catalyze a detectable reaction to indicate the binding of the reagents to their respective targets.

In some embodiments, the kits further comprise sample processing materials comprising a high molecular weight gel filtration composition (e.g., agarose such as SEPHAROSE) in a low volume (e.g., 1 ml, 3 ml, 5 ml, 10 ml) vertical column for rapid preparation of a microparticle-enriched sample from plasma. For instance, the microparticle-enriched sample can be prepared at the point of care before freezing and shipping to an analytical laboratory for further processing.

In some embodiments, the kits further comprise instructions for assessing risk of placenta accreta, in particular, placenta accreta. As used herein, the term “instructions” refers to directions for using the reagents contained in the kit for detecting the presence (including determining the expression level) of a protein(s) of interest in a sample from a subject. The proteins of interest may comprise one or more biomarkers of placenta accreta.

In another embodiment, a kit comprises one or more containers wherein each container containing one or a plurality of stable isotope standard (SIS) peptides corresponding to peptide biomarkers, e.g., peptides produced from protease (e.g., trypsin) digestion of biomarker proteins. In another embodiment, a majority or all of the SIS peptides correspond to the biomarker peptides. In another embodiment, the kit further comprises the biomarker peptides which the SIS peptides correspond.

In another embodiment, provided is a composition of matter that includes protein biomarkers of placenta accreta and, for a plurality of those biomarkers, a corresponding stable isotope standard peptide. This can be prepared by combining a sample comprising proteins isolated from microparticles, with stable isotope standard peptides.

### X. Systems

Provided herein also are systems comprising a computer comprising a processor and memory. The computer can be configured to receive into memory one or more measurements of one or more biomarkers provided herein that are measured from a sample. The memory can include computer readable instructions which, when executed, classify the sample as at risk of placenta accreta or not at risk of placenta accreta. The computer system can be operatively coupled to a computer network with the aid of a communications interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The system can include a first computer connected with a second computer through a communications network, such as, a high-speed transmission network including, without limitation, Digital Subscriber Line (DSL), Cable Modem, Fiber, Wireless, Satellite and, Broadband over Powerlines (BPL). Accordingly, results providing classification of a sample as at increased risk or as not at increased risk of placenta accreta can be transmitted from a transmitting computer to a remote receiving computer, such as located at the office of a healthcare provider or to a mobile device, such as a smart phone.

### EXAMPLES

### Example 1

Maternal EDTA plasma samples were collected from patients who were >18 years of age, receiving prenatal care and planning on delivery at a hospital in the second and third trimesters at a median of about 26 (+/−2) and about 35 (+/−2) weeks' gestation, respectively. Pregnancy dating was confirmed by ultrasound at ≤12 weeks gestation. The samples were aliquoted and stored at −80 degrees centigrade. 35 Placenta accreta spectrum, referred to herein as “PAS”, cases and 70 controls were analyzed including 27 cases of grade 1 PAS, 7 cases of grade 2 PAS, and 1 case of grade 3 PAS as defined by the International Federation of Gynaecology and Obstetrics (“FIGO”) (Table 1.1).

TABLE 1.1
FIGO clinical and histologic criteria for PAS

| | Clinical Criteria | Histologic Criteria |
|---|---|---|
| Grade 1 | Attempts at manual removal | Hysterectomy specimen |
| | of adherent placenta results | with extended areas of |
| | in heavy bleeding requiring | absent decidua between |
| | mechanical or surgical | villous tissue and |
| | measures | myometrium |
| Grade 2 | Macroscopic findings: | Hysterectomy specimen |
| | hypervascularity, placental | shows placental villi within |
| | bulge, “dimple sign” | muscular fibers |
| Grade 3 | Macroscopic findings of | Hysterectomy specimen |
| | Grade 2 AND placental villi | showing villous tissue |
| | seen to be invading through | breaching the uterine serosa |
| | uterine surface +/− invasion | |
| | into other pelvic structures | |

Cases were defined as subjects with clinical or histologic grade 1 to 3 PAS consistent with the 2019 FIGO PAS classification (Table 1.1), delivery >23 weeks gestation and inclusion in the LIFECODES biobank. Prospective cases were first identified in the electronic medical record using the following word searches for records between 2007 and 2020: “adhere-” in operative reports and discharge summaries as well as “-creta,” “hyst-” or “previa” in pathology reports. The medical record for each prospective case was independently reviewed by two obstetricians within the institution's multidisciplinary PAS team. The higher grade between the clinical and histologic grades was designated as the assigned grade. Disagreement in either inclusion status or assigned PAS grade were adjudicated by a review committee. Subjects identified to have a PAS diagnosis were cross-referenced with the LIFECODES biobank for inclusion. Controls were defined as subjects without a diagnosis of PAS and randomly matched 2:1. Cases and controls were matched by gestational age of sampling (+1 week) and number of fetuses. Exclusion criteria were defined as current cancer diagnosis, use of immunomodulating medication or documented fetal chromosomal abnormality. Univariate analyses were conducted with chi-square tests and continuous variables were compared with Wilcoxon tests using SAS 9.4. All tests were two-tailed; P<0.05 was used to define statistical significance.

Compared to controls, cases were more likely to be of older maternal age and have placenta previa at delivery. Twenty (57.1%) of the PAS cases did not have a history of cesarean section nor placenta previa at delivery. Twenty-three (65.7%) of the PAS cases were determined by clinical criteria rather than histologic criteria. Notably, only 11% of grade 1 PAS cases were detected by ultrasound prior to delivery, compared to 57% and 100% of grade 2 and 3 PAS cases, respectively (Table 1.2).

TABLE 1.2
Characteristics and grading determination of PAS cases.

| | Cases⁺ | | Controls⁺ | | |
|---|---|---|---|---|---|
| | n = 35 | | n = 70 | | p-value |
| Maternal age (yrs) | 36.9 | (+2.9) | 32.9 | (+2.7) | <0.01 |
| # prior cesarean | | | | | 0.33 |
| sections | | | | | |
| 0 | 23 | (65.7) | 55 | (78.6) | |
| 1 | 7 | (20.0) | 10 | (14.3) | |
| >2 | 5 | (14/3) | 5 | (7.1) | |
| Placenta previa at | 8 | (22.9) | 6 | (8.6) | <0.01 |

| delivery | | | |
|---|---|---|---|
| PAS diagnosed by | | | N/A |
| ultrasound | | | |
| Grade 1 (n = 27) | 3 | (11.1) | |
| Grade 2 (n = 7) | 4 | (57.1) | |
| Grade 3 (n = 1) | 1 | (100.0) | |
| PAS grade and | | | |
| determination | | | |
| Grade 1 | 27 | (77.1) | |
| Clinical | 21 | | |
| Histologic | 0 | | |
| Both concordant | 6 | | |
| Grade 2 | 7 | (20.0) | |
| Clinical | 2 | | |
| Histologic | 1 | | |
| Both concordant | 4 | | |
| Grade 3 | 1 | (2.9) | |

| | |
|---|---|
| Clinical | 0 |
| Histologic | 0 |
| Both concordant | 1 |

⁺Data displayed as median (+IQR) or n (%)

To enrich each sample for circulating microparticles (“CMP”), Size Exclusion Chromatography (SEC) was used for CMP isolation. Anonymized EDTA plasma samples identified only by a study number that was agnostic to case or control status were randomly assorted and shipped on dry ice to NX Prenatal, Inc. (Houston, TX) where CMP protein enrichment was carried out by SEC and isocratically eluted using the NeXosome Elution Reagent. This involved NeXosome Isolation Columns manufactured by AmericanBio, Inc. (Canton, MA). Briefly, these columns were packed by AmericanBio with Sepharose 4B-CL (4% agarose, particle size 45-165 μm) from Cytiva (Marlborough, MA) to a total packed volume of 10 mL and delivered to NX Prenatal. Once received by NX Prenatal, the columns were stored at 2-8° C. until use. Prior to using the columns for CMP isolation, the columns were allowed to equilibrate to room temperature (overnight) and subsequently washed with NeXosome Elution Regent. EDTA plasma samples were thawed and 0.5 mL of plasma was applied and allowed to incorporate into the NeXosome Isolation Column. The plasma samples were not filtered, diluted, or pretreated prior to application to the columns. Following the incorporation of the sample into the column, the NeXosome Elution Reagent was added and 0.5 mL column fractions were collected. The eluted fractions yielded two peaks. The CMPs were captured in the column void volume and resolved from the high abundant soluble protein peak. Samples were processed according to a randomization scheme. Each CMP-containing fraction (0.5 mL aliquots of each fraction) was pooled within each individual sample and a total protein measurement was performed, using the Pierce BCA Protein Assay Kit (ThermoFisher Scientific). An aliquot containing a total protein of 200 μg from each individual CMP isolate pool was then transferred to 2-mL microcentrifuge tubes (VWR, Radnor, PA) and stored at −80° C. pending completion of all CMP isolate processing. All CMP isolates were then shipped on dry ice to BGI Americas Corporation (Cambridge, MA) for proteomic analysis.

A total of 158 Plasma enriched exosome samples were individually processed for LC-MS/MS analysis. 100 μL of enriched exosomes were mixed with 700 μL of lysis buffer that contained 9M urea, at pH 8.5, and 0.5% Rapigest (SKU: 186001861, Waters™). Samples were water-bath sonicated for 30 minutes followed by spinning at high-speed (14,000 rpm) in a centrifuge for 10 minutes. The protein concentration of samples was measured by the BCA assay (Cat No: A53225, ThermoFisher Scientific) post sample lysis.

50 μg of each sample was taken from the lysate and normalized to the same volume with lysis buffer. Samples were reduced in 10 mM DTT for 25 minutes at 60° C., then the reduced samples were alkylated in 20 mM IAM (iodoacetamide) in a dark environment for 20 minutes at room temperature. Excess IAM in the samples was quenched by adding 100 mM DTT. DI water and HEPE buffer at pH 8.5 were added to each sample so that the final urea concentration was diluted to 1.6 M, and a final pH of 8, for enzymatic digestion. 1 μg of Try/LysC (Cat No: A41007, ThermoFisher Scientific) was added to each sample. The samples were incubated overnight at 37° C. for 12 hours. An additional 1 μg of Tryp/LysC was added to each sample the next day and they were incubated for another 4 hours to complete the enzymatic digestion.

10% TFA was added into the digested samples (peptides) to produce a final concentration of 1% TFA—the pH was tested and the samples were acidic. Then, acidified samples were passed through a 10 mg SEK PAK column (Cat No: 60108-302, ThermoFisher Scientific) for desalting. 20% of the desalted peptides of each sample was taken and pooled together to create a composite “library” of peptides. The library samples were then fractionated into 96 fractions with a high pH, reverse-phase, offline HPLC fractionator (Vanquish™, ThermoFisher Scientific). The mobile phase A was made up of DI H2O with 20 mM formic acetate, pH 9.3; the mobile phase B was made up of acetonitrile (Optima™, LC/MS grade, Fisher Chemical™) with 20 mM formic acetate, pH 9.3. The gradient of separation is displayed in Table 1.3. 96 fractions were then combined into 24 fractions and readied for liquid chromatography mass spectrometry (LC/MS) analysis.

TABLE 1.3
High-pH, Reverse-Phase HPLC Fractionation Gradient Information

| Time[min] | Flow[ml/min] | % B |
|---|---|---|
| 0.00 | 0.500 | 2.0 |
| 1.00 | 0.500 | 6.0 |
| 12.00 | 0.500 | 20.0 |
| 30.00 | 0.500 | 28.0 |
| 50.00 | 0.500 | 65.0 |
| 53.00 | 0.500 | 98.0 |
| 57.00 | 0.500 | 98.0 |
| 59.00 | 0.500 | 2.0 |
| 60.00 | 0.500 | 2.0 |

All fractionated samples were analyzed by nanoflow HPLC (Ultimate 3000, Thermo Fisher Scientific) followed by Thermo Orbitrap mass spectrometer (Tribrid Eclipse) analysis. A Nanospray Flex™ Ion Source (Thermo Fisher Scientific) was equipped with Column Oven (PRSO-V2, Sonation) to heat up the nanocolumn (PicoFrit, 100 μm×250 mm×15 μm tip, New Objective) for peptide separation. The nanoLC method is water acetonitrile based that was 150 minutes long with a 0.300 L/min flowrate. For each sample injection, all peptides were first engaged on a trap column (Cat. No: 160454, Thermo Fisher) and then were delivered to the separation nanocolumn by the mobile phase. The specifics of the gradient used are provided in Table 1.4.

TABLE 1.4
High pH Reverse Phase HPLC Fractionation Gradient Information

| Time[min] | Flow[ml/min] | % B |
|---|---|---|
| 0.00 | 0.500 | 2.0 |
| 1.00 | 0.500 | 6.0 |
| 12.00 | 0.500 | 20.0 |
| 30.00 | 0.500 | 28.0 |
| 50.00 | 0.500 | 65.0 |
| 53.00 | 0.500 | 98.0 |
| 57.00 | 0.500 | 98.0 |
| 59.00 | 0.500 | 2.0 |
| 60.00 | 0.500 | 2.0 |

For the DDA library construction, a DIA library-specific DDA, MS2-based mass spectrometry method on Eclipse was used to sequence fractionated peptides that were eluted from the nanocolumn. For the full MS spectrum, a resolution of 120,000 was used with a scan range of 375 m/z-1500 m/z. For the dd-MS (MS2), a resolution of 15,000 was used, and the isolation window is 1.6 Da. ‘Standard’ AGC target and ‘Auto’ Max Ion injection times (Max IT) were selected for both MS1 and MS2 acquisition. The collision energy (NCE) was set to 35%, and the total cycle time is 1 second. For DIA analytical samples, a high-resolution, full MS scan, followed by two segment DIA methods, was used for the DIA data acquisition. For the full MS scan, a resolution of 120,000 was used for the range of 400 m/z-1200 m/z with a ‘Standard’ AGC target and 50 ms Max IT. For both DIA segments, the details of the isolation windows (IW) and the precursor mass ranges are shown in Table 1.5 and Table 1.6. For the DIA fragments scan, a resolution of 30,000 was used for the range of 110 m/z-1,800 m/z with a ‘Standard’ AGC target and ‘Auto’ Max IT.

TABLE 1.5
DIA segment 1 Precursor Scan Range Information.
Segment 1 (400-800 m/z, IW 15 m/z, Overlap 1 m/z)

| | |
|---|---|
| 399.5-415.5 | 609.5-625.5 |
| 414.5-430.5 | 624.5-640.5 |
| 429.5-445.5 | 639.5-655.5 |
| 444.5-460.5 | 654.5-670.5 |
| 459.5-475.5 | 669.5-685.5 |
| 474.5-490.5 | 684.5-700.5 |
| 489.5-505.5 | 699.5-715.5 |
| 504.5-520.5 | 714.5-730.5 |
| 519.5-535.5 | 729.5-745.5 |
| 534.5-550.5 | 744.5-760.5 |
| 549.5-565.5 | 759.5-775.5 |
| 564.5-580.5 | 774.5-790.5 |
| 579.5-595.5 | 789.5-800.5 |
| 594.5-610.5 | |

TABLE 1.6
DIA segment 2 Precursor Scan Range Information.
Segment 2 (800-1200 m/z, IW 25 m/z, Overlap 1 m/z)

| | |
|---|---|
| 799.5-825.5 | 999.5-1025.5 |
| 824.5-850.5 | 1024.5-1050.5 |
| 849.5-875.5 | 1049.5-1075.5 |
| 874.5-900.5 | 1074.5-1100.5 |
| 899.5-925.5 | 1099.5-1125.5 |
| 924.5-950.5 | 1024.5-1150.5 |
| 949.5-975.5 | 1049.5-1175.5 |
| 974.5-1000.5 | 1074.5-1200.5 |

The initial process is based on the sample data generated from a high-resolution mass spectrometer. The DDA data was identified by the Andromeda search engine within MaxQuant, and Spectronaut™ was used for the identification of results for spectral library construction. MaxQuant was used for the identification of DDA data, which served as a spectrum library for the subsequent DIA analysis. The analysis pipeline used raw data as input files and set corresponding parameters and human databases (UP000005640), then the identification and quantitative analysis was performed. The identified peptides satisfied a FDR of ≤1% to construct the final spectral library. For this DIA dataset, Spectronaut™ was employed to construct spectral library information to complete deconvolution and extraction, then the mProphet algorithm was used to complete an analytical quality control (1% FDR) to obtain reliable quantitative results. GO, COG, and Pathway functional annotation analysis and time series analysis were also performed in the pipeline described above. MStats, the core algorithm of which is a linear mixed effect model, was used to process the DIA quantification results data according to the predefined comparison group, and then a significance test was performed based on the model. Thereafter, differential protein screening was executed and a fold change of ≥2 and an adj P-value of <0.05 was defined as a significant difference. Based on the quantitative comparison results, the differential proteins between comparison groups were identified; finally a function enrichment analysis, a protein-protein interaction (PPI) examination, and a subcellular localization analysis of the differential proteins were carried out. The sample classification analyses were then implemented as described below.

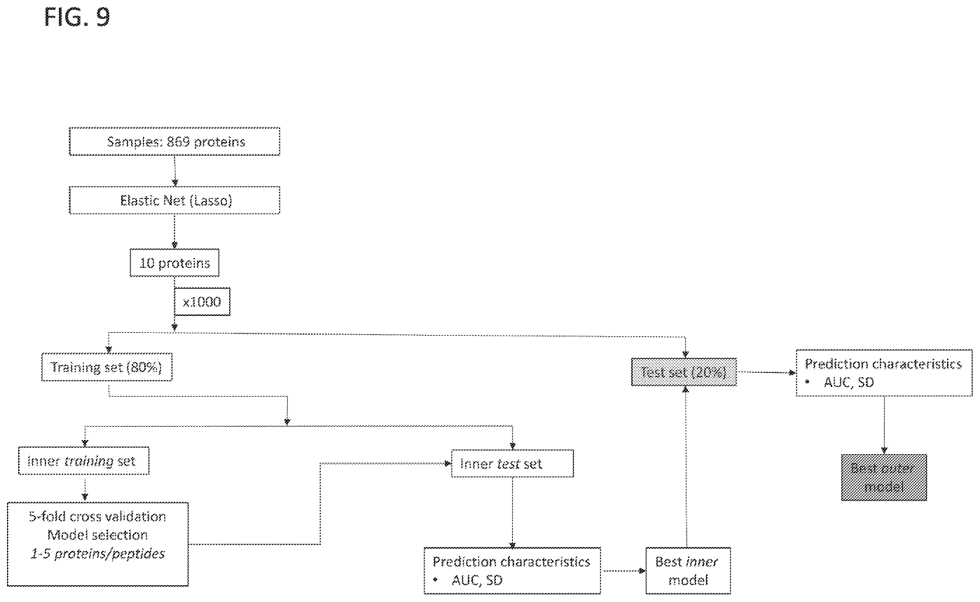

PAS was classified using regularized (L1) regression to define a restricted set of candidate CMP proteins from the superset of all identified proteins. To select the putative panel from the restricted set of candidate CMP proteins, a cross-validation procedure using logistic regression was chosen. The sample was randomly divided into a training and validation set (80% vs. 20%). The proteins in the training set were then ranked by their Akaike information criterion (AIC) using an ensemble feature selection procedure. The top 10 proteins were then passed to the glmulti package in R version 3.6.3 where the training set was subjected to fivefold cross-validation. To avoid overfitting, given the limited sample size, the model was restricted to no more than 5 predictors. The model with the greatest area under the curve (AUC) and the lowest standard deviation of the AUC was then tested against the set-aside, external validation set. The AUC and standard deviation of the AUC of this external validation set was then recorded and the workflow re-iterated for a total of 1000 iterations (FIG. 9). The models were then ranked by their mean AUC and mean standard deviation of the AUC. The workflow was then repeated with randomly permuted sample labels. Predictive statistics for the observed versus permuted data were then compared.

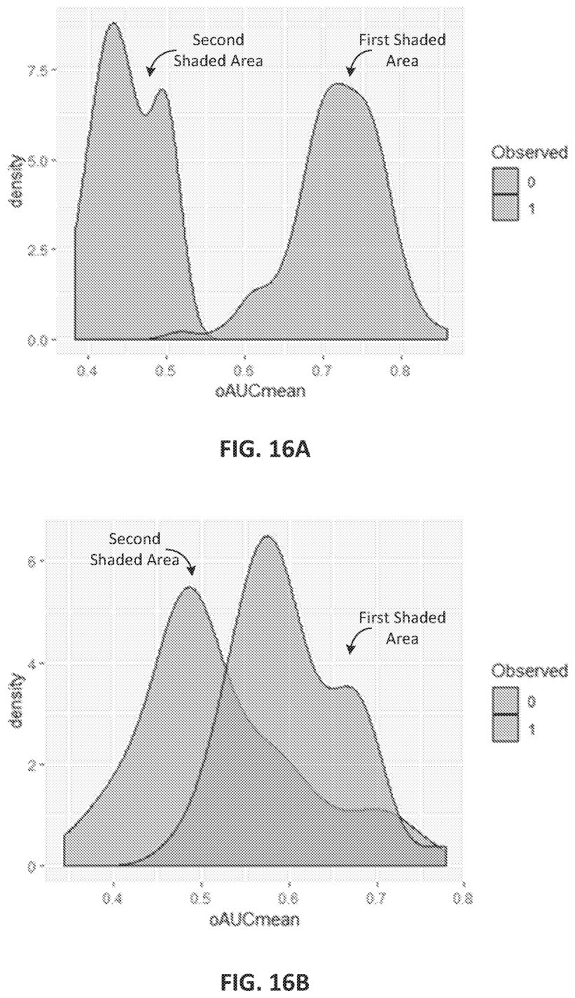

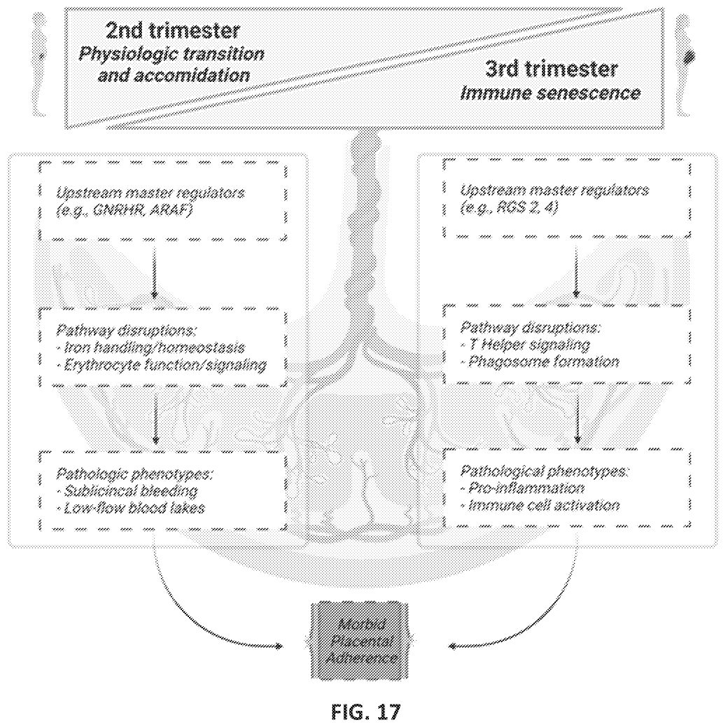

To establish a panel of CMP proteins that would serve as a classifier for the risk of PAS in the second and third trimesters, a two-step iterative workflow using regularized (L1) regression followed by a cross-validation procedure using logistic regression was used. The two-step workflow was also repeated with randomly permuted sample labels to simulate random chance. In second trimester samples, the mean of all area under the curves (AUCs) for the observed (e.g., first shaded area) versus permuted (e.g., second shaded area) panels was significantly different (0.72 vs. 0.45; p<2.20e-16; FIG. 16A). The top performing panel of markers distinguished PAS from controls with a mean AUC of 0.83. CMP proteins of this panel included: isthmin-2 (ISM2), sulfhydryl oxidase 1 (QSOX1), histone H4 (H4), hemoglobin subunit gamma-2 (HBG2) and cartilage acidic protein 1 (CRAC1). Within third trimester samples, the mean of all AUCs for the observed (e.g., first shaded area) versus permuted (e.g., second shaded area) panels were also significantly different (0.60 vs 0.52; p=2.79e-5; FIG. 16B). The top performing panel distinguished PAS from controls with a mean AUC of 0.78. CMP proteins in this panel included ISM2, ubiquitin carboxyl-terminal hydrolase 1 (UBP1), immunoglobulin lambda variables 10-54 (LVX54) and Ig-like domain-containing protein. Additionally, the mean of all AUC in the second trimester was significantly greater than that in the third trimester (p=1.59e-12).

To determine the biological function of those proteins identified as differentially expressed by PAS status in the differential expression analysis, an Ingenuity Pathway Analysis was also applied. Core analysis with the Ingenuity Knowledge Base reference set was used. Direct and indirect relationships were considered for network and regulatory analyses. IPA is a curated bioinformatic repository of functionally annotated analytes which allows for functional annotation, canonical pathway and network analyses, and upstream regulator analysis. Significantly (P<0.05) over-represented canonical pathways, upstream regulators, and molecular and cellular functions were identified. Only relevant pathways and biological functions containing two or more overlapping hits were included, while only upstream regulators with a predicted activation state (activated, inhibited) were included. Overlap ratios were calculated as the percent of overlap between differentially expressed proteins and the target pathway.

To determine the biological function of those proteins identified as differentially expressed by PAS status in the differential expression analysis, an Ingenuity Pathway Analysis was also applied. This revealed significant over-representation of canonical pathways, upstream regulators, and molecular and cellular functions. In the second trimester, proteomic changes in PAS yielded significant over-representation of several canonical pathways including iron homeostasis signaling and erythropoietin signaling (Table 1.7).

TABLE 1.7
Canonical pathways significantly over-represented by differentially
expressed proteins in second trimester placenta accreta cases.

| Ingenuity | | | |
|---|---|---|---|
| Canonical Pathways | -log(p-value) | Overlap ratio | Molecules |
| Erythropoietin | 4.33 | 0.0169 | HBA1/HBA2, HBB, |
| Signaling Pathway | | | HBG1 |
| Iron homeostasis | 4.64 | 0.0216 | HBA1/HBA2, HBB, |
| signaling pathway | | | HBG1 |

Master upstream regulators included seven targets predicted to be significantly activated and six predicted to be significantly inhibited (Table 1.8).

TABLE 1.8
Select master upstream regulators of target molecules
in second trimester placenta accreta dataset.

| | | Predicted | | |
|---|---|---|---|---|
| Master | Molecule | Activation | p-value of | |
| Regulator | Type | State | overlap | Target molecules in dataset |
| signal | complex | Activated | 0.0000173 | ALB, ANGPTL2, GRN, HBA1/HBA2, |
| peptidase | | | | HBB, NID1, VNN1 |
| GNRHR | G-protein | Activated | 0.000143 | GRN, HBA1/HBA2, HBB, VNN1 |
| | coupled | | | |
| | receptor | | | |
| DRD3 | G-protein | Activated | 0.000148 | GRN, HBA1/HBA2, HBB, VNN1 |
| | coupled | | | |
| | receptor | | | |
| ARAF | kinase | Activated | 0.000408 | GRN, HBA1/HBA2, HBB, VNN1 |
| G protein | group | Activated | 0.000471 | GRN, HBA1/HBA2, HBB, VNN1 |
| alpha | | | | |
| F10 | peptidase | Activated | 0.000649 | ANGPTL2, HBA1/HBA2, HBB, VNN1 |
| BDKRB2 | G-protein | Activated | 0.000826 | GRN, HBA1/HBA2, HBB, VNN1 |
| | coupled | | | |
| | receptor | | | |
| MAP2K7 | kinase | Inhibited | 0.000139 | ALB, ANGPTL2, HBA1/HBA2, HBB, |
| | | | | HBG1 |
| CDK5R1 | kinase | Inhibited | 0.000269 | ANGPTL2, GRN, HBA1/HBA2, HBB, |
| | | | | VNN1 |
| SERPINB1 | other | Inhibited | 0.000691 | ANGPTL2, GRN, HBA1/HBA2, HBB, |
| | | | | NID1, VNN1 |

IPA molecular and cellular functions analyses revealed 43 select annotated functions which were significantly over-represented based on differentially-expressed molecular hits from second trimester placenta accreta analyses (Table 1.9). Cellular and molecular functions around iron handling and erythrocyte function agreed with canonical iron homeostasis and erythropoietin signaling pathways.

TABLE 1.9
Select over-represented molecular and cellular functions
of proteins altered in second trimester placenta accreta

| Diseases or | | |
|---|---|---|
| Functions Annotation | p-value | Molecules |
| Heinz body anemia | 1.59E−07 | HBA1/HBA2, HBB |
| Mineral deficiency | 2.90E−07 | ALB, HBA1/HBA2, HBB |
| Iron deficiency anemia of | 4.77E−07 | HBA1/HBA2, HBB |
| pregnancy | | |
| Refractory iron deficiency | 4.77E−07 | HBA1/HBA2, HBB |
| Hereditary persistence of fetal | 1.59E−06 | HBB, HBG1 |
| hemoglobin | | |
| Transport of oxygen | 2.38E−06 | HBA1/HBA2, HBB |
| Biosynthesis of hydrogen | 1.99E−05 | ALB, HBA1/HBA2, HBB |
| peroxide | | |
| Catabolismof hydrogen peroxide | 3.01E−05 | HBA1/HBA2, HBB |
| Hemolysis | 3.59E−05 | ALB, HBA1/HBA2, HBB |
| Degranulation of phagocytes | 9.34E−05 | ALB, GRN, HBB, VNN1 |
| Degranulation of myeloid cells | 9.86E−05 | ALB, GRN, HBB, VNN1 |
| Familial hemolytic anemia | 1.29E−04 | HBA1/HBA2, HBB |
| Peripheral vascular disease | 1.49E−04 | ALB, GRN, HBA1/HBA2, |
| | | HBB |
| Aggregation of blood platelets | 1.60E−04 | ALB, ANGPTL2, HBB |
| Polycythemia | 1.90E−04 | GRN, HBA1/HBA2, HBB |
| Receptor-mediated endocytosis | 2.10E−04 | ALB, HBA1/HBA2, HBB |
| Synthesis of reactive oxygen | 2.30E−04 | ALB, GRN, HBA1/HBA2, |
| species | | HBB |
| Quantity of reticulocytes | 3.26E−04 | HBA1/HBA2, HBB |
| Endocytosis | 4.02E−04 | ALB, GRN, HBA1/HBA2, |
| | | HBB |
| Degranulation of neutrophils | 9.46E−04 | GRN, HBB, VNN1 |
| Formation of epithelial tissue | 9.64E−04 | GRN, NID1 |
| Hypertension | 1.02E−03 | ALB, ANGPTL2, |
| | | HBA1/HBA2, HBG1 |
| Ischemic stroke | 1.11E−03 | ALB, GRN |
| Production of reactive oxygen | 1.54E−03 | GRN, HBA1/HBA2, HBB |
| species | | |
| Production of superoxide | 1.95E−03 | HBA1/HBA2, HBB |
| Quantity of myeloid cells | 5.34E−03 | GRN, HBA1/HBA2, HBB |
| Consumption of oxygen | 5.88E−03 | HBA1/HBA2, HBB |
| Cell movement of myeloid cells | 5.88E−03 | ALB, ANGPTL2, GRN |
| Generation of reactive oxygen | 6.20E−03 | ALB, HBA1/HBA2 |
| species | | |
| Innate immune response | 7.24E−03 | GRN, VNN1 |
| Coagulation of blood | 1.01E−02 | HBB, HBG1 |
| Severe inflammatory disorder | 1.05E−02 | GRN, HBG1 |
| Synthesis of nitric oxide | 1.18E−02 | ALB, ANGPTL2 |
| Inflammatory response | 1.48E−02 | ANGPTL2, GRN, VNN1 |
| Cell movement of neutrophils | 1.73E−02 | ALB, GRN |
| Apoptosis | 2.49E−02 | ALB, GRN, HBA1/HBA2, |
| | | HBB, VNN1 |
| Quantity of metal ion | 2.73E−02 | GRN, HBA1/HBA2 |
| Abnormal morphology of | 2.75E−02 | GRN, NID1 |
| epithelial tissue | | |
| Bleeding | 2.95E−02 | ALB, GRN |
| Development of vasculature | 3.62E−02 | ANGPTL2, GRN, NID1 |
| Preeclampsia | 3.73E−02 | ANGPTL2, HBG1 |
| Chronic inflammatory disorder | 3.77E−02 | ALB, GRN, HBA1/HBA2 |
| Development of epithelial tissue | 4.45E−02 | GRN, NID1 |

As with second trimester data, IPA Core Analysis revealed significant over-representation of canonical pathways, upstream regulators, and molecular and cellular functions in the third trimester in PAS. Canonical pathway analysis of third trimester proteomic changes in PAS revealed significant over-representation of pathways including immune and extracellular signaling pathways, specifically involving IL-15 (Table 1.10).

TABLE 1.10
Canonical Pathway analysis of Third Trimester Changes

| Ingenuity | -log(p- | Overlap | |
|---|---|---|---|
| Canonical Pathways | value) | Ratio | Molecules |
| Communication between | 2.9 | 0.00431 | IGHV3-20, IGKV3D-11, |
| Innate and Adaptive | | | IGKV4-1, IGLV2-18 |
| Immune Cells | | | |
| B Cell Receptor Signaling | 3.54 | 0.00634 | IGHV3-20, IGKV3D-11, |
| | | | IGKV4-1, IGLV2-18 |
| Integrin Signaling | 3.72 | 0.0141 | ARPC5, ILK, PARVB |
| IL-15 Signaling | 3.84 | 0.00758 | IGHV3-20, IGKV3D-11, |
| | | | IGKV4-1, IGLV2-18 |

Master upstream regulators included three predicted to be significantly activated and two significantly inhibited (Table 1.11).

TABLE 1.11
Select master upstream regulators of target molecules
in third trimester placenta accreta dataset.

| | | Predicted | p-value | |
|---|---|---|---|---|
| Master | Molecule | Activation | of | Target molecules |
| Regulator | Type | State | overlap | in dataset |
| DVL1 | other | Activated | 0.000188 | GAPDH, HSPG2, ILK, |
| | | | | MPO, PARVB |
| Histone | group | Activated | 0.00121 | ARPC5, GAPDH, |
| h3 | | | | HSPG2, ILK, MPO |
| ADCY | group | Activated | 0.00168 | ARPC5, GAPDH, |
| | | | | HSPG2, PARVB |
| RGS (2, 4, | group | Inhibited | 0.0000907 | GAPDH, HSPG2, ILK, |
| 7, 16, 18) | | | | MPO, SPP2 |

Molecular and cellular functional analyses revealed 24 select annotated functions which were significantly over-represented based on differentially expressed molecular hits from third trimester analyses in PAS (Table 1.12).

TABLE 1.12
Select over-represented molecular and cellular functions
of proteins altered in third trimester placenta accreta

| Diseases or Functions Annotation | p-Value | Molecules |
|---|---|---|
| Classical complement pathway | 0.0000102 | IGKV4-1, C1QB, IGKV3D-11 |
| Receptor-mediated endocytosis | 0.000487 | IGKV4-1, HSPG2, IGKV3D-11 |
| Cell movement | 0.00278 | IGKV4-1, MPO, HSPG2, GAPDH, |
| | | IGKV3D-11, PARVB, ILK |
| Reorganization of actin cytoskeleton | 0.00342 | PARVB, ILK |
| Attachment of cells | 0.0035 | HSPG2, ILK |
| Apoptosis of cardiomyocytes | 0.00764 | MPO, GAPDH |
| Cellular infiltration | 0.0079 | MPO, GAPDH, ILK |
| Leukocyte migration | 0.0087 | IGKV4-1, MPO, IGKV3D-11, ILK |
| Migration of connective tissue cells | 0.00895 | HSPG2, ILK |
| Aggregation of blood platelets | 0.00908 | MPO, ILK |
| Degranulation of cells | 0.00916 | SPP2, ARPC5, MPO |
| DNA damage | 0.0159 | MPO, GAPDH |
| Organization of cellular membrane | 0.0165 | ARPC5, ILK |
| Cell death of epithelial cell lines | 0.0213 | MPO, G APDH |
| Cell death of kidney cell lines | 0.0213 | GAPDH, ILK |
| Familial heart disease | 0.0242 | HSPG2, ILK |
| Acute respiratory disorder | 0.0258 | MPO, GAPDH |
| Immune mediated inflammatory disease | 0.0267 | MPO, CIQB, GAPDH, ILK |
| Degranulation of neutrophils | 0.0286 | ARPC5, MPO |
| Cell movement of neutrophils | 0.0288 | MPO, ILK |
| Adhesion of immune cells | 0.0305 | MPO, ILK |
| Organization of cytoskeleton | 0.0322 | ARPC5, GAPDH, PARVB, ILK |
| Disorder of pregnancy | 0.0337 | MPO, GAPDH, ILK |
| Migration of cells | 0.0376 | IGKV4-1, MPO, HSPG2, IGKV3D- |
| | | 11, ILK |

There was some agreement, particularly around immune signaling and cytoskeletal and cell growth functions, between these cellular and molecular functions and the canonical pathways revealed by IPA.

### EXEMPLARY EMBODIMENTS

The following exemplary embodiments are provided herein.

### Set I:

Embodiment I-1. A method of preparing a peptide sample comprising:
(a) providing a blood, serum or plasma sample from a pregnant subject at around 24 weeks or 34 weeks of pregnancy;
(b) enriching the sample for microparticles by loading the sample on a size-exclusion column and eluting the microparticles from the column using water as the mobile phase, to produce a microparticle-enriched fraction;
(c) preparing a microparticle-associated peptide fraction from the microparticle-enriched fraction by contacting with a protease;
(d) separating the microparticle-associated peptides by mass spectrometry; and
(e) measuring, based on a mass spectrometry signal, one or more peptides corresponding to each of one or more protein biomarkers selected from:
   (i) a protein biomarker of Table 1, Table 3 or Table 5, wherein the blood sample is collected at about 24 weeks of pregnancy; and
   (ii) a protein biomarker of Table 2, Table 4 or Table 6, wherein the blood sample is collected at about 34 weeks of pregnancy.

Embodiment I-2. The method of embodiment I-1, wherein the one or more protein biomarkers is a plurality of protein biomarkers.

Embodiment I-3. The method of embodiment I-1, wherein the biomarkers comprise a panel of no more than any of 10, 9, 8, 7, 6, 5, 4 or 3 protein biomarkers.

Embodiment I-4. The method of embodiment I-1, wherein the biomarkers comprise, consist essentially of or consist of a panel of biomarkers selected from:
(i) a biomarker panel of Table 7, wherein the blood sample is collected at about 24 weeks of pregnancy; and
(ii) a protein biomarker of Table 8, wherein the blood sample is collected at about 34 weeks of pregnancy.

Embodiment I-5. The method of embodiment I-1, wherein measuring the one or more peptides comprises measuring a surrogate biomarker of any of FIGS. 10A-10C, FIGS. 11A-11C, FIGS. 12A-12H, FIGS. 13A-13H, FIGS. 14A-14D, FIGS. 15A-15B.

Embodiment I-6. The method of embodiment I-1, wherein the pregnant subject has one or more risk factors for placenta accreta.

Embodiment I-7. The method of embodiment I-1, wherein the pregnant subject is primigravida, multigravida, primiparous or multiparous.

Embodiment I-8. The method of embodiment I-1, wherein the blood sample is plasma or serum.

Embodiment I-9. The method of embodiment I-1, wherein the water is deionized distilled water (“ddH2O”).

Embodiment I-10. The method of embodiment I-1, wherein the size-exclusion chromatography is performed with an agarose solid phase and an aqueous liquid phase.

Embodiment I-11. The method of embodiment I-1, wherein the preparing step further comprises using ultrafiltration or reverse-phase chromatography.

Embodiment I-12. The method of embodiment I-1, wherein the preparing step further comprises denaturation using urea, reduction using dithiothreitol, alkylation using iodoacetamine, and digestion using trypsin after the size exclusion chromatography.

Embodiment I-13. The method of embodiment I-1, wherein the microparticles are further purified to enrich for placental-derived exosomes or vascular endothelial-derived exosomes.

Embodiment I-14. The method of embodiment I-1, wherein mass spectrometry comprises liquid chromatography/mass spectrometry (LC/MS), e.g., liquid chromatography/triple quadrupole mass spectrometry.

Embodiment I-15. The method of embodiment I-1, wherein the mass spectrometry comprises multiple reaction monitoring.

Embodiment I-16. The method of embodiment I-1, wherein the peptide(s) are selected from:
(i) a biomarker panel of Table 7, wherein the blood sample is collected at about 24 weeks of pregnancy; and
(ii) a protein biomarker of Table 8, wherein the blood sample is collected at about 34 weeks of pregnancy.

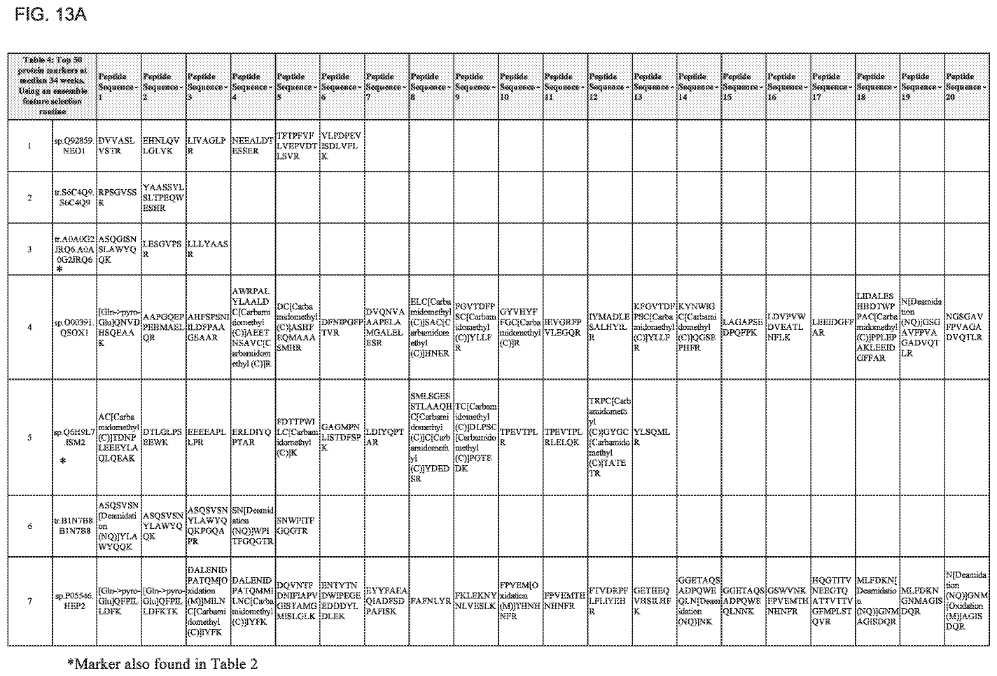

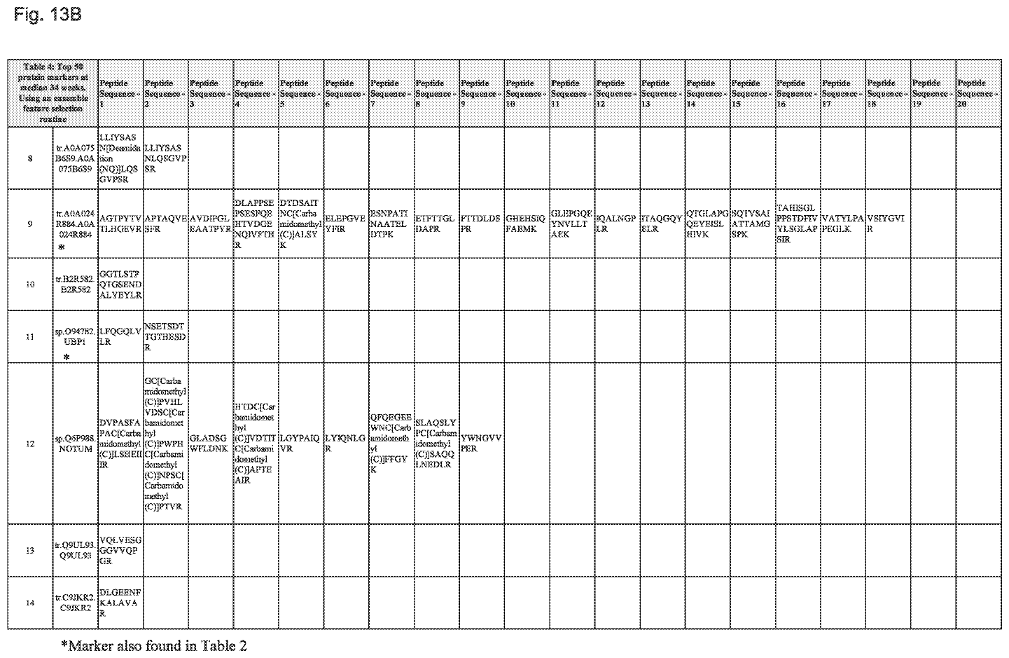

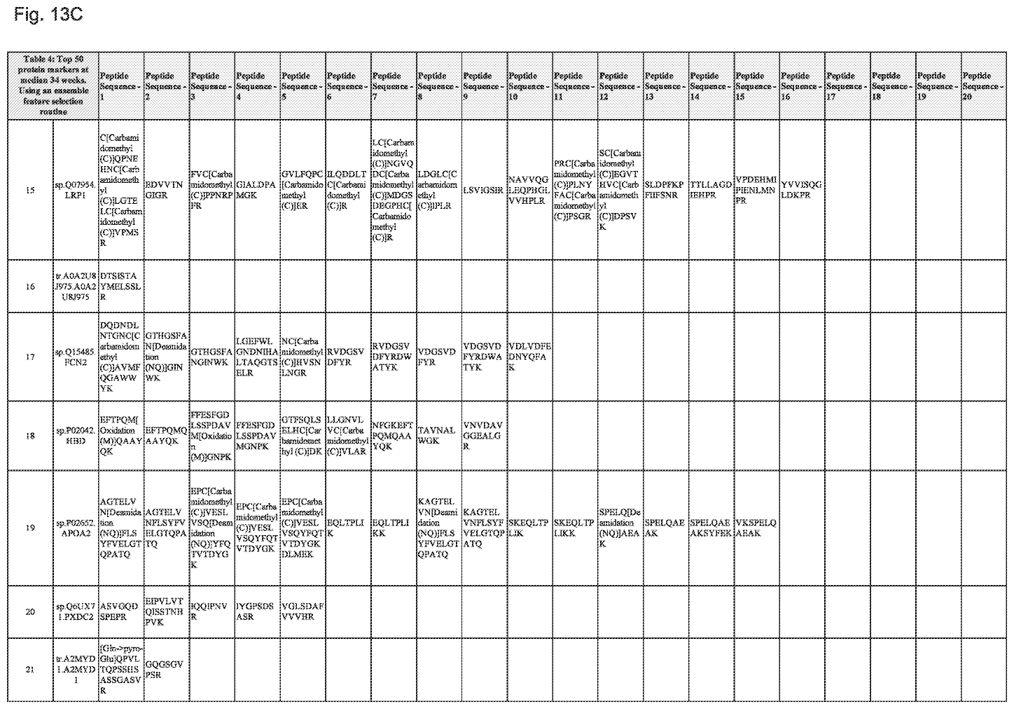

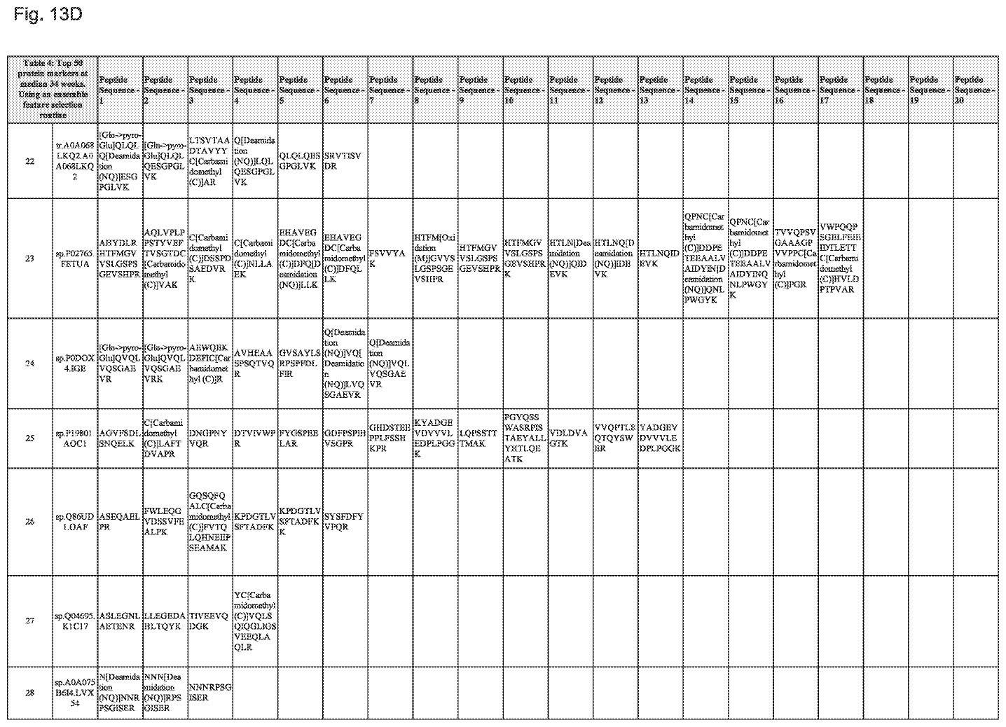

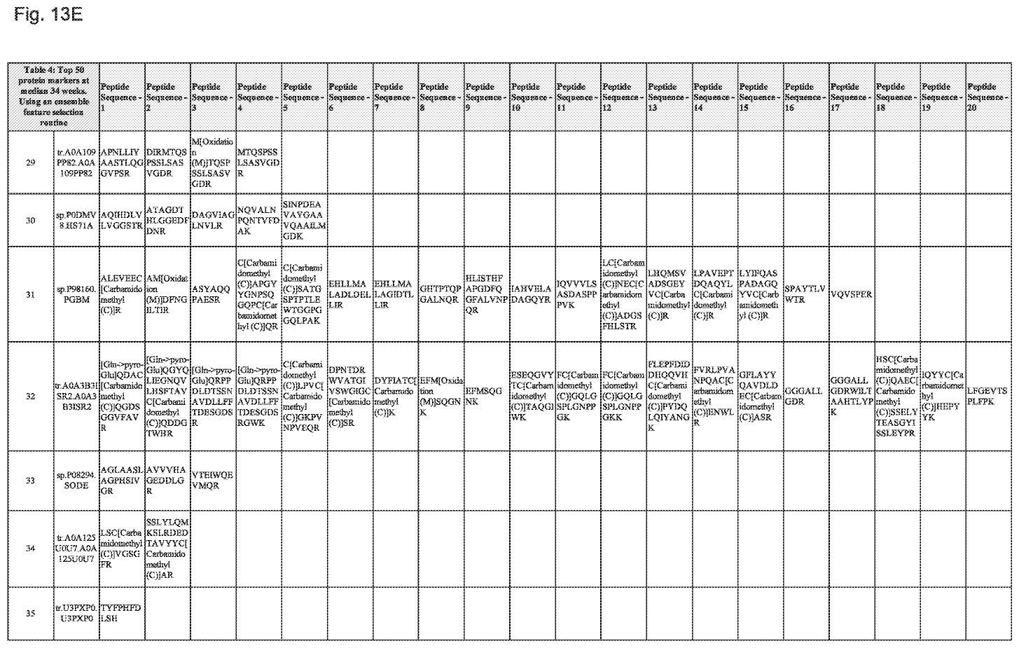

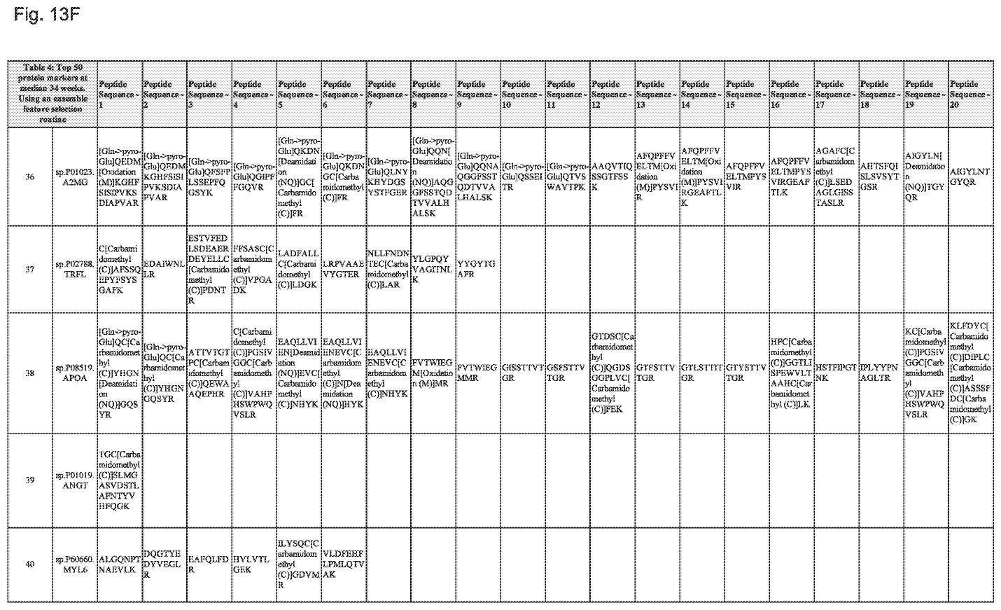

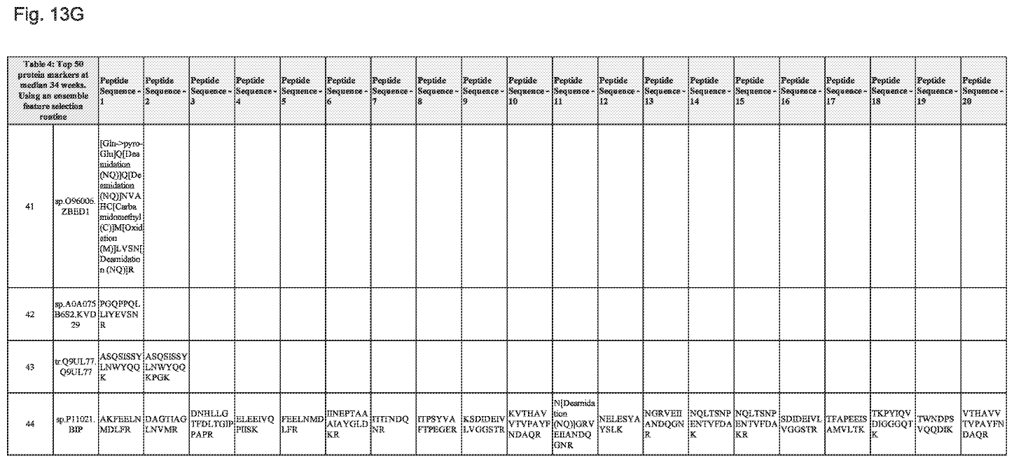

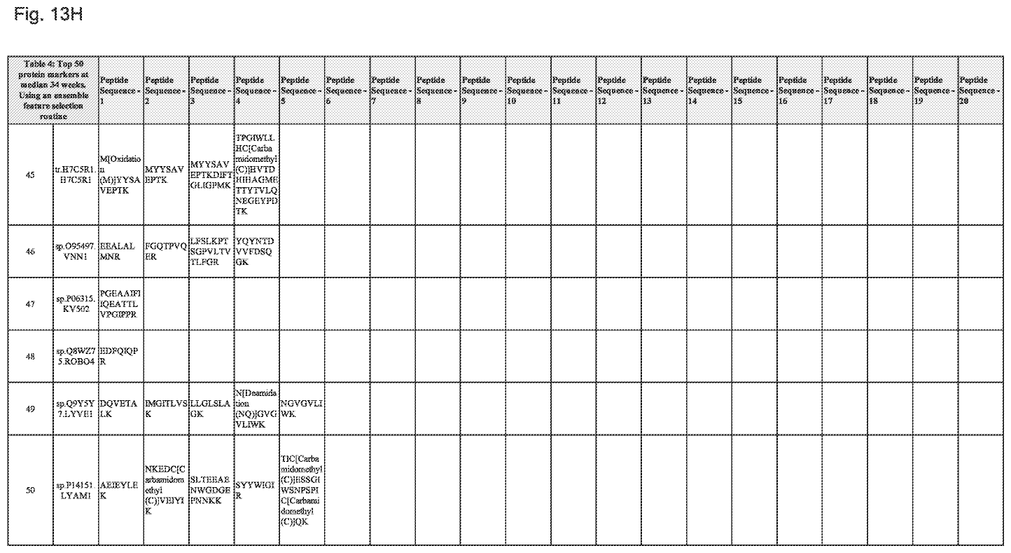

Embodiment I-17. A panel comprising a plurality of substantially pure protein biomarkers or surrogate biomarkers selected from:
(i) a protein biomarker of Table 1, Table 3 or Table 5;
(ii) a protein biomarker of Table 2, Table 4 or Table 6;
(iii) a surrogate biomarker of FIGS. 10A-10C, FIGS. 11A-11C or FIGS. 12A-12H; and
(iv) a surrogate biomarker of FIGS. 13A-13H, FIGS. 14A-14D or FIGS. 15A-15B.

Embodiment I-18. The panel of embodiment I-17, further comprising a stable isotope standard peptide paired with each of the surrogate biomarkers.

Embodiment I-19. A kit comprising one or a plurality of containers, wherein each container comprises one or more of each of a plurality of Stable Isotopic Standards, each stable isotopic standard corresponding to a surrogate peptide for a biomarker from a panel of biomarkers selected from:
(i) a protein biomarker of Table 1, Table 3, or Table 5;
(ii) a protein biomarker of Table 2, Table 4, or Table 6
(iii) a surrogate biomarker of FIGS. 10A-10C, FIGS. 11A-11C or FIGS. 12A-12H; and
(iii) a surrogate biomarker of FIGS. 13A-13H, FIGS. 14A-14D or FIGS. 15A-15B.

Embodiment I-20. A composition comprising one or a plurality of pairs of polypeptides, each pair comprising a protein biomarkers or surrogate biomarkers selected from:

(i) a protein biomarker of Table 1, Table 3, or Table 5;

(ii) a protein biomarker of Table 2, Table 4, or Table 6

(iii) a surrogate biomarker of FIGS. 10A-10C, FIGS. 11A-11C or FIGS. 12A-12H; and
(iii) a surrogate biomarker of FIGS. 13A-13H, FIGS. 14A-14D or FIGS. 15A-15B.

Embodiment I-21. A computer readable medium in tangible, non-transitory form comprising code to implement a classification rule generated by a method as described herein.

Embodiment I-22. A system comprising:
(a) a computer comprising:
   (i) a processor; and
   (ii) a memory, coupled to the processor, the memory storing a module comprising:
      (1) test data for a sample from a subject including values indicating a measure of one or more protein biomarkers in the fraction, wherein the protein biomarkers are selected from
         (i) a protein biomarker of Table 1, Table 3, or Table 5, wherein the sample is collected at about 20 weeks of pregnancy; and
         (ii) a protein biomarker of Table 2, Table 4, or Table 6, wherein the sample is collected at about 37 weeks of pregnancy;
      (2) a classification rule which, based on values including the measurements, classifies the subject as being at increased risk of placenta accreta, wherein the classification rule is configured to have a sensitivity of at least 75%, at least 85% or at least 95%; and
      (3) computer executable instructions for implementing the classification rule on the test data.

Embodiment I-23. The system of embodiment I-22, wherein the protein biomarker is a surrogate biomarker selected from:
(iii) a surrogate biomarker of FIGS. 10A-10C, FIGS. 11A-11C or FIGS. 12A-12H; and
(iii) a surrogate biomarker of FIGS. 13A-13H, FIG. 14A-14D or FIG. 15A-15B.

Embodiment I-24. A method comprising:
(a) at a computer system comprising a processor and a memory, coupled to the processor, the memory storing a module comprising:
   (1) test data for a sample from a subject including values indicating a measure of one or more protein biomarkers in the fraction, wherein the protein biomarkers are selected from
      (i) a protein biomarker of Table 1, Table 3, or Table 5, wherein the sample is collected at about 24 weeks of pregnancy; and
      (ii) a protein biomarker of Table 2, Table 4, or Table 6, wherein the sample is collected at about 34 weeks of pregnancy;
   (2) a classification rule to be executed by the processor, which, based on values including the measurements, classifies the subject as being at increased risk of placenta accreta, wherein the classification rule is configured to have a sensitivity of at least 75%, at least 85% or at least 95%;
(b) accessing the text data; and
(c) executing the classification rule on the test data.

Embodiment I-25. A method of assessing risk of placenta accreta in a pregnant subject, the method comprising:
(a) preparing a microparticle-enriched fraction from a blood sample from a pregnant subject;
(b) determining a measure of one or more microparticle-associated protein biomarkers in the fraction, wherein protein biomarkers are selected from:
   (i) a protein biomarker of Table 1, Table 3, or Table 5, wherein the blood sample is collected at about 24 weeks of pregnancy; and
   (ii) a protein biomarker of Table 2, Table 4, or Table 6, wherein the blood sample is collected at about 34 weeks of pregnancy;
(c) assessing risk of placenta accreta based on the one or more measures.

Embodiment I-26. The method of embodiment I-25, wherein the protein biomarker is a surrogate biomarker selected from:
(iii) a surrogate biomarker of FIGS. 10A-10C, FIGS. 11A-11C or FIGS. 12A-12H; and
(iii) a surrogate biomarker of FIGS. 13A-13H, FIGS. 14A-14D or FIGS. 15A-15B.

Embodiment I-27. The method of embodiment I-25, wherein determining a quantitative measure comprises contacting the sample with one or more capture reagents, each capture reagent specifically binding one of the protein biomarkers, and detecting binding between the capture reagent in the protein biomarker.

Embodiment I-28. The method of embodiment I-27, comprising performing an immunoassay.

Embodiment I-29. The method of embodiment I-28, wherein the immunoassay is selected from the group consisting of enzyme immunoassay (EIA), enzyme-linked immunosorbent assay (ELISA), and radioimmunoassay (RIA).

Embodiment I-30. The method of embodiment I-25, wherein the assessing comprises executing a classification rule, which rule classifies the subject at being at risk of placenta accreta, and wherein execution of the classification rule produces a correlation between placenta accreta or term birth with a p value of less than at least 0.05.

Embodiment I-31. The method of embodiment I-25, wherein the assessing comprises executing a classification rule, which rule classifies the subject at being at risk of placenta accreta, and wherein execution of the classification rule produces a receiver operating characteristic (ROC) curve, wherein the ROC curve has an area under the curve (AUC) of at least 0.6, at least 0.7, at least 0.8 or at least 0.9.

Embodiment I-32. The method of embodiment I-25, wherein values on which the classification rule classifies a subject further include at least one of: placenta previa, previous cesarean delivery, endometrial ablation, in vitro fertilization, prior uterine infection, or previous uterine surgery.

Embodiment I-33. The method of any of the preceding embodiments, wherein the classification rule employs cut-off, linear regression (e.g., multiple linear regression (MLR), partial least squares (PLS) regression, principal components regression (PCR)), binary decision trees (e.g., recursive partitioning processes such as CART-classification and regression trees), artificial neural networks such as back propagation networks, discriminant analyses (e.g., Bayesian classifier or Fischer analysis), logistic classifiers, and support vector classifiers (e.g., support vector machines).

Embodiment I-34. The method of embodiment I-25, wherein the classification rule is configured to have a sensitivity, specificity, positive predictive value, or negative predictive value of at least 70%, least 80%, at least 90% or at least 95%.

Embodiment I-35. The method of embodiment I-25, wherein assessing an increased risk of placenta accreta comprises determining that the protein biomarker (if upregulated) is above or (if down regulated) is below a threshold level.

Embodiment I-36. The method of embodiment I-35, wherein the threshold level represents a level at least one, at least two or at least three z scores from a measure of central tendency (e.g., mean, median or mode) for the protein determined from at least 50, at least 100 or at least 200 control subjects.

Embodiment I-37. The method of embodiment I-25, wherein the assessing comprises comparing the measure of each protein in the panel to a reference standard.

Embodiment I-38. The method of embodiment I-25, further comprising communicating the risk of placenta accreta for a pregnant subject to a health care provider.

Embodiment I-39. A method of treating placenta accreta in a pregnant subject, the method comprising:
(a) assessing risk of placenta accreta for a pregnant subject according to the method of any one of embodiments I-25 to I-38; and
(b) administering a therapeutic intervention to the subject effective to decrease the risk of placenta accreta and/or reduce neonatal complications of placenta accreta.

Embodiment I-40. The method of embodiment I-39, wherein treating comprises a therapeutic intervention selected from the group consisting of:
(i) referring the subject to a medical center with advanced multidisciplinary surgical expertise and experience;
(ii) surgical uterine conservation; and
(iii) performing a Cesarean hysterectomy, performing a prophylactic embolization, inserting a uterine balloon tamponade, a temporal internal iliac occlusion balloon catheter, a ureteral stents, administering methotrexate, leaving a portion of the placenta in-situ, and referring the subject to bed rest to prevent preterm labo.

Embodiment I-41. A method comprising administering to a pregnant subject determined to have an increased risk of placenta accreta by a method as described herein, a therapeutic intervention effective to reduce the risk of placenta accreta.

Embodiment I-42. A method of administering to a pregnant subject an effective amount of a treatment designed to reduce the risk of placenta accreta, wherein the subject has an altered quantitative measure as compared to a reference standard of any one of the panels of protein biomarkers selected from:
(i) a protein biomarker of Table 1, Table 3 or Table 5, wherein the blood sample is collected at about 24 weeks of pregnancy; and
(ii) a protein biomarker of Table 2, Table 4 or Table 6, wherein the blood sample is collected at about 34 weeks of pregnancy.

Embodiment I-43. The method of embodiment I-42, wherein the protein biomarker is a surrogate biomarker selected from:
(i) a surrogate biomarker of FIGS. 10A-10C, FIGS. 11A-11C or FIGS. 12A-12H; and
(ii) a surrogate biomarker of FIGS. 13A-13H, FIGS. 14A-14D or FIGS. 15A-15B.

Embodiment I-44. A method comprising:
a) measuring, via mass spectrometry, masses of more than 100, more than 1000, more than 10,000 or more than 100,000 peptides from a biological sample comprising peptide fragments of proteins, to produce a dataset comprising more than 100, more than 1000, more than 10,000 or more than 100,000 data entries;
b) at a computer system comprising one or more processors and memory storing programs foe execution by the one or more processors;
   (1) identifying from among the data entries, based on the masses, peptides corresponding to:
      (i) a protein biomarker of Table 1, Table 3 or Table 5, wherein the biological sample is collected at about 24 weeks of pregnancy; and
      (ii) a protein biomarker of Table 2, Table 4 or Table 6, wherein the biological sample is collected at about 34 weeks of pregnancy;
   (2) executing a classification rule on the measures of the identified peptides, which classification rule classifies the sample as being from subject at increased risk of placenta accreta.

### Set II:

Embodiment II-1. A method of assessing the risk of placenta accreta in a pregnant subject, comprising:
(a) providing a sample from a pregnant subject between about 20 weeks of pregnancy to about 37 weeks of pregnancy;
(b) preparing a microparticle-associated peptide fraction from the sample;
(c) measuring a plurality of protein biomarkers in the fraction; and
(d) executing a classification rule on one or more measurement values of (c), wherein the classification rule classifies the sample as being from a subject at increased risk of placenta accreta.

Embodiment II-2. The method of embodiment II-1, wherein the protein biomarkers comprise a panel of no more than any of 10, 9, 8, 7, 6, 5, 4 or 3 protein biomarkers.

Embodiment II-3. The method of embodiment II-1, wherein the protein biomarkers comprise, consist essentially of or consist of a panel of biomarkers selected from:
(i) a biomarker panel of Table 7; and
(ii) a protein biomarker of Table 8.

Embodiment II-4. The method of embodiment II-1, wherein the plurality of protein biomarkers comprise:
(i) a plurality of protein biomarkers from Table 1, Table 3, and Table 5;
(ii) a plurality of protein biomarkers from Table 2, Table 4, and Table 6;
(iii) a plurality of protein biomarkers from Table 9;
(iv) two or more of isthmin-2 (ISM2), sulfhydryl oxidase 1 (QSOX1), histone H4 (H4), hemoglobin subunit gamma-2 (HBG2) and cartilage acidic protein 1 (CRAC1); or
(v) two or more of isthmin-2 (ISM2), ubiquitin carboxyl-terminal hydrolase 1 (UBP1), immunoglobulin lambda variables 10-54 (LVX54) and Ig-like domain-containing protein.

Embodiment II-5. The method of embodiment II-1, wherein measuring the plurality of biomarkers comprises measuring the relevant surrogate biomarkers of FIGS. 10A-10C, FIGS. 11A-11C, FIGS. 12A-12H, FIGS. 13A-13H, FIGS. 14A-14D, and FIGS. 15A-15B.

Embodiment II-6. The method of embodiment II-1, wherein the pregnant subject has one or more risk factors for placenta accreta.

Embodiment II-7. The method of embodiment II-1, wherein the pregnant subject is primigravida, multigravida, primiparous or multiparous.

Embodiment II-8. The method of embodiment II-1, wherein the sample is a blood sample.

Embodiment II-9. The method of embodiment II-1, wherein the sample is plasma or serum.

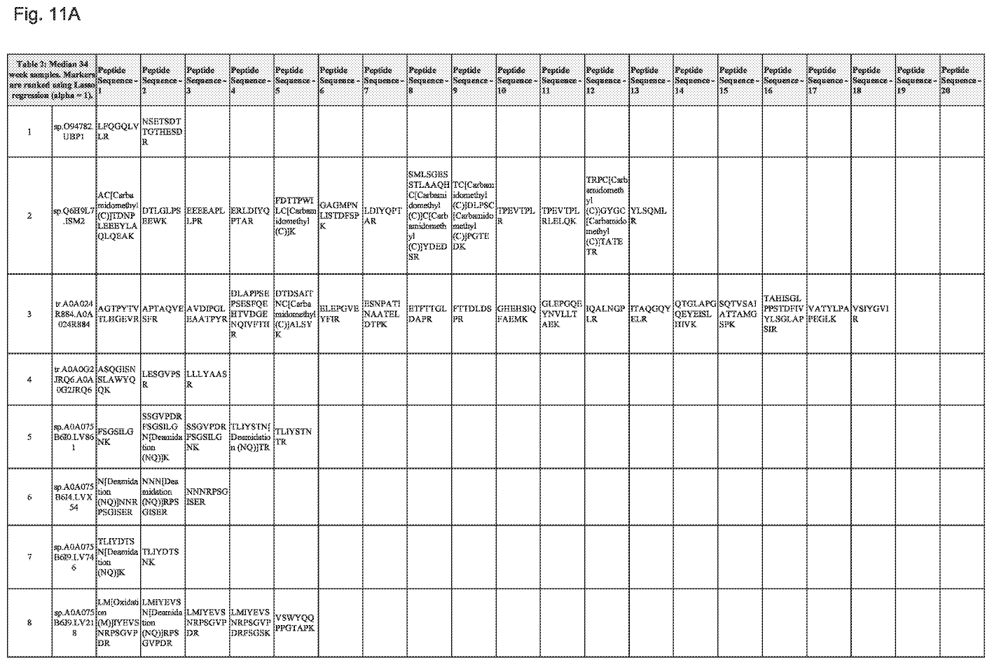

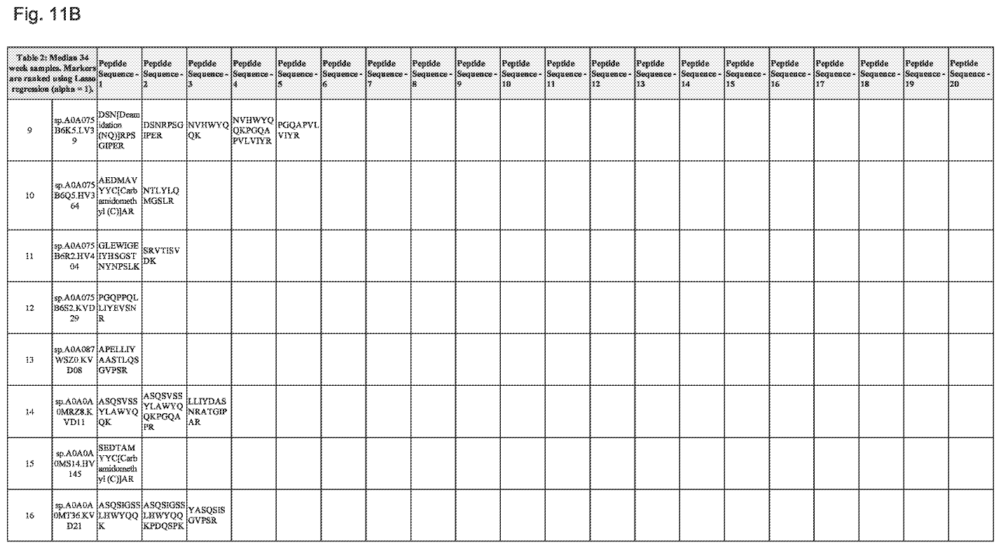

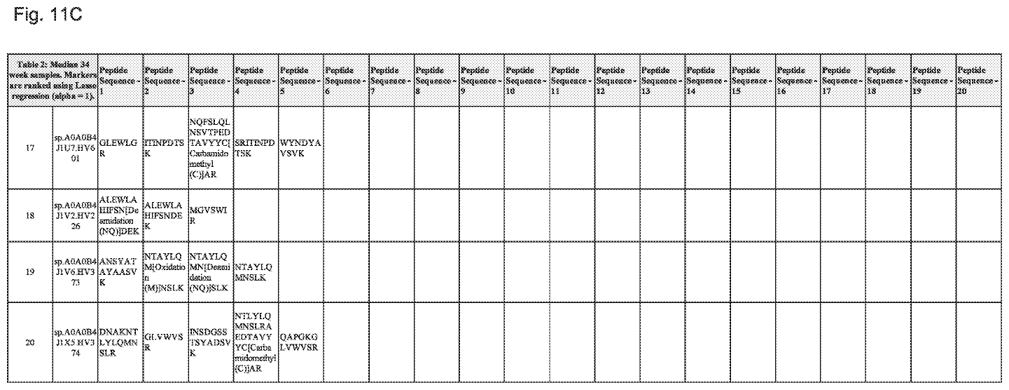

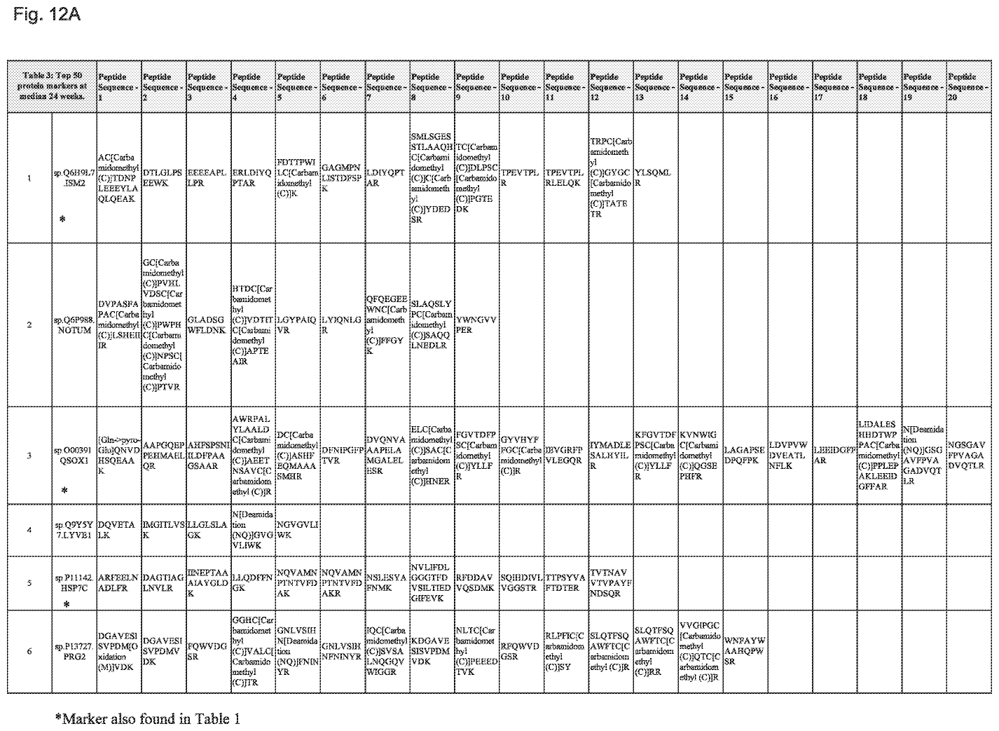

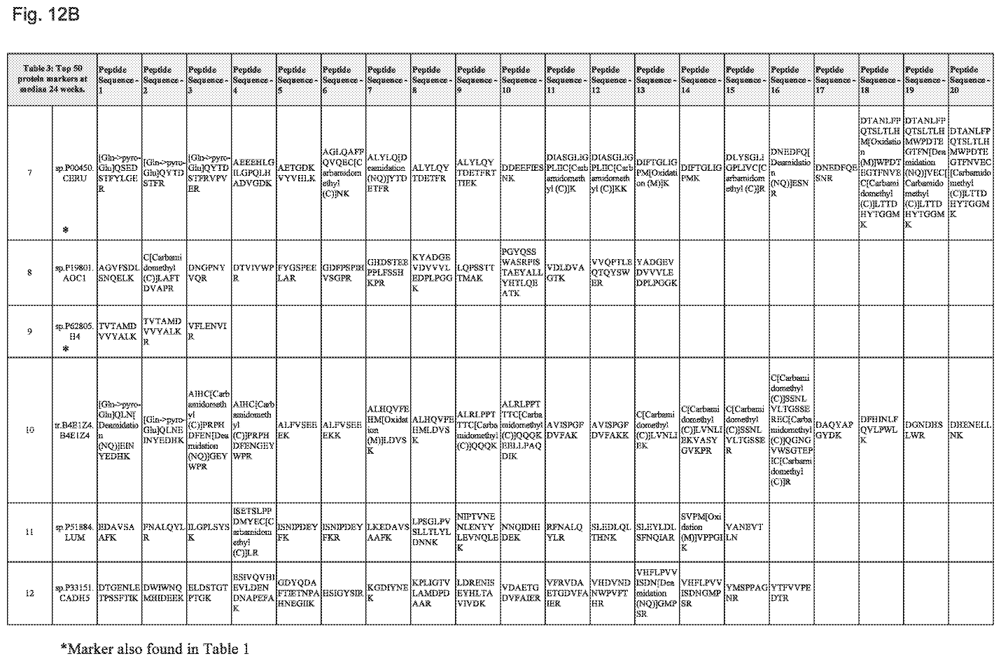

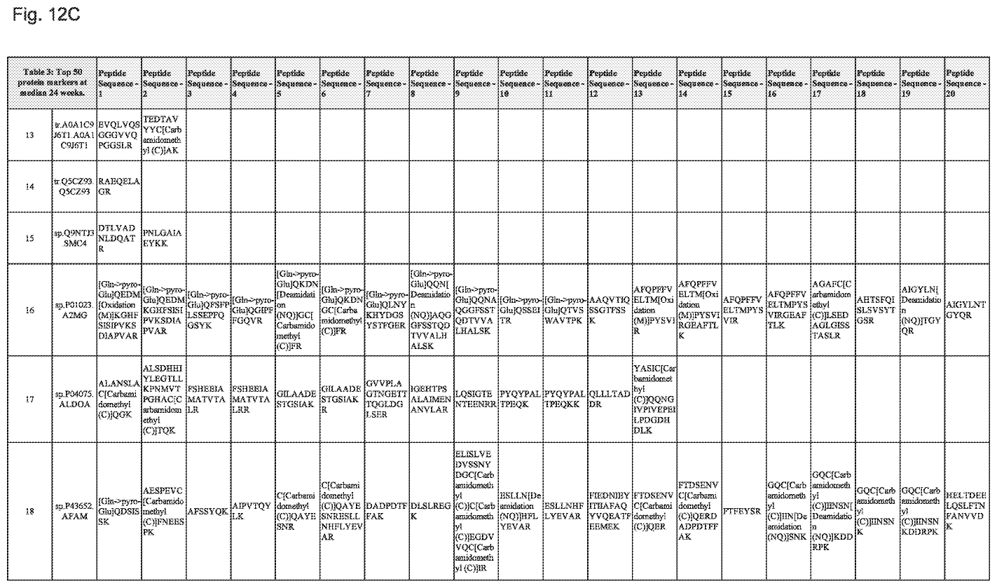

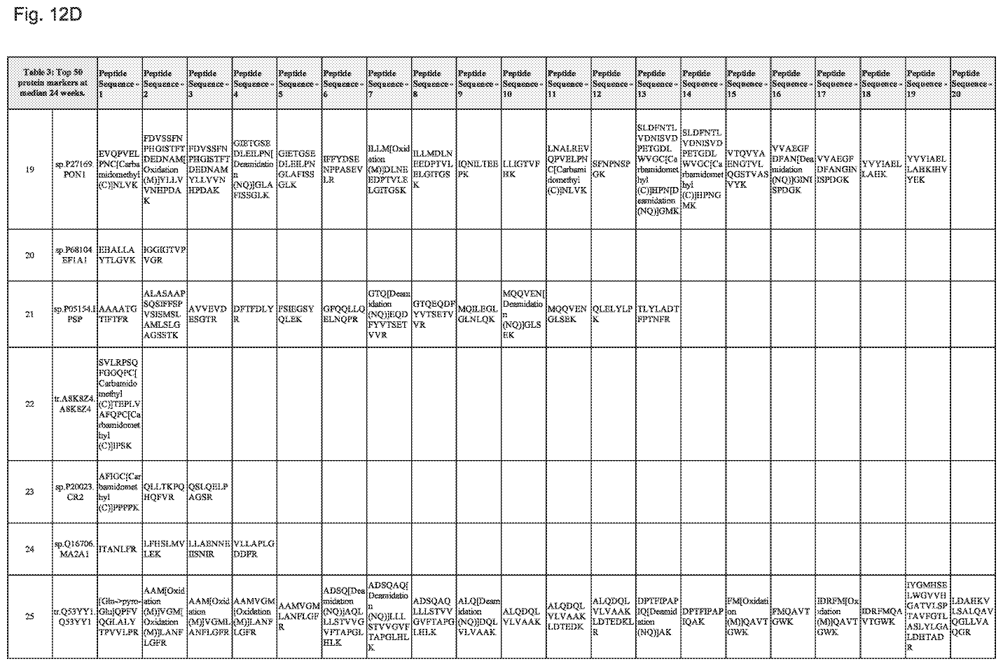

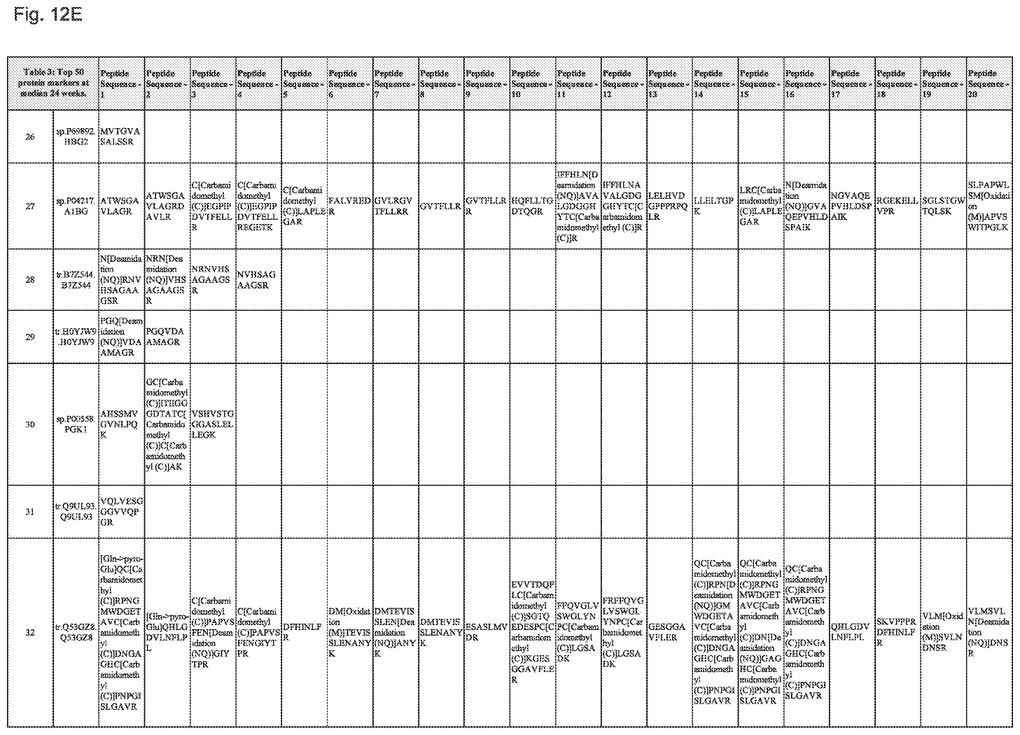

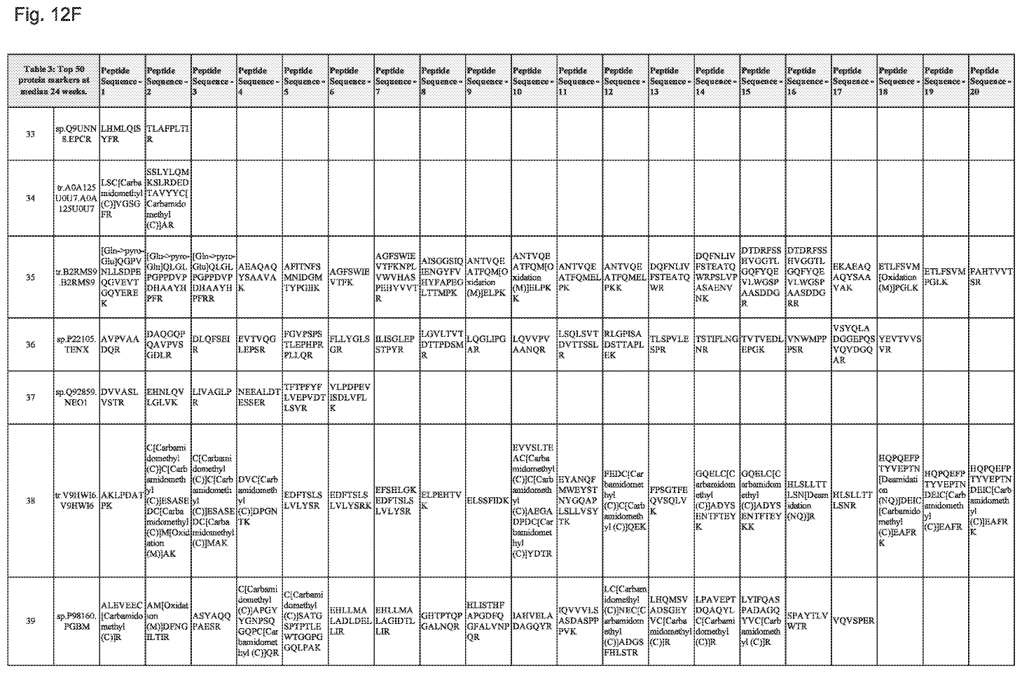

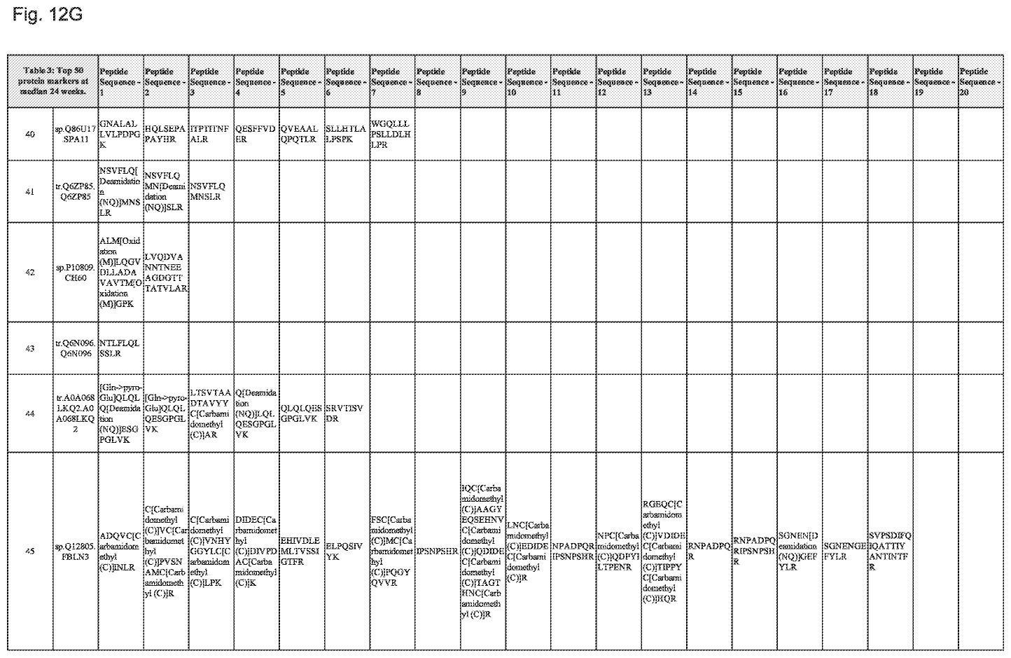

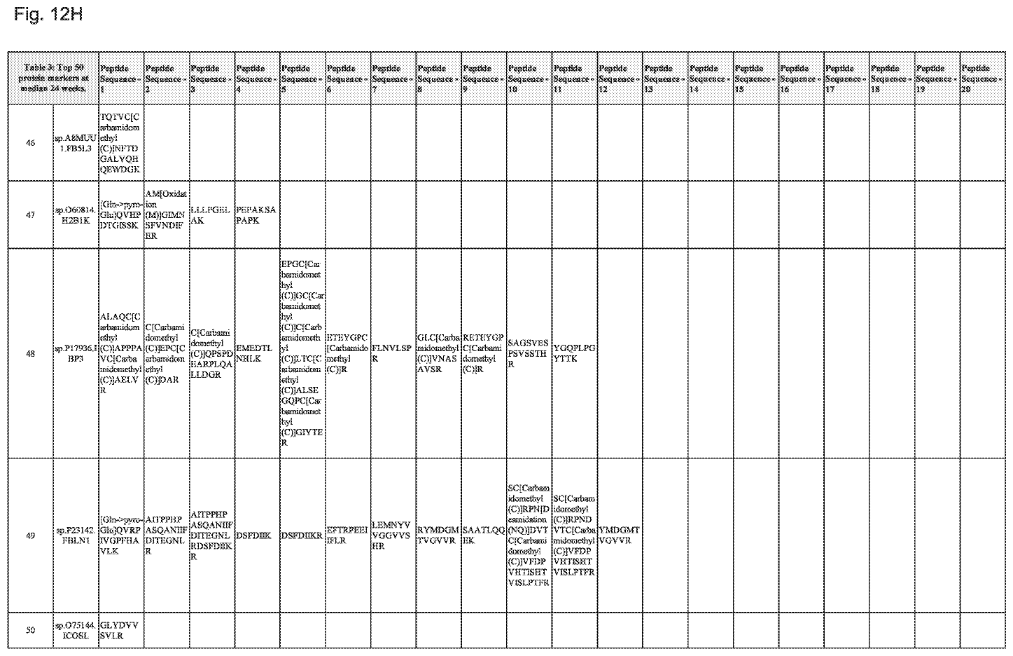

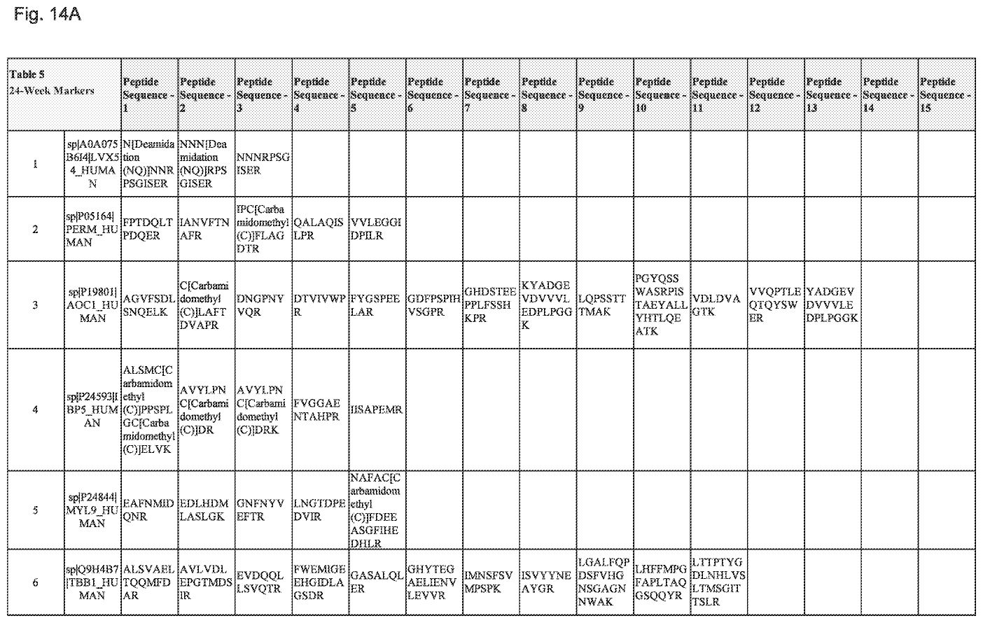

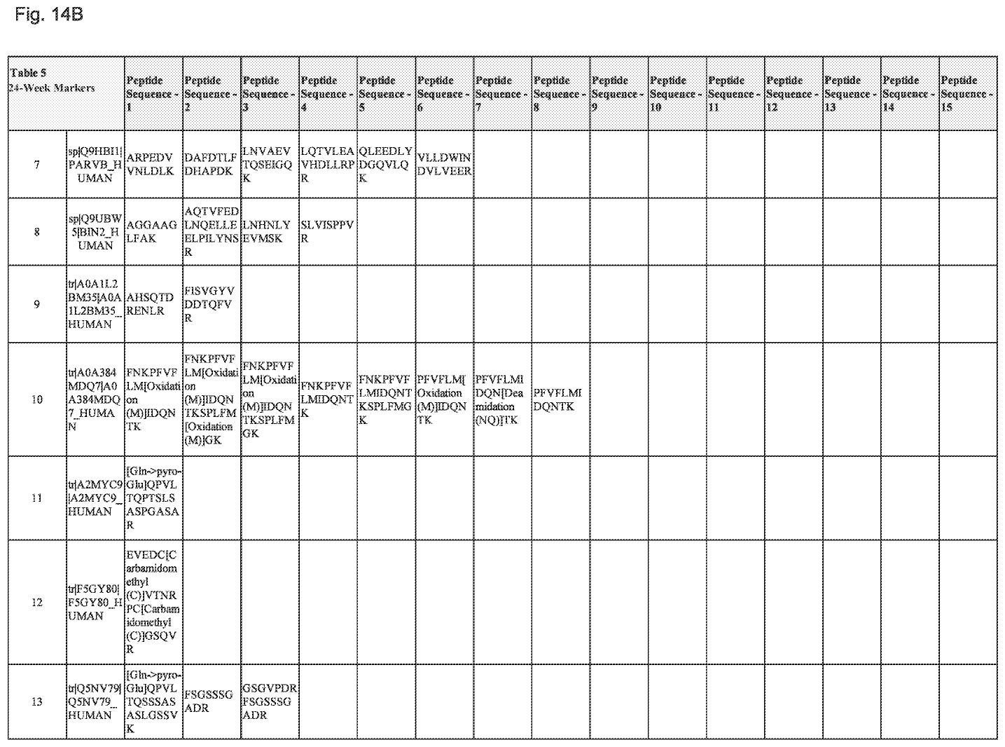

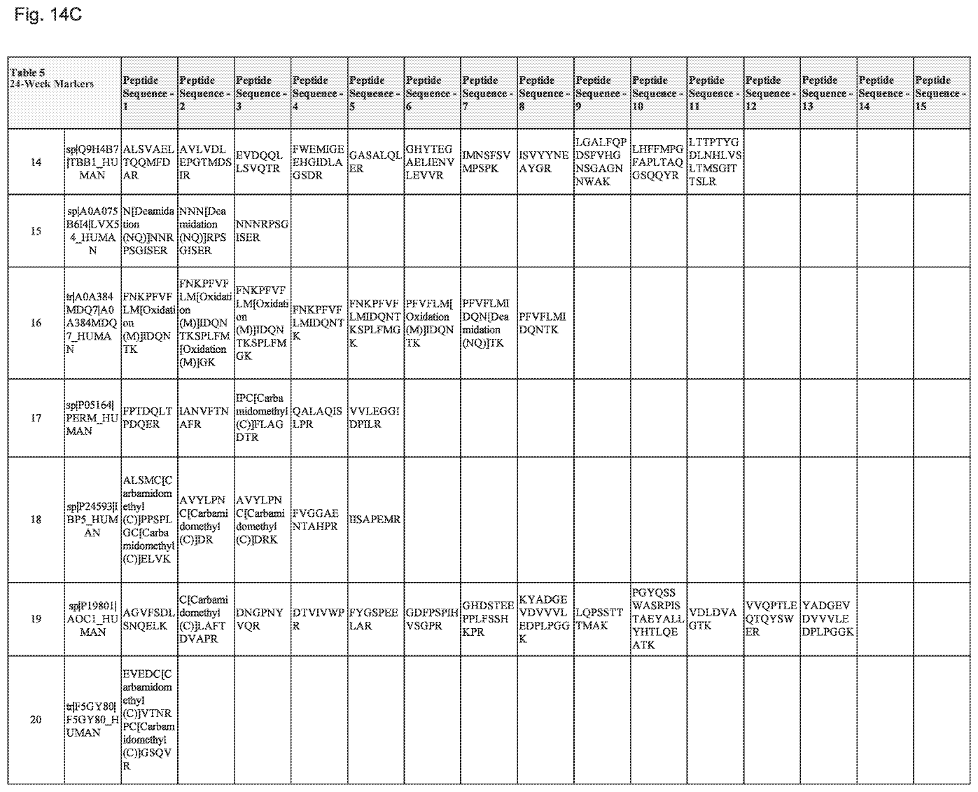

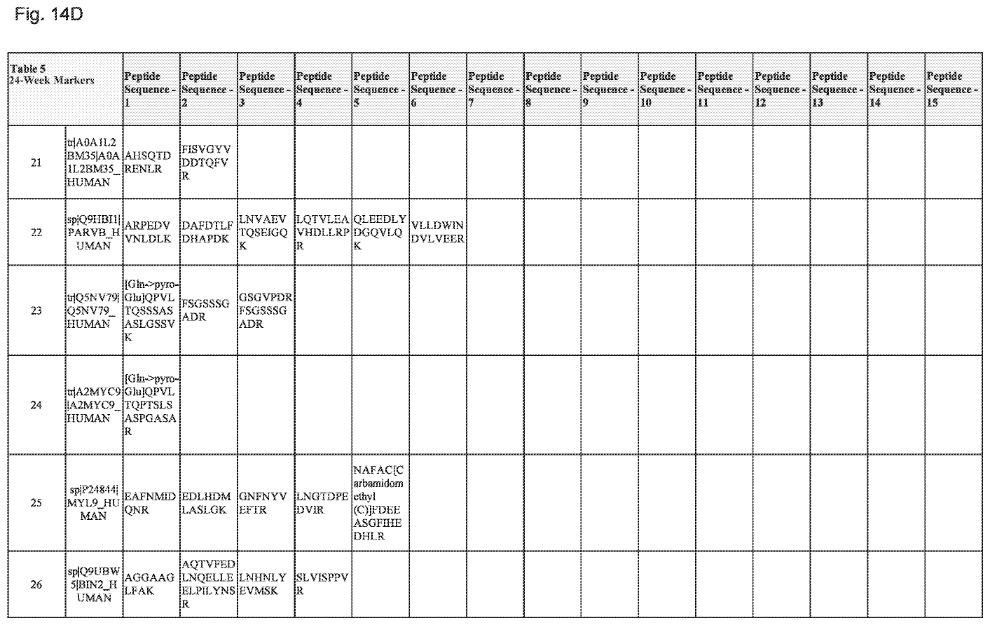

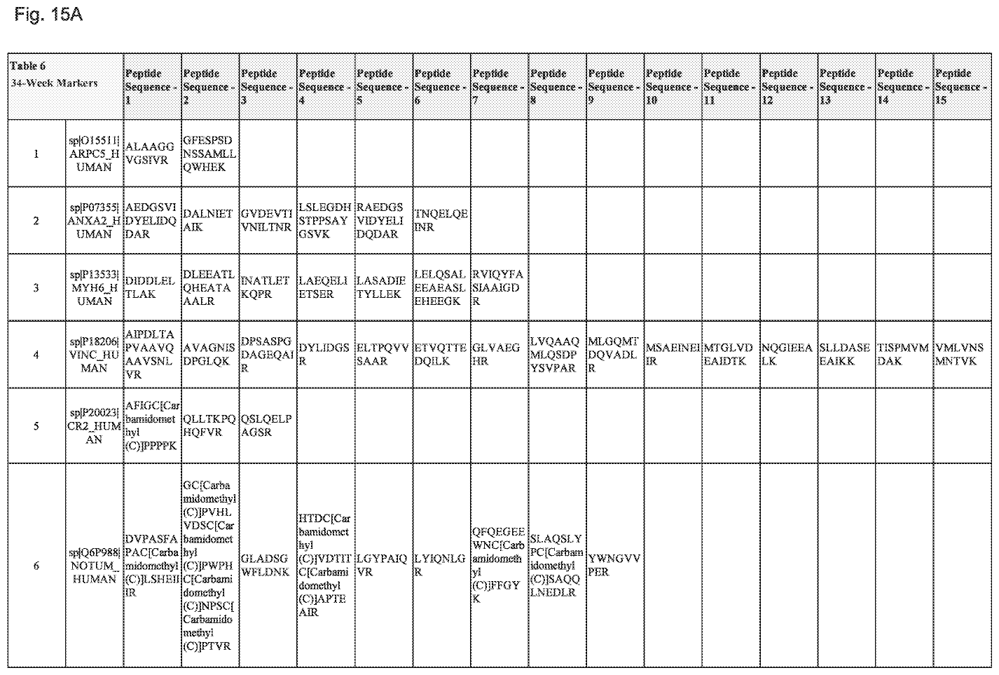

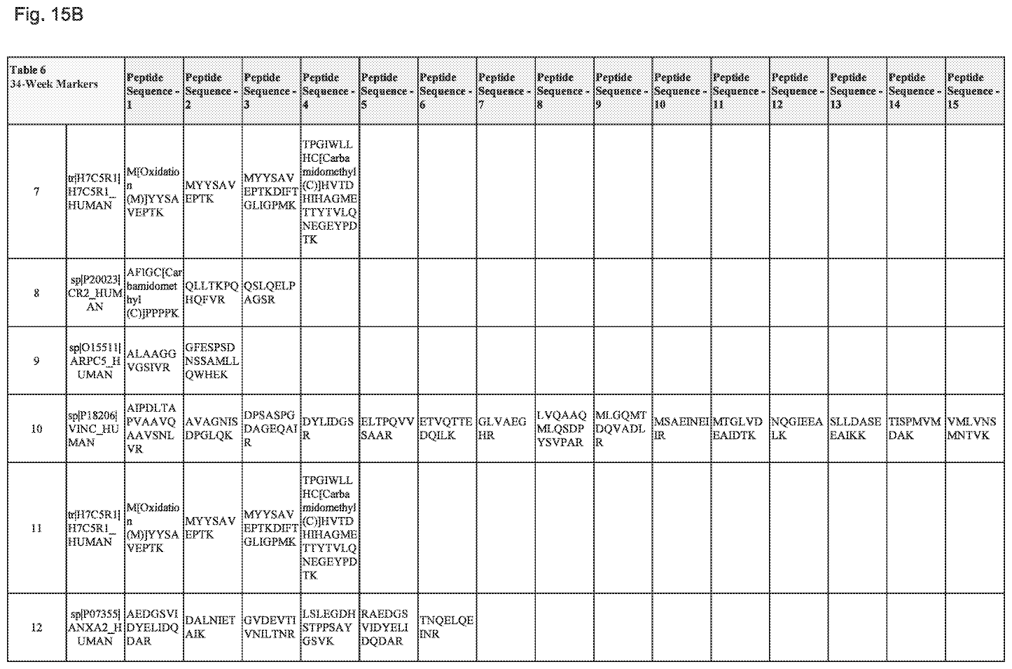

Embodiment II-10. A panel comprising a plurality of substantially pure protein biomarkers or surrogate biomarkers selected from:
(i) a protein biomarker of Table 1, Table 3, or Table 5;
(ii) a protein biomarker of Table 2, Table 4, or Table 6
(iii) a protein biomarker of Table 9;
(iv) a surrogate biomarker of FIGS. 10A-10C, FIGS. 11A-11C, or FIGS. 12A-12H;
(v) a surrogate biomarker of FIGS. 13A-13H, FIGS. 14A-14D, or FIGS. 15A-15B;
(vi) isthmin-2 (ISM2), sulfhydryl oxidase 1 (QSOX1), histone H4 (H4), hemoglobin subunit gamma-2 (HBG2) and cartilage acidic protein 1 (CRAC1); and
(vii) isthmin-2 (ISM2), ubiquitin carboxyl-terminal hydrolase 1 (UBP1), immunoglobulin lambda variables 10-54 (LVX54) and Ig-like domain-containing protein.

Embodiment II-11. The panel of embodiment II-10, further comprising a stable isotope standard peptide paired with each of the surrogate biomarkers of FIGS. 10A-10C, FIGS. 11A-11C, FIGS. 12A-12H, FIGS. 13A-13H, FIGS. 14A-14D, and FIGS. 15A-15B.

Embodiment II-12. A method of preparing a peptide sample, comprising:
(a) providing a sample from a pregnant subject between about 20 weeks of pregnancy to about 37 weeks of pregnancy;
(b) enriching the sample for microparticles by loading the sample on a size-exclusion column and eluting the microparticles from the column using water as a mobile phase, to produce a microparticle-enriched fraction;
(c) preparing a microparticle-associated peptide fraction from the microparticle-enriched fraction by contacting the microparticle-enriched fraction with a protease;
(d) separating the microparticle-associated peptides by mass spectrometry; and
(e) measuring, based on a mass spectrometry signal, one or more peptides corresponding to one or more protein biomarkers.

Embodiment II-13. The method of embodiment II-12, wherein the one or more protein biomarkers includes a plurality of protein biomarkers.

Embodiment II-14. The method of embodiment II-12, wherein the protein biomarkers comprise a panel of no more than any of 10, 9, 8, 7, 6, 5, 4 or 3 protein biomarkers.

Embodiment II-15. The method of embodiment II-12, wherein the protein biomarkers comprise, consist essentially of or consist of a panel of biomarkers selected from:
(i) a biomarker panel of Table 7; and
(ii) a protein biomarker of Table 8.

Embodiment II-16. The method of embodiment II-12, wherein the protein biomarkers comprise:
(i) one or more of isthmin-2 (ISM2), sulfhydryl oxidase 1 (QSOX1), histone H4 (H4), hemoglobin subunit gamma-2 (HBG2) and cartilage acidic protein 1 (CRAC1); or
(ii) one or more of isthmin-2 (ISM2), ubiquitin carboxyl-terminal hydrolase 1 (UBP1), immunoglobulin lambda variables 10-54 (LVX54) and Ig-like domain-containing protein.

Embodiment II-17. The method of embodiment II-12, wherein measuring the one or more peptides comprises measuring a surrogate biomarker of any of FIGS. 10A-10C, FIGS. 11A-11C, FIGS. 12A-12H, FIGS. 13A-13H, FIGS. 14A-14D, and FIGS. 15A-15B.

Embodiment II-18. The method of embodiment II-12, wherein the pregnant subject has one or more risk factors for placenta accreta.

Embodiment II-19. The method of embodiment II-12, wherein the pregnant subject is primigravida, multigravida, primiparous or multiparous.

Embodiment II-20. The method of embodiment II-12, wherein the blood sample is plasma or serum.

Embodiment II-21. The method of embodiment II-12, wherein the water is deionized distilled water.

Embodiment II-22. The method of embodiment II-12, wherein the size-exclusion column comprises an agarose solid phase and an aqueous liquid phase.

Embodiment II-23. The method of embodiment II-12, wherein preparing the microparticle-associated peptide fraction further comprises using ultrafiltration or reverse-phase chromatography.

Embodiment II-24. The method of embodiment II-12, wherein preparing the microparticle-associated peptide fraction further comprises denaturation of the microparticle-enriched fraction using urea, reduction of the microparticle-enriched fraction using dithiothreitol, alkylation of the microparticle-enriched fraction using iodoacetamine, and digestion of the microparticle-enriched fraction using trypsin.

Embodiment II-25. The method of embodiment II-12, wherein enriching the sample for microparticles includes further purifying the microparticles to enrich for placental-derived exosomes or vascular endothelial-derived exosomes.

Embodiment II-26. The method of embodiment II-12, wherein separating the microparticle-associated peptides by mass spectrometry comprises separating the microparticle-associated peptides by liquid chromatography/mass spectrometry (LC/MS) including liquid chromatography/triple quadrupole mass spectrometry.

Embodiment II-27. The method of embodiment II-12, wherein separating the microparticle-associated peptides by mass spectrometry includes the mass spectrometry comprising multiple reaction monitoring.

Embodiment II-28. The method of embodiment II-12, wherein the one or more peptides are selected from:
(i) a biomarker panel of Table 7, wherein the blood sample is collected at about 20 weeks of pregnancy; and
(ii) a protein biomarker of Table 8, wherein the blood sample is collected at about 37 weeks of pregnancy.

Embodiment II-29. A kit comprising one or a plurality of containers, wherein each container comprises one or more of each of a plurality of Stable Isotopic Standards, wherein each stable isotopic standard corresponds to a surrogate peptide for a biomarker from a panel of biomarkers selected from:
(i) a protein biomarker of Table 1, Table 3, or Table 5;
(ii) a protein biomarker of Table 2, Table 4, or Table 6;
(iii) a protein biomarker of Table 9;
(iv) a surrogate biomarker of FIGS. 10A-10C, FIGS. 11A-11C, or FIGS. 12A-12H;
(v) a surrogate biomarker of FIGS. 13A-13H, FIGS. 14A-14D, or FIGS. 15A-15B;
(vi) isthmin-2 (ISM2), sulfhydryl oxidase 1 (QSOX1), histone H4 (H4), hemoglobin subunit gamma-2 (HBG2) and cartilage acidic protein 1 (CRAC1); and
(vi) isthmin-2 (ISM2), ubiquitin carboxyl-terminal hydrolase 1 (UBP1), immunoglobulin lambda variables 10-54 (LVX54) and Ig-like domain-containing protein.

Embodiment II-30. A composition comprising one or a plurality of pairs of polypeptides, wherein each pair of polypeptides comprise one or more protein biomarkers or one or more surrogate biomarkers selected from:
(i) a protein biomarker of Table 1, Table 3, or Table 5;
(ii) a protein biomarker of Table 2, Table 4, or Table 6
(iii) a protein biomarker of Table 9;
(iv) a surrogate biomarker of FIGS. 10A-10C, FIGS. 11A-11C, or FIGS. 12A-12H;
(v) a surrogate biomarker of FIGS. 13A-13H, FIGS. 14A-14D, or FIGS. 15A-15B;
(vi) isthmin-2 (ISM2), sulfhydryl oxidase 1 (QSOX1), histone H4 (H4), hemoglobin subunit gamma-2 (HBG2) and cartilage acidic protein 1 (CRAC1); and
(vii) isthmin-2 (ISM2), ubiquitin carboxyl-terminal hydrolase 1 (UBP1), immunoglobulin lambda variables 10-54 (LVX54) and Ig-like domain-containing protein.

Embodiment II-31. A computer readable medium in tangible, non-transitory form comprising code implementing one or more classification rules generated by analysis of one or more datasets of biomarker measurements derived from one or more pregnant subjects classified into a first group at risk for placenta accreta or a second group not at risk of placenta accreta.

Embodiment II-32. A system comprising:
(a) a computer comprising:
   (i) a processor; and
   (ii) a memory, coupled to the processor, the memory storing a module comprising:
      (1) test data for a sample from a subject including one or more values, wherein each value indicates a measurement of one or more protein biomarkers in a fraction of microparticle-associated peptides, wherein the one or more protein biomarkers are selected from:
         (i) a protein biomarker of Table 1, Table 3, or Table 5, wherein the blood sample is collected at about 20 weeks of pregnancy;
         (ii) a protein biomarker of Table 2, Table 4, or Table 6, wherein the blood sample is collected at about 37 weeks of pregnancy;
         (iii) a protein biomarker of Table 9;
         (iv) one or more of isthmin-2 (ISM2), sulfhydryl oxidase 1 (QSOX1), histone H4 (H4), hemoglobin subunit gamma-2 (HBG2) and/or cartilage acidic protein 1 (CRAC1); and
         (v) one or more of isthmin-2 (ISM2), ubiquitin carboxyl-terminal hydrolase 1 (UBP1), immunoglobulin lambda variables 10-54 (LVX54) and/or Ig-like domain-containing protein; and
      (2) a classification rule which, based on the one or more values wherein each value indicates the measurement, classifies the subject as being at increased risk of placenta accreta, wherein the classification rule is configured to have a sensitivity value of at least 75%, at least 85% or at least 95%; and
      (3) computer executable instructions for implementing the classification rule on the test data.

Embodiment II-33. The system of embodiment II-32, wherein the protein biomarker is a surrogate biomarker selected from:
(i) a surrogate biomarker of FIGS. 10A-10C, FIGS. 11A-11C, or FIGS. 12A-12H; and
(ii) a surrogate biomarker of FIGS. 13A-13H, FIGS. 14A-14D, or FIGS. 15A-15B.

Embodiment II-34. A method comprising:
(a) using a computer system to compile test data, the computer system comprising a processor and a memory, coupled to the processor, the memory storing a module comprising:
   (1) test data for a sample from a subject including values indicating one or more measurement values of one or more protein biomarkers of the disclosure in a fraction of microparticle-associated peptides;
   (2) a classification rule to be executed by the processor, which, based on values including the one or more measurement values, classifies the subject as being at increased risk of placenta accreta, wherein the classification rule is configured to have a sensitivity of at least 75%, at least 85% or at least 95%;
(b) accessing the test data; and
(c) executing the classification rule on the test data.

Embodiment II-35. A method of assessing risk of placenta accreta in a pregnant subject, the method comprising:
(a) preparing a microparticle-enriched fraction from a blood sample from a pregnant subject;
(b) determining a quantitative measure of one or more microparticle-associated protein biomarkers in the microparticle-enriched fraction, wherein the one or more microparticle-associated protein biomarkers are selected from:
   (i) a protein biomarker of Table 1, Table 3, or Table 5, wherein the blood sample is collected at about 20 weeks of pregnancy;
   (ii) a protein biomarker of Table 2, Table 4, or Table 6, wherein the blood sample is collected at about 37 weeks of pregnancy;
   (iii) a protein biomarker of Table 9;
   (iv) one or more of isthmin-2 (ISM2), sulfhydryl oxidase 1 (QSOX1), histone H4 (H4), hemoglobin subunit gamma-2 (HBG2) and/or cartilage acidic protein 1 (CRAC1); and
   (v) one or more of isthmin-2 (ISM2), ubiquitin carboxyl-terminal hydrolase 1 (UBP1), immunoglobulin lambda variables 10-54 (LVX54) and/or Ig-like domain-containing protein; and
(c) assessing risk of placenta accreta based on the one or more quantitative measures.

Embodiment II-36. The method of embodiment II-35, wherein the protein biomarker is a surrogate biomarker selected from:
(i) a surrogate biomarker of FIGS. 10A-10C, FIGS. 11A-11C, or FIGS. 12A-12H;
(ii) a surrogate biomarker of FIGS. 13A-13H, FIGS. 14A-14D, or FIGS. 15A-15B;
(iii) a surrogate biomarker of isthmin-2 (ISM2), sulfhydryl oxidase 1 (QSOX1), histone H4 (H4), hemoglobin subunit gamma-2 (HBG2) and/or cartilage acidic protein 1 (CRAC1); and
(iv) a surrogate biomarker of isthmin-2 (ISM2), ubiquitin carboxyl-terminal hydrolase 1 (UBP1), immunoglobulin lambda variables 10-54 (LVX54) and/or Ig-like domain-containing protein.

Embodiment II-37. The method of embodiment II-35, wherein determining the quantitative measure of one or more microparticle-associated protein biomarkers comprises contacting the sample with one or more capture reagents, each capture reagent specifically binding one of the protein biomarkers, and detecting binding between the capture reagent and the protein biomarker.

Embodiment II-38. The method of embodiment II-37, wherein determining the quantitative measure of one or more microparticle-associated protein biomarkers comprises performing an immunoassay.

Embodiment II-39. The method of embodiment II-38, wherein the immunoassay is selected from the group consisting of an enzyme immunoassay (EIA), an enzyme-linked immunosorbent assay (ELISA), and a radioimmunoassay (RIA).

Embodiment II-40. The method of embodiment II-35, wherein the assessing risk of placenta accreta comprises executing a classification rule, wherein the classification rule classifies the subject at being at risk of placenta accreta, and wherein execution of the classification rule produces a correlation between placenta accreta or term birth with a p value of less than at least 0.05.

Embodiment II-41. The method of embodiment II-35, wherein the assessing risk of placenta accreta comprises executing a classification rule, wherein the classification rule classifies the subject at being at risk of placenta accreta, and wherein execution of the classification rule produces a receiver operating characteristic (ROC) curve, wherein the ROC curve has an area under the curve (AUC) of at least 0.6, at least 0.7, at least 0.8 or at least 0.9.

Embodiment II-42. The method of embodiment II-35, wherein the classification rule classifies a subject based on one or more values wherein the one or more values further include at least one of: placenta previa, previous cesarean delivery, endometrial ablation, in vitro fertilization, prior uterine infection, or previous uterine surgery.

Embodiment II-43. The method of embodiments II-25 to II-32, wherein the classification rule employs cut-off, linear regression including multiple linear regression, partial least squares regression, principal components regression, binary decision trees including recursive partitioning processes further including classification and regression trees, artificial neural networks including back propagation networks, discriminant analyses further includingBayesian classifier or Fischer analysis, logistic classifiers, and support vector classifiers including support vector machines.

Embodiment II-44. The method of embodiment II-35, wherein the classification rule is configured to have a sensitivity value, a specificity value, a positive predictive value, or a negative predictive value of at least 70%, least 80%, at least 90% or at least 95%.

Embodiment II-45. The method of embodiment II-35, wherein assessing risk of placenta accreta comprises determining that the protein biomarker, if upregulated, is above a threshold level or if down regulated, is below the threshold level.

Embodiment II-46. The method of embodiment II-45, wherein the threshold level represents a level at least one, at least two or at least three z scores from a measure of central tendency including a mean, a median or a mode for the protein biomarker determined from at least 50, at least 100 or at least 200 control subjects.

Embodiment II-47. The method of embodiment II-35, wherein the assessing risk of placenta accreta comprises comparing the one or more quantitative measures of each protein biomarker in the panel to a reference standard.

Embodiment II-48. The method of embodiment II-35, further comprising communicating the risk of placenta accreta for a pregnant subject to a health care provider.

Embodiment II-49. A method of treating placenta accreta in a pregnant subject, the method comprising:
(a) assessing risk of placenta accreta for a pregnant subject according to the method of any one of embodiments II-1 to II-9, and II-35 to II-48; and
(b) administering a therapeutic intervention to the subject effective to decrease the risk of placenta accreta and/or reduce neonatal complications of placenta accreta.

Embodiment II-50. The method of embodiment II-49, wherein administering the therapeutic intervention comprises a therapeutic intervention selected from the group consisting of:
(i) referring the subject to a medical center having advanced multidisciplinary surgical expertise and experience;
(ii) planning surgical uterine conservation; and
(iii) performing a Cesarean hysterectomy, performing a prophylactic embolization, inserting a uterine balloon tamponade, a temporal internal iliac occlusion balloon catheter, a ureteral stents, administering methotrexate, leaving a portion of the placenta in-situ, and referring the subject to bed rest to prevent preterm labor.

Embodiment II-51. A method comprising administering to a pregnant subject determined to have an increased risk of placenta accreta by a method according to any one of embodiments II-1 to II-9, and II-35 to II-48, a therapeutic intervention effective to reduce the risk of placenta accreta.

Embodiment II-52. A method of administering to a pregnant subject an effective amount of a treatment designed to reduce the risk of placenta accreta, wherein the subject has an altered quantitative measure as compared to a reference standard of any one of a panel of protein biomarkers selected from:
(i) a protein biomarker of Table 1, Table 3, or Table 5, wherein the blood sample is collected at about 20 weeks of pregnancy;
(ii) a protein biomarker of Table 2, Table 4, or Table 6, wherein the blood sample is collected at about 37 weeks of pregnancy;
(iii) a protein biomarker of Table 9;
(iv) one or more of isthmin-2 (ISM2), sulfhydryl oxidase 1 (QSOX1), histone H4 (H4), hemoglobin subunit gamma-2 (HBG2) and/or cartilage acidic protein 1 (CRAC1); and
(v) one or more of isthmin-2 (ISM2), ubiquitin carboxyl-terminal hydrolase 1 (UBP1), immunoglobulin lambda variables 10-54 (LVX54) and/or Ig-like domain-containing protein.

Embodiment II-53. The method of embodiment II-52, wherein the protein biomarker is a surrogate biomarker selected from:
(i) a surrogate biomarker of FIGS. 10A-10C, FIGS. 11A-11C, or FIGS. 12A-12H;
(ii) a surrogate biomarker of FIGS. 13A-13H, FIGS. 14A-14D, or FIGS. 15A-15B;
(iii) a surrogate biomarker of isthmin-2 (ISM2), sulfhydryl oxidase 1 (QSOX1), histone H4 (H4), hemoglobin subunit gamma-2 (HBG2) and/or cartilage acidic protein 1 (CRAC1); and
(iv) a surrogate biomarker of isthmin-2 (ISM2), ubiquitin carboxyl-terminal hydrolase 1 (UBP1), immunoglobulin lambda variables 10-54 (LVX54) and/or Ig-like domain-containing protein.

Embodiment II-54. A method comprising:
a) measuring, via mass spectrometry, masses of more than 100, more than 1000, more than 10,000 or more than 100,000 peptides from a biological sample comprising peptide fragments of proteins, to produce a dataset comprising more than 100, more than 1000, more than 10,000 or more than 100,000 data entries;
b) using a computer system comprising one or more processors and memory storing programs for execution by the one or more processors in
   (1) identifying from among the data entries, based on the masses, peptides corresponding to:
      (i) a protein biomarker of Table 1, Table 3, or Table 5, wherein the biological sample is collected at about 20 weeks of pregnancy; and
      (ii) a protein biomarker of Table 2, Table 4, or Table 6, wherein the biological sample is collected at about 37 weeks of pregnancy;
      (iii) a protein biomarker of Table 9;
      (iv) one or more of isthmin-2 (ISM2), sulfhydryl oxidase 1 (QSOX1), histone H4 (H4), hemoglobin subunit gamma-2 (HBG2) and/or cartilage acidic protein 1 (CRAC1); and
      (v) one or more of isthmin-2 (ISM2), ubiquitin carboxyl-terminal hydrolase 1 (UBP1), immunoglobulin lambda variables 10-54 (LVX54) and/or Ig-like domain-containing protein.
   (2) executing a classification rule on one or more measurement values of the identified peptides, wherein the classification rule classifies the sample as being from a subject at increased risk of placenta accreta.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination. Unless otherwise specified, the term “about” in reference to a value refers to 90% to 110% of that value or 95% to 105% of that value.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

1. A method of assessing the risk of placenta accreta in a pregnant subject, comprising:
(a) providing a sample from a pregnant subject between about 20 weeks of pregnancy to about 37 weeks of pregnancy;
(b) preparing a microparticle-associated peptide fraction from the sample;
(c) measuring a plurality of protein biomarkers in the fraction; and
(d) executing a classification rule on one or more measurement values of (c), wherein the classification rule classifies the sample as being from a subject at increased risk of placenta accreta.

2. The method of claim 1, wherein the protein biomarkers comprise a panel of no more than any of 10, 9, 8, 7, 6, 5, 4 or 3 protein biomarkers.

3. The method of claim 1, wherein the protein biomarkers comprise, consist essentially of or consist of a panel of biomarkers selected from:
(i) a biomarker panel of Table 7; and
(ii) a protein biomarker of Table 8.

4. The method of claim 1, wherein the plurality of protein biomarkers comprise:
(i) a plurality of protein biomarkers from Table 1, Table 3, and Table 5;
(ii) a plurality of protein biomarkers from Table 2, Table 4, and Table 6;
(iii) a plurality of protein biomarkers from Table 9;
(iv) two or more of isthmin-2 (ISM2), sulfhydryl oxidase 1 (QSOX1), histone H4 (H4), hemoglobin subunit gamma-2 (HBG2) and cartilage acidic protein 1 (CRAC1); or
(v) two or more of isthmin-2 (ISM2), ubiquitin carboxyl-terminal hydrolase 1 (UBP1), immunoglobulin lambda variables 10-54 (LVX54) and Ig-like domain-containing protein.

5. The method of claim 1, wherein measuring the plurality of biomarkers comprises measuring the relevant surrogate biomarkers of FIGS. 10A-10C, FIGS. 11A-11C, FIGS. 12A-12H, FIGS. 13A-13H, FIGS. 14A-14D, and FIGS. 15A-15B.

6. The method of claim 1, wherein the pregnant subject has one or more risk factors for placenta accreta.

7. The method of claim 1, wherein the pregnant subject is primigravida, multigravida, primiparous or multiparous.

8. The method of claim 1, wherein the sample is a blood sample.

9. The method of claim 1, wherein the sample is plasma or serum.

10.-28. (canceled)

29. A kit comprising one or a plurality of containers, wherein each container comprises one or more of each of a plurality of Stable Isotopic Standards, wherein each stable isotopic standard corresponds to a surrogate peptide for a biomarker from a panel of biomarkers selected from:
(i) a protein biomarker of Table 1, Table 3, or Table 5;
(ii) a protein biomarker of Table 2, Table 4, or Table 6;
(iii) a protein biomarker of Table 9;
(iv) a surrogate biomarker of FIGS. 10A-10C, FIGS. 11A-11C, or FIGS. 12A-12H;
(v) a surrogate biomarker of FIGS. 13A-13H, FIGS. 14A-14D, or FIGS. 15A-15B;
(vi) isthmin-2 (ISM2), sulfhydryl oxidase 1 (QSOX1), histone H4 (H4), hemoglobin subunit gamma-2 (HBG2) and cartilage acidic protein 1 (CRAC1); and
(vii) isthmin-2 (ISM2), ubiquitin carboxyl-terminal hydrolase 1 (UBP1), immunoglobulin lambda variables 10-54 (LVX54) and Ig-like domain-containing protein.

30.-34. (canceled)

35. A method of assessing risk of placenta accreta in a pregnant subject, the method comprising:
(a) preparing a microparticle-enriched fraction from a blood sample from a pregnant subject;
(b) determining a quantitative measure of one or more microparticle-associated protein biomarkers in the microparticle-enriched fraction, wherein the one or more microparticle-associated protein biomarkers are selected from:
(i) a protein biomarker of Table 1, Table 3, or Table 5, wherein the blood sample is collected at about 20 weeks of pregnancy;
(ii) a protein biomarker of Table 2, Table 4, or Table 6, wherein the blood sample is collected at about 37 weeks of pregnancy;
(iii) a protein biomarker of Table 9;
(iv) one or more of isthmin-2 (ISM2), sulfhydryl oxidase 1 (QSOX1), histone H4 (H4), hemoglobin subunit gamma-2 (HBG2) and/or cartilage acidic protein 1 (CRAC1); and
(v) one or more of isthmin-2 (ISM2), ubiquitin carboxyl-terminal hydrolase 1 (UBP1), immunoglobulin lambda variables 10-54 (LVX54) and/or Ig-like domain-containing protein; and
(c) assessing risk of placenta accreta based on the one or more quantitative measures.

36. The method of claim 35, wherein the protein biomarker is a surrogate biomarker selected from:
(i) a surrogate biomarker of FIGS. 10A-10C, FIGS. 11A-11C, or FIGS. 12A-12H;
(ii) a surrogate biomarker of FIGS. 13A-13H, FIGS. 14A-14D, or FIGS. 15A-15B;
(iii) a surrogate biomarker of isthmin-2 (ISM2), sulfhydryl oxidase 1 (QSOX1), histone H4 (H4), hemoglobin subunit gamma-2 (HBG2) and/or cartilage acidic protein 1 (CRAC1); and
(iv) a surrogate biomarker of isthmin-2 (ISM2), ubiquitin carboxyl-terminal hydrolase 1 (UBP1), immunoglobulin lambda variables 10-54 (LVX54) and/or Ig-like domain-containing protein.

37. The method of claim 35, wherein determining the quantitative measure of one or more microparticle-associated protein biomarkers comprises contacting the sample with one or more capture reagents, each capture reagent specifically binding one of the protein biomarkers, and detecting binding between the capture reagent and the protein biomarker.

38. The method of claim 37, wherein determining the quantitative measure of one or more microparticle-associated protein biomarkers comprises performing an immunoassay.

39. The method of claim 38, wherein the immunoassay is selected from the group consisting of an enzyme immunoassay (EIA), an enzyme-linked immunosorbent assay (ELISA), and a radioimmunoassay (RIA).

40. The method of claim 35, wherein the assessing risk of placenta accreta comprises executing a classification rule, wherein the classification rule classifies the subject at being at risk of placenta accreta, and wherein execution of the classification rule produces a correlation between placenta accreta or term birth with a p value of less than at least 0.05.

41. The method of claim 35, wherein the assessing risk of placenta accreta comprises executing a classification rule, wherein the classification rule classifies the subject at being at risk of placenta accreta, and wherein execution of the classification rule produces a receiver operating characteristic (ROC) curve, wherein the ROC curve has an area under the curve (AUC) of at least 0.6, at least 0.7, at least 0.8 or at least 0.9.

42. The method of claim 35, wherein the classification rule classifies a subject based on one or more values wherein the one or more values further include at least one of: placenta previa, previous cesarean delivery, endometrial ablation, in vitro fertilization, prior uterine infection, or previous uterine surgery.

43. The method of claim 35, wherein the classification rule employs cut-off, linear regression including multiple linear regression, partial least squares regression, principal components regression, binary decision trees including recursive partitioning processes further including classification and regression trees, artificial neural networks including back propagation networks, discriminant analyses further including Bayesian classifier or Fischer analysis, logistic classifiers, and support vector classifiers including support vector machines.

44. The method of claim 35, wherein the classification rule is configured to have a sensitivity value, a specificity value, a positive predictive value, or a negative predictive value of at least 70%, least 80%, at least 90% or at least 95%.

45. The method of claim 35, wherein assessing risk of placenta accreta comprises determining that the protein biomarker, if upregulated, is above a threshold level or if down regulated, is below the threshold level.

46. The method of claim 45, wherein the threshold level represents a level at least one, at least two or at least three z scores from a measure of central tendency including a mean, a median or a mode for the protein biomarker determined from at least 50, at least 100 or at least 200 control subjects.

47. The method of claim 35, wherein the assessing risk of placenta accreta comprises comparing the one or more quantitative measures of each protein biomarker in the panel to a reference standard.

48. (canceled)

49. A method of treating placenta accreta in a pregnant subject, the method comprising:
(a) determining, according to the method of claim 35 that a pregnant subject is at increased risk of placenta accreta; and
(b) administering a therapeutic intervention to the subject effective to decrease the risk of placenta accreta and/or reduce neonatal complications of placenta accreta.

50. The method of claim 49, wherein administering the therapeutic intervention comprises a therapeutic intervention selected from the group consisting of:
(i) referring the subject to a medical center having advanced multidisciplinary surgical expertise and experience;
(ii) planning surgical uterine conservation; and
(iii) performing a Cesarean hysterectomy, performing a prophylactic embolization, inserting a uterine balloon tamponade, a temporal internal iliac occlusion balloon catheter, a ureteral stents, administering methotrexate, leaving a portion of the placenta in-situ, and referring the subject to bed rest to prevent preterm labor.

51.-54. (canceled)

55. A machine learning method for generating a classifier for classifying risk of placenta accreta comprising:
(a) providing a dataset comprising biomarker measurements derived from biological samples from subjects in the second trimester of pregnancy, wherein the subjects are classed into (i) subjects at increased risk of placenta accreta and (ii) subjects not at increased risk of placenta accreta;
(b) analyzing the dataset using a machine learning algorithm to generate a classifier that classifies a sample as being at increased risk of placenta accreta or not at increased risk of placenta accreta.
